(19) United States
(12) Patent Application Publication
(10) Pub. No.: US 2022/0378890 A1
(43) Pub. Date: Dec. 1, 2022

(54) IMMUNOGENIC EGFR PEPTIDE COMPOSITIONS AND THEIR USE IN THE TREATMENT OF CANCER

(71) Applicant: Board of Regents, The University of Texas System, Austin, TX (US)

(72) Inventors: Gregory LIZEE, Houston, TX (US); Fenge LI, Houston, TX (US)

(21) Appl. No.: 17/764,104

(22) PCT Filed: Sep. 25, 2020
(86) PCT No.: PCT/US20/52930

§ 371 Date: Mar. 25, 2022

Related U.S. Application Data
(60) Provisional application No. 62/906,688, filed on Sep. 26, 2019.

(51) Int. Cl. A61K 39/00; A61K 31/4745; A61K 39/395; C12N 5/0783; A61P 35/00; A61P 11/00

(52) U.S. Cl. CPC A61K 39/001104; A61K 31/4745; A61K 39/3955; C12N 5/0638; A61P 35/00; A61P 11/00; A61K 2039/5154; A61K 2039/86; C12N 2502/1121

(57) ABSTRACT

Provided are compositions including EGFR mutant peptides that bind to HLA class I and/or HLA class II complexes and compositions comprising a plurality of such peptides. Methods for treating EGFR-mutant cancers with peptides of the embodiments are likewise provided. Methods for expanding related populations of immune effector cells, such as T cells, are also provided.

### CROSS-REFERENCE TO RELATED APPLICATIONS

This application is an international application under the Patent Cooperation Treaty which claims priority to U.S. Provisional Patent Application No. 62/906,688, filed 26 Sep. 2019, the content of which is hereby incorporated by reference is its entirety.

### REFERENCE TO SEQUENCE LISTING SUBMITTED ELECTRONICALLY

Pursuant to the EFS-Web legal framework and 37 CFR §§ 1.821-825 (see MPEP § 2442.03(a)), a Sequence Listing in the form of an ASCII-compliant text file (entitled “Sequence_Listing_3000072-001977_ST25.txt” created on 22 Sep. 2020, and 243,949 bytes in size) is submitted concurrently with the instant application, and the entire contents of the Sequence Listing are incorporated herein by reference.

### BACKGROUND OF THE INVENTION

### 1. Field of the Invention

The present invention relates generally to the field of immunology and medicine. More particularly, it concerns cancer-specific peptides that bind to HLA class I and HLA class II molecules.

### 2. Description of Related Art

T cell based therapies have shown significant promise as a method for treating many cancers; unfortunately, this approach has also been hindered by a paucity of immunogenic antigen targets for common cancers and potential toxicity to non-cancerous tissues. These T cell based therapies can include ACT (adoptive cell transfer) and vaccination approaches. ACT generally involves which involves infusing a large number of autologous activated tumor-specific T cells into a patient, e.g., to treat a cancer. Generally, to develop effective anti-tumor T cell responses, the following three steps are normally required: priming and activating antigen-specific T cells, migrating activated T cells to tumor site, and recognizing and killing tumor by antigen-specific T cells. The choice of target antigen is important for induction of effective antigen-specific T cells.

Neoantigen peptides derived from protein-coding tumor mutations that are displayed at the tumor cell surface by human leukocyte antigen (HLA) molecules could serve as promising antigens for generating an effective immune response. However, as a consequence of HLA diversity and the private nature of most tumor-associated mutations, few neoantigens are shared between patients, and vaccine-induced clinical responses have remained rare event. Thus, method for identification and validation of novel epitopes for cancers expressing neoantigens is needed.

### BRIEF SUMMARY OF THE INVENTION

In some embodiments, the present disclosure provides methods of treating a subject having a EGFR-mutant cancer comprising administering to the subject at least a first HLA-binding peptide from EGFR and at least a first EGFR inhibitor, said peptide comprising a mutated amino acid sequence relative to wild type human EGFR that matches the mutation in the cancer of the subject. In some aspects, the HIA-binding peptide from EGFR binds to a HLA class I molecule. In some aspects, the HLA class I-binding peptide is 8, 9, 10, 11, 12 or 13 amino acids in length. In further aspects, the HLA class I-binding peptide is 9, 10 or 11 amino acids in length. In some aspects, the HLA-binding peptide from EGFR binds to a HLA class II molecule. In some aspects, the HLA class II-binding peptide is 13-30 amino acids in length. In further aspects, the HLA class II-binding peptide is 15-23 amino acids in length.

In some aspects, the methods comprise administering at least a first and a second HLA-binding peptide from EGFR, wherein said peptides each comprise a mutated amino acid sequence relative to wild type human EGFR that matches the mutation in the cancer of the subject. In some aspects, the first HLA-binding peptide from EGFR binds to a HLA class I molecule and the second HLA-binding peptide from EGFR binds to a HLA class II molecule. In some aspects, the methods comprise administering a plurality of HIA-binding peptides from EGFR, wherein said peptides each comprise a mutated amino acid sequence relative to wild type human EGFR that matches the mutation in the cancer of the subject. In some aspects, the plurality of HLA-binding peptides comprises peptides that bind to both HLA class I and HLA class II molecules. In some aspects, the methods comprise administering 2 to 30 different HLA-binding peptides to the subject. In further aspects, the methods comprise administering 5 to 30 different HLA-binding peptides to the subject.

In some aspects, the HLA genotype of the subject has been determined. In some aspects, the HLA-binding peptide from EGFR is predicted to bind to a HLA type carried by the subject. In some aspects, HLA-binding peptide from EGFR is predicted to bind to a HLA type carried by the subject using an automated analysis program. In some aspects, the EGFR-mutant cancer expresses an EGFR polypeptide having an amino acid substitution, deletion or insertion relative to wild type EGFR. In some aspects, the EGFR-mutant cancer comprises a mutation selected from the group consisting of E709K, E709A, E709H, G719A, G719S, G719C, S768I, H773L, T790M, L858R, L861Q, 709_710del>D, 745_749del, 745_750del, 746_750del, 746_751del, 746_751del>A, 746_752del>V, 747_750del, 747_751del, 747_751del>P, 747_753del, 747_753del>S, 747_753del>Q, 747_750del>P, 747_752del, 751_759del>N, 752_759del, 744_745ins>KIPVAI, 745_746ins>IPVAIK, 745_746ins>VPVAIK, 745_746ins>TPVAIK, 763_764ins>FQEA, 764_765ins>HH, 766_767ins>AI, 768_770dupSVD, 769_770ins>ASV, 770_771ins>G, 770_771ins>NPG, 770.771ins>SVD, 771_772ins>N, 771_772ins>H, 772_773ins>NP, 772_773ins>PR, 773_774ins>H, 773_774ins>PH, 773_774ins>NPH, 773_774ins>AH, 774_775ins>HV, and 775_776ins>YVMA. In some aspects, the EGFR-mutant cancer comprises a mutation selected from the group consisting of G719A, G719S, G719C, S768I, H773L, T790M, L858R, L861Q, 709_710del>D, 746_750del, 746_752del>V, 747_751del, 747_751del>P, 747_753del>S, 747_750del, 747_752del, 744_745ins>KIPVAI, 745_746ins>IPVAIK, 763_764ins>FQEA, 768_770dupSVD, 769_770ins>ASV, 770_771ins>SVD, 772_773ins>PR, 773_774ins>H, 773_774ins>PH, 773_774ins>NPH, and 773_774ins>AH. In some aspects, the EGFR-mutant cancer comprises a mutation selected from the group consisting of L858R, H773L, T790M, E709V, 747_751Del, V774M and S768I.

In some aspects, the EGFR inhibitor is administered before said HLA-binding peptide from EGFR. In other aspects, the EGFR inhibitor is administered after or essentially simultaneously with said HLA-binding peptide(s) from EGFR. In some aspects, the HLA-binding peptide(s) from mutant EGFR are administered in conjunction with TLR ligand. In some aspects, the TLR ligand is a TLR2, TLR3, TLR4, TLR7, TLR8 or TLR9 agonist. In some aspects, the TLR ligand is a TLR7 agonist. In some aspects, the TLR7 agonist is CL075, CL097, CL264, CL307, GS-9620, Poly(dT), imiquimod, gardiquimod, resiquimod (R848), loxoribine or a ssRNA oligonucleotide. In some aspects, the TLR7 agonist is imiquimod. In some aspects, the EGFR inhibitor is a tyrosine kinase inhibitor. In some aspects, the EGFR inhibitor is an EGFR binding antibody. In some aspects, the EGFR inhibitor is osimertinib, erlotinib, gefitinib, cetuximab, matuzumab, panitumumab, AEE788, CI-1033, HKI-272, HKI-357 or EKB-569.

In some aspects, the cancer is lung cancer. In some aspects, the lung cancer is non-small cell lung cancer. In some aspects, the lung cancer is a metastatic lung cancer. In some aspects, the lung cancer is a lung adenocarcinoma. In some aspects, the cancer is EGFR inhibitor resistant.

In another embodiment, the present disclosure provides immunogenic compositions comprising at least a first and a second HLA-binding peptide from EGFR, said first and second peptides comprising a mutated amino acid sequence relative to wild type human EGFR that matches a mutation in a human EGFR-mutant cancer, wherein the first HLA-binding peptide from EGFR binds to a HLA class I molecule and the second HLA-binding peptide from EGFR binds to a HLA class II molecule. In some aspects, the HLA-binding peptides are formulated in a pharmaceutically acceptable carrier. In some aspects, the pharmaceutically acceptable carrier is an aqueous carrier. In some aspects, the pharmaceutically acceptable carrier is a salt solution. In some aspects, the pharmaceutically acceptable carrier is a saline solution, preferably an isotonic saline solution. In some aspects, the HLA class I-binding peptide is 8, 9, 10, 11, 12 or 13 amino acids in length. In further aspects, the HLA class I-binding peptide is 9, 10 or 11 amino acids in length. In some aspects, the HLA class II-binding peptide is 13-30 amino acids in length. In further aspects, the HLA class I-binding peptide is 15-23 amino acids in length. In some aspects, the compositions comprise a plurality of HLA-binding peptides from EGFR wherein said peptides each comprise a mutated amino acid sequence relative to wild type human EGFR that matches a EGFR mutation in a human EGFR-mutant cancer. In some aspects, the compositions comprise 2 to 30 different HLA-binding peptides to the subject. In some aspects, the compositions comprise at least two HLA class I-binding peptides and at least one HLA class II-binding peptide. In some aspects, the compositions comprise at least one HLA class I-binding peptide and at least two HLA class II-binding peptides. In some aspects, the compositions comprise at least two HLA class I-binding peptides and at least two HLA class II-binding peptides. In some aspects, the compositions comprise at least 3 HLA class I-binding peptides and at least 3 HLA class II-binding peptides.

In some aspects, the HLA-binding peptides from EGFR comprise an amino acid substitution, deletion or insertion relative to wild type EGFR. In some aspects, the HLA-binding peptides from EGFR comprise an amino acid substitution relative to wild type EGFR. In some aspects, the HLA-binding peptides comprises a mutation selected from the group consisting of E709K, E709A, E709H, G719A, G719S, G719C, S768I, H773L, T790M, L858R, L861Q, 709_710del>D, 745_749del, 745_750del, 746_750del, 746_751del, 746_751del>A, 746_752del>V, 747_750del, 747_751del, 747_751del>P, 747_753del, 747_753del>S, 747_753del>Q, 747_750del>P, 747_752del, 751_759del>N, 752_759del, 744_745ins>KIPVAI, 745_746ins>IPVAIK, 745_746ins>VPVAIK, 745_746ins>TPVAIK, 763_764ins>FQEA, 764_765ins>HH, 766_767ins>AI, 768_770dupSVD, 769_770ins>ASV, 770_771ins>G, 770_771ins>NPG, 770_771ins>SVD, 771_772ins>N, 771_772ins>H, 772_773ins>NP, 772_773ins>PR, 773_774ins>H, 773_774ins>PH, 773_774ins>NPH, 773_774ins>AH, 774_775ins>HV, and 775_776ins>YVMA relative to wild type human EGFR. In some aspects, the HLA-binding peptides comprises a mutation selected from the group consisting of G719A, G719S, G719C, S768I, H773L, T790M, L858R, L861Q, 709_710del>D, 746_750del, 746_752del>V, 747_751del, 747_751del>P, 747_753del>S, 747_750del>P, 747_752del, 744_745ins>KIPVAI, 745_746ins>IPVAIK, 763_764ins>FQEA, 768_770dupSVD, 769_770ins>ASV, 770_771ins>SVD, 772_773ins>PR, 773_774ins>H, 773_774ins>PH, 773_774ins>NPH, and 773_774ins>AH relative to wild type human EGFR. In some aspects, the HLA-binding peptides comprises a mutation selected from the group consisting of L858R, H773L, T790M, E709V, 747_751Del, V774M and S768I relative to wild type human EGFR.

In some aspects, the compositions further comprise an EGFR inhibitor. In some aspects, the compositions further comprise a TLR ligand. In some aspects, the TLR ligand is a TLR2, TLR 2, TLR4, TLR7, TLR8 or TLR9 agonist. In some aspects, the compositions further comprise a TLR-7 agonist. In some aspects, the TLR7 agonist is CL075, CL097, CL264, CL307, GS-9620, Poly(dT), imiquimod, gardiquimod, resiquimod (R848), loxoribine or a ssRNA oligonucleotide. In further aspects, the TLR7 agonist is imiquimod. In some aspects, the HLA-binding peptides are in complex with HLA class I and/or HLA class II molecules. In some aspects, the compositions further comprise an antigen presenting cell. In some aspects, the antigen presenting cell comprises a dendritic cell. In some aspects, the HLA-binding peptides are comprised in a liposome, lipid-containing nanoparticle, or in a lipid-based carrier. In some aspects, the compositions further comprise an adjuvant component.

In still another embodiment, the present disclosure provides methods of treating a subject comprising administering an effective amount of a composition of the present disclosure to the subject (e.g., a composition comprising), wherein the subject has an EGFR-mutant cancer and wherein the HLA-binding peptide from EGFR in the composition comprising mutations matching those from the EGFR-mutant cancer in the subject. In some aspects, the methods further comprise sequencing the EGFR gene in the cancer of the subject. In some aspects, the HLA genotype of the subject has been determined. In some aspects, HLA-binding peptides from EGFR are predicted to bind to a HLA class I or HLA class II type carried by the subject. In further aspects, EGFR-mutant peptides included in the composition are selected using an algorithm that predicts HLA binding to HLA types expressed in the subject. In some aspects, the peptides included in the composition are selected based on predicted HLA affinity and/or predicted HLA ranking. In further aspects, the peptides included in the composition are selected based on predicted HLA affinity and predicted HLA ranking. In further aspects, the algorithm used for selecting HLA-binding peptides is selected from NetMHC4.0, NetMHCpan3.0, NetMHCpan4.0, NetMHCII2.2 and/or NetMHCII2.3. (See, e.g., Andreatta et al., 2015 and Jensen et al., 2018, each of which is incorporated herein by reference).

In some aspects, the methods further comprise administering an EGFR inhibitor to the subject. In some aspects, the EGFR inhibitor is administered before said HLA-binding peptides from mutated EGFR. In some aspects, the EGFR inhibitor is administered after or essentially simultaneously with said HLA-binding peptides from mutated EGFR. In some aspects, the EGFR inhibitor is an EGFR binding antibody. In some aspects, the EGFR inhibitor is Osimertinib, erlotinib, gefitinib, cetuximab, matuzumab, panitumumab, AEE788; CI-1033, HKI-272, HKI-357 or EKB-569. In some aspects, the EGFR inhibitor is a tyrosine kinase inhibitor. In some aspects, the HLA-binding peptide from EGFR is administered in conjunction with a TLR ligand. In some aspects, the TLR ligand is a TLR2, TLR 2, TLR4, TLR7, TLR8 or TLR9 agonist, preferably a TLR7 agonist. In some aspects, the TLR7 agonist is CL075, CL097, CL264, CL307, GS-9620, Poly(dT), imiquimod, gardiquimod, resiquimod (R848), loxoribine or a ssRNA oligonucleotide. In further aspects, the TLR7 agonist is imiquimod.

In some aspects, the cancer is lung cancer. In some aspects, the lung cancer is non-small cell lung cancer. In some aspects, the lung cancer is a metastatic lung cancer. In some aspects, the lung cancer is a lung adenocarcinoma. In some aspects, the cancer is EGFR inhibitor resistant. In some aspects, the composition is administered by parenteral administration, intravenous injection, intramuscular injection, inhalation, or subcutaneous injection. In some aspects, the composition is formulated in an aqueous carrier. In some aspects, the aqueous carrier is a salt solution. In some aspects, the aqueous carrier is an isotonic saline solution. In some aspects, the composition is administered at least two, three, four or five times. In some aspects, there are 2, 3, 4, 5, 6 or 7 days between administrations. In some aspects, the methods further comprise administering a further anti-cancer therapy. In some aspects, the further anti-cancer therapy is selected from the group consisting of a chemotherapy, a radiotherapy, an immunotherapy, or a surgery. In some aspects, the immunotherapy comprises at least one immune checkpoint inhibitor. In some aspects, the immune checkpoint inhibitor is an anti-PD1 or anti-CTLA-4 monoclonal antibody. In some aspects, the immunotherapy is a combination of immune checkpoint inhibitors.

In yet another embodiment, the present disclosure provides methods of producing EGFR-mutant cancer-specific immune effector cells comprising obtaining a starting population of immune effector cells and contacting the starting population of immune effector cells with a composition of the present disclosure, thereby generating EGFR-mutant cancer-specific immune effector cells. In some aspects, contacting is further defined as co-culturing the starting population of immune effector cells with antigen presenting cells (APCs), wherein the APCs present the HLA-binding peptides of the present disclosure on their surface. In some aspects, the APCs are dendritic cells. In some aspects, the immune effector cells are T cells, peripheral blood lymphocytes, NK cells, invariant NK cells, NKT cells. In some aspects, the immune effector cells have been differentiated from mesenchymal stem cell (MSC) or induced pluripotent stem iPS) cells. In some aspects, the T cell is a CD8⁺ T cell, CD4⁺ T cell, or γδ T cell. In some aspects, the starting population of T cells are CD8⁺ T cells or CD4⁺ T cells. In some aspects, the T cells are cytotoxic T lymphocytes (CTLs). In some aspects, obtaining comprises isolating the starting population of immune effector cells from peripheral blood mononuclear cells (PBMCs).

In still another embodiment, the present disclosure provides EGFR-mutant cancer-specific T cells produced by a method of the present disclosure.

In yet another embodiment, the present disclosure provides pharmaceutical compositions comprising the EGFR-mutant cancer-specific T cells produced by a method of the present disclosure.

In still another aspect, the present disclosure provides methods of treating cancer in a subject comprising administering an effective amount of the EGFR-mutant cancer-specific T cells of the present disclosure to the subject.

In yet another aspect, the present disclosure provides compositions comprising an effective amount of the EGFR-mutant cancer-specific T cells of the present disclosure for the treatment of cancer in a subject.

As used herein, “essentially free,” in terms of a specified component, is used herein to mean that the specified component has not been purposefully formulated into a composition and/or is present only as a contaminant or in trace amounts.

As used herein, “a” or “an” may mean one or more than one.

As used herein, the term “or” means “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1H show personalized peptide vaccine (PPV) trial design and patient outcomes. (FIG. 1A) Neoantigen peptide vaccine manufacturing pipeline leading to immunization of patients with advanced non-small cell lung cancer (NSCLC). DNA from lung tumor biopsies was sequenced using a panel of 508 tumor-associated genes while high resolution HLA typing was performed on patient peripheral blood. Neoantigen vaccine peptides were selected largely based on HLA class I and class II binding predictions (see Methods). Each patient was immunized weekly with a saline-based mixture of short and long neoantigen peptides divided into 2 cocktails and administered into opposite extremities for 12 weeks. Arrows represent weeks when vaccination was received. Blood was drawn at weeks 0, 4, 8 and 12 as indicated with syringe symbols. (FIG. 1B) Clinical event timeline for the 24 NSCLC patients who received PPV. Dark bars (labelled Neoantigen vaccine), duration of PPV immunization. Thin light bars (labelled EGFR inhibitor), duration of EGFR inhibitor therapy. (FIG. 1C) 14 PPV patients were divided into 3 groups based on EGFR mutation status and use of EGFR inhibitor during vaccination. (FIGS. 1D-1F) Numbers of vaccine peptides, numbers of mutations targeted, and predicted vaccine peptide binding affinities stratified by patient cohort, clinical response and progression-free survival. Black dots indicate non-EGFR neoantigen peptides and gray dots indicate EGFR neoantigen peptides, with percentages of the latter listed at bottom. (FIG. 1G) Survival analysis of the three patient groups showed that patients in Group 3 experienced significantly longer overall survival compared with the other groups (Group 2 vs. Group 3, P=0.038). (FIG. 1H) Comparison of progression-free survival between patients experiencing an objective clinical response (CR/PR, n=7), stable disease (SD, n=9), or progressive disease (PD. n=8). Survival analyses were performed using a Log-rank (Mantel-Cox) Test. Two-tailed unpaired t test or Mann-Whitney U test was used to analyze the statistical significance between groups. P<0.05 was considered significantly different.

FIG. 2 shows immunization time course of the 24 PPV patients. Each square represents one week. Dark squares and arrows represent weeks when vaccination was received and white squares represent weeks when no vaccine was administered. According to the trial design, patients were to receive weekly vaccinations for a minimum of 12 weeks. All patients completed 12 weeks of vaccination, with the exception of Pts. 9, 19, and 20, who expired during the initial trial period due to advanced disease. Patients were given the opportunity to continue immunizations beyond 12 weeks if they desired.

FIG. 3 shows treatment and clinical outcomes of the 24 PPV patients separated into groups.

FIGS. 4A & 4B show a summary of clinical baseline characteristics by group. (FIG. 4A) Clinical and demographic characteristics of PPV groups at baseline are indicated, with statistical comparisons of groups PPV-1 (EGFR-WT, PPV only), PPV-2 (EGFR mutant. PPV only) and PPV-3 (EGFR mutant, PPV+EGFRi). SQ, squamous cell carcinoma; AD, adenocarcinoma. Continuous data were shown as mean+standard deviation (SD). (FIG. 4B) EGFR inhibitor treatment history of PPV-2 and PPV-3 patients. TKI, tyrosine kinase inhibitor. Two-tailed unpaired t test or Chi-square test was used to analyze the statistical significance between groups. P<0.05 was considered significantly different.

FIGS. 5A-5C show clinical responses of PPV patients by group. (FIG. 5A) Response summary of immunized patients by group showing progression-free survival, overall survival, and clinical response as assessed using RECIST1.1 criteria 12-18 weeks following initiation of PPV. (FIG. 5B) Measurements of the overall tumor burden (sum of all target lesions) of PPV patients over the course of treatment. The clinical response of each patient is as follows: Pt.1—PD; Pt.4—PD; Pt. 6—SD; Pt.7—SD; Pt.9—SD; Pt.10—PD; Pt. 19—PD; Pt.11—CR or PR; Pt.14—CR or PR; Pt.16—SD; Pt.17—CR or PR; Pt.18—PD; Pt.20—PD; Pt. 24—PD; Pt.3—SD; Pt.5—CR or PR; Pt.8—CR or PR; Pt.12—CR or PR; Pt.13—SD; Pt. 15—SD; Pt.21—SD; Pt.22—CR or PR; Pt.23—PD. * Patient 1 developed pleural effusion at 12 wks. ** Follow-up CT scan of patient 11 was taken at 24 wks. No tumor measurements are shown for Patient 2 due to their having no measurable tumors. Follow-up CT scans were not available for patients 13 and 15, but other clinical follow-up information regarding response and survival was obtained. PPV, personalized peptide vaccine; PPV-1, PPV Group 1; PPV-2, PPV Group 2; PPV-3, PPV Group 3. MUT, mutated; CR, complete response; PR, partial response; PD, progressive disease; SD, stable disease. (FIG. 5C) List of treatment-related adverse events.

FIGS. 6A-6C show representative CT scans of selected PPV patients, including pre-vaccine trial EGFR inhibitor failures. (FIG. 6A) Serial CT scans showing that pleural effusion of Patient 2 (Group 1) disappeared 10 weeks after PPV treatment. (FIG. 6B) CT scans of Patient 23 showing progression of liver metastases during PPV treatment. Patient 24 similarly demonstrated lung tumor progression II weeks after the start of immunization. (FIG. 6C) Serial CT scans depicting pre-PPV trial EGFR inhibitor failures in Patients 17, 5, 8 and 12 followed by clinical objective responses after starting PPV treatment.

FIGS. 7A-7F show patient clinical responses following personalized neoantigen peptide vaccination. (FIG. 7A) CT scans showing regression of two lung lesions from complete responder Patient 17. (FIG. 7B) Tissue biopsy confirmed that the remaining lung CT signal was comprised of only fibrotic tissue containing no viable tumor cells. (FIG. 7C) Patient 17 bone metastases evaluated by T2 weighted magnetic resonance imaging (MRI) disappeared 18 weeks after the start of neoantigen vaccination. This bone metastasis was considered as non-targeted lesion according to RECIST (version 1, bone lesion measurability). (FIG. 7D) Two additional patients in Group 2 had objective clinical responses to PPV. Patient 11 experienced lung tumor regression in addition to resolution of obstructive atelectasis 24 weeks after PPV initiation (white arrow, right), while a pneumothorax showed no change (gray arrow). Neck metastases of Patient 14 showed significant regression 12 weeks after the start of PPV treatment (right). (FIG. 7E) CT scans showing lung tumor regressions in Patients 5, 8, 12 and 22, all of whom had partial clinical responses following PPV treatment. (FIG. 7F) Change in overall tumor burden of PPV study patients 3 to 4 months post-PPV compared with pre-treatment baseline. *Patient 1 developed pleural effusion at 12 wks. CR, complete response; PR, partial response.

FIGS. 8A-8E show survival analysis of different patient groups. (FIG. 8A) Progression-free survival (PFS) analysis of PPV patients by group. (FIG. 8B) Overall survival (OS) analysis of PPV patients by group. (FIG. 8C) Comparison of PFS between patients with disease control (CR+PR+SD; n=16) or with PD (n=8ted EGFR patients, Group 2 patients, or Group 3 patients. And Comparison of overall survival between patients experiencing an objective clinical response (CR/PR, n=7), stable disease (SD, n=9), or progressive disease (PD, n=8). (FIG. 8D) PFS comparison between patients who had either used or not used the third-generation TKI Osimertinib, as shown for all 16 patients, Group 2 patients, or Group 3 patients. (FIG. 8E) Overall survival comparison between patients that had used or not used Osimertinib for all 16 mutated EGFR patients. Survival analyses were performed using a Log-rank (Mantel-Cox) Test, with P<0.05 considered significantly different (bold). 95% confidence intervals (CI) is for hazard ratio.

FIGS. 9A-9C show vaccine peptide analysis by group, clinical response and progression-free survival. (FIG. 9A) Number of different HLA class I and class I molecules engaged by the vaccine peptides, as predicted by HLA peptide binding affinity. Each dot represents one PPV patient. (FIG. 9B) Number of administered vaccine peptides restricted to HLA class I (short) or HLA class II (long). Each dot or circle represents one PPV patient. (FIG. 9C) Peptide Delta Score of vaccine peptides. Delta Score is calculated by subtracting the mutant neoantigen peptide predicted binding affinity from the corresponding wild-type peptide binding affinity. Each dot represents one vaccine peptide. Gray, EGFR neoantigen peptides; Black, non-EGFR neoantigen peptides. Using a two-tailed unpaired t test, no significant differences were found between groups, with P<0.05 considered significantly different. MUT, mutated. WT, wild-type. PD, progressive disease; SD, stable disease; PR, partial response; CR, complete response.

FIGS. 10A & 10B (FIG. 10A) Factors associated with survival of personalized peptide vaccine patients, as determined by univariate analysis. (FIG. 10B) Kaplan-Meier analysis showing that pleural effusion and tumor burden were two risk factors impacting overall survival of PPV trial patients. Survival analyses were performed using a Log-rank (Mantel-Cox) Test, with P<0.05 considered significant (bold). SD, standard deviation; EGFRi; EGFR inhibitor; ECOG, Eastern Cooperative Oncology Group; PS, performance status; *SQ, squamous cell carcinoma; AD, adenocarcinoma. EGFRi failure category 1, failed first generation EGFRi; 2/3, failed second or third-generation EGFRi.

FIGS. 11A-11I show EGFR neoantigen peptides are immunogenic, shared and show distinctive HLA class I binding preferences. (FIG. 11A) Interferon-gamma (IFN-γ) ELISA assays performed on peptide pool-stimulated patient PBMC supernatants showed vaccine peptide pool-specific responses primarily in 6 PPV patients: 5, 8, 14, 17, 21, and 22. Five of the six patients had experienced objective clinical responses following PPV (Pt.11, Pt.14, Pt.17, Pt.5, Pt.8, Pt.12, Pt.22). (FIG. 11B) Deconvolution of individual vaccine peptide reactivities by IFN-γ ELISA for Patients 5, 8, 14 and 17 revealed dominant immune responses in Patients 5, 8, and 14 against the HLA-A*1101 restricted EGFR-L858R peptide KITDFGRAK (SEQ ID NO: 4). Complete responder Patient 17 similarly showed a dominant response against the HLA*C1502 restricted EGFR-T790M peptide LTSTVQLIM (SEQ ID NO: 65). Individual peptide reactivities for other PPV study patients are shown in FIG. 12. (FIGS. 11C & 11D) Summaries of IFN-γ ELISPOT assay and HLA tetramer-based staining determined that PBMC frequencies of HLA-A*I 101/KITDFGRAK (SEQ ID NO: 4)-specific CD8⁺ T cells in Patients 5, 8 and 14, and HLA-C*1502/LTSTVQLIM (SEQ ID NO: 65)-specific CD8⁺ T cells in Patient 17 increased significantly over the course of PPV. (FIG. 11E) ELISPOT assay showed post-PPV PBMC from Patients 5, 8, 14, and 17 specifically recognized mutated EGFR neoantigen peptides but not the corresponding wild type (WT) EGFR peptides. (FIG. 11F) EGFR protein sequences and predicted HLA peptide binding affinities of the mutant EGFR-L858R (boxed region of larger peptide SEQ ID NO: 980) and T790M (boxed region of larger peptide SEQ ID NO: 982) peptides and corresponding WT epitopes (boxed region of larger peptide SEQ ID NO: 979 and boxed region of larger peptide SEQ ID NO: 981, respectively). (FIGS. 11G & 11H) Neoantigens derived from the most prevalent EGFR mutations, L858R (light gray) and Exon 19 deletions (Ex19del, medium gray), show distinctive binding preferences for HLA class I allotypes within the A3 superfamily, whereas other less prevalent EGFR point mutations (S768I, T790M, and L861Q, dark gray) show binding preferences for A2 and C3 superfamily members. (FIG. 11I) Expanded view showing individual HLA class I allotypes with the highest number of predicted binding EGFR neoantigens (<500 nM affinity) for the most prevalent shared EGFR mutations in lung cancer. Black arrows indicate the A*1101-restricted KITDFGRAK peptide (SEQ ID NO: 4) and C*1502-restricted LTSTVQLIM peptide (SEQ ID NO: 65). Two-tailed unpaired t tests were used to analyze the statistical significance between groups. P≤0.05 was considered significantly different.

FIGS. 12A & 12B show ELISA-based immune monitoring of PPV-induced immune responses. (FIG. 12A) Results of IFN-γ ELISA assay measuring peripheral blood reactivity in response to individual vaccine peptides. Peptide numbers correspond to those listed in Table 5. Fold change is measured relative to the no peptide control (No) at the indicated time points. I.C., irrelevant peptide control. Clinical responders are indicated as either (CR) or (PR). (FIG. 12B) Summary figure showing the total vaccine peptide-specific immune reactivity for each patient (ComboScore, see Methods) along with their associated group, histology, and clinical outcome.

FIGS. 13A-13H show ELISPOT-based ex vivo peripheral blood immune monitoring of vaccine-induced responses. IFN-γ ELISPOT assay results show PPV-induced immune reactivity against selected vaccine peptides (2.5×10⁵ cell per well) in eight immunized patients, including 7 clinical responders. The EGFR(L858R) NeoAg peptide KITDFGRAK (SEQ ID NO: 4) elicited dominant IFN-γ responses in three different responding Pts. 5, 8, and 14 (FIGS. 13A, 13B, and 13D); however, no immune response could be detected against this vaccine peptide in SD Pt. 16 (FIG. 13E). Complete responding Pt. 17 generated a dominant immune response against the LTSTVQLIM (SEQ ID NO: 65) NeoAg peptide containing the shared EGFR(T790M) mutation (FIG. 13F). Two additional PR patients generated CD8+ T cell responses against private mutation-encoding NeoAgs: the AQP12A(L28R) peptide KARLPVGAY (SEQ ID NO: 875) in Pt. 11 (FIG. 13C) and the FGFR1(R734W) peptide YMMMWDCWHAV (SEQ ID NO: 964) in Pt. 22 (FIG. 13G). Immune responses against long, CD4+ restricted EGFR NeoAg peptides were also elicited in 3 of the patients: the EGFR(L858R)-containing peptide HVKITDFGRAKLLGAEE (SEQ ID NO: 826) in Pts. 8 and 16 (FIGS. 13B & 13E), and the H773UV774M NeoAg peptide MASVDNPLMCRLLGICL (SEQ ID NO: 958) in Pt. 22 (FIG. 13G). Also depicted are line graph summaries showing the number IFN-γ spots per 10⁶ PBMC (normalized) for each vaccine peptide that elicited a response. Peptide identification numbers (P1, P2, etc.) correspond to those listed in Table 5 and also correspond to SEQ ID NO: 874 (RLPVGAYEV); SEQ ID NO: 880 (VMASVDNPL); SEQ ID NO: 1 (HVKITDFGR); SEQ ID NO: 16 (VKITDFGRAK); SEQ ID NO: 32 (AIKESPKANK); SEQ ID NO: 914 (KIPVAIKESPKANKEIL); SEQ ID NO: 687 (STVQLIMQL); SEQ ID NO: 956 (NPLMCRLLGI); SEQ ID NO: 15 (TDFGRAKLL); and SEQ ID NO: 884 (RLSISFENLDTAKKKLP). Two-tailed unpaired t test was used to analyze the statistical significance between groups. P<0.05 was considered significantly different. *P<0.05, **P<0.01, ***P<0.01.

FIGS. 14A-14C show HLA/peptide tetramer-based immune monitoring of vaccine-induced CD8+θT-cell responses. (FIG. 14A) Listing of custom synthesized EGFR neoantigen tetramers used for immune monitoring analyses: KITDFGRAK (SEQ ID NO: 4); VKITDFGRAK (SEQ ID NO: 16); HVKITDFGR (SEQ ID NO: 1); AIKESPKANK (SEQ ID NO: 32); AIKTSPKANK (SEQ ID NO: 20); LTSTVQLIM (SEQ ID NO: 65). (FIGS. 14B & 14C) Tetramer staining results of ex vivo pre- and post-vaccine PBMCs drawn at the time points indicated. (FIG. 14B) EGFR neoantigen-specific CD8+ T cell populations were observed for PPV Pts. 5, 8, 14, and 16(L858R; SEQ ID NO: 4) and 17 (T790M; SEQ ID NO: 65). (FIG. 14C) Shown are examples of negative EGFR neoantigen tetramer staining of PBMC drawn from vaccinated Pts. 3, 5, 8, 14 and 16.

FIGS. 15A-15C show HLA class I and class II superfamily peptide binding analysis of shared EGFR neoantigens. HLA binding prediction was performed for EGFR neoantigen peptides containing the 10 most prevalent EGFR mutations in lung cancer, including five shared point mutations (S768L, H773L, T790M, L858R, and L861Q) and five common Exon 19 deletions (legend). (FIG. 15A) Number of 9, 10, or 11-mer EGFR neoantigen peptides predicted to bind to the 100 most prevalent HLA class I allotypes worldwide with predicted binding affinity of 500 nM or less. Allotypes are divided into HLA superfamilies, revealing distinct superfamily binding preferences for different shared EGFR mutations. (FIG. 15B) Number of 9, 10, or 11-mer EGFR neoantigen peptides predicted to bind to HLA class I allotypes with predicted binding affinity of 5000 nM or less. (FIG. 15C) Number of 17-mer EGFR neoantigen peptides predicted to bind to HLA class II allotypes with predicted binding affinity of 500 nM or less. Binding predictions were performed using NetMHCpan4.0 for HLA class I peptides and NetMHCII2.3 for HLA class II peptides. HLA class I and 11 superfamily groupings were adapted from Sidney et al., 2008, Harjanto et al., 2014, and Jensen et al., 2018. BX, unclassified HLA-B allotypes.

FIGS. 16A-16H show neoantigen vaccination induced increased frequencies and numbers of EGFR-L858R neoantigen-specific T cell clones in peripheral blood and tumor. (FIGS. 16A & 16B) Percent change in TCRVβ-CDR3 clonality score in post-PPV patient PBMC, stratified by patient group and progression-free survival. (FIG. 16C) HLA-A* 1101/KITDFGRAK (SEQ ID NO: 4) tetramer staining and flow sorting of CD8⁺Tetramer⁺ (Tet) T cells from 10-month post-PPV PBMC of Patient 5. Sorted Tet⁺ cells underwent single-cell TCRα/β sequencing. (FIG. 16D) TCRVβ-CDR3 sequencing was performed on Patient 5 PBMC and tumor biopsies taken pre- or post-PPV treatment. Venn diagrams and dot plots show the numbers and frequencies of CDR3 clones that overlap between pairs of samples. 52 high-confidence Tet⁺ Vβ-CDR3 clones sorted from post-PPV showed a high degree of overlap with both PBMC and TIL CDR3 clones (gray dots). PPV induced significant increases in the frequency of neoantigen-specific Tet⁺ clones (small boxes), and 13 new Tet⁺ clones also appeared post-PPV (light gray dots). Immunization also induced a population of other CDR3 clones in both PBMC and TIL (black arrows) (FIG. 16E) PBMC and TIL frequencies of the top 10 pre-existing Tet⁺ CDR3 clones at time points prior to and post-PPV. (FIG. 16F) PBMC and TIL frequencies of 12 vaccine-induced Tet⁺ CDR3 clones prior to and post-PPV. (FIG. 16G) Single-cell sequencing of sorted Tet+ clones facilitated cloning of Vα-N1 and Vβ-N1 with the HLA-A * 110/KITDFGRAK (SEQ ID NO: 4) tetramer. A549 cells were also engineered to express HLA-A*1101 and/or a KITDFGRAK (SEQ ID NO: 4) peptide-encoding minigene. (FIG. 16H) TCR-N1 engineered T cells co-cultured with A11. KIT-transduced A549 cells produced significantly more IFN-γ compared to control T cells, as determined by IFN-γ ELISA (top) (P<0.001). TCR-N1 T cells did not recognize control parental A549, A549-A11, or A549.KIT target cells, demonstrating neoantigen-specific reactivity (bottom). Statistical comparisons were measured compared to control. Two-tailed unpaired t test or Mann-Whitney U test was used to analyze the statistical significance between groups. P<0.05 was considered significantly different. **, P<0.01; ***, P<0.001.

FIGS. 17A-17G. (FIG. 17A) 52 TCR-VβCDR3 clones identified through single-cell TCR sequencing of KITDFGRAK (SEQ ID NO: 4) tetramer-positive CD8+ (Tet+) T cells from 10 Mo PBMC show changes in PMBC frequency during PPV treatment of patient 5. Y-axis shows CDR3 frequency within sorted Tet+ cells, X-axis shows the CDR3 frequency within PBMC at the indicated time points. Correlation coefficients are shown. (FIG. 17B) Maximum fold expansion of 52 Tet+ CDR3 clones in peripheral blood during the course of PPV treatment. Induced clones were not detectable in pre-treatment PBMC. (FIG. 17C) Similar analysis as FIG. 17 A, except the 52 Tet+ clones are compared with CDR3 frequencies in tumor samples taken pre- or 12 Mo post-PPV. (FIG. 17D) EGFR inhibitor Osimertinib treatment of H1975 (EGFR-L858R/T790M) and H1299 (EGFR-WT) lung cancer cells showed significantly decreased phospho-EGFR and phospho-ERK in H1975 cells as shown by Western blot. EGFRi concentration in μM is shown. C1 and C2 are untreated control cells. (FIG. 17E) Heat map showing RNA transcript changes in H1975 and H1299 cells following exposure to EGFRi (24 h) or EGFR (24 h) plus IFN-γ (12 h). (FIG. 17F) Gene signature changes in Jak-Stat, TNFα, and TRAIL signaling following EGFRi or EGFRi+IFNγ treatment of H1975 cells (circles) and H1299 cells (squares). (FIG. 17G) Gene set enrichment analysis of RNAseq following EGFRi treatment of H1975 cells.

FIGS. 18A-18M show immunomodulation by EGFR inhibitors promotes immune cell infiltration, tumor antigen presentation, and T-cell activation. H1975 (EGFR-L858R/T790M) and H1299 (EGFR-WT) cell lines were treated with EGFR inhibitor (EGFRi) Osimertinib, and RNAseq analysis was performed at 0, 12, or 24 hours post-treatment. RNAseq was also performed on 4 patient tumors, 2 on-EGFRi and 2 off-EGFRi. (FIG. 18A) Relative transcript expression levels of genes associated with cell division, cell cycle, apoptosis and cell survival decreased in 111975 cells following EGFRi treatment, a trend mirrored in the on-EGFRi patient tumor samples. (FIG. 18C) Gene expression pathway changes in EGFRi-treated H1975 and H1299 cell lines. In H1975 cells, HLA expression, STAT signaling, TRAIL signaling, Apoptosis, TNF-alpha signaling, and NF-kappaB signaling were upregulated; and EGFR signaling, MAPK signaling, PI3K signaling, Cell cycle, MYC signaling, and EMT signature were downregulated after 24 h EGFRi treatment. In H1299 cells, TRAIL signaling, Apoptosis, STAT signaling, and EMT signature were upregulated; and TNF-alpha signaling, NFkappaB signaling, HLA expression, EGFR signaling, MAPK signaling, PI3K signaling, Cell cycle, MYC signaling were downregulated after 24 h EGFRi treatment. (FIG. 18B) EGFRi upregulated expression of immune-related genes associated with antigen presentation and immune cell trafficking in H1975 cells. (FIG. 18D) EGFRi treatment of H1975 cells (circles) and H1299 cells (squares) downregulated genes associated with EGFR signaling and proliferation rate while upregulating genes associated with TRAIL signaling. (FIGS. 18F & 18G) Luminex analysis of H1975 cell supernatants confirmed changes of 10 chemokines and cytokines at the protein level. Statistical comparisons were measured compared to control. (FIG. 18H) Migration assay showed that EGFRi treatment of H1975 cells increased the migration of PBMC monocytes and CD4⁺ T cells, and activated CD8⁺ tumor infiltrating lymphocytes (TIL) towards H1975 cell supernatants. (FIG. 18I) HLA class I surface expression increased in H1975 but not H1299 cells following EGFRi treatment. (FIG. 18J) Tumor antigen-specific CD8⁺ T-cells showed significantly increased IFN-γ secretion in response to recognition of cognate antigen on EGFRi-treated H1975 cells compared to untreated cells. (FIG. 18E) Patients on-EGFRi treatment demonstrated similar changes in EGFR signaling, proliferation rate, and TRAIL signaling as EGFRi-treated H1975 cells. (FIGS. 18K & 18L) Immune cell content of patient tumor specimens was imputed from RNAseq data using a method that considers immune cell-type specific gene expression (Methods). (FIG. 18M) Proposed mechanistic model to explain how EGFRi treatment may synergize with PPV to enhance immune cell trafficking and T-cell activation at the tumor site. Two-tailed unpaired t test or Mann-Whitney U test was used to analyze the statistical significance between groups. P<0.05 was considered significantly different. *, P<0.05; **, P<0.01.

### DETAILED DESCRIPTION OF THE INVENTION

### I. The Present Invention

In some aspects, peptides derived from mutated EGFR neoantigens are provided that are recognized by, and bind to, HLA class I and/or HLA class II molecules. In particular, peptides are identified that are predicted to bind to specific HLA molecules carrier by a cancer patient having a given EGFR mutant cancer. These peptides, or a cocktail of such peptides (e.g., including both HLA class I and HLA class II-binding peptides), may be employed to stimulate an effective immune response against the EGFR mutant cancer. Thus, such peptides and peptide cocktails may be administered directly to a subject to simulate an immune response in vivo in a subject having an EGFR-mutant cancer, such as a lung cancer. Alternatively, peptides of the embodiments can be used to activate and expand immune effector cells (such as T-cells) ex vivo for use in anti-cancer immunotherapy composition.

### II. Definitions

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.01%. Most preferred is a composition in which no amount of the specified component can be detected with standard analytical methods.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

“Treatment” and “treating” refer to administration or application of a therapeutic agent to a subject or performance of a procedure or modality on a subject for the purpose of obtaining a therapeutic benefit of a disease or health-related condition. For example, a treatment may include administration of a T cell therapy.

“Subject” and “patient” are used interchangeably to refer to either a human or non-human, such as primates, mammals, and vertebrates. In particular embodiments, the subject is a human.

The term “therapeutic benefit” or “therapeutically effective” as used throughout this application refers to anything that promotes or enhances the well-being of the subject with respect to the medical treatment of this condition. This includes, but is not limited to, a reduction in the frequency or severity of the signs or symptoms of a disease. For example, treatment of cancer may involve, for example, a reduction in the size of a tumor, a reduction in the invasiveness of a tumor, reduction in the growth rate of the cancer, or prevention of metastasis. Treatment of cancer may also refer to prolonging survival of a subject with cancer.

An “anti-cancer” agent is capable of negatively affecting a cancer cell/tumor in a subject, for example, by promoting killing of cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of a subject with cancer.

The term “antibody” herein is used in the broadest sense and specifically covers monoclonal antibodies (including full length monoclonal antibodies), polyclonal antibodies, multispecific antibodies (e.g., bispecific antibodies), and antibody fragments so long as they exhibit the desired biological activity.

The phrases “pharmaceutical or pharmacologically acceptable” refers to molecular entities and compositions that do not produce an adverse, allergic, or other untoward reaction when administered to an animal, such as a human, as appropriate. The preparation of a pharmaceutical composition comprising an antibody or additional active ingredient will be known to those of skill in the art in light of the present disclosure. Moreover, for animal (e.g., human) administration, it will be understood that preparations should meet sterility, pyrogenicity, general safety, and purity standards as required by FDA Office of Biological Standards.

As used herein, “pharmaceutically acceptable carrier” includes any and all aqueous solvents (e.g., water, alcoholic/aqueous solutions, saline solutions, parenteral vehicles, such as sodium chloride, Ringer's dextrose, etc.), non-aqueous solvents (e.g., propylene glycol, polyethylene glycol, vegetable oil, and injectable organic esters, such as ethyloleate), dispersion media, coatings, surfactants, antioxidants, preservatives (e.g., antibacterial or antifungal agents, anti-oxidants, chelating agents, and inert gases), isotonic agents, absorption delaying agents, salts, drugs, drug stabilizers, gels, binders, excipients, disintegration agents, lubricants, sweetening agents, flavoring agents, dyes, fluid and nutrient replenishers, such like materials and combinations thereof, as would be known to one of ordinary skill in the art. The pH and exact concentration of the various components in a pharmaceutical composition are adjusted according to well-known parameters.

The term “unit dose” or “dosage” refers to physically discrete units suitable for use in a subject, each unit containing a predetermined quantity of the therapeutic composition calculated to produce the desired responses discussed above in association with its administration, i.e., the appropriate route and treatment regimen. The quantity to be administered, both according to number of treatments and unit dose, depends on the effect desired. The actual dosage amount of a composition of the present embodiments administered to a patient or subject can be determined by physical and physiological factors, such as body weight, the age, health, and sex of the subject, the type of disease being treated, the extent of disease penetration, previous or concurrent therapeutic interventions, idiopathy of the patient, the route of administration, and the potency, stability, and toxicity of the particular therapeutic substance. For example, a dose may also comprise from about 1 μg/kg/body weight to about 1000 mg/kg/body weight (this such range includes intervening doses) or more per administration, and any range derivable therein. In nonlimiting examples of a derivable range from the numbers listed herein, a range of about 5 μg/kg/body weight to about 100 mg/kg/body weight, about 5 μg/kg/body weight to about 500 mg/kg/body weight, etc., can be administered. The practitioner responsible for administration will, in any event, determine the concentration of active ingredient(s) in a composition and appropriate dose(s) for the individual subject. In some embodiments, the dosage of antigen-specific T cell infusion may comprise about 100 million to about 30 billion cells, such as 10, 15, or 20 billion cells.

The term “immune checkpoint” refers to a molecule such as a protein in the immune system which provides signals to its components in order to balance immune reactions. Known immune checkpoint proteins comprise CTLA-4, PD1 and its ligands PD-L1 and PD-L2 and in addition LAG-3, BTLA, B7H3, B7H4, TIM3, KIR. The pathways involving LAG3, BTLA, B7H3, B7H4, TIM3, and KIR are recognized in the art to constitute immune checkpoint pathways similar to the CTLA-4 and PD-1 dependent pathways (see e.g. Pardoll, 2012; Mellman et al., 2011.

An “immune checkpoint inhibitor” refers to any compound inhibiting the function of an immune checkpoint protein. Inhibition includes reduction of function and full blockade. In particular, the immune checkpoint protein is a human immune checkpoint protein. Thus, the immune checkpoint protein inhibitor in particular is an inhibitor of a human immune checkpoint protein.

As used herein, a “protective immune response” refers to a response by the immune system of a mammalian host to a cancer. A protective immune response may provide a therapeutic effect for the treatment of a cancer, e.g., decreasing tumor size or increasing survival.

As used herein, the term “antigen” is a molecule capable of being bound by an antibody or T-cell receptor. An antigen may generally be used to induce a humoral immune response and/or a cellular immune response leading to the production of B and/or T lymphocytes.

The terms “tumor-associated antigen,” “tumor antigen” and “cancer cell antigen” are used interchangeably herein. In each case, the terms refer to proteins, glycoproteins or carbohydrates that are specifically or preferentially expressed by cancer cells.

The term “chimeric antigen receptors (CARs),” as used herein, may refer to artificial T-cell receptors, chimeric T-cell receptors, or chimeric immunoreceptors, for example, and encompass engineered receptors that graft an artificial specificity onto a particular immune effector cell. CARs may be employed to impart the specificity of a monoclonal antibody onto a T cell, thereby allowing a large number of specific T cells to be generated, for example, for use in adoptive cell therapy. In specific embodiments, CARs direct specificity of the cell to a tumor associated antigen, for example. In some embodiments, CARs comprise an intracellular activation domain, a transmembrane domain, and an extracellular domain comprising a tumor associated antigen binding region. In particular aspects, CARs comprise fusions of single-chain variable fragments (scFv) derived from monoclonal antibodies, fused to CD3-zeta a transmembrane domain and endodomain. The specificity of other CAR designs may be derived from ligands of receptors (e.g., peptides) or from pattern-recognition receptors, such as Dectins. In certain cases, the spacing of the antigen-recognition domain can be modified to reduce activation-induced cell death. In certain cases, CARs comprise domains for additional co-stimulatory signaling, such as CD3ζ, FcR, CD27. CD28, CD137, DAP10, and/or OX40. In some cases, molecules can be co-expressed with the CAR, including co-stimulatory molecules, reporter genes for imaging (e.g., for positron emission tomography), gene products that conditionally ablate the T cells upon addition of a pro-drug, homing receptors, chemokines, chemokine receptors, cytokines, and cytokine receptors.

A polynucleotide or polynucleotide region (or a polypeptide or polypeptide region) has a certain percentage (for example, 80%, 85%, 90%, or 95%) of “sequence identity” or “homology” to another sequence means that, when aligned, that percentage of bases (or amino acids) are the same in comparing the two sequences. This alignment and the percent homology or sequence identity can be determined using software programs known in the art, for example those described in CURRENT PROTOCOLS IN MOLECULAR BIOLOGY (F. M. Ausubel el al., eds., 1987) Supplement 30, section 7.7.18, Table 7.7.1. Preferably, default parameters are used for alignment. A preferred alignment program is BLAST, using default parameters. In particular, preferred programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR.

### III. EGFR Mutant Peptides

Embodiments of the present disclosure concern tumor antigen-specific peptides, such as to the EGFR peptides that include a mutant EGFR sequence (e.g., a peptide having an insertion, substitution or deletion relative to a wildtype EGR sequence). In particular embodiments, the tumor antigen-specific peptides have the amino acid sequence of a EGFR mutant peptides. In some aspects, the peptide is no more than 50, 45, 40, 35, 30, 25, 20 or 15 amino acids in length. In certain aspects, such as any of those in Tables 1-3 is fused to polypeptide having a non-EGFR amino acid sequence (i.e., a heterologous polypeptide). In some aspects the tumor antigen-specific peptide may have an amino acid sequence with at least 80, 85, 90, 95, 96, 97, 98, 99, or 100 percent sequence identity with the peptide sequence of those in Tables 1-3 or a sequence according to those in Tables 1-3, but including 1, 2 or 3 amino acid substitutions or deletions relative to those in Tables 1-3.

As used herein, the term “peptide” encompasses amino acid chains comprising 7-35 amino acids, preferably 8-35 amino acid residues, and even more preferably 8-25 amino acids, or 7, 8, 9, 10, 11, 12, 13, 14, 15, 16, 17, 18, 19, 20, 21, 22, 23, 24, 25, 26, 27, 28, 29, 30, 31, 32, 33, 34, or 35 amino acids in length, or any range derivable therein. For example, EGFR mutant peptides of the present disclosure may, in some embodiments, comprise or consist of the sequence of any one of those in Tables 1-3. As used herein, an “antigenic peptide” is a peptide which, when introduced into a vertebrate, can stimulate the production of antibodies in the vertebrate, i.e., is antigenic, and wherein the antibody can selectively recognize and/or bind the antigenic peptide. An antigenic peptide may comprise an immunoreactive EGFR mutant peptides, and may comprise additional sequences. The additional sequences may be derived from a native antigen and may be heterologous, and such sequences may, but need not, be immunogenic. In some embodiments, a tumor antigen-specific peptide (e.g., EGFR mutant peptides) can selectively bind with Specific HLA class I or HLA class II complexes. In certain embodiments, the EGFR mutant peptides are 8, 9, 10, 11, 12, 13, 14, 15, 16, 17, 18, 19, 20, 21, 22, 23, 24, 25, 26, 27, 28, 29, 30, 31, 32, 33, 34, or 35 amino acids in length, or any range derivable therein. Preferably, the tumor antigen-specific peptide (e.g., EGFR mutant peptides) is from 8 to 35 amino acids in length. In some embodiments, the tumor antigen-specific peptide (e.g., EGFR mutant peptides) is from 8 to 10 amino acids in length.

As would be appreciated by one of skill in the art, MHC molecules can bind peptides of varying sizes, but typically not full-length proteins. While MHC class I molecules have been traditionally described to bind to peptides of 8-11 amino acids long, it has been shown that peptides 15 amino acids in length can bind to MHC class I molecules by bulging in the middle of the binding site or extending out of the MHC class I binding groove (Guo et al., 1992; Burrows et al., 2006; Samino et al., 2006; Stryhn el al, 2000; Collins et al., 1994; Blanchard and Shastri, 2008). Further, recent studies also demonstrated that longer peptides may be more efficiently endocytosed, processed, and presented by antigen-presenting cells (Zwaveling et al., 2002; Bijker et al., 2007; Melief and van der Burg, 2008; Quintarelli et al., 2011). As demonstrated in Zwaveling el al. (2002) peptides up to 35 amino acids in length may be used to selectively bind a class II MHC and are effective. As would be immediately appreciated by one of skill, a naturally occurring full-length tumor antigen, such as mutant EGFR, would not be useful to selectively bind a class II MHC such that it would be endocytosed and generate proliferation of T cells. Generally, the naturally occurring full-length tumor antigen proteins do not display these properties and would thus not be useful for these immunotherapy purposes.

In certain embodiments, a tumor antigen-specific peptide (e.g., EGFR mutant peptides) is immunogenic or antigenic. As shown in the below examples, various tumor antigen-specific peptides (e.g., EGFR mutant peptides) of the present disclosure can promote the proliferation of T cells. It is anticipated that such peptides may be used to induce some degree of protective immunity.

A tumor antigen-specific peptide (e.g., EGFR mutant peptides) may be a recombinant peptide, synthetic peptide, purified peptide, immobilized peptide, detectably labeled peptide, encapsulated peptide, or a vector-expressed peptide (e.g., a peptide encoded by a nucleic acid in a vector comprising a heterologous promoter operably linked to the nucleic acid). In some embodiments, a synthetic tumor antigen-specific peptide (e.g., EGFR mutant peptides) may be administered to a subject, such as a human patient, to induce an immune response in the subject. Synthetic peptides may display certain advantages, such as a decreased risk of bacterial contamination, as compared to recombinantly expressed peptides. A tumor antigen-specific peptide (e.g., EGFR mutant peptides) may also be comprised in a pharmaceutical composition such as, e.g., a vaccine composition, which is formulated for administration to a mammalian or human subject.

A. HLA Class I and H Library Peptides Example Procedures for Peptide Selection

Table 1 below shows an example of a final peptide composition of EGFR mutant peptides predicted to bind to specific HLA class I complexes. The peptides have been ranked based on predicted binding characteristics. In this case, the example patient has a L858R mutation and 2 different specific HLA-A, HLA-B and HLA-C genes.

Example Procedure for Selecting MIA Class I Peptides (HLA Class II Peptides can be Selected Using the Same Procedure):
1. Patient's tumor is analyzed genetically to determine if they have an EGFR mutation.
2. Patient's HLA class I and HLA class II typing is performed to determine which HLA molecules they express.
3. If patient contains an EGFR mutation, HLA peptide binding prediction is performed to determine whether peptides containing the EGFR mutation are capable of binding to any of the HLA class I or HLA class II molecules. For the Top 20 most prevalent EGFR mutations, those predicted peptides are listed in the tables included.
4. Patient example in Table 1: EGFR sequencing showed that the patient had an L858R EGFR mutation, and HLA class I typing was HLA-A*0101, A*0301, B*0702, B*4601, C*0102, and C*0701.
5. From the specific peptide lists (See Tables 2 & 3), obtain all peptides that are listed for those 6 HLA types and combine them into one list.
6. Score the peptides individually according to their predicted affinity (in nM) and predicted percentile as follows:

| Affinity ranking: | | Priority | Score | |
|---|---|---|---|---|
| <1000 | nM | high priority | 3 | points |
| 1001-5000 | nM | medium | 2 | pts. |
| 5001-12000 | nM | low | 1 | pt. |
| >12001 | nM | very low | 0 | pts. |

| Percentile ranking: | | |
|---|---|---|
| <1.0 | high priority | 3 pts |
| 1.0-3.0 | medium | 2 pts |
| 3.0-7.0 | low | 1 pt |
| >7.0 | very low | 0 pts |

7. Sum the affinity scores (max=3) and percentile scores (max=3) together to obtain the final total score for each peptide (maximum total score=6).
8. Rank the peptides according to their total score.
9. Peptides with the highest total scores are prioritized to include in the immunogenic composition (vaccine)

The HLA Class I Library contains the top 20 EGFR mutations and covers ˜95% of EGFRmut patients (see Table 2 below). There are 364 total EGFR mutant peptides in library, including 342 mutation-specific peptides and 16 peptides shared by 2 mutations. The library contains 100 HLA class I allotypes (HLA-A, 30; HLA-B, 48; and HLA-C, 22)

The HLA Class II Library comes the top 20 EGFR mutations and contains 429 total EGFR mutant peptides in the library (see Table 3 below). The top 20 EFGR mutation are A763_Y764insFQEA, D770_N771insSVD, E746_A750del, E746_S752del>V, G719A, G719C, G719S, H773_V774insH, I744_K745insKIPVAI, L747_A750del>P, L747_P753del>S, L747_S752del, L747_T751del, L747_T751del>P, L858R, L861Q, P772_H773insPR, S768I, T790M, and V769_D770insASV.

TABLE 1
Example HL A Class I Library of peptides and associated characteristics.

| HLA | Neoantigen | Affinity | Percentile | Affinity | Percentile | Affinity | Percentile | Total | SEQ ID |
|---|---|---|---|---|---|---|---|---|---|
| class I | Peptide | (nM) | rank | Priofity | Priority | scare | score | score | NO |
| A*0301 | RITDFGRAK | 103 | 0.37 | high | high | 3 | 3 | 6 | 4 |
| C*0102 | ITDFGRAKL | 1826 | 0.14 | medium | high | 2 | 3 | 5 | 3 |
| A*0301 | VKITDFGRAR | 888 | 1.71 | high | medium | 3 | 2 | 5 | 16 |
| A*0101 | ITDFGRAKL | 3125 | 1.07 | medium | medium | 2 | 2 | 4 | 3 |
| A*0101 | ITDFGRAKLL | 3387 | 1.13 | medium | medium | 2 | 2 | 4 | 12 |
| A*0101 | ITDFGRAKLLGA | 4886 | 1.51 | medium | medium | 2 | 2 | 4 | 17 |
| A*0301 | RAKLLGAEER | 1971 | 2.73 | medium | medium | 2 | 2 | 4 | 2 |
| C*0102 | ITDFGRAKLL | 8109 | 1.37 | low | medium | 1 | 2 | 3 | 12 |
| A*0301 | RITDFGRAKL | 3454 | 3.74 | medium | tow | 2 | 1 | 3 | 10 |
| A*0301 | HVKITDFGRAK | 3827 | 3.97 | medium | low | 2 | 1 | 3 | 9 |
| C*0102 | RITDFGRAKL | 12062 | 2.61 | very low | medium | 0 | 2 | 2 | 10 |
| 8*0702 | KITDFGRAKL | 5092 | 3.43 | low | low | 1 | 1 | 2 | 10 |
| C*0701 | ITDFGRAKL | 8774 | 3.58 | tow | tow | 1 | 1 | 2 | 3 |
| 8*0702 | ITDFGRAKL | 6083 | 3.91 | low | low | 1 | 1 | 2 | 3 |
| C*0701 | ITDFGRAKLL | 10076 | 4.33 | tow | tow | 1 | 1 | 2 | 12 |
| C*0701 | FGRAKLLGA | 10891 | 4.81 | low | low | 1 | 1 | 2 | 6 |
| 8*0702 | RAKLLGAEE | 9823 | 5.87 | tow | tow | 1 | 1 | 2 | 8 |
| 8*0702 | FGRAKLLGA | 11811 | 6.98 | low | low | 1 | 1 | 2 | 6 |
| B*4601 | ITDFGRAKL | 24630 | 5.06 | very low | tow | 0 | 1 | 1 | 3 |
| A*0101 | KITDFGRAKL | 15530 | 5.62 | very tow | low | 0 | 1 | 1 | 10 |
| 8*4601 | FGRAKLLGA | 26586 | 6.29 | very low | tow | 0 | 1 | 1 | 6 |
| A*0301 | AVKITDFGR | 9015 | 7.10 | low | very low | 1 | 0 | 1 | 1 |

TABLE 2
HLA Class I Library of peptides and associated characteristics.

| Peptide | | Peptide | Mutation | Peptide | Minimum | Minimum | SEQ ID |
|---|---|---|---|---|---|---|---|
| ID | Mutation | length | position | mt | affinity | ranking | NO |
| 1875 | L858R | 9 | 9 | HVKITDFGR | 19 | 0.08 | 1 |
| 1826 | L858R | 10 | 1 | RAKLLGAEEK | 48 | 0.25 | 2 |
| 1872 | L858R | 9 | 6 | ITDFGRAKL | 64 | 0.04 | 3 |
| 1873 | L858R | 9 | 7 | KITDFGRAK | 98 | 0.37 | 4 |
| 1827 | L858R | 10 | 10 | QHVKITDFGR | 217 | 0.80 | 5 |
| 1869 | L858R | 9 | 3 | FGRAKLLGA | 329 | 0.55 | 6 |
| 1839 | L858R | 11 | 2 | GRAKLLGAEEK | 388 | 1.03 | 7 |
| 1867 | L858R | 9 | 1 | RAKLLGAEE | 418 | 1.77 | 8 |
| 1846 | L858R | 11 | 9 | HVKITDFGRAK | 425 | 1.80 | 9 |
| 1833 | L858R | 10 | 7 | KITDFGRAKL | 492 | 1.10 | 10 |
| 1850 | L858R | 12 | 12 | TPQHVKITDFGR | 577 | 1.65 | 11 |
| 1832 | L858R | 10 | 6 | ITDFGRAKLL | 591 | 0.22 | 12 |
| 1847 | L858R | 12 | 1 | RAKLLGAEEKEY | 598 | 1.09 | 13 |
| 1835 | L858R | 10 | 9 | HVKITDFGRA | 694 | 2.64 | 14 |
| 1871 | L858R | 9 | 5 | TDFGRAKLL | 766 | 0.15 | 15 |
| 1834 | L858R | 10 | 8 | VKITDFGRAK | 825 | 1.60 | 16 |
| 1855 | L858R | 12 | 6 | ITDFGRAKLLGA | 882 | 1.51 | 17 |
| 1868 | L858R | 9 | 5 | GRAKLLGAE | 1148 | 2.15 | 18 |
| 487 | E746_A750del | 10 | 7 | IPVAIKTSPK | 71 | 0.27 | 19 |
| 484 | E746_A750del | 10 | 4 | AIKTSPKANK | 133 | 0.47 | 20 |
| 498 | E746_A750del | 11 | 8 | KJPVAIKTSPK | 141 | 0.49 | 21 |
| 481 | E746_A750del | 10 | 10 | KVKIPVAIKT | 190 | 0.92 | 22 |
| 522 | E746_A750del | 9 | 6 | PVAIKTSPK | 555 | 1.12 | 23 |
| 510 | E746_A750del | 12 | 9 | VKIPVAIKTSPK | 577 | 1.33 | 24 |
| 511 | E746_A750del | 8 | 2 | KTSPKANK | 641 | 2.48 | 25 |
| 497 | E746_A750del | 11 | 7 | IPVAIKTSPKA | 644 | 0.66 | 26 |
| 523 | E746_A750del | 9 | 7 | IPVAIKTSP | 970 | 0.85 | 27 |
| 495 | E746_A750del | 11 | 5 | VAIKTSPKANK | 1296 | 2.89 | 28 |
| 482 | E746_A750del | 10 | 2 | KTSPKANKEI | 1384 | 1.57 | 29 |
| 521 | E746_A750del | 9 | 5 | VAIKTSPKA | 1759 | 1.88 | 30 |
| 492 | E746_A750del | 11 | 2 | KTSPKANKEIL | 2737 | 2.23 | 31 |
| 1740 | L747_T751del | 10 | 5 | AIKESPKANK | 171 | 0.57 | 32 |
| 1743 | L747_T751del | 10 | 8 | IPVAIKESPK | 464 | 1.15 | 33 |
| 1754 | L747_T751del | 11 | 9 | KIPVAIKFSPK | 560 | 1.54 | 34 |
| 1753 | L747_T751del | 11 | 8 | IPVAIKESPKA | 1306 | 1.06 | 35 |
| 1746 | L747_T751del | 11 | 11 | KVKIPVAIKIS | 1495 | 4.72 | 36 |
| 1751 | L747_T751del | 11 | 6 | VAIKESPKANK | 1789 | 3.40 | 37 |
| 1738 | L747_T751del | 10 | 3 | KESPKANKEI | 1792 | 1.07 | 38 |
| 1779 | L747_T751del | 9 | 8 | IPVAIKESP | 1955 | 1.43 | 39 |
| 1748 | L747_T751del | 11 | 3 | KESPKANKEIL | 1968 | 1.43 | 40 |
| 1755 | L747_T751del | 12 | 10 | VKIPVAIKESPK | 2445 | 3.76 | 41 |
| 1737 | L747_T751del | 10 | 2 | ESPKANKEIL | 2500 | 2.25 | 42 |
| 1778 | L747_T751del | 9 | 7 | PVAIKESPK | 2638 | 3.72 | 43 |
| 1760 | L747_T751del | 12 | 4 | IKESPKANKEIL | 3351 | 3.14 | 44 |
| 1777 | L747_T751del | 9 | 6 | VAIKESPKA | 3645 | 4.07 | 45 |
| | L747_P753del>S | 9 | 5 | AIKESKANK | 79 | 0.40 | 46 |
| | 1747_P753del>S | 12 | 11 | KVKIPVAIKESK | 134 | 0.64 | 47 |
| | L747_P753del>S | 10 | 8 | IPVAIKESKA | 211 | 0.27 | 48 |
| | L747_P753del>S | 10 | 3 | KESKANKEIL | 230 | 0.44 | 49 |
| | L747_P753del>S | 9 | 3 | KESKANKEL | 361 | 0.39 | 50 |
| | 1747_P753del>S | 10 | 9 | KIPVAIKESK | 400 | 1.25 | 51 |
| | L747_P753del>S | 10 | 6 | VAIKESKANK | 722 | 1.43 | 52 |
| | L747_P753del>S | 11 | 11 | KVKIPVAIKES | 1495 | 4.72 | 53 |
| | 1747_P753del>S | 12 | 1 | SKANKEILDEAY | 1765 | 4.61 | 54 |
| | L747_P753del>S | 9 | 8 | IPVAIKESK | 2523 | 2.49 | 55 |
| | 1747_P753del>S | 11 | 5 | AIKESKANKEL | 2920 | 1.57 | 56 |
| | L747_P753del>S | 12 | 8 | IPVAIKESKANK | 3799 | 3.95 | 57 |
| | L747_P753del>S | 10 | 4 | IKESKANKEI | 4716 | 1.60 | 58 |
| 2391 | T790M | 10 | 5 | VQLIMQLMPF | 5 | 0.02 | 59 |
| 2386 | T790M | 10 | 1 | MQLMPFGCLL | 8 | 0.03 | 60 |
| 2427 | T790M | 9 | J | MQLMPFGCL | 10 | 0.05 | 61 |
| 2430 | T790M | 9 | 4 | QUMQLMPF | 11 | 0.06 | 62 |
| 2399 | T790M | 11 | 2 | IMQLMPFGCLL | 31 | 0.33 | 63 |
| 2407 | T790M | 12 | 1 | MQLMPFGCLLDY | 40 | 0.27 | 64 |
| 2435 | T790M | 9 | 9 | LTSTVQLIM | 49 | 0.09 | 65 |
| 2412 | T790M | 12 | 3 | LIMQLMPFGCLL | 61 | 0.66 | 66 |
| 2388 | T790M | 10 | 2 | IMQLMPFGCL | 83 | 0.58 | 67 |
| 2433 | T790M | 9 | 7 | STVQLIMQL | 89 | 0.34 | 68 |
| 2421 | T790M | 8 | 3 | LIMQLMPF | 159 | 0.67 | 69 |
| 2403 | T790M | 11 | 6 | TVQLIMQLMPF | 209 | 1.63 | 70 |
| 2423 | T790M | 8 | 5 | VQLIMQLM | 268 | 2.30 | 71 |
| 2387 | T790M | 10 | 10 | CLTSTVQLIM | 326 | 1.10 | 77 |
| 2416 | T790M | 12 | 7 | STVQLIMQLMPP | 350 | 0.87 | 73 |
| 2394 | T790M | 10 | 8 | TSTVQLIMQL | 377 | 1.70 | 74 |
| 2400 | T790M | 11 | 3 | LIMQLMPFGCL | 492 | 2.09 | 75 |
| 2393 | T790M | 10 | 7 | STVQLIMQLM | 542 | 0.21 | 76 |
| 2390 | T790M | 10 | 4 | QLIMQLMPFG | 583 | 1.88 | 77 |
| 2418 | T790M | 12 | 9 | LTSTVQLIMQLM | 588 | 1.54 | 78 |
| 2406 | T790M | 11 | 9 | LTSTVQLIMQL | 657 | 1.69 | 79 |
| 2398 | T790M | 11 | 11 | ICLTSTVQLIM | 907 | 2.09 | 80 |
| 2395 | T790M | 10 | 9 | LTSTVQLIMQ | 1125 | 2.42 | 81 |
| 2432 | T790M | 9 | 6 | TVQLIMQLM | 1312 | 0.62 | 82 |
| 1878 | L861Q | 10 | 2 | KQLGAEEKEY | 76 | 0.66 | 83 |
| 1924 | L861Q | 9 | 8 | TDFGLAKQL | 714 | 0.69 | 84 |
| 1877 | L861Q | 10 | 10 | KITDFGLAKQ | 753 | 1.74 | 85 |
| 1903 | L861Q | 12 | 4 | LAKQLGAEEKEY | 805 | 2.16 | 86 |
| 1885 | L861Q | 10 | 9 | ITDFGLAKQL | 1059 | 0.36 | 87 |
| 1880 | L861Q | 10 | 4 | LAKQLGAEEK | 1355 | 3.21 | 88 |
| 1892 | L861Q | 11 | 5 | GLAKQLGAEEK | 1607 | 2.44 | 89 |
| 1922 | L861Q | 9 | 6 | FGLAKQLGA | 1745 | 4.34 | 90 |
| 1887 | L861Q | 11 | 10 | KITDFGLAKQL | 1925 | 4.62 | 91 |
| 1917 | L861Q | 9 | 1 | QLGAEEKEY | 3461 | 3.83 | 92 |
| 1908 | L861Q | 12 | 9 | ITDFGLAKQLGA | 4074 | 1.30 | 93 |
| 2232 | S768I | 10 | 10 | ILDEAYVMAI | 6 | 0.11 | 94 |
| 2247 | S768I | 11 | 5 | YVMAIVDNPHV | 8 | 0.13 | 95 |
| 2235 | S768I | 10 | 4 | VMAIVDNPHV | 17 | 0.60 | 96 |
| 2242 | S768I | 11 | 10 | ILDEAYVMAIV | 25 | 0.66 | 97 |
| 2255 | S768I | 12 | 12 | KEILDEAYVMAI | 54 | 0.21 | 98 |
| 2245 | S768I | 11 | 3 | MAIVDNPHVCR | 78 | 0.54 | 99 |
| 2274 | S768I | 9 | 3 | MAIVDNPHV | 93 | 0.18 | 100 |
| 2260 | S768I | 12 | 6 | AYVMAIVDNPHV | 105 | 1.34 | 101 |
| 2233 | S768I | 10 | 2 | AIVDNPHVCR | 134 | 0.82 | 102 |
| 2254 | S768I | 12 | 11 | EILDEAYVMAIV | 150 | 0.93 | 103 |
| 2258 | S768I | 12 | 4 | VMAIVDNPHVCR | 168 | 0.88 | 104 |
| 2243 | S768I | 11 | 11 | EILDEAYVMAI | 198 | 2.45 | 105 |
| 2272 | S768I | 9 | 1 | IVDNPHVCR | 224 | 0.92 | 106 |
| 2770 | S768I | 9 | 8 | DEAYVMAIV | 242 | 0.16 | 107 |
| 2259 | S768I | 12 | 5 | YVMAIVDNPHVC | 266 | 2.39 | 108 |
| 2236 | S768I | 10 | 5 | YVMAIVDNPH | 326 | 0.80 | 109 |
| 2231 | S768I | 10 | 1 | IVDNPHVCRL | 332 | 0.14 | 110 |
| 2275 | $7681 | 9 | 4 | VMAIVDNPH | 333 | 1.42 | ill |
| 2240 | S768I | 10 | 9 | LDEAYVMAIV | 523 | 0.46 | 112 |
| 2271 | S768I | 8 | 8 | DEAYVMAI | 543 | 0.33 | 113 |
| 2270 | S768I | 8 | 7 | EAYVMAIV | 900 | 0.16 | 114 |
| 2241 | S768I | 11 | 1 | IVDNPHVCRLL | 958 | 0.33 | 115 |
| 2276 | S768I | 9 | 5 | YVMAIVDNP | 1129 | 3.22 | 116 |
| 2244 | S768I | 11 | 2 | AIVDNPHVCRL | 1268 | 3.33 | 117 |
| 2280 | S768I | 9 | 9 | LDEAYVMAI | 1333 | 0.35 | 118 |
| 2257 | S768I | 12 | 3 | MAIVDNPHVCRL | 1619 | 2.82 | 119 |
| 2266 | S768I | 8 | 3 | MAIVDNPH | 2620 | 2.49 | 120 |
| 2256 | S768I | 12 | 2 | AIVDNPHVCRLL | 2631 | 3.60 | 121 |
| 706 | G719A | 9 | 5 | IKVLASGAF | 4 | 0.03 | 122 |
| 661 | G719A | 10 | 1 | ASGAFGTVYK | 8 | 0.01 | 123 |
| 664 | G719A | 10 | 3 | VLASGAFGTV | 8 | 0.15 | 124 |
| 667 | G719A | 10 | 6 | KIKVLASGAF | 15 | 0.17 | 125 |
| 663 | G719A | 10 | 2 | LASGAFGTVY | 26 | 0.07 | 126 |
| 702 | G719A | 9 | 1 | ASGAFGTVY | 28 | 0.20 | 127 |
| 707 | G719A | 9 | 6 | KIKVLASGA | 30 | 0.15 | 128 |
| 704 | G719A | 9 | 3 | VLASGAFGT | 31 | 1.31 | 129 |
| 703 | G719A | 9 | 2 | LASGAFGTV | 41 | 0.18 | 130 |
| 679 | G719A | 11 | 7 | KKIKVLASGAF | 41 | 0.74 | 131 |
| 687 | G719A | 12 | 3 | VLASGAFGTVYK | 64 | 0.30 | 132 |
| 705 | G719A | 9 | 4 | KVLASGAFG | 68 | 0.35 | 133 |
| 674 | G719A | 11 | 7 | LASGAFGTVYK | 94 | 0.55 | 134 |
| 688 | G719A | 12 | 4 | KVLASGAFGTVY | 95 | 0.24 | 135 |
| 675 | G719A | 11 | 3 | VLASGAFGTVY | 112 | 0.58 | 136 |
| 676 | G719A | 11 | 4 | KVLASGAFGTV | 119 | 1.65 | 137 |
| 692 | G719A | 12 | 8 | FKKIKVLASGAF | 126 | 1.65 | 138 |
| 666 | G719A | 10 | 5 | IKVLASGAFG | 193 | 2.20 | 139 |
| 697 | G719A | 8 | 4 | KVLASGAF | 207 | 2.22 | 140 |
| 662 | G719A | 10 | 10 | TEFKKIKVLA | 259 | 0.24 | 141 |
| 671 | G719A | 11 | 1 | ASGAFGTVYKG | 502 | 1.76 | 142 |
| 668 | G719A | 10 | 7 | KKIKVLASGA | 590 | 2.33 | 143 |
| 709 | G719A | 9 | 8 | FKKIKVLAS | 981 | 1.31 | 144 |
| 685 | G7I9A | 12 | 12 | KETEFKKIKVLA | 1688 | 1.35 | 145 |
| 701 | G719A | 8 | 8 | FKKIKVLA | 2264 | 2.66 | 146 |
| 682 | G719A | 12 | 1 | ASGAFGTVYKGL | 2279 | 3.86 | 147 |
| 680 | G719A | 11 | 8 | FKKIKVLASGA | 2591 | 4.27 | 148 |
| 756 | G719C | 9 | 5 | IKVLCSGAF | 5 | 0.04 | 149 |
| 714 | G719C | 10 | 3 | VLCSGAFGTV | 20 | 0.49 | 150 |
| 711 | G719C | 10 | 1 | CSGAFGTVYK | 23 | 0.13 | 151 |
| 717 | G719C | 10 | 6 | KIKVLCSGAF | 26 | 0.25 | 152 |
| 729 | G719C | 11 | 7 | KKIKVLCSGAF | 57 | 0.96 | 153 |
| 752 | G719C | 9 | 1 | CSGAFGTVY | 63 | 0.14 | 154 |
| 757 | G719C | 9 | 6 | KIKVLCSGA | 65 | 0.34 | 155 |
| 737 | G719C | 12 | 3 | VLCSGAFGTVYK | 92 | 0.37 | 156 |
| 755 | G719C | 9 | 4 | KVLCSGAFG | 101 | 0.50 | 157 |
| 738 | G719C | 12 | 4 | KVLCSGAFGTVY | 135 | 0.35 | 158 |
| 754 | G719C | 9 | 3 | VLCSGAFGT | 159 | 3.34 | 159 |
| 726 | G719C | 11 | 4 | KVLCSGAFGTV | 162 | 1.56 | 160 |
| 713 | G719C | 10 | 2 | LCSGAFGTVY | 176 | 0.41 | 161 |
| 747 | G7190 | 8 | 4 | KVLCSGAF | 264 | 2.49 | 162 |
| 742 | G719C | 12 | 8 | FKKIKVLCSGAF | 273 | 1.71 | 163 |
| 725 | G719C | 11 | 3 | VLCSGAFGTVY | 462 | 0.89 | 164 |
| 716 | G719C | 10 | 5 | IKVLCSGAFG | 481 | 3.78 | 165 |
| 724 | G719C | 11 | 2 | LCSGAFGTVYK | 676 | 2.06 | 166 |
| 718 | G719C | 10 | 7 | KKIKVLCSGA | 933 | 3.31 | 167 |
| 712 | G719C | 10 | 10 | TEFKKIKVLC | 1083 | 1.02 | 168 |
| 728 | G719C | 11 | 6 | KIKVLCSGAFG | 1216 | 4.03 | 169 |
| 753 | G719C | 9 | 7 | LCSGAFGTV | 1813 | 4.03 | 170 |
| 806 | G719S | 9 | 5 | IKVLSSGAF | 4 | 0.02 | 171 |
| 761 | G719S | 10 | 1 | SSGAFGTVYK | 7 | 0.01 | 172 |
| 764 | G719S | 10 | 3 | VLSSGAFGTV | 14 | 0.33 | 173 |
| 767 | G719S | 10 | 6 | KIKVLSSGAF | 16 | 0.14 | 174 |
| 807 | G719S | 9 | 6 | KIKVLSSGA | 21 | 0.09 | 175 |
| 763 | G719S | 10 | 2 | LSSGAFGTVY | 34 | 0.07 | 176 |
| 802 | G7I9S | 9 | 1 | SSGAFGTVY | 35 | 0.22 | 177 |
| 779 | G719S | 11 | 7 | KKIKVLSSGAF | 40 | 0.73 | 178 |
| 803 | G719S | 9 | 2 | LSSGAFGTV | 54 | 0.17 | 179 |
| 774 | G719S | 11 | 2 | LSSGAFGTVYK | 55 | 0.34 | 180 |
| 787 | G719S | 12 | 3 | VLSSGAFGTVYK | 71 | 0.31 | 181 |
| 805 | G719S | 9 | 4 | KVLSSGAFG | 84 | 0.42 | 182 |
| 804 | G719S | 9 | 3 | VLSSGAFGT | 86 | 2.19 | 183 |
| 788 | G719S | 12 | 4 | KVLSSGAFGTVY | 87 | 0.22 | 184 |
| 792 | G719S | 12 | 8 | FKKIKVLSSGAF | 96 | 1.38 | 185 |
| 766 | G719S | 10 | 5 | IKVLSSGAFG | 174 | 2.05 | 186 |
| 776 | G719S | 11 | 4 | KVLSSGAFGTV | 185 | 1.72 | 187 |
| 775 | G7198 | 11 | 3 | VLSSGAFGTVY | 186 | 0.53 | 188 |
| 797 | G719S | 8 | 4 | KVLSSGAF | 245 | 2.42 | 189 |
| 768 | G719S | 10 | 7 | KKIKVLSSGA | 368 | 1.59 | 190 |
| 771 | G719S | 11 | 1 | SSGAFGTVYKG | 482 | 1.72 | 191 |
| 809 | G719S | 9 | 8 | FKKIKVLSS | 1302 | 1.65 | 192 |
| 762 | G719S | 10 | 10 | TEFKKIKVLS | 1616 | 1.04 | 193 |
| | E746_S752del>V | 10 | 7 | IPVAIKVPKA | 44 | 0.05 | 194 |
| | E746_S752del>V | 12 | 10 | KVKIPVAIKVPK | 59 | 0.30 | 195 |
| | E746_S752del>V | 9 | J | VPKANKEIL | 59 | 0.22 | 196 |
| | E746_S752del>V | 9 | 4 | AIKVPKANK | 60 | 0.31 | 197 |
| | E746_S752del>V | 10 | 10 | KVKIPVAIKV | 69 | 0.35 | 198 |
| | E746_S752del>V | 10 | 8 | KIPVAIKVPK | 81 | 0.36 | 199 |
| | E746_S752del>V | 10 | 2 | KVPKANKEIL | 41.0 | 0.69 | 200 |
| | E746_S752del>y | 10 | 5 | VAIKVPKANK | 450 | 0.93 | 201 |
| | E746_S752del>V | 9 | 7 | IPVAIKVPK | 814 | 0.97 | 202 |
| | E746_S752del>V | 9 | 9 | VKIPVAIKV | 1072 | 1.45 | 203 |
| | E746_S752del>V | 12 | 12 | GEKVKIPVAIKV | 1166 | 1.21 | 204 |
| | E746_S752del>V | 11 | 9 | VKIPVAIKVPK | 2442 | 3.06 | 205 |
| | E746_S752del>V | 11 | 3 | IKVPKANKEIL | 2716 | 2.73 | 206 |
| | E746_S752del>V | 9 | 2 | KVPKANKEI | 2720 | 1.72 | 207 |
| | L747_A750del>P | 9 | 5 | AIKEPTSPK | 23 | 0.10 | 208 |
| | L747_A7S0del>P | 10 | 6 | VAIKEPTSPK | 75 | 0.16 | 209 |
| | 1747_A750del>P | 9 | 8 | IPVAIKEPT | 140 | 0.17 | 210 |
| | L747_A750del>P | 12 | 5 | AIKEPTSPKANK | 237 | 0.72 | 211 |
| | L747_A750del>P | 12 | 8 | IPVAIKEPTSPK | 356 | 0.96 | 212 |
| | L747_A750deP>P | 10 | 5 | AIKEPTSPKA | 479 | 1.62 | 213 |
| | L747_A750del>P | 10 | 8 | IPVAIKEPTS | 931 | 0.79 | 214 |
| | L747_A750del>P | 11 | 7 | PVAIKEPTSPK | 1907 | 2.75 | 215 |
| | L747_A750del>P | 11 | 11 | KVKIPVAIKEP | 2335 | 6.30 | 216 |
| | L747_A750del>P | 10 | 9 | KIPVAIKEPT | 3463 | 2.27 | 217 |
| | L747_A750del>P | 9 | 6 | VAIKEPSPK | 154 | 0.79 | 218 |
| | 1747_A750del>P | 9 | 8 | IPVAIKEPS | 201 | 0.21 | 219 |
| | L747_A750del>P | 9 | 5 | AIKEPSPKA | 226 | 1.06 | 220 |
| | 1747_A750del>P | 11 | 5 | AIKEPSPKANK | 358 | 0.96 | 221 |
| | L747_A750del>P | 12 | 9 | KIPVAIKEPSPK | 495 | 1.30 | 222 |
| | L747_A750del>P | 12 | 8 | IPVAIKEPSPKA | 1079 | 0.97 | 223 |
| | L747_A750del>P | 12 | 11 | KVKIPVAIKEPS | 1085 | 3.70 | 224 |
| | L747_A750del>P | 12 | 6 | VAIKEPSPKANK | 1867 | 3.47 | 225 |
| 1699 | L747_S752del | 10 | 9 | KIPVAIKEPK | 218 | 1.06 | 226 |
| 1693 | L747_S752del | 10 | 3 | KEPKANKEIL | 410 | 0.69 | 227 |
| 1696 | L747_S752del | 10 | 6 | VAIKTPKANK | 500 | 1.02 | 228 |
| 1728 | L747_S752del | 9 | 2 | EPKANKEIL | 590 | 0.64 | 229 |
| 1734 | L747_S752del | 9 | 8 | IPVAIKEPK | 1415 | 1.76 | 230 |
| 1729 | L747_S752del | 9 | 3 | KEPKANKEI | 1882 | 1.59 | 231 |
| 1146 | I744_K745insKIPVAI | 9 | 4 | VAIKIPVAI | 11 | 0.04 | 232 |
| 1148 | I744_K745insKIPVAI | 9 | 6 | IPVAIKIPV | 18 | 0.03 | 233 |
| 1091 | I744_K745insKIPVAI | 10 | 6 | IPVAIKIPVA | 32 | 0.04 | 234 |
| 1145 | I744_K745insKIPVAI | 9 | 3 | AIKIPVAIK | 39 | 0.20 | 235 |
| 1086 | I744_K745insKIPVAI | 10 | 1 | KIPVAIKELR | 83 | 0.60 | 236 |
| 1094 | I744_K745insKIPVAI | 10 | 9 | KVKIPVAIKI | 87 | 0.44 | 237 |
| 1092 | I744_K745insKIPVAI | 10 | 7 | KIPVAIKIPV | 100 | 0.36 | 238 |
| 1130 | I744_K745insKIPVAI | 8 | 0 | IPVAIKEL | 122 | 0.51 | 239 |
| 1125 | I744_K745insKIPVAI | 12 | 9 | KVKIPVAIKIPV | 149 | 0.73 | 240 |
| 1112 | I744_K745insKIPVAI | 12 | −3 | AIKELREATSPK | 158 | 0.54 | 241 |
| 1098 | I744_K745insKIPVAI | 11 | −4 | IKELREATSPK | 161 | 0.55 | 242 |
| 1089 | I744_K745insKIPVAI | 10 | 4 | VAIKIPVAIK | 214 | 0.70 | 243 |
| 1119 | I744_K745insKIPVAI | 12 | 3 | AIKIPVAIKELR | 247 | 1.39 | 244 |
| 1106 | I744_K745insKIPVAI | 11 | 6 | IPVA1KIPVA1 | 316 | 0.57 | 245 |
| 1142 | I744_K745insKIPVAI | 9 | 0 | IPVAIKELR | 545 | 1.32 | 246 |
| 1087 | I744_K745insKIPVAI | 10 | 2 | IKIPVAIKEL | 568 | 1.80 | 247 |
| 1143 | I744_K745insKIPVAI | 9 | 1 | KIPVAIKEL | 600 | 0.34 | 248 |
| 1150 | I744_K745insKIPVAI | 9 | 8 | VKIPVAIKI | 648 | 0.57 | 249 |
| 1099 | I744_K745insKIPVAI | 11 | 0 | IPVAIKELREA | 754 | 0.67 | 250 |
| 1139 | I744_K745insKIPVAI | 9 | −2 | VAIKELREA | 786 | 2.16 | 251 |
| 1108 | I744_K745insKIPVAI | 11 | 8 | VKIPVAIKIPV | 886 | 1.18 | 252 |
| 1117 | I744_K745insKIPVAI | 12 | 11 | GEKVKIPVAIKI | 899 | 0.86 | 253 |
| 1123 | I744_K745insKIPVAI | 12 | 7 | KIPVAIKIPVAI | 1975 | 2.95 | 254 |
| 1102 | I744_K745insKIPVAI | 11 | 2 | IKIPVAIKELR | 2076 | 4.51 | 255 |
| 1135 | I744_K745insKIPVAI | 8 | 5 | PVAIKIPV | 2479 | 2.08 | 256 |
| 1107 | I744_K745insKIPVAI | 11 | 7 | KIPVAIKIPVA | 2509 | 1.71 | 257 |
| 1090 | I744_K745insKIPVAI | 10 | 5 | PVAIKIPVAI | 3025 | 3.28 | 258 |
| 1133 | I744_K745insKIPVAI | 8 | 3 | AIKIPVAI | 3372 | 3.79 | 259 |
| 1120 | I744_K745insKIPVAI | 12 | 4 | VAIKIPVAIKEL | 3482 | 3.55 | 260 |
| 4 | A763_Y764insFQEA | 10 | 1 | FQEAYVMASV | 5 | 0.05 | 261 |
| 62 | A763_Y764insFQEA | 9 | 6 | ILDEAFQEA | 5 | 0.16 | 262 |
| 24 | A763_Y764insFQEA | 11 | 6 | ILDEAFQEAYV | 6 | 0.18 | 263 |
| 59 | A763_Y764insFQEA | 9 | 3 | EAFQEAYVM | 13 | 0.05 | 264 |
| 36 | A763_Y764insFQEA | 12 | 3 | EAFQEAYVMASV | 19 | 0.15 | 265 |
| 56 | A763_Y764insFQEA | 9 | 0 | QEAYVMASV | 36 | 0.07 | 266 |
| 40 | A763_Y764insFQEA | 12 | 7 | EILDEAFQEAYV | 46 | 1.27 | 267 |
| 8 | A763_Y764insFQEA | 10 | 4 | DEAFQEAYVM | 52 | 0.04 | 268 |
| 39 | A763_Y764insFQEA | 12 | 6 | ILDEAFQEAYVM | 77 | 0.49 | 269 |
| 49 | A763_Y764insFQEA | 8 | 4 | DEAFQEAY | 77 | 0.05 | 270 |
| 20 | A763_Y764insFQEA | 11 | 2 | AFQEAYVMASV | 78 | 0.91 | 271 |
| 10 | A763_Y764insFQEA | 10 | 6 | ILDEAFQEAY | 79 | 0.06 | 272 |
| 7 | A763_Y764insFQEA | 10 | 3 | EAFQEAYVMA | 97 | 0.71 | 273 |
| 29 | A763_Y764insFQEA | 12 | −2 | AYVMASVDNPHV | 134 | 1.43 | 274 |
| 41 | A763_Y764insFQEA | 12 | 8 | KEILDEAFQEAY | 204 | 0.30 | 275 |
| 26 | A763_Y764insFQEA | 11 | 8 | KEILDEAFQEA | 235 | 0.33 | 276 |
| 11 | A763_Y764insFQEA | 10 | 7 | EILDEAFQEA | 267 | 2.61 | 277 |
| 43 | A763_Y764insFQEA | 8 | −1 | EAYVMASV | 392 | 0.12 | 278 |
| 19 | A763_Y764insFQEA | 11 | 11 | KANKEILDEAF | 473 | 1.38 | 279 |
| 53 | A763_Y764insFQEA | 8 | 8 | KEILDEAF | 544 | 1.58 | 280 |
| 47 | A763_Y764insFQEA | 8 | 2 | AFQEAYVM | 749 | 1.61 | 281 |
| 48 | A763_Y764insFQEA | 8 | 3 | EAFQEAYV | 1273 | 0.22 | 282 |
| 23 | A763_Y764insFQEA | 11 | 5 | LDEAFQEAYVM | 1608 | 0.77 | 283 |
| 65 | A763_Y764insFQEA | 9 | 9 | NKEILDEAF | 1716 | 1.38 | 284 |
| 3 | A763_Y764insFQEA | 10 | 0 | QEAYVMASVD | 2016 | 1.45 | 285 |
| 61 | A763_Y764insFQEA | 9 | 5 | LDEAFQEAY | 2035 | 1.00 | 286 |
| 60 | A763_Y764insFQEA | 9 | 4 | DEAFQEAYV | 2071 | 0.94 | 287 |
| 58 | A763_Y764insFQEA | 9 | 2 | AFQEAYVMA | 2295 | 2.35 | 288 |
| 9 | A763_Y764insFQEA | 10 | 5 | LDEAFQEAYV | 2759 | 1.11 | 289 |
| 22 | A763_Y764insFQEA | 11 | 4 | DEAFQEAYVMA | 2805 | 1.19 | 290 |
| 2493 | V769_D770insASV | 9 | 7 | YVMASVASV | 2 | 0.01 | 291 |
| 2446 | V769_D770insASV | 10 | 8 | AYVMASVASV | 7 | 0.12 | 292 |
| 2445 | V769_D770insASV | 10 | 7 | YVMASVASVD | 14 | 0.34 | 293 |
| 2441 | V769_D770insASV | 10 | 3 | SVASVDNPHV | 18 | 0.14 | 294 |
| 2460 | V769_D770insASV | 11 | 9 | EAYVMASVASV | 22 | 0.19 | 295 |
| 2468 | V769_D770insASV | 12 | 3 | SVASVDNPHVCR | 32 | 0.38 | 296 |
| 2482 | V769_D770insASV | 8 | 6 | VMASVASV | 40 | 0,98 | 297 |
| 2464 | V769_D770insASV | 12 | 10 | DEAYVMASVASV | 58 | 0.83 | 298 |
| 2438 | V769_D770insASV | 10 | 1 | ASVDNPHVCR | 103 | 0.77 | 299 |
| 2486 | V769_D770insASV | 9 | 0 | SVDNPHVCR | 129 | 0.69 | 300 |
| 2458 | V769_D770insASV | 11 | 7 | YVMASVASVDN | 142 | 1.83 | 301 |
| 2495 | V769_D770insASV | 9 | 9 | EAYVMASVA | 211 | 0.07 | 302 |
| 2439 | V769_D770insASV | 10 | 10 | DEAYVMASVA | 226 | 0.23 | 303 |
| 2489 | V769_D770insASV | 9 | 3 | SVASVDNPH | 338 | 1.70 | 304 |
| 2437 | V769_D770insASV | 10 | 0 | SVDNPHVCRL | 824 | 0.29 | 305 |
| 2485 | V769_D770insASV | 9 | −1 | VDNPHVCRL | 1410 | 0.37 | 306 |
| 2488 | V769_D770insASV | 9 | 2 | VASVDNPHV | 1878 | 1.57 | 307 |
| 2447 | V769_D770insASV | 10 | 9 | EAYVMASVAS | 1981 | 1.92 | 308 |
| 2449 | V769_D770insASV | 11 | 0 | SVDNPHVCRLL | 2087 | 0.63 | 309 |
| 2443 | V769_D770insASV | 10 | 5 | MASVASVDNP | 3002 | 4.71 | 310 |
| 2436 | V769_D770insASV | 10 | −1 | VDNPHVCRLL | 3323 | 1.07 | 311 |
| 2451 | V769_D770insASV | 11 | 10 | DEAYVMASVAS | 3435 | 1.42 | 312 |
| 289 | D770_N771insSVD | 9 | 8 | YVMASVDSV | 2 | 0.01 | 313 |
| 242 | D770_N771insSVD | 10 | 9 | AYVMASVDSV | 22 | 0.28 | 314 |
| 241 | D770_N771insSVD | 10 | 8 | YVMASVDSVD | 46 | 0.58 | 315 |
| 246 | D770_N771insSVD | 11 | 10 | EAYVMASVDSV | 64 | 0.52 | 316 |
| 235 | D770_N771insSVD | 10 | 2 | DSVDNPHVCR | 71 | 0.50 | 317 |
| 260 | D770_N771insSVD | 12 | 11 | DEAYVMASVDSV | 223 | 1.74 | 318 |
| 237 | D770_N771insSVD | 10 | 4 | SVDSVDNPHV | 669 | 1.24 | 319 |
| 285 | D770_N771insSVD | 9 | 4 | SVDSVDNPH | 3937 | 2.08 | 320 |
| 2184 | P772_H773insPR | 9 | 8 | MASVDNPPR | 20 | 0.24 | 321 |
| 2144 | P772_H773insPR | 11 | 10 | YVMASVDNPPR | 34 | 0.40 | 322 |
| 2166 | P772_H773insPR | 12 | 9 | VMASVDNPPRHV | 40 | 1.24 | 323 |
| 2141 | P772_H773insPR | 10 | 9 | VMASVDNPPR | 90 | 0.33 | 324 |
| 2135 | P772_H773insPR | 10 | 3 | NPPRHVCRLL | 148 | 0.48 | 325 |
| 2179 | P772_H773insPR | 9 | 3 | NPPRHVCRL | 202 | 0.67 | 326 |
| 2133 | P772_H773insPR | 10 | 10 | YVMASVDNPP | 305 | 4.32 | 327 |
| 2176 | P772_H773insPR | 9 | 0 | RHVCRLLGI | 309 | 0.36 | 328 |
| 2164 | P772_H773insPR | 12 | 7 | ASVDNPPRHVCR | 419 | 1.89 | 329 |
| 2157 | P772_H773insPR | 12 | 11 | AYVMASVDNPPR | 431 | 2.03 | 330 |
| 2180 | P772_H773insPR | 9 | 4 | DNPPRHVCR | 462 | 0.86 | 331 |
| 2178 | P772_H773insPR | 9 | 2 | PPRHVCRLL | 628 | 1.23 | 332 |
| 2154 | P772_H773insPR | 12 | 0 | RHVCRLLGICLT | 662 | 1.52 | 333 |
| 2162 | P772_H773insPR | 12 | 5 | VDNPPRHVCRLL | 715 | 0.89 | 334 |
| 2185 | P772_H773insPR | 9 | 9 | VMASVDNPP | 719 | 6.64 | 335 |
| 2150 | P772_H773insPR | 11 | 6 | SVDNPPRHVCR | 842 | 2.01 | 336 |
| 2182 | P772_H773insPR | 9 | 6 | SVDNPPRHV | 1095 | 0.37 | 337 |
| 2142 | P772_H773insPR | 11 | 0 | RHVCRLLGICL | 1201 | 1.39 | 338 |
| 2140 | P772_H773insPR | 10 | 8 | MASVDNPPRH | 1606 | 3.33 | 339 |
| 2139 | P772_H773insPR | 10 | 7 | ASVDNPPRHV | 3279 | 2.87 | 340 |
| 2152 | P772_H773insPR | 11 | 8 | MASVDNPPRHV | 4122 | 3.33 | 341 |
| 939 | H773_V774insH | 12 | 11 | YVMASVDNPHHV | 9 | 0.25 | 342 |
| 927 | H773_V774insH | 11 | 10 | VMASVDNPHHV | 22 | 0.76 | 343 |
| 923 | H773_V774insH | 10 | 7 | SVDNPHHVCR | 37 | 0.30 | 344 |
| 960 | H773_V774insH | 9 | 4 | NPHHVCRLL | 43 | 0.19 | 345 |
| 925 | H773_V774insH | 10 | 9 | MASVDNPHHV | 124 | 0.84 | 346 |
| 958 | H773_V774insH | 9 | 2 | HHVCRLLGI | 136 | 0.04 | 347 |
| 935 | H773_V774insH | 11 | 8 | ASVDNPHHVCR | 227 | 1.32 | 348 |
| 948 | H773_V774insH | 12 | 9 | MASVDNPHHVCR | 240 | 0.81 | 349 |
| 964 | H773_V774insH | 9 | 8 | ASVDNPHHV | 241 | 0.50 | 350 |
| 929 | H773_V774insH | 11 | 7 | HHVCRLLGICL | 407 | 0.39 | 351 |
| 952 | H773_V774insH | 8 | 4 | NPHHVCRL | 729 | 1.11 | 352 |
| 965 | H773_V774insH | 9 | 9 | MASVDNPHH | 870 | 1.23 | 353 |
| 916 | H773_V774insH | 10 | 1 | HVCRLLGICL | 964 | 2.39 | 354 |
| 946 | H773_V774insH | 12 | 7 | SVDNPHHVCRLL | 1142 | 0.94 | 355 |
| 933 | H773_V774insH | 11 | 6 | VDNPHHVCRLL | 1389 | 1.90 | 356 |
| 917 | H773_V774insH | 10 | 10 | VMASVDNPHH | 1457 | 3.40 | 357 |
| 953 | H773_V774insH | 8 | 5 | DNPHHVCR | 1762 | 2.05 | 358 |
| 921 | H773_V774insH | 10 | 5 | DNPHHVCRLL | 2011 | 2.46 | 359 |
| 931 | H773_V774insH | 11 | 4 | NPHHVCRLLGI | 2249 | 1.40 | 360 |
| 920 | H773_V774insH | 10 | 4 | NPHHVCRLLG | 2653 | 2.69 | 361 |
| 926 | H773_V774insH | 11 | 1 | HVCRLLGICLT | 2689 | 3.62 | 362 |
| 934 | H773_V774insH | 11 | 7 | SVDNPHIVCRL | 3112 | 0.90 | 363 |
| 922 | H773_V774insH | 10 | 6 | VDNPUIIVCRL | 4364 | 1.50 | 364 |

TABLE 3
HLA Class II Library of peptides and associated characteristics.

| | Peptide | Mutation | Peptide | Minimum | Minimum | SEQ NO |
|---|---|---|---|---|---|---|
| Mutation | length | position | mt | affinity | ranking | ID |
| A763_Y764insFQBA | 21 | −1 | EAYVMASVDNPHVCRLLGICL | 30 | 0.17 | 365 |
| A763_Y764insFQEA | 21 | −2 | AYVMASVDNPHVCRLLGICLT | 31 | 0.25 | 366 |
| A763_Y764insFQEA | 21 | 0 | QEAYVMASVDNPHVCRLLGIC | 32 | 0.17 | 367 |
| A763_Y764insFQEA | 21 | 1 | FQEAYVMASVDNPHVCRLLGI | 33 | 0.17 | 368 |
| A763_Y764insFQEA | 21 | 10 | ANKEILDEAFQEAYVMASVDN | 20 | 0.6 | 369 |
| A763_Y764insFQEA | 21 | 11 | KANKEILDEAFQEAYVMASVD | 19 | 0.6 | 370 |
| A763_Y764insFQEA | 21 | 12 | PKANKEILDEAFQEAYVMASV | 22 | 2.5 | 371 |
| A763_Y764insFQEA | 21 | 13 | SPKANKEILDEAFQEAYVMAS | 38 | 3 | 372 |
| A763_Y764insFQEA | 21 | 14 | TSPKANKEILDEAFQEAYVMA | 53 | 3.5 | 373 |
| A763_Y764insFQEA | 21 | 15 | ATSPKANKEILDEAFQEAYVM | 57 | 3.5 | 374 |
| A763_Y764insFQEA | 21 | 16 | EATSPKANKEILDEAFQEAYV | 57 | 4.5 | 375 |
| A763_Y764insFQEA | 21 | 17 | REATSPKANKEILDEAFQEAY | 62 | 4 | 376 |
| A763_Y764insFQEA | 21 | 18 | LREATSPKANKEILDEAFQEA | 79 | 4.5 | 377 |
| A763_Y764insFQEA | 21 | 19 | ELREATSPKANKEILDEAFQE | 206 | 7.5 | 378 |
| A763_Y764insFQEA | 21 | 2 | AFQEAYVMASVDNPHVCRLLG | 26 | 0.17 | 379 |
| A763_Y764insFQEA | 21 | 20 | KELREATSPKANKEILDEAFQ | 108 | 3.5 | 380 |
| A763_Y764insFQEA | 21 | 21 | IKELREATSPKANKEILDEAF | 68 | 2.5 | 381 |
| A763_Y764insFQEA | 21 | 3 | EAFQEAYVMASVDNPHVCRLL | 24 | 0.17 | 382 |
| A763_Y764insFQEA | 21 | 4 | DEAFQEAYVMASVDNPHVCRL | 23 | 0.2 | 383 |
| A763_Y764insFQEA | 21 | 5 | LDEAFQEAYVMASVDNPHVCR | 22 | 0.3 | 384 |
| A763_Y764insFQEA | 21 | 6 | ILDEAFQEAYVMASVDNPHVC | 18 | 0.5 | 385 |
| A763_Y764insFQEA | 21 | 7 | EILDEAFQEAYVMASVDNPHV | 17 | 0.7 | 386 |
| A763_Y764insFQEA | 21 | 8 | KEILDEAFQEAYVMASVDNPH | 18 | 0.7 | 387 |
| A763_Y764insFQEA | 21 | 9 | NKEILDEAFQEAYVMASVDNP | 18 | 0.6 | 388 |
| D770_N771insSVD | 21 | −1 | DNPHVCRLLGICLTSTVQLIT | 7 | 0.5 | 389 |
| D770_N771insSVD | 21 | 0 | VDNPHVCRLLGICLTSTVQLI | 8 | 0.6 | 390 |
| D770_N771insSVD | 21 | 1 | SVDNPHVCRLLGICLTSTVQL | 10 | 1.1 | 391 |
| D770_N771insSVD | 21 | 10 | EAYVMASVDSVDNPHVCRLLG | 27 | 1 | 392 |
| D770_N771insSVD | 21 | 11 | DEAYVMASVDSVDNPHVCRLL | 28 | 0.9 | 393 |
| D770_N771insSVD | 21 | 12 | LDEAYVMASVDSVDNPHVCRL | 26 | 0.8 | 394 |
| D770_N771insSVD | 21 | 13 | ILDEAYVMASVDSVDNPHVCR | 24 | 0.7 | 395 |
| D770_N771insSVD | 21 | 54 | EILDEAYVMASVDSVDNPHVC | 23 | 0.7 | 396 |
| D770_N771insSVD | 21 | 15 | KEILDEAYVMASVDSVDNPHV | 23 | 0.6 | 397 |
| D770_N771insSVD | 21 | 16 | NKEILDEAYVMASVDSVDNPH | 23 | 0.6 | 398 |
| D770_N771insSVD | 21 | 17 | ANKEILDEAYVMASVDSVDNP | 24 | 0.5 | 399 |
| D770_N771insSVD | 21 | 18 | KANKEILDEAYVMASVDSVDN | 29 | 0.5 | 400 |
| D770_N771insSVD | 21 | 19 | PKANKEILDEAYVMASVDSVD | 31 | 0.5 | 401 |
| D770_N771insSVD | 21 | 2 | DSVDNPHVCRLLGICLTSTVQ | 19 | 1 | 402 |
| D770_N771insSVD | 21 | 20 | SPKANKEILDEAYVMASVDSV | 40 | 0.9 | 403 |
| D770_N771insSVD | 21 | 21 | TSPKANKEILDEAYVMASVDS | 64 | 0.9 | 404 |
| D770_N771insSVD | 21 | 2 | VDSVDNPHVCRLLGICLTSTV | 20 | 1 | 405 |
| D770_N771insSVD | 21 | 4 | SVDSVDNPHVCRLLGICLTST | 69 | 1 | 406 |
| D770_N771insSVD | 21 | 5 | ASVDSVDNPHVCRLLGICLTS | 131 | 1 | 407 |
| D770_N771insSVD | 21 | 6 | MASVDSVDNPHVCRLLGICLT | 141 | 1.1 | 408 |
| D770_N771insSVD | 21 | 7 | VMASVDSVDNPHVCRLLGICL | 102 | 1.1 | 409 |
| D770_N771insSVD | 21 | 8 | YVMASVDSVDNPHVCRLLGIC | 39 | 1 | 410 |
| D770_N771insSVD | 21 | 9 | AYVMASVDSVDNPHVCRLLGI | 28 | 1.3 | 411 |
| E746_A750del | 21 | 10 | KVKIPVAIKTSPKANKEILDE | 25 | 1.7 | 412 |
| E746_A750del | 21 | 11 | EKVKIPVAIKTSPKANKEILD | 23 | 1.4 | 413 |
| E746_A750del | 21 | 12 | GEKVKIPVAlKTSPKANKEIL | 22 | 1.2 | 414 |
| E746_A750del | 21 | 13 | EGEKVKIPVAIKTSPKANKEI | 24 | 1.7 | 415 |
| E746_A750del | 21 | 14 | PEGEKVKIPVAIKTSPKANKE | 25 | 2.5 | 416 |
| E746_A750del | 21 | 15 | IPEGEKVKIPVAIKTSPKANK | 26 | 3 | 417 |
| E746_A750del | 21 | 16 | WIPEGEKVKIPVAIKTSPKAN | 40 | 4 | 418 |
| E746_A750del | 21 | 17 | LWIPEGEKVKIPVAIKTSPKA | 51 | 5 | 419 |
| E746_A750del | 21 | 18 | GLWIPEGEKVKIPVAIKTSPK | 58 | 5.5 | 420 |
| E746_A750del | 21 | 19 | KGLWIPEGEKVKIPVAIKTSP | 62 | 6 | 421 |
| E746_A750del | 21 | 2 | KTSPKANKEILDEAYVMASVD | 76 | 0.9 | 422 |
| E746_A750del | 21 | 20 | YKGLWIPEGEKVKIPVAIKTS | 68 | 7 | 423 |
| E746_A750del | 21 | 21 | VYKGLWIPEGEKVKIPVAIKT | 76 | 7.5 | 424 |
| E746_A750del | 21 | 3 | IKTSPKANKEILDEAYVMASV | 104 | 11 | 425 |
| E746_A750del | 21 | 4 | AIKTSPKANKEILDEAYVMAS | 93 | 9.5 | 426 |
| E746_A750del | 21 | 5 | VAIKTSPKANKEILDEAYVMA | 86 | 9 | 427 |
| E746_A750del | 21 | 6 | PVAIKTSPKANKEILDEAYVM | 52 | 8 | 428 |
| E746_A750del | 21 | 7 | IPVAIKTSPKANKEILDEAYV | 44 | 7 | 429 |
| E746_A750del | 21 | 8 | KIPVAIKTSPKANKELLDEAY | 36 | 6 | 430 |
| E746_A750del | 21 | 9 | VKIPVAIKTSPKANKEILDEA | 34 | 4.5 | 431 |
| E746_S752del>V | 21 | 4 | AIKVPKANKEILDEAYVMASV | 105 | 10 | 432 |
| E746_S752del>V | 21 | 13 | EGEKVKIPVAIKVPKANKEIL | 19 | 2.5 | 433 |
| E746_S752del>V | 21 | 11 | EKVKIPVAIKVPKANKEILDE | 21 | 3 | 434 |
| E746_S752del>V | 21 | 12 | GEKVKIPVAIKVPKANKEILD | 19 | 2.5 | 435 |
| E746_S752del>V | 21 | 18 | GLWIPEGEKVKIPVAIKVPKA | 22 | 3 | 436 |
| E746_S752del>V | 21 | 3 | IKVPKANKEILDEAYVMASVD | 76 | 0.9 | 437 |
| E746_S752del>V | 21 | 15 | IPEGEKVKIPVAIKVPKANKE | 20 | 3 | 438 |
| E746_S752del>V | 21 | 7 | IPVAIKVPKANKEILDEAYVM | 54 | 17 | 439 |
| E746_S752del>V | 21 | 19 | KGLWIPEGEKVKIPVAIKVPK | 27 | 3.5 | 440 |
| E746_S752del>V | 21 | 8 | KIPVAIKVPKANKEILDEAYV | 54 | 15 | 441 |
| E746_S752del>V | 21 | 10 | KVKIPVAIKVPKANKEILDEA | 24 | 4 | 442 |
| E746_S752del>V | 21 | ? | KVPKANKEILDEAYVMASVDN | 71 | 5 | 443 |
| E746_S752del>V | 21 | 17 | LWIPEGEKVKIPVAIKVPKAN | 19 | 3 | 444 |
| E746_S752del>V | 21 | 14 | PEGEKVK1PVAIKVPKANKEI | 19 | 3 | 445 |
| E746_S752del>V | 21 | 6 | PVAIKVPKANKEILDEAYVMA | 65 | 17 | 446 |
| E746_S752del>V | 21 | 5 | VAIKVPKANKEILDEAYVMAS | 83 | 15 | 447 |
| E746_S752del>V | 21 | 9 | VKIPVAIKVPKANKEILDEAY | 33 | 13 | 448 |
| E746_S752del>V | 21 | 1 | VPKANKEILDEAYVMASVDNP | 74 | 0.9 | 449 |
| E746_S752del>V | 21 | 21 | VYKGLWIPEGEKVKIPVAIKV | 30 | 7 | 450 |
| E746_S752del>V | 21 | 16 | W1PEGEKVKIPVAIKVPKANK | 20 | 3 | 451 |
| E746_S752del>V | 21 | 20 | YKGLWIPEGEKVKIPVAIKVP | 29 | 5 | 452 |
| G719A | 21 | 1 | ASGAFGTVYKGLWIPEGEKVK | 55 | 0.7 | 453 |
| G719A | 21 | 10 | TEFKKIKVLASGAFGTVYKGL | 12 | 0.03 | 454 |
| G719A | 21 | 11 | ETEFKKIKVLASGAFGTVYKG | 11 | 0.02 | 455 |
| G719A | 21 | 12 | KETEFKKIKVLASGAFGTVYK | 11 | 0.01 | 456 |
| G719A | 21 | 13 | LKETEFKKIKVLASGAFGTVY | 11 | 0.01 | 457 |
| G719A | 21 | 14 | ILKETEFKKIKVLASGAFGTV | 10 | 0.01 | 458 |
| G719A | 21 | 15 | RILKETEFKKIKVLASGAFGT | 8 | 0.01 | 459 |
| G719A | 21 | 16 | LRILKETEFKKIKVLASGAFG | 7 | 0.01 | 460 |
| G719A | 21 | 17 | LLRILKETEFKKIKVLASGAF | 6 | 0.01 | 461 |
| G719A | 21 | 18 | ALLRILKETEFKKIKVLASGA | 7 | 0.02 | 462 |
| G719A | 21 | 19 | QALLRILKETEFKKIKVLASG | 7 | 0.05 | 463 |
| G719A | 21 | 2 | LASGAFGTVYKGLWIPEGEKV | 33 | 3 | 464 |
| G719A | 21 | 20 | NQALLRILKETEFKKIKVLAS | 6 | 0.3 | 465 |
| G719A | 21 | 21 | PNQALLRILKETEFKKIKVLA | 7 | 0.8 | 466 |
| G719A | 21 | 3 | VLASGAFGTVYKGLWIPEGEK | 24 | 1.5 | 467 |
| G719A | 21 | 4 | KVLASGAFGTVYKGLWIPEGE | 21 | 0.4 | 468 |
| G719A | 21 | 5 | IKVLASGAFGTVYKGLWIPEG | 19 | 0.3 | 469 |
| G719A | 21 | 6 | KIKVLASGAFGTVYKGLWIPE | 19 | 0.25 | 470 |
| G719A | 21 | 7 | KKIKVLASGAFGTVYKGLWIP | 19 | 0.25 | 471 |
| G719A | 21 | 8 | FKKIKVLASGAFGTVYKGLWI | 19 | 0.3 | 472 |
| G719A | 21 | 9 | EFKKIKVLASGAFGTVYKGLW | 15 | 0.12 | 473 |
| G719C | 21 | 1 | CSGAFGTVYKGLWIPEGEKVK | 57 | 0.7 | 474 |
| G719C | 21 | 10 | TEFKKIKVLCSGAFGTVYKGL | 10 | 0.01 | 475 |
| G719C | 21 | 11 | ETEFKKIKVLCSGAFGTVYKG | 9 | 0.01 | 476 |
| G719C | 21 | 12 | KETEFKKIKVLCSGAFGTVYK | 8 | 0.01 | 477 |
| G719C | 21 | 13 | LKETEFKKIKVLCSGAFGTVY | 8 | 0.01 | 478 |
| G719C | 21 | 14 | ILKETEFKKIKVLCSGAFGTV | 8 | 0.01 | 479 |
| G719C | 21 | 15 | RILKETEFKKIKVLCSGAFGT | 8 | 0.01 | 480 |
| G719C | 21 | 16 | LRILKETEFKKIKVLCSGAFG | 7 | 0.01 | 481 |
| G719C | 21 | 17 | LLRILKETEFKKIKVLCSGAF | 7 | 0.01 | 482 |
| G719C | 21 | 18 | ALLRILKETEFKKIKVLCSGA | 7 | 0.01 | 483 |
| G719C | 21 | 19 | QALLRILKETEFKKIKVLCSG | 7 | 0.03 | 484 |
| G719C | 21 | 2 | LCSGAFGTVYKGLWIPEGEKV | 44 | 3 | 485 |
| G719C | 21 | 20 | NQALLRILKETEFKKIKVLCS | 7 | 0.15 | 486 |
| G719C | 21 | 21 | PNQALLRILKETEFKKIKVLC | 7 | 0.6 | 487 |
| G719C | 21 | 3 | VLCSGAFGTVYKGLWIPEGEK | 40 | 2.5 | 488 |
| G719C | 21 | 4 | KVLCSGAFGTVYKGLWIPEGE | 27 | 1.6 | 489 |
| G719C | 21 | 5 | IKVLCSGAFGTVYKGLWIPEG | 26 | 1.5 | 490 |
| G719C | 21 | 6 | KIKVLCSGAFGTVYKGLWIPE | 26 | 1.5 | 491 |
| G719C | 21 | 7 | KKIKVLCSGAFGTVYKGLWIP | 26 | 1.5 | 492 |
| G719C | 21 | 8 | FKKIKVLCSGAFGTVYKGLW1 | 27 | 1.2 | 493 |
| G719C | 21 | 9 | EFKKIKVLCSGAFGTVYKGLW | 16 | 0.04 | 494 |
| G719S | 21 | 1 | SSGAFGTVYKGLWIPEGEKVK | 57 | 0.7 | 495 |
| G719S | 21 | 10 | TEFKKIKVLSSGAFGTVYKGL | 13 | 0.02 | 496 |
| G719S | 21 | 11 | ETEFKKIKVLSSGAFGTVYKG | 11 | 0.01 | 497 |
| G719S | 21 | 12 | KETEFKKIKVLSSGAFGTVYK | 10 | 0.01 | 498 |
| G719S | 21 | 13 | LKETEFKKIKVLSSGAFGTVY | 10 | 0.01 | 499 |
| G719S | 21 | 14 | ILKETEFKKIKVLSSGAFGTV | 10 | 0.01 | 500 |
| G719S | 21 | 15 | RILKETEFKKIKVLSSGAFGT | 9 | 0.01 | 501 |
| G719S | 21 | 16 | LRILKETEFKKIKVLSSGAFG | 8 | 0.01 | 502 |
| G719S | 21 | 17 | LLRILKETEFKKIKVLSSGAF | 7 | 0.01 | 503 |
| G719S | 21 | 18 | ALLRILKETEFKKIKVLSSGA | 7 | 0.02 | 504 |
| G719S | 21 | 19 | QALLRILKETEFKKIKVLSSG | 7 | 0.04 | 505 |
| G719S | 21 | 2 | LSSGAFGTVYKGLWIPEGEKV | 39 | 3 | 506 |
| G719S | 21 | 20 | NQALLRILKETEFKKIKVLSS | 7 | 0.25 | 507 |
| G719S | 21 | 21 | PNQALLRILKETEFKKIKVLS | 7 | 1 | 508 |
| G719S | 21 | 3 | VLSSGAFGTVYKGLWIPEGEK | 29 | 1.7 | 509 |
| G719S | 21 | 4 | KVLSSGAFGTVYKGLWIPEGE | 17 | 0.7 | 510 |
| G719S | 21 | 5 | IKVLSSGAFGTVYKGLWIPEG | 15 | 0.5 | 511 |
| G719S | 21 | 6 | KIKVLSSGAFGTVYKGLWIPE | 15 | 0.5 | 512 |
| G719S | 21 | 77 | KKIKVLSSGAFGTVYKGLWIP | 15 | 0.5 | 513 |
| G719S | 21 | 8 | FKKIKVLSSGAFGTVYKGLWI | 15 | 0.5 | 514 |
| G719S | 21 | 9 | EFKKIKVLSSGAFGTVYKGLW | 14 | 0.09 | 515 |
| H773_v774insH | 21 | 1 | HVCRLLGICLTSTVQLITQLM | 7 | 0.4 | 516 |
| H773_v774insH | 21 | 10 | VMASVDNPHHVCRLLGICLTS | 69 | 3.5 | 517 |
| H773_v774insH | 21 | 11 | YVMASVDNPHHVCRLLGICLT | 55 | 0.7 | 518 |
| H773_v774insH | 21 | 12 | AYVMASVDNPHHVCRLLGICL | 39 | 0.3 | 519 |
| H773_v774insH | 21 | 13 | EAYVMASVDNPHHVCRLLGIC | 36 | 0.25 | 520 |
| H773_v774insH | 21 | 14 | DEAYVMASVDNPHHVCRLLGI | 35 | 0.2 | 521 |
| H773_v774insH | 21 | 15 | LDEAYVMASVDNPHHVCRLLG | 35 | 0.25 | 522 |
| H773_v774insH | 21 | 16 | ILDEAYVMASVDNPHHVCRLL | 36 | 0.25 | 523 |
| H773_v774insH | 21 | 17 | EILDEAYVMASVDNPHHVCRL | 37 | 0.25 | 524 |
| H773_v774insH | 21 | 18 | KEILDEAYVMASVDNPHHVCR | 37 | 0.25 | 525 |
| H773_v774insH | 21 | 19 | NKEILDEAYVMASVDNPHHVC | 38 | 0.4 | 526 |
| H773_v774insH | 21 | 2 | HHVCRLLGICLTSTVQLITQL | 7 | 0.4 | 527 |
| H773_v774insH | 21 | 20 | ANKEILDEAYVMASVDNPHHV | 39 | 0.5 | 528 |
| H773_v774insH | 21 | 21 | KANKEILDEAYVMASVDNPHH | 44 | 1 | 529 |
| H773_v774insH | 21 | 3 | PHHVCRLLGICLTSTVQLITQ | 7 | 0.4 | 530 |
| H773_v774insH | 21 | 4 | NPHHVCRLLGICLTSTVQLIT | 7 | 0.4 | 531 |
| H773_v774insH | 21 | 5 | DNPHHVCRLLGICLTSTVQLI | 8 | 0.5 | 532 |
| H773_v774insH | 21 | 6 | VDNPHHVCRLLGICLTSTVQL | 11 | 1.4 | 533 |
| H773_v774insH | 21 | 7 | SVDNPHHVCRLLGICLTSTVQ | 22 | 3 | 534 |
| H773_v774insH | 21 | 8 | ASVDNPHHVCRLLGICL1STV | 21 | 3 | 535 |
| H773_v774insH | 21 | 9 | MASVDNPHHVCRLLGICLTST | 63 | 3.5 | 536 |
| I744_K745insKIPVAI | 21 | −1 | PVAIKELREATSPKANKEILD | 31 | 1.6 | 537 |
| I744_K745insKIPVAI | 21 | −2 | VAIKELREATSPKANKEILDE | 34 | 1.7 | 538 |
| I744_K745insKIPVAI | 21 | −3 | AIKELREATSPKANKEILDEA | 46 | 1.9 | 539 |
| I744_K745insKIPVAI | 21 | −4 | 1KELREATSPKANKEILDEAY | 68 | 2.5 | 540 |
| I744_K745insKIPVAI | 21 | 0 | IPVAIKELREATSPKANKEIL | 29 | 1.6 | 541 |
| I744_K745insKIPVAI | 21 | 1 | KIPVAIKELREATSPKANKEI | 29 | 1.7 | 542 |
| I744_K745insKIPVAI | 21 | 10 | EKVKIPVAIKIPVAIKELREA | 17 | 0.8 | 543 |
| I744_K745insKIPVAI | 21 | 11 | GEKVKIPVAIK1PVAIKELRE | 16 | 0.8 | 544 |
| I744_K745insKIPVAI | 21 | 12 | EGEKVKIPVAIKIPVAIKELR | 15 | 0.8 | 545 |
| I744_K745insKIPVAI | 21 | 13 | PEGEKVKIPVAIKIPVAIKEL | 15 | 1 | 546 |
| I744_K745insKIPVAI | 21 | 14 | IPEGEKVKIPVAIKIPVAIKE | 16 | 1.7 | 547 |
| I744_K745insKIPVAI | 21 | 15 | WIPEGEKVKIPVAIKIPVAIK | 16 | 1.9 | 548 |
| I744_K745insKIPVAI | 21 | 16 | LWIPEGEKVKIPVAIKIPVAI | 15 | 1.9 | 549 |
| I744_K745insKIPVAI | 21 | 17 | GLWIPEGEKVKIPVAIKIPVA | 21 | 2.5 | 550 |
| I744_K745insKIPVAI | 21 | 18 | KGLWIPEGEKVKIPVAIKIPV | 24 | 3 | 551 |
| I744_K745insKIPVAI | 21 | 19 | YKGLWIPEGEKVKIPVAIKIP | 30 | 4.5 | 552 |
| I744_K745insKIPVAI | 21 | 2 | IKJPVAIKELREATSPKANKE | 30 | 1.9 | 553 |
| I744_K745insKIPVAI | 21 | 20 | VYKGLWIPEGEKVKIPVAIKI | 31 | 7 | 554 |
| I744_K745insKIPVAI | 21 | 3 | AIKIPVAIKELREATSPKANK | 27 | 2.5 | 555 |
| I744_K745insKIPVAI | 21 | 4 | VAIKIPVAIKELREATSPKAN | 30 | 3.5 | 556 |
| I744_K745insKIPVAI | 21 | 5 | PVAIKIPVAIKELREATSPKA | 30 | 3.5 | 557 |
| I744_K745insKIPVAI | 21 | 6 | IPVAIKIPVAIKELREATSPK | 34 | 3.5 | 558 |
| I744_K745insKIPVAI | 21 | 7 | KIPVAIKIPVAIKELREATSP | 26 | 2.5 | 559 |
| I744_K745insKIPVAI | 21 | 8 | VKIPVAIKIPVAIKELREATS | 21 | 1.7 | 560 |
| I744_K745insKIPVAI | 21 | 9 | KVKIPVAIKIPVAIKELREAT | 18 | 3 | 561 |
| L747_A750del>P | 21 | 5 | AIKEPTSPKANKEILDEAYVM | 533 | 24 | 562 |
| L747_A750del>P | 21 | 14 | EGEKVKIPVAIKEPTSPKANK | 70 | 7 | 563 |
| L747_A750del>P | 21 | 12 | EKVKIPVAIKEPTSPKANKEI | 80 | 8 | 564 |
| L747_A750del>P | 21 | 2 | EPTSPKANKEILDEAYVMASV | 104 | 11 | 565 |
| L747_A750del>P | 21 | 13 | GEKVKIPVAIKEPTSPKANKE | 71 | 7 | 566 |
| L747_A750del>P | 21 | 19 | GLWIPEGEKVKIPVAiKEPTS | 70 | 7 | 567 |
| L747_A750del>P | 21 | 4 | IKEPTSPKANKEILDEAYVMA | 478 | 18 | 568 |
| L747_A750del>P | 21 | 16 | IPEGEKVKIPVAlKEPTSPKA | 70 | 7 | 569 |
| L747_A750del>P | 21 | 8 | IPVAIKEPTSPKANKEILDEA | 216 | 31 | 570 |
| L747_A750del>P | 21 | 3 | KEPTSPKANKEILDEAYVMAS | 343 | 16 | 571 |
| L747_A750del>P | 21 | 20 | KGLWIPEGEKVKIPVAIKEPT | 74 | 7.5 | 572 |
| L747_A750del>P | 21 | 9 | KIPVAIKEPTSPKANKEILDE | 190 | 29 | 573 |
| L747_A750del>P | 21 | 11 | KVKIPVAIKEPTSPKANKEIL | 112 | 12 | 574 |
| L747_A750del>P | 21 | 18 | LWIPEGEKVKIPVAIKEPTSP | 69 | 7 | 575 |
| L747_A750del>P | 21 | 15 | PEGEKVK1PVAIKEPTSPKAN | 69 | 7 | 576 |
| L747_A750del>P | 21 | 1 | PTSPKANKEILDEAYVMASVD | 75 | 0.9 | 577 |
| L747_A750del>P | 21 | 7 | PVAIKEPTSPKANKEILDEAY | 334 | 33 | 578 |
| L747_A750del>P | 21 | 6 | VAIKEPTSPKANKEILDEAYV | 672 | 35 | 579 |
| L747_A750del>P | 21 | 10 | VKIPVAIKEPTSPKANKEILD | 173 | 28 | 580 |
| L747_A750del>P | 21 | 17 | WIPEGEKVKIPVAIKEPTSPK | 68 | 7 | 581 |
| L747_A750del>P | 21 | 21 | YKGLWIPEGEKVKIPVAIKEP | 89 | 9 | 582 |
| L747_A750del>P | 21 | 5 | AIKESKANKEILDEAYVMASV | 100 | 10 | 583 |
| L747_A750del>P | 21 | 14 | EGEKVKIPVAIKESKANKEIL | 44 | 4 | 584 |
| L747_P753del>S | 21 | 12 | EKVKIPVAIKESKANKEILDE | 50 | 5 | 585 |
| L747_P753del>S | 21 | 2 | ESKANKEILDEAYVMASVDNP | 73 | 0.9 | 586 |
| L747_P753del>S | 21 | 13 | GEKVKIPVAIKESKANKEILD | 44 | 4 | 587 |
| L747_P753del>S | 21 | 19 | GLWIPEGEKVKIPVAIKESKA | 56 | 5.5 | 588 |
| L747_P753del>S | 21 | 4 | IKESKANKEILDEAYVMASVD | 74 | 0.9 | 589 |
| L747_P753del>S | 21 | 16 | IPEGEKVKIPVAIKESKANKE | 47 | 4.5 | 590 |
| L747_P753del>S | 21 | 8 | 1PVAIKESKANKEILDEAYVM | 84 | 22 | 591 |
| L747_P753del>S | 21 | 3 | KESKANKEILDEAYVMASVDN | 69 | 1 | 592 |
| L747_P753del>S | 21 | 20 | KGLWIPEGEKVKIPVAIKESK | 62 | 6 | 593 |
| L747_P753del>S | 21 | 9 | KIPVAIKESKANKEILDEAYV | 113 | 27 | 594 |
| L747_P753del>S | 21 | 11 | KVKIPVAIKESKANKEILDEA | 66 | 6.5 | 595 |
| L747_P753del>S | 21 | 18 | LWIPEGEKVKIPVAIKESKAN | 49 | 4.5 | 596 |
| L747_P753del>S | 21 | 15 | PEGEKVKIPVAIKESKANKEI | 46 | 4.5 | 597 |
| L747_P753del>S | 21 | 7 | PVAIKESKANKEILDEAYVMA | 87 | 18 | 598 |
| L747_P753del>S | 21 | 1 | SKANKEILDEAYVMASVDNPH | 60 | 1.1 | 599 |
| L747_P753del>S | 21 | 6 | VAIKESKANKEILDEAYVMAS | 110 | 15 | 600 |
| L747_P753del>S | 21 | 10 | VKIPVAIKESKANKEILDEAY | 104 | 23 | 601 |
| L747_P753del>S | 21 | 17 | WiPEGEKVKIPVAIKESKANK | 47 | 4.5 | 602 |
| L747_P753del>S | 21 | 21 | YKGLWIPEGEKVKIPVAIKES | 77 | 8 | 603 |
| L747_S752del | 21 | 10 | VKIPVAIKEPKANKEILDEAY | 176 | 26 | 604 |
| L747_S752del | 21 | 11 | KVKIPVAIKEPKANKEILDEA | 96 | 10 | 605 |
| L747_S752del | 21 | 12 | EKVKIPVAIKEPKANKEILDE | 70 | 7 | 606 |
| L747_S752del | 21 | 13 | GEKVKIPVAIKEPKANKEILD | 62 | 6 | 607 |
| L747_S752del | 21 | 14 | EGEKVKIPVAIKEPKANKEIL | 62 | 6 | 608 |
| L747_S752del | 21 | 15 | PEGEKVKIPVAIKEPKANKEI | 62 | 6 | 609 |
| L747_S752del | 21 | 16 | IPEGEKVKIPVAIKEPKANKE | 61 | 6 | 610 |
| L747_S752del | 21 | 17 | WIPEGEKVKIPVAIKEPKANK | 60 | 6 | 611 |
| L747_S752del | 21 | 18 | LWIPEGEKVKIPVAIKEPKAN | 61 | 6 | 612 |
| L747_S752del | 21 | 19 | GLWIPEGEKVKIPVAIKEPKA | 65 | 6.5 | 613 |
| L747_S752del | 21 | 2 | EPKANKEILDEAYVMASVDNP | 74 | 0.9 | 614 |
| L747_S752del | 21 | 20 | KGLWIPEGEKVKIPVAIKEPK | 72 | 7.5 | 615 |
| L747_S752del | 21 | 21 | YKGLWIPEGEKVKIPVAIKEP | 89 | 9 | 616 |
| L747_S752del | 21 | 3 | KEPKANKEILDEAYVMASVDN | 71 | 1 | 617 |
| L747_S752del | 21 | 4 | IKEPKANKEILDEAYVMASVD | 76 | 0.9 | 618 |
| L747_S752del | 21 | 5 | AIKEPKANKEILDEAYVMASV | 104 | 10 | 619 |
| L747_S752del | 21 | 6 | VAIKEPKANKEILDEAYVMAS | 264 | 15 | 620 |
| L747_S752del | 21 | 7 | PVAIKEPKANKEILDEAYVMA | 184 | 18 | 621 |
| L747_S752del | 21 | 8 | IPVAIKEPKANKElLDEAYVM | 156 | 24 | 622 |
| L747_S752del | 21 | 9 | KIPVAIKEPKANKEILDEAYV | 208 | 31 | 623 |
| L747_T751del | 21 | 10 | VKIPVAIKESPKANKEILDEA | 97 | 13 | 624 |
| L747_T751del | 21 | 11 | KVKIPVAIKESPKANKEILDE | 45 | 4 | 625 |
| L747_T751del | 21 | 12 | EKVKIPVAIKESPKANKEILD | 39 | 3.5 | 626 |
| L747_T751del | 21 | 13 | GEKVKIPVAIKESPKANKEIL | 38 | 3 | 627 |
| L747_T751del | 21 | 14 | EGEKVKIPVAIKESPKANKEI | 43 | 4 | 628 |
| L747_T751del | 21 | 15 | PEGEKVKIPVAIKESPKANKE | 49 | 4.5 | 629 |
| L747_T751del | 21 | 16 | IPEGEKVKIPVAIKESPKANK | 50 | 5 | 630 |
| L747_T751del | 21 | 17 | WIPEGEKVKIPVAIKESPKAN | 55 | 5.5 | 631 |
| L747_T751del | 21 | 18 | LWIPEGEKVKIPVAIKESPKA | 59 | 6 | 632 |
| L747_T751del | 21 | 19 | GLWIPEGEKVKIPVAIKESPK | 65 | 6.5 | 633 |
| L747_T751del | 21 | 2 | ESPKANKEILDEAYVMASVDN | 71 | 1 | 634 |
| L747_T751del | 21 | 20 | KGLWIPEGEKVKIPVAIKESP | 71 | 7 | 635 |
| L747_T751del | 21 | 21 | YKGLWIPEGEKVKIPVA1KES | 77 | 8 | 636 |
| L747_T751del | 21 | 3 | KESPKANKEILDEAYVMASVD | 76 | 0.9 | 637 |
| L747_T751del | 21 | 4 | IKESPKANKEILDEAYVMASV | 105 | 10 | 638 |
| L747_T751del | 21 | 5 | AIKESPKANKEILDEAYVMAS | 248 | 15 | 639 |
| L747_T751del | 21 | 6 | VAIKESPKANKEILDEAYVMA | 160 | 19 | 640 |
| L747_T751del | 21 | 7 | PVAIKESPKANKEILDEAYVM | 119 | 24 | 641 |
| L747_T751del | 21 | 8 | IFVAIKESPKANKEIIDEAYV | 116 | 24 | 642 |
| L747_T751del | 21 | 9 | KIPVAIKESPKANKEILDEAY | 106 | 20 | 643 |
| L747_T751del>P | 21 | 5 | AIKEPSPKANKEILDEAYVMA | 323 | 19 | 644 |
| L747_T751del>P | 21 | 14 | EGEKVKIPVAIKEPSPKANKE | 66 | 7 | 645 |
| L747_T751del>P | 21 | 12 | EKVKIPVAIKEPSPKANKEIL | 77 | 8 | 646 |
| L747_T751del>P | 21 | 2 | EPSPKANKEILDEAYVMASVD | 76 | 0.9 | 647 |
| L747_T751del>P | 21 | 13 | GEKVKIPVAIKEPSPKANKEI | 66 | 6.5 | 648 |
| L747_T751del>P | 21 | 19 | GLWIPEGEKVKIPVAIKEPSP | 74 | 7.5 | 649 |
| L747_T751del>P | 21 | 4 | IKEPSPKANKEILDEAYVMAS | 337 | 16 | 650 |
| L747_T751del>P | 21 | 16 | IPEGEKVKIPVAIKEPSPKAN | 67 | 6.5 | 651 |
| L747_T751del>P | 21 | 8 | IPVAIKEPSPKANKEILDEAY | 116 | 27 | 652 |
| L747_T751del>P | 21 | 3 | KEPSPKANKEILDEAYVMASV | 105 | 10 | 653 |
| L747_T751del>P | 21 | 20 | KGLWIPEGEKVK1PVAIKEPS | 75 | 7.5 | 654 |
| L747_T751del>P | 21 | 9 | KIPVAIKEPSPKANKEILDEA | 106 | 20 | 655 |
| L747_T751del>P | 21 | 11 | KVKIPVAIKEPSPKANKEILD | 90 | 11 | 656 |
| L747_T751del>P | 21 | 18 | LWIPEGEKVKIPVAIKEPSPK | 68 | 7 | 657 |
| L747_T751del>P | 21 | 15 | PEGEKVKIPVAIKEPSPKANK | 67 | 7 | 658 |
| L747_T751del>P | 21 | 1 | PSPKANKEILDEAYVMASVDN | 71 | 1 | 659 |
| L747_T751del>P | 21 | 7 | PVAIKEPSPKANKEILDEAYV | 151 | 29 | 660 |
| L747_T751del>P | 21 | 6 | VAIKEPSPKANKEILDEAYVM | 215 | 24 | 661 |
| L747_T751del>P | 21 | 10 | VKIPVAIKEPSPKANKEILDE | 97 | 19 | 662 |
| L747_T751del>P | 21 | 17 | WIPEGEKVKIPVAIKEPSPKA | 67 | 7 | 663 |
| L747_T751del>P | 21 | 21 | YKGLWIPEGEKVKIPVAIKEP | 89 | 9 | 664 |
| L858R | 21 | 1 | RAKLLGAEEKEYHAEGGKVPI | 167 | 16 | 665 |
| L858R | 21 | 10 | QHVKITDFGRAKLLGAEEKEY | 76 | 5.5 | 666 |
| L858R | 21 | 11 | PQHVKITDFGRAKLLGAEEKE | 47 | 5.5 | 667 |
| L858R | 21 | 12 | TPQHVKITDFGRAKLLGAEEK | 48 | 5.5 | 668 |
| L858R | 21 | 13 | KTPQHVKITDFGRAKLLGAEE | 52 | 5.5 | 669 |
| L858R | 21 | 14 | VKTPQHVKITDFGRAKLLGAE | 33 | 5 | 670 |
| L858R | 21 | 15 | LVKTPQHVKITDFGRAKLLGA | 25 | 3 | 671 |
| L858R | 21 | 16 | VLVKTPQHVKITDFGRAKLLO | 15 | 0.7 | 672 |
| L858R | 21 | 17 | NVLVKTPQHVKITDFGRAKLL | 10 | 0.5 | 673 |
| L858R | 21 | 18 | RNVLVKTPQHVKITDFGRAKL | 8 | 0.6 | 674 |
| L858R | 21 | 19 | ARNVLVKTPQHVKITDFGRAK | 8 | 0.8 | 675 |
| L858R | 21 | 2 | GRAKLLGAEEKEYHAEGGKVP | 261 | 16 | 676 |
| L858R | 21 | 20 | AARNVLVKTPQHVKITDFGRA | 7 | 0.9 | 677 |
| L858R | 21 | 21 | LAARNVLVKTPQHVKITDFGR | 8 | 0.8 | 678 |
| L858R | 21 | 2 | FGRAKLLGAEEKEYHAEGGKV | 208 | 16 | 679 |
| L858R | 21 | 4 | DFGRAKLLGAEEKEYHAEGGK | 171 | 16 | 680 |
| L858R | 21 | 5 | TDFGRAKLLGAEEKEYHAEGG | 112 | 15 | 681 |
| L858R | 21 | 6 | ITDFGRAKLLGAEEKEYHAEG | 92 | 13 | 682 |
| L858R | 21 | 7 | KITDFGRAKLLGAEEKEYHAE | 82 | 12 | 683 |
| L858R | 21 | 8 | VKITDFGRAKLLGAEEKEYHA | 78 | 8 | 684 |
| L858R | 21 | 9 | HVKITDFGRAKLLGAEEKEYH | 76 | 5.5 | 685 |
| L861Q | 21 | 1 | QLGAEEKEYHAEGGKVPIKWM | 26 | 4 | 686 |
| L861Q | 21 | 10 | KITDFGLAKQLGAEEKEYHAE | 96 | 7.5 | 687 |
| L861Q | 21 | 11 | VKITDFGLAKQLGAEEKEYHA | 71 | 7 | 688 |
| L861Q | 21 | 12 | HVKITDFGLAKQLGAEEKEYH | 57 | 6 | 689 |
| L861Q | 21 | 13 | QHVKITDFGLAKQLGAEEKEY | 52 | 6 | 690 |
| L861Q | 21 | 14 | PQHVKITDFGLAKQLGAEEKE | 52 | 6 | 691 |
| L861Q | 21 | 15 | TPQHVKITDFGLAKQLGAEEK | 53 | 6.5 | 692 |
| L861Q | 21 | 16 | KTPQHVKITDFGLAKQLGAEE | 65 | 7.5 | 693 |
| L861Q | 21 | 17 | VKTPQHVKITDFGLAKQLGAE | 52 | 8.5 | 694 |
| L861Q | 21 | 18 | LVKTPQHVKITDFGLAKQLGA | 37 | 11 | 695 |
| L861Q | 21 | 19 | VLVKTPQHVKITDFGLAKQLG | 18 | 1.6 | 696 |
| L861Q | 21 | 2 | KQLGAEEKEYHAEGGKVPIKW | 26 | 4 | 697 |
| L861Q | 21 | 20 | NVLVKTPQHVKITDFGLAKQL | 12 | 0.9 | 698 |
| L861Q | 21 | 21 | RNVLVKTPQHVKTTDFGLAKQ | 9 | 0.9 | 699 |
| L861Q | 21 | 3 | AKQLGAEEKEYHAEGGKVPIK | 36 | 6.5 | 700 |
| L861Q | 21 | 4 | LAKQLGAEEKEYHAEGGKVPI | 179 | 25 | 701 |
| L861Q | 21 | 5 | GLAKQLGAEEKEYHAEGGKVP | 334 | 35 | 702 |
| L861Q | 21 | 6 | FGLAKQLGAEEKEYHAEGGKV | 247 | 15 | 703 |
| L861Q | 21 | 7 | DFGLAKQLGAEEKEYHAEGGK | 173 | 8 | 704 |
| L861Q | 21 | 8 | TDFGLAKQLGAEEKEYHAEGG | 141 | 7.5 | 705 |
| L861Q | 21 | 9 | ITDFGLAKQLGAEEKEYHAEG | 126 | 7.5 | 706 |
| P772_H773insPR | 21 | 0 | RHVCRLLGICLTSTVQLITQL | 7 | 0.3 | 707 |
| P772_H773insPR | 21 | 1 | PRHVCRLLGICLTSTVQLITQ | 7 | 0.4 | 708 |
| P772_H773insPR | 21 | 10 | YVMASVDNPPRHVCRLLGICL | 28 | 1.5 | 709 |
| P772_H773insPR | 21 | 11 | AYVMASVDNPPRHVCRLLGIC | 7 | 0.9 | 710 |
| P772_H773insPR | 21 | 12 | EAYVMASVDNPPRHVCRLLGI | 26 | 0.7 | 711 |
| P772_H773insPR | 21 | 13 | DEAYVMASVDNPPRHVCRLLG | 26 | 0.7 | 712 |
| P772_H773insPR | 21 | 14 | LDEAYVMASVDNPPRHVCRLL | 27 | 0.7 | 713 |
| P772_H773insPR | 21 | 15 | ILDEAYVMASVDNPPRHVCRL | 26 | 0.7 | 714 |
| P772_H773insPR | 21 | 16 | EILDEAYVMASVDNPPRHVCR | 27 | 0.7 | 715 |
| P772_H773insPR | 21 | 17 | KEILDEAYVMASVDNPPRHVC | 28 | 0.8 | 716 |
| P772_H773insPR | 21 | 18 | NKEILDEAYVMASVDNPPRHV | 31 | 0.9 | 717 |
| P772_H773insPR | 21 | 19 | ANKEILDEAYVMASVDNPPRH | 44 | 12 | 718 |
| P772_H773insPR | 21 | 2 | PPRHVCRLLGICLTSTVQLIT | 7 | 0.4 | 719 |
| P772_H773insPR | 21 | 20 | KANKEILDEAYVMASVDNPPR | 49 | 1.1 | 720 |
| P772_H773insPR | 21 | 21 | PKANKEILDEAYVMASVDNPP | 55 | 1 | 721 |
| P772_H773insPR | 21 | 3 | NPPRHVCRLLGICLTSTVQLI | 8 | 0.5 | 722 |
| P772_H773insPR | 21 | 4 | DNPPRHVCRLLGICLTSTVQL | 11 | 1.2 | 723 |
| P772_H773insPR | 21 | 5 | VDNPPRHVCRLLGICLTSTVQ | 22 | 3 | 724 |
| P772_H773insPR | 21 | 6 | SVDNPPRHVCRLLGICLTSTV | 22 | 4.5 | 725 |
| P772_H773insPR | 21 | 7 | ASVDNPPRHVCRLLGICLTST | 56 | 4 | 726 |
| P772_H773insPR | 21 | 8 | MASVDNPPRHVCRLLGICLTS | 40 | 4.5 | 727 |
| P772_H773insPR | 21 | 9 | VMASVDNPPRHVCRLLGICLT | 32 | 5 | 728 |
| S768I | 21 | 1 | IVDNPHVCRLLGICLTSTVQL | 10 | 1.3 | 729 |
| S768I | 21 | 10 | ILDEAYVMAIVDNPHVCRLLG | 15 | 0.04 | 730 |
| S768I | 21 | 11 | EILDEAYVMAIVDNPHVCRLL | 15 | 0.04 | 731 |
| S768I | 21 | 12 | KEILDEAYVMAIVDNPHVCRL | 17 | 0.05 | 732 |
| S768I | 21 | 13 | NKEILDEAYVMAIVDNPHVCR | 20 | 0.06 | 733 |
| S768I | 21 | 14 | ANKEILDEAYVMAIVDNPHVC | 26 | 0.12 | 734 |
| S768I | 21 | 15 | KANKEILDEAYVMAIVDNPHV | 33 | 0.3 | 735 |
| S768I | 21 | 16 | PKANKEILDEAYVMAIVDNPH | 47 | 1 | 736 |
| S768I | 21 | 17 | SPKANKEILDEAYVMA1VDNP | 71 | 0.8 | 737 |
| S768I | 21 | 18 | TSPKANKEILDEAYVMAIVDN | 123 | 0.6 | 738 |
| S768I | 21 | 19 | ATSPKANKEILDEAYVMAIVD | 137 | 0.9 | 739 |
| S768I | 21 | 2 | AIVDNPHVCRLLGICLTSTVQ | 12 | 0.4 | 740 |
| S768I | 21 | 20 | EATSPKANKEILDEAYVMAIV | 209 | 10 | 741 |
| S768I | 21 | 21 | REATSPKANKEILDEAYVMAI | 266 | 13 | 742 |
| S768I | 21 | 3 | MAIVDNPHVCRLLGICLTSTV | 9 | 0.5 | 743 |
| S768I | 21 | 4 | VMAIVDNPHVCRLLGICLTST | 17 | 0.25 | 744 |
| S768I | 21 | 5 | YVMAIVDNPHVCRLLGICLTS | 15 | 0.06 | 745 |
| S768I | 21 | 6 | AYVMAIVDNPHVCRLLGICLT | 14 | 0.04 | 746 |
| S768I | 21 | 7 | EAYVMAIVDNPHVCRLLGICL | 14 | 0.04 | 747 |
| S768I | 21 | 8 | DEAYVMAIVDNPHVCRLLGIC | 15 | 0.04 | 748 |
| S768I | 21 | 9 | LDEAYVMAIVDNPHVCRLLGI | 15 | 0.04 | 749 |
| T790M | 21 | 1 | MQLMPFGCLLDYVREHKDNIG | 83 | 5.5 | 750 |
| T790M | 21 | 10 | CLTSTVQLIMQLMPFGCLLDY | 10 | 1.8 | 751 |
| T790M | 21 | 51 | ICLTSTVQLIMQLMPFGCLLD | 11 | 1.9 | 752 |
| T790M | 21 | 12 | GICLTSTVQLIMQLMPFGCLL | 11 | 1.7 | 753 |
| T790M | 21 | 13 | LGICLTSTVQLIMQLMPFGCL | 9 | 1 | 754 |
| T790M | 21 | 14 | LLGICLTSTVQLIMQLMPFGC | 7 | 0.7 | 755 |
| T790M | 21 | 15 | RLLGICLTSTVQLIMQLMPFG | 7 | 1.2 | 756 |
| T790M | 21 | 16 | CRLLGICLTSTVQLIMQLMPF | 7 | 0.6 | 757 |
| T790M | 21 | 17 | VCRLLGICLTSTVQLIMQLMP | 7 | 0.3 | 758 |
| T790M | 21 | 18 | HVCRLLGICLTSTVQLIMQLM | 7 | 0.4 | 759 |
| T790M | 21 | 19 | PHVCRLLGICLTSTVQLIMQL | 7 | 0.3 | 760 |
| T790M | 21 | 2 | IMQLMPFGCLLDYVREHKDNI | 29 | 5.5 | 761 |
| T790M | 21 | 20 | NPHVCRLLGICLTSTVQLIMQ | 7 | 0.4 | 762 |
| T790M | 21 | 21 | DNPHVCRLLGICLTSTVQLIM | 7 | 0.5 | 763 |
| T790M | 21 | 3 | LIMQLMPFGCLLDYVREHKDN | 16 | 3 | 764 |
| T790M | 21 | 4 | QLIMQLMPFGCLLDYVREHKD | 9 | 2.5 | 765 |
| T790M | 21 | 5 | VQLIMQLMPFGCLLDYVREHK | 9 | 1.7 | 766 |
| T790M | 21 | 6 | TVQLIMQLMPFGCLLDYVREH | 9 | 2.5 | 767 |
| T790M | 21 | 7 | STVQLIMQLMPFGCLLDYVRE | 10 | 2.5 | 768 |
| T790M | 21 | 8 | TSTVQLIMQLMPFGCLLDYVR | 10 | 2.5 | 769 |
| T790M | 21 | 9 | LTSTVQLIMQLMPFGCLLDYV | 10 | 2 | 770 |
| V769_D770insASV | 21 | −1 | VDNPHVCRLLGICLTSTVQLI | 8 | 0.6 | 771 |
| V769_D770insASV | 21 | 0 | SVDNPHVCRLLGICLTSTVQL | 10 | 1.1 | 777 |
| V769_D770insASV | 21 | 1 | ASVDNPHVCRLLGICLTSTVQ | 17 | 1.1 | 773 |
| V769_D770insASV | 21 | 10 | DEAYVMASVASVDNPHVCRLL | 9 | 0.09 | 774 |
| V769_D770insASV | 21 | 11 | LDEAYVMASVASVDNPHVCRL | 9 | 0.09 | 775 |
| V769_D770insASV | 21 | 12 | ILDEAYVMASVASVDNPHVCR | 9 | 0.09 | 776 |
| V769_D770insASV | 21 | 13 | EILDEAYVMASVASVDNPHVC | 9 | 0.09 | 777 |
| V769_D770insASV | 21 | 14 | KEILDEAYVMASVASVDNPHV | 9 | 0.09 | 778 |
| V769_D770insASV | 21 | 15 | NKEILDEAYVMASVASVDNPH | 10 | 0.08 | 779 |
| V769_D770insASV | 21 | 16 | ANKEILDEAYVMASVASVDNR | 10 | 0.07 | 780 |
| V769_D770insASV | 21 | 17 | KANKEILDEAYVMASVASVDN | 10 | 0.08 | 781 |
| V769_D770insASV | 21 | 18 | PKANKEILDEAYVMASVASVD | 10 | 0.08 | 782 |
| V769_D770insASV | 21 | 19 | SPKANKEILDEAYVMASVASV | 10 | 0.2 | 783 |
| V769_D770insASV | 21 | 2 | VASVDNPHVCRLLGICLTSTV | 14 | 1.1 | 784 |
| V769_D770insASV | 21 | 20 | TSPKANKEILDEAYVMASVAS | 38 | 0.7 | 785 |
| V769_D770insASV | 21 | 21 | ATSPKANKEILDEAYVMASVA | 76 | 14 | 786 |
| V769_D770insASV | 21 | 3 | SVASVDNPHVCRLLGICLTST | 55 | 1.1 | 787 |
| V769_D770insASV | 21 | 4 | ASVASVDNPHVCRLLGICLTS | 50 | 1.1 | 788 |
| V769_D770insASV | 21 | 5 | MASVASVDNPHVCRLLGICLT | 46 | 1.1 | 789 |
| V769_D770insASV | 21 | 6 | VMASVASVDNPHVCRLLGICL | 43 | 0.7 | 790 |
| V769_D770insASV | 21 | 7 | YVMASVASVDNPHVCRLLGIC | 23 | 0.5 | 791 |
| V769_D770insASV | 21 | 8 | AYVMASVASVDNPHVCRLLGI | 12 | 0.3 | 792 |
| V769_D770insASV | 21 | 9 | EAYVMASVASVDNPHVCRLLG | 9 | 0.09 | 793 |

B. Cell Penetrating Peptides

In some embodiments, an immunotherapy may utilize a tumor antigen-specific peptide (e.g., EGFR mutant peptides) of the present disclosure that is associated with a cell penetrator, such as a liposome or a cell penetrating peptide (CPP). Antigen presenting cells (such as dendritic cells) pulsed with peptides may be used to enhance antitumour immunity (Celluzzi et al., 1996; Young et al., 1996). Liposomes and CPPs are described in further detail below. In some embodiments, an immunotherapy may utilize a nucleic acid encoding a tumor antigen-specific peptide (e.g., EGFR mutant peptides) of the present disclosure, wherein the nucleic acid is delivered, e.g., in a viral vector or non-viral vector.

A tumor antigen-specific peptide (e.g., EGFR mutant peptides) may also be associated with or covalently bound to a cell penetrating peptide (CPP). Cell penetrating peptides that may be covalently bound to a tumor antigen-specific peptide (e.g., EGFR mutant peptides) include, e.g., HIV Tat, herpes virus VP22, the Drosophila Antennapedia homeobox gene product, signal sequences, fusion sequences, or protegrin I. Covalently binding a peptide to a CPP can prolong the presentation of a peptide by dendritic cells, thus enhancing antitumour immunity (Wang and Wang, 2002). In some embodiments, a tumor antigen-specific peptide (e.g., the EGFR mutant peptide) of the present disclosure (e.g., comprised within a peptide or polyepitope string) may be covalently bound (e.g., via a peptide bond) to a CPP to generate a fusion protein. In other embodiments, a tumor antigen-specific peptide (e.g., EGFR mutant peptides) or nucleic acid encoding a tumor antigen-specific peptide may be encapsulated within or associated with a liposome, such as a mulitlamellar, vesicular, or multivesicular liposome, an exocytic vesicle or exosome.

As used herein, “association” means a physical association, a chemical association or both. For example, an association can involve a covalent bond, a hydrophobic interaction, encapsulation, surface adsorption, or the like.

As used herein, “cell penetrator” refers to a composition or compound which enhances the intracellular delivery of the peptide/polyepitope string to the antigen presenting cell. For example, the cell penetrator may be a lipid which, when associated with the peptide, enhances its capacity to cross the plasma membrane. Alternatively, the cell penetrator may be a peptide. Cell penetrating peptides (CPPs) are known in the art, and include, e.g., the Tat protein of HIV (Frankel and Pabo, 1988), the VP22 protein of HSV (Elliott and O'Hare, 1997) and fibroblast growth factor (Lin et al., 1995).

Cell-penetrating peptides (or “protein transduction domains”) have been identified from the third helix of the Drosophila Antennapedia homeobox gene (Antp), the HIV Tat, and the herpes virus VP22, all of which contain positively charged domains enriched for arginine and lysine residues (Schwarze et al., 2000; Schwarze et al., 1999). Also, hydrophobic peptides derived from signal sequences have been identified as cell-penetrating peptides. (Rojas el al., 1996; Rojas et al., 1998; Du et al., 1998). Coupling these peptides to marker proteins such as β-galactosidase has been shown to confer efficient internalization of the marker protein into cells, and chimeric, in-frame fusion proteins containing these peptides have been used to deliver proteins to a wide spectrum of cell types both in vitro and in vivo (Drin et al., 2002). Fusion of these cell penetrating peptides to a tumor antigen-specific peptide (e.g., EGFR mutant peptides) in accordance with the present disclosure may enhance cellular uptake of the polypeptides.

In some embodiments, cellular uptake is facilitated by the attachment of a lipid, such as stearate or myristilate, to the polypeptide. Lipidation has been shown to enhance the passage of peptides into cells. The attachment of a lipid moiety is another way that the present disclosure increases polypeptide uptake by the cell. Cellular uptake is further discussed below.

A tumor antigen-specific peptide (e.g., EGFR mutant peptides) of the present disclosure may be included in a liposomal vaccine composition. For example, the liposomal composition may be or comprise a proteoliposomal composition. Methods for producing proteoliposomal compositions that may be used with the present disclosure are described, e.g., in Neelapu et al. (2007) and Popescu et al. (2007). In some embodiments, proteoliposomal compositions may be used to treat a melanoma.

By enhancing the uptake of a tumor antigen-specific polypeptide, it may be possible to reduce the amount of protein or peptide required for treatment. This in turn can significantly reduce the cost of treatment and increase the supply of therapeutic agent. Lower dosages can also minimize the potential immunogenicity of peptides and limit toxic side effects.

In some embodiments, a tumor antigen-specific peptide (e.g., EGFR mutant peptides) may be associated with a nanoparticle to form nanoparticle-polypeptide complex. In some embodiments, the nanoparticle is a liposomes or other lipid-based nanoparticle such as a lipid-based vesicle (e.g., a DOTAP:cholesterol vesicle). In other embodiments, the nanoparticle is an iron-oxide based superparamagnetic nanoparticles. Superparamagnetic nanoparticles ranging in diameter from about 10 to 100 nm are small enough to avoid sequestering by the spleen, but large enough to avoid clearance by the liver. Particles this size can penetrate very small capillaries and can be effectively distributed in body tissues. Superparamagnetic nanoparticles-polypeptide complexes can be used as MRI contrast agents to identify and follow those cells that take up the tumor antigen-specific peptide (e.g., EGFR mutant peptides). In some embodiments, the nanoparticle is a semiconductor nanocrystal or a semiconductor quantum dot, both of which can be used in optical imaging. In further embodiments, the nanoparticle can be a nanoshell, which comprises a gold layer over a core of silica. One advantage of nanoshells is that polypeptides can be conjugated to the gold layer using standard chemistry. In other embodiments, the nanoparticle can be a fullerene or a nanotube (Gupta el al., 2005).

Peptides are rapidly removed from the circulation by the kidney and are sensitive to degradation by proteases in serum. By associating a tumor antigen-specific peptide (e.g., EGFR mutant peptides) with a nanoparticle, the nanoparticle-polypeptide complexes of the present disclosure may protect against degradation and/or reduce clearance by the kidney. This may increase the serum half-life of polypeptides, thereby reducing the polypeptide dose need for effective therapy. Further, this may decrease the costs of treatment, and minimizes immunological problems and toxic reactions of therapy.

C. Polyepitope Strings

In some embodiments, a tumor antigen-specific peptide (e.g., EGFR mutant peptides) is included or comprised in a polyepitope string. A polyepitope string is a peptide or polypeptide containing a plurality of antigenic epitopes from one or more antigens linked together. A polyepitope string may be used to induce an immune response in a subject, such as a human subject. Polyepitope strings have been previously used to target malaria and other pathogens (Baraldo et al.. 2005; Moorthy et al., 2004; Baird et al., 2004). A polyepitope string may refer to a nucleic acid (e.g., a nucleic acid encoding a plurality of antigens including EGFR mutant peptides) or a peptide or polypeptide (e.g., containing a plurality of antigens including EGFR mutant peptides). A polyepitope string may be included in a cancer vaccine composition.

D. Biological Functional Equivalents

A tumor antigen-specific peptide (e.g., EGFR mutant peptides) of the present disclosure may be modified to contain amino acid substitutions, insertions and/or deletions that do not alter their respective interactions with an HLA class protein, such as HLA-A*0101, binding regions. Such a biologically functional equivalent of a tumor antigen-specific peptide (e.g., EGFR mutant peptides) could be a molecule having like or otherwise desirable characteristics, e.g., binding of Specific HLA class I or HLA class II complexes. As a nonlimiting example, certain amino acids may be substituted for other amino acids in a tumor antigen-specific peptide (e.g., EGFR mutant peptides) disclosed herein without appreciable loss of interactive capacity, as demonstrated by detectably unchanged peptide binding to HLA. In some embodiments, the tumor antigen-specific peptide has a substitution mutation at an anchor residue, such as a substitution mutation at one, two, or all of positions: 1 (P1), 2 (P2), and/or 9 (P9). It is thus contemplated that a tumor antigen-specific peptide (e.g., EGFR mutant peptides) disclosed herein (or a nucleic acid encoding such a peptide) which is modified in sequence and/or structure, but which is unchanged in biological utility or activity remains within the scope of the compositions and methods disclosed herein.

It is also well understood by the skilled artisan that, inherent in the definition of a biologically functional equivalent peptide, is the concept that there is a limit to the number of changes that may be made within a defined portion of the molecule while still maintaining an acceptable level of equivalent biological activity. Biologically functional equivalent peptides are thus defined herein as those peptides in which certain, not most or all, of the amino acids may be substituted. Of course, a plurality of distinct peptides with different substitutions may easily be made and used in accordance with the present disclosure.

The skilled artisan is also aware that where certain residues are shown to be particularly important to the biological or structural properties of a peptide, e.g., residues in specific epitopes, such residues may not generally be exchanged. This may be the case in the present disclosure, as a mutation in an tumor antigen-specific peptide (e.g., the EGFR mutant peptide) disclosed herein could result in a loss of species-specificity and in turn, reduce the utility of the resulting peptide for use in methods of the present disclosure. Thus, peptides which are antigenic (e.g., bind specifically to HLA class I or HLA class II complexes) and comprise conservative amino acid substitutions are understood to be included in the present disclosure. Conservative substitutions are least likely to drastically alter the activity of a protein. A “conservative amino acid substitution” refers to replacement of amino acid with a chemically similar amino acid, i.e., replacing nonpolar amino acids with other nonpolar amino acids; substitution of polar amino acids with other polar amino acids, acidic residues with other acidic amino acids, etc.

Amino acid substitutions, such as those which might be employed in modifying a tumor antigen-specific peptide (e.g., EGFR mutant peptides) disclosed herein are generally based on the relative similarity of the amino acid side-chain substituents, for example, their hydrophobicity, hydrophilicity, charge, size, and the like. An analysis of the size, shape and type of the amino acid side-chain substituents reveals that arginine, lysine and histidine are all positively charged residues; that alanine, glycine and serine are all a similar size; and that phenylalanine, tryptophan and tyrosine all have a generally similar shape. Therefore, based upon these considerations, arginine, lysine and histidine; alanine, glycine and serine; and phenylalanine, tryptophan and tyrosine; are defined herein as biologically functional equivalents. In some embodiments, the mutation may enhance TCR-pMHC interaction and/or peptide-MHC binding.

The present disclosure also contemplates isoforms of the tumor antigen-specific peptides (e.g., EGFR mutant peptides) disclosed herein. An isoform contains the same number and kinds of amino acids as a peptide of the present disclosure, but the isoform has a different molecular structure. The isoforms contemplated by the present disclosure are those having the same properties as a peptide of the present disclosure as described herein.

Nonstandard amino acids may be incorporated into proteins by chemical modification of existing amino acids or by de novo synthesis of a peptide disclosed herein. A nonstandard amino acid refers to an amino acid that differs in chemical structure from the twenty standard amino acids encoded by the genetic code.

In select embodiments, the present disclosure contemplates a chemical derivative of a tumor antigen-specific peptide (e.g., EGFR mutant peptides) disclosed herein. “Chemical derivative” refers to a peptide having one or more residues chemically derivatized by reaction of a functional side group, and retaining biological activity and utility. Such derivatized peptides include, for example, those in which free amino groups have been derivatized to form specific salts or derivatized by alkylation and/or acylation, p-toluene sulfonyl groups, carbobenzoxy groups, t-butyloxycarbonyl groups, chloroacetyl groups, formyl or acetyl groups among others. Free carboxyl groups may be derivatized to form organic or inorganic salts, methyl and ethyl esters or other types of esters or hydrazides and preferably amides (primary or secondary). Chemical derivatives may include those peptides which comprise one or more naturally occurring amino acids derivatives of the twenty standard amino acids. For example, 4-hydroxyproline may be substituted for serine; and ornithine may be substituted for lysine.

It should be noted that all amino-acid residue sequences are represented herein by formulae whose left and right orientation is in the conventional direction of amino-terminus to carboxy-terminus. Furthermore, it should be noted that a dash at the beginning or end of an amino acid residue sequence indicates a peptide bond to a further sequence of one or more amino-acid residues. The amino acids described herein are preferred to be in the “L” isomeric form. However, residues in the “D” isomeric form can be substituted for any L-amino acid residue, as long as the desired functional properties set forth herein are retained by the protein.

Preferred tumor antigen-specific peptides (e.g., EGFR mutant peptides) or analogs thereof preferably specifically or preferentially bind a Specific HLA class I or HLA class II complexes. Determining whether or to what degree a particular tumor antigen-specific peptide or labeled peptide, or an analog thereof, can bind an Specific HLA class I or HLA class II complexes and can be assessed using an in vitro assay such as, for example, an enzyme-linked immunosorbent assay (ELISA), immunoblotting, immunoprecipitation, radioimmunoassay (RIA), immunostaining, latex agglutination, indirect hemagglutination assay (IHA), complement fixation, indirect immunofluorescent assay (FA), nephelometry, flow cytometry assay, chemiluminescence assay, lateral flow immunoassay, u-capture assay, mass spectrometry assay, particle-based assay, inhibition assay and/or an avidity assay.

E. Nucleic Acids Encoding a Tumor Antigen-Specific Peptide

In an aspect, the present disclosure provides a nucleic acid encoding an isolated antigen-specific peptide comprising a sequence that has at least about 90%, 91%, 92%, 93%, 94%, 95%, 96%, 97%, 98%, 99%, or 100% sequence identity to a peptide selected from those in Tables 1-3, or the peptide may have 1, 2, 3, or 4 point mutations (e.g., substitution mutations) as compared to a peptide selected from those in Tables 1-3. As stated above, such a tumor antigen-specific peptide may be, e.g., from 8 to 35 amino acids in length, or any range derivable therein.

Some embodiments of the present disclosure provide recombinantly-produced tumor antigen-specific peptides (e.g., EGFR mutant peptides) which can specifically bind a Specific HLA class I or HLA class II complexes. Accordingly, a nucleic acid encoding a tumor antigen-specific peptide may be operably linked to an expression vector and the peptide produced in the appropriate expression system using methods well known in the molecular biological arts. A nucleic acid encoding a tumor antigen-specific peptide disclosed herein may be incorporated into any expression vector which ensures good expression of the peptide. Possible expression vectors include but are not limited to cosmids, plasmids, or modified viruses (e.g. replication defective retroviruses, adenoviruses and adeno-associated viruses), so long as the vector is suitable for transformation of a host cell.

A recombinant expression vector being “suitable for transformation of a host cell” means that the expression vector contains a nucleic acid molecule of the present disclosure and regulatory sequences selected on the basis of the host cells to be used for expression, which is operatively linked to the nucleic acid molecule. The terms, “operatively linked” or “operably linked” are used interchangeably and are intended to mean that the nucleic acid is linked to regulatory sequences in a manner which allows expression of the nucleic acid.

Accordingly, the present disclosure provides a recombinant expression vector comprising nucleic acid encoding a tumor antigen-specific peptide, and the necessary regulatory sequences for the transcription and translation of the inserted protein-sequence. Suitable regulatory sequences may be derived from a variety of sources, including bacterial, fungal, or viral genes (e.g., see the regulatory sequences described in Goeddel (1990).

Selection of appropriate regulatory sequences is generally dependent on the host cell chosen, and may be readily accomplished by one of ordinary skill in the art. Examples of such regulatory sequences include: a transcriptional promoter and enhancer or RNA polymerase binding sequence, a ribosomal binding sequence, including a translation initiation signal. Additionally, depending on the host cell chosen and the vector employed, other sequences, such as an origin of replication, additional DNA restriction sites, enhancers, and sequences conferring inducibility of transcription may be incorporated into the expression vector. It will also be appreciated that the necessary regulatory sequences may be supplied by the native protein and/or its flanking regions.

A recombinant expression vector may also contain a selectable marker gene which facilitates the selection of host cells transformed or transfected with a recombinant tumor antigen-specific peptides (e.g., EGFR mutant peptides) disclosed herein. Examples of selectable marker genes are genes encoding a protein such as G418 and hygromycin which confer resistance to certain drugs, β-galactosidase, chloramphenicol acetyltransferase, or firefly luciferase. Transcription of the selectable marker gene is monitored by changes in the concentration of the selectable marker protein such as β-galactosidase, chloramphenicol acetyltransferase, or firefly luciferase. If the selectable marker gene encodes a protein conferring antibiotic resistance such as neomycin resistance transformant cells can be selected with G418. Cells that have incorporated the selectable marker gene will survive, while the other cells die. This makes it possible to visualize and assay for expression of a recombinant expression vector, and in particular, to determine the effect of a mutation on expression and phenotype. It will be appreciated that selectable markers can be introduced on a separate vector from the nucleic acid of interest.

Recombinant expression vectors can be introduced into host cells to produce a transformant host cell. The term “transformant host cell” is intended to include prokaryotic and eukaryotic cells which have been transformed or transfected with a recombinant expression vector of the present disclosure. The terms “transformed with”, “transfected with”, “transformation” and “transfection” are intended to encompass introduction of nucleic acid (e.g. a vector) into a cell by one of many possible techniques known in the art. Suitable host cells include a wide variety of prokaryotic and eukaryotic host cells. For example, the proteins of the present disclosure may be expressed in bacterial cells such as E. coli, insect cells (using baculovirus), yeast cells or mammalian cells.

A nucleic acid molecule of the present disclosure may also be chemically synthesized using standard techniques. Various methods of chemically synthesizing polydeoxy-nucleotides are known, including solid-phase synthesis which, like peptide synthesis, has been fully automated in commercially available DNA synthesizers (See e.g., U.S. Pat. Nos. 4,598,049; 4,458,066; 4,401,796; and 4,373,071).

### IV. Antigen-Specific Cell Therapy

Embodiments of the present disclosure concern obtaining and administering antigen-specific cells (e.g., autologous or allogeneic T cells (e.g., regulatory T cells, CD4 T cells, CD8⁺ T cells, or gamma-delta T cells), NK cells, invariant NK cells, NKT cells, mesenchymal stem cell (MSC)s, or induced pluripotent stem (iPS) cells) to a subject as an immunotherapy to target cancer cells. In particular, the cells are antigen-specific T cells (e.g., mutant EGFR-specific T cells). Several basic approaches for the derivation, activation and expansion of functional anti-tumor effector cells have been described in the last two decades. These include: autologous cells, such as tumor-infiltrating lymphocytes (TILs); T cells activated ex-vivo using autologous DCs, lymphocytes, artificial antigen-presenting cells (APCs) or beads coated with T cell ligands and activating antibodies, or cells isolated by virtue of capturing target cell membrane; allogeneic cells naturally expressing anti-host tumor T cell receptor (TCR); and non-tumor-specific autologous or allogeneic cells genetically reprogrammed or “redirected” to express tumor-reactive TCR or chimeric TCR molecules displaying antibody-like tumor recognition capacity known as “T-bodies”. These approaches have given rise to numerous protocols for T cell preparation and immunization which can be used in the methods described herein.

B. T Cell Preparation

In some embodiments, the T cells are derived from the blood, bone marrow, lymph, umbilical cord, or lymphoid organs. In some aspects, the cells are human cells. The cells typically are primary cells, such as those isolated directly from a subject and/or isolated from a subject and frozen. In some embodiments, the cells include one or more subsets of T cells or other cell types, such as whole T cell populations, CD4⁺ cells, CD8⁺ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. With reference to the subject to be treated, the cells may be allogeneic and/or autologous. In some aspects, such as for off-the-shelf technologies, the cells are pluripotent and/or multipotent, such as stem cells, such as induced pluripotent stem cells (iPSCs). In some embodiments, the methods include isolating cells from the subject, preparing, processing, culturing, and/or engineering them, as described herein, and re-introducing them into the same patient, before or after cryopreservation.

Among the sub-types and subpopulations of T cells (e.g., CD4⁺ and/or CD8⁺ T cells) are naive T (T_{N}) cells, effector T cells (T_{EFF}), memory T cells and sub-types thereof, such as stem cell memory T (TSC_{M}), central memory T (TC_{M}), effector memory T (T_{EM}), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells.

In some embodiments, one or more of the T cell populations is enriched for or depleted of cells that are positive for a specific marker, such as surface markers, or that are negative for a specific marker. In some cases, such markers are those that are absent or expressed at relatively low levels on certain populations of T cells (e.g., non-memory cells) but are present or expressed at relatively higher levels on certain other populations of T cells (e.g., memory cells).

In some embodiments, T cells are separated from a PBMC sample by negative selection of markers expressed on non-T cells, such as B cells, monocytes, or other white blood cells, such as CD14. In some aspects, a CD4⁺ or CD8⁺ selection step is used to separate CD4⁺ helper and CD8⁺ cytotoxic T cells. Such CD4⁺ and CD8⁺ populations can be further sorted into sub-populations by positive or negative selection for markers expressed or expressed to a relatively higher degree on one or more naive, memory, and/or effector T cell subpopulations.

In some embodiments, CD8⁺ T cells are further enriched for or depleted of naive, central memory, effector memory, and/or central memory stem cells, such as by positive or negative selection based on surface antigens associated with the respective subpopulation. In some embodiments, enrichment for central memory T (T_{CM}) cells is carried out to increase efficacy, such as to improve long-term survival, expansion, and/or engraftment following administration, which in some aspects is particularly robust in such sub-populations. See Terakura et al., 2012; Wang el al., 2012.

In some embodiments, the T cells are autologous T cells. In this method, tumor samples are obtained from patients and a single cell suspension is obtained. The single cell suspension can be obtained in any suitable manner, e.g., mechanically (disaggregating the tumor using, e.g., a gentleMACS™ Dissociator, Miltenyi Biotec, Auburn, Calif.) or enzymatically (e.g., collagenase or DNase). Single-cell suspensions of tumor enzymatic digests are cultured in interleukin-2 (IL-2). The cells are cultured until confluence (e.g., about 2×10⁶ lymphocytes), e.g., from about 5 to about 21 days, preferably from about 10 to about 14 days.

The cultured T cells can be pooled and rapidly expanded. Rapid expansion provides an increase in the number of antigen-specific T-cells of at least about 50-fold (e.g., 50-, 60-, 70-, 80-, 90-, or 100-fold, or greater) over a period of about 10 to about 14 days. More preferably, rapid expansion provides an increase of at least about 200-fold (e.g., 200-, 300-, 400-, 500-, 600-, 700-, 800-, 900-, or greater) over a period of about 10 to about 14 days.

Expansion can be accomplished by any of a number of methods as are known in the art. For example, T cells can be rapidly expanded using non-specific T cell receptor stimulation in the presence of feeder lymphocytes and either interleukin-2 (IL-2) or interleukin-15 (IL-15), with IL-2 being preferred. The non-specific T cell receptor stimulus can include around 30 ng/ml of OKT3, a mouse monoclonal anti-CD3 antibody (available from Ortho-McNeil®, Raritan, N.J.). Alternatively, T cells can be rapidly expanded by stimulation of PBMCs in vitro with one or more antigens (including antigenic portions thereof, such as epitope(s), or a cell) of the cancer, which can be optionally expressed from a vector, such as an human leukocyte antigen A2 (HLA-A2) binding peptide, in the presence of a T cell growth factor, such as 300 IU/ml IL-2 or IL-15, with IL-2 being preferred. The in vitro-induced T cells are rapidly expanded by re-stimulation with the same antigen(s) of the cancer pulsed onto HLA-A2-expressing antigen-presenting cells. Alternatively, the T cells can be re-stimulated with irradiated, autologous lymphocytes or with irradiated HLA-A2+ allogeneic lymphocytes and IL-2, for example.

The autologous T cells can be modified to express a T cell growth factor that promotes the growth and activation of the autologous T cells. Suitable T cell growth factors include, for example, IL-2, IL-7, IL-15, and IL-12. Suitable methods of modification are known in the art. See, for instance, Sambrook et al., 2001; and Ausubel et al., 1994. In particular aspects, modified autologous T cells express the T cell growth factor at high levels. T cell growth factor coding sequences, such as that of IL-12, are readily available in the art, as are promoters, the operable linkage of which to a T cell growth factor coding sequence promote high-level expression.

C. Antigen-Presenting Cells

Antigen-presenting cells, which include macrophages, B lymphocytes, and dendritic cells, are distinguished by their expression of a particular MHC molecule. APCs internalize antigen and re-express a part of that antigen, together with the MHC molecule on their outer cell membrane. The major histocompatibility complex (MHC) is a large genetic complex with multiple loci. The MHC loci encode two major classes of MHC membrane molecules, referred to as class I and class II MHCs. T helper lymphocytes generally recognize antigen associated with MHC class II molecules, and T cytotoxic lymphocytes recognize antigen associated with MHC class I molecules. In humans the MHC is referred to as the HLA complex and in mice the H-2 complex. In some aspects, EGFR mutant peptides of the embodiments are expressed in antigen presenting cells. Such cells provide engineered APCs that can be used to specifically propagate immune effector cells specific for the mutant EGER antigen of interest.

In some cases, aAPCs are useful in preparing therapeutic compositions and cell therapy products of the embodiments. For general guidance regarding the preparation and use of antigen-presenting systems, see, e.g., U.S. Pat. Nos. 6,225,042, 6,355,479, 6,362,001 and 6,790,662; U.S. Patent Application Publication Nos. 2009/0017000 and 2009/0004142; and International Publication No. WO2007/103009.

aAPC systems may comprise at least one exogenous assisting molecule. Any suitable number and combination of assisting molecules may be employed. The assisting molecule may be selected from assisting molecules such as co-stimulatory molecules and adhesion molecules. Exemplary co-stimulatory molecules include CD86, CD64 (FcγRI), 41BB ligand, and IL-21. Adhesion molecules may include carbohydrate-binding glycoproteins such as selectins, transmembrane binding glycoproteins such as integrins, calcium-dependent proteins such as cadherins, and single-pass transmembrane immunoglobulin (Ig) superfamily proteins, such as intercellular adhesion molecules (ICAMs), which promote, for example, cell-to-cell or cell-to-matrix contact. Exemplary adhesion molecules include LFA-3 and ICAMs, such as ICAM-1. Techniques, methods, and reagents useful for selection, cloning, preparation, and expression of exemplary assisting molecules, including co-stimulatory molecules and adhesion molecules, are exemplified in, e.g., U.S. Pat. Nos. 6,225,042, 6,355,479, and 6,362,001.

### V. Methods of Treatment

Provided herein are methods for treating or delaying progression of cancer in an individual comprising administering to the individual an effective amount an antigen-specific cell therapy, such as a mutant EGFR-specific T cell therapy. Adoptive T cell therapies with genetically engineered TCR-transduced T cells (conjugate TCR to other bio reactive proteins (e.g., anti-CD3) are also provided herein. In further embodiments, methods are provided for the treatment of cancer comprising immunizing a subject with a purified tumor antigen or an immunodominant tumor antigen-specific peptide.

The EGFR mutant peptides provided herein can be utilized to develop cancer vaccines or immunogens (e.g., a peptide or modified peptide mix with adjuvant, coding polynucleotide and corresponding expression products such as inactive virus or other microorganisms vaccine). These peptide specific vaccines or immunogens can be used for immunizing cancer patients directly to induce anti-tumor immuno-response in vivo, or for expanding antigen specific T cells in vitro with peptide or coded polynucleotide loaded APC stimulation. These large number of T cells can be adoptively transferred to patients to induce tumor regression.

Examples of cancers contemplated for treatment include lung cancer, head and neck cancer, breast cancer, pancreatic cancer, prostate cancer, renal cancer, bone cancer, testicular cancer, cervical cancer, gastrointestinal cancer, lymphomas, pre-neoplastic lesions in the lung, colon cancer, melanoma, and bladder cancer.

In some embodiments, T cells are autologous. However, the cells can be allogeneic. In some embodiments, the T cells are isolated from the patient themself, so that the cells are autologous. If the T cells are allogeneic, the T cells can be pooled from several donors. The cells are administered to the subject of interest in an amount sufficient to control, reduce, or eliminate symptoms and signs of the disease being treated.

In some embodiments, the subject can be administered nonmyeloablative lymphodepleting chemotherapy prior to the T cell therapy. The nonmyeloablative lymphodepleting chemotherapy can be any suitable such therapy, which can be administered by any suitable route. The nonmyeloablative lymphodepleting chemotherapy can comprise, for example, the administration of cyclophosphamide and fludarabine, particularly if the cancer is melanoma, which can be metastatic. An exemplary route of administering cyclophosphamide and fludarabine is intravenously. Likewise, any suitable dose of cyclophosphamide and fludarabine can be administered. In particular aspects, around 60 mg/kg of cyclophosphamide is administered for two days after which around 25 mg/m² fludarabine is administered for five days.

In certain embodiments, a T-cell growth factor that promotes the growth and activation of the autologous T cells is administered to the subject either concomitantly with the autologous T cells or subsequently to the autologous T cells. The T-cell growth factor can be any suitable growth factor that promotes the growth and activation of the autologous T-cells. Examples of suitable T-cell growth factors include IL-2, IL-7, IL-15, and IL-12, which can be used alone or in various combinations, such as IL-2 and IL-7, IL-2 and IL-15, IL-7 and IL-15, IL-2, IL-7 and IL-15, IL-12 and IL-7, IL-12 and IL-15, or IL-12 and IL2. IL-12 is a preferred T-cell growth factor.

The T cell may be administered intravenously, intramuscularly, subcutaneously, topically, orally, transdermally, intraperitoneally, intraorbitally, by implantation, by inhalation, intrathecally, intraventricularly, or intranasally. The appropriate dosage of the T cell therapy may be determined based on the type of disease to be treated, severity and course of the disease, the clinical condition of the individual, the individual's clinical history and response to the treatment, and the discretion of the attending physician.

Intratumoral injection, or injection into the tumor vasculature is specifically contemplated for discrete, solid, accessible tumors. Local, regional or systemic administration also may be appropriate. For tumors of >4 cm, the volume to be administered will be about 4-10 ml (in particular 10 ml), while for tumors of <4 cm, a volume of about 1-3 ml will be used (in particular 3 ml). Multiple injections delivered as single dose comprise about 0.1 to about 0.5 ml volumes.

B. Pharmaceutical Compositions

Also provided herein are pharmaceutical compositions and formulations comprising antigen-specific immune cells (e.g., T cells) or receptors (e.g., TCR) and a pharmaceutically acceptable carrier. A vaccine composition for pharmaceutical use in a subject may comprise a tumor antigen peptide (e.g., a EGFR mutant peptide) composition disclosed herein and a pharmaceutically acceptable carrier.

Pharmaceutical compositions and formulations as described herein can be prepared by mixing the active ingredients (such as an antibody or a polypeptide) having the desired degree of purity with one or more optional pharmaceutically acceptable carriers (Remington's Pharmaceutical Sciences 22^{nd} edition, 2012), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Exemplary pharmaceutically acceptable carriers herein further include interstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

C. Combination Therapies

In certain embodiments, the compositions and methods of the present embodiments involve an antigen-specific immune cell population or TCR in combination with at least one additional therapy. The additional therapy may be radiation therapy, surgery (e.g., lumpectomy and a mastectomy), chemotherapy, gene therapy, DNA therapy, viral therapy, RNA therapy, immunotherapy, bone marrow transplantation, nanotherapy, monoclonal antibody therapy, or a combination of the foregoing. The additional therapy may be in the form of adjuvant or neoadjuvant therapy.

In some embodiments, the additional therapy is the administration of small molecule enzymatic inhibitor or anti-metastatic agent. In some embodiments, the additional therapy is the administration of side-effect limiting agents (e.g., agents intended to lessen the occurrence and/or severity of side effects of treatment, such as anti-nausea agents, etc.). In some embodiments, the additional therapy is radiation therapy. In some embodiments, the additional therapy is surgery. In some embodiments, the additional therapy is a combination of radiation therapy and surgery. In some embodiments, the additional therapy is gamma irradiation. In some embodiments, the additional therapy is therapy targeting PBK/AKT/mTOR pathway, HSP90 inhibitor, tubulin inhibitor, apoptosis inhibitor, and/or chemopreventative agent. The additional therapy may be one or more of the chemotherapeutic agents known in the art.

An immune cell therapy may be administered before, during, after, or in various combinations relative to an additional cancer therapy, such as immune checkpoint therapy. The administrations may be in intervals ranging from concurrently to minutes to days to weeks. In embodiments where the immune cell therapy is provided to a patient separately from an additional therapeutic agent, one would generally ensure that a significant period of time did not expire between the time of each delivery, such that the two compounds would still be able to exert an advantageously combined effect on the patient. In such instances, it is contemplated that one may provide a patient with the antibody therapy and the anti-cancer therapy within about 12 to 24 or 72 h of each other and, more particularly, within about 6-12 h of each other. In some situations it may be desirable to extend the time period for treatment significantly where several days (2, 3, 4, 5, 6, or 7) to several weeks (1, 2, 3, 4, 5, 6, 7, or 8) lapse between respective administrations.

Various combinations may be employed. For the example below an antigen-specific immune cell therapy, peptide, or TCR is “A” and an anti-cancer therapy is “B”:

| | | | | | | | |
|---|---|---|---|---|---|---|---|
| A/B/A | B/A/B | B/B/A | A/A/B | A//B/B | B/A/A | A/B/B/B | B/A/B/B |

| | | | | | |
|---|---|---|---|---|---|
| B/B/B/A | B/B/A/B | A/A/B/B | A/B/A/B | A/B/B/A | B/B/A/A |
| B/A/B/A | B/A/A/B | A/A/A/B | B/A/A/A | A/B/A/A | A/A/B/A |

Administration of any compound or therapy of the present embodiments to a patient will follow general protocols for the administration of such compounds, taking into account the toxicity, if any, of the agents. Therefore, in some embodiments there is a step of monitoring toxicity that is attributable to combination therapy.

2. Chemotherapy

A wide variety of chemotherapeutic agents may be used in accordance with the present embodiments. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis.

Examples of chemotherapeutic agents include alkylating agents, such as thiotepa and cyclosphosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trietylenephosphoramide, triethiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TMI); eleutherobin; pancratistatin; a sarcodictyin; spongistatin; nitrogen mustards, such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as the enediyne antibiotics (e.g., calicheamicin, especially calicheamicin gammaII and calicheamicin omegaII); dynemicin, including dynemicin A; bisphosphonates, such as clodronate; an esperamicin; as well as neocarzinostatin chromophore and related chromoprotein enediyne antibiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenisher, such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSKpolysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluoromethylornithine (DMFO); retinoids, such as retinoic acid; capecitabine; carboplatin, procarbazine, plicomycin, gemcitabine, navelbine, farnesyl-protein transferase inhibitors, transplatinum, and pharmaceutically acceptable salts, acids, or derivatives of any of the above.

3. Radiotherapy

Other factors that cause DNA damage and have been used extensively include what are commonly known as γ-rays, X-rays, and/or the directed delivery of radioisotopes to tumor cells. Other forms of DNA damaging factors are also contemplated, such as microwaves, proton beam irradiation (U.S. Pat. Nos. 5,760,395 and 4,870,287), and UV-irradiation. It is most likely that all of these factors affect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

4. Immunotherapy

The skilled artisan will understand that additional immunotherapies may be used in combination or in conjunction with methods of the embodiments. In the context of cancer treatment, immunotherapeutics, generally, rely on the use of immune effector cells and molecules to target and destroy cancer cells. Rituximab (RITUXAN®) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve as a targeting agent. Alternatively, the effector may be a lymphocyte carrying a surface molecule that interacts, either directly or indirectly, with a tumor cell target. Various effector cells include cytotoxic T cells and NK cells.

Antibody-drug conjugates have emerged as a breakthrough approach to the development of cancer therapeutics. Cancer is one of the leading causes of deaths in the world. Antibody-drug conjugates (ADCs) comprise monoclonal antibodies (MAbs) that are covalently linked to cell-killing drugs. This approach combines the high specificity of MAbs against their antigen targets with highly potent cytotoxic drugs, resulting in “armed” MAbs that deliver the payload (drug) to tumor cells with enriched levels of the antigen. Targeted delivery of the drug also minimizes its exposure in normal tissues, resulting in decreased toxicity and improved therapeutic index. The approval of two ADC drugs, ADCETRIS® (brentuximab vedotin) in 2011 and KADCYLA® (trastuzumab emtansine or T-DM1) in 2013 by FDA validated the approach. There are currently more than 30 ADC drug candidates in various stages of clinical trials for cancer treatment (Leal et al., 2014). As antibody engineering and linker-payload optimization are becoming more and more mature, the discovery and development of new ADCs are increasingly dependent on the identification and validation of new targets that are suitable to this approach and the generation of targeting MAbs. Two criteria for ADC targets are upregulated/high levels of expression in tumor cells and robust internalization.

In one aspect of immunotherapy, the tumor cell must bear some marker that is amenable to targeting, i.e., is not present on the majority of other cells. Many tumor markers exist and any of these may be suitable for targeting in the context of the present embodiments. Common tumor markers include CD20, carcinoembryonic antigen, tyrosinase (p97), gp68, TAG-72, HMFG, Sialyl Lewis Antigen, MucA, MucB, PLAP, laminin receptor, erb B, and p155. An alternative aspect of immunotherapy is to combine anticancer effects with immune stimulatory effects. Immune stimulating molecules also exist including: cytokines, such as IL-2, IL-4, IL-12, GM-CSF, gamma-IFN, chemokines, such as MIP-1, MCP-1, IL-8, and growth factors, such as FLT3 ligand.

Examples of immunotherapies currently under investigation or in use are immune adjuvants, e.g., Mycobacterium bovis, Plasmodium falciparum, dinitrochlorobenzene, and aromatic compounds (U.S. Pat. Nos. 5,801,005 and 5,739,169; Hui and Hashimoto, 1998; Christodoulides et al., 1998); cytokine therapy, e.g., interferons α, β, and γ, IL-1, GM-CSF, and TNF (Bukowski et al., 1998; Davidson el al., 1998; Hellstrand et al., 1998); gene therapy, e.g., TNF, IL-1, IL-2, and p53 (Qin et al., 1998. Austin-Ward and Villaseca, 1998; U.S. Pat. Nos. 5,830,880 and 5,846,945); and monoclonal antibodies, e.g., anti-CD20, anti-ganglioside GM2, and anti-p185 (Hollander, 2012; Hanibuchi et al., 1998; U.S. Pat. No. 5,824,311). It is contemplated that one or more anti-cancer therapies may be employed with the antibody therapies described herein.

In some embodiments, the immunotherapy may be an immune checkpoint inhibitor. Immune checkpoints either turn up a signal (e.g., co-stimulatory molecules) or turn down a signal. Inhibitory immune checkpoints that may be targeted by immune checkpoint blockade include adenosine A2A receptor (A2AR), B7-H3 (also known as CD276), B and T lymphocyte attenuator (BTLA), cytotoxic T-lymphocyte-associated protein 4 (CTLA-4, also known as CD152), indoleamine 2,3-dioxygenase (IDO), killer-cell immunoglobulin (KIR), lymphocyte activation gene-3 (LAG3), programmed death 1 (PD-1), T-cell immunoglobulin domain and mucin domain 3 (TIM-3) and V-domain Ig suppressor of T cell activation (VISTA). In particular, the immune checkpoint inhibitors target the PD-1 axis and/or CTLA-4.

The immune checkpoint inhibitors may be drugs such as small molecules, recombinant forms of ligand or receptors, or, in particular, are antibodies, such as human antibodies (e.g., International Patent Publication WO2015016718; Pardoll, Nat Rev Cancer, 12(4): 252-64, 2012; both incorporated herein by reference). Known inhibitors of the immune checkpoint proteins or analogs thereof may be used, in particular chimerized, humanized or human forms of antibodies may be used. As the skilled person will know, alternative and/or equivalent names may be in use for certain antibodies mentioned in the present disclosure. Such alternative and/or equivalent names are interchangeable in the context of the present disclosure. For example, it is known that lambrolizumab is also known under the alternative and equivalent names MK-3475 and pembrolizumab.

In some embodiments, the PD-1 binding antagonist is a molecule that inhibits the binding of PD-1 to its ligand binding partners. In a specific aspect, the PD-1 ligand binding partners are PDL1 and/or PDL2. In another embodiment, a PDL1 binding antagonist is a molecule that inhibits the binding of PDL1 to its binding partners. In a specific aspect, PDL1 binding partners are PD-1 and/or B7-1. In another embodiment, the PDL2 binding antagonist is a molecule that inhibits the binding of PDL2 to its binding partners. In a specific aspect, a PDL2 binding partner is PD-1. The antagonist may be an antibody, an antigen binding fragment thereof, an immunoadhesin, a fusion protein, or oligopeptide. Exemplary antibodies are described in U.S. Pat. Nos. 8,735,553, 8,354,509, and 8,008,449, all incorporated herein by reference. Other PD-1 axis antagonists for use in the methods provided herein are known in the art such as described in U.S. Patent Application No. US20140294898, US2014022021, and US20110008369, all incorporated herein by reference.

In some embodiments, the PD-1 binding antagonist is an anti-PD-1 antibody (e.g., a human antibody, a humanized antibody, or a chimeric antibody). In some embodiments, the anti-PD-1 antibody is selected from the group consisting of nivolumab, pembrolizumab, and CT-011. In some embodiments, the PD-1 binding antagonist is an immunoadhesin (e.g., an immunoadhesin comprising an extracellular or PD-1 binding portion of PDL1 or PDL2 fused to a constant region (e.g., an Fc region of an immunoglobulin sequence). In some embodiments, the PD-1 binding antagonist is AMP-224. Nivolumab, also known as MDX-1106-04, MDX-1106, ONO-4538, BMS-936558, and OPDIVO®, is an anti-PD-1 antibody described in WO2006/121168. Pembrolizumab, also known as MK-3475, Merck 3475, lambrolizumab, KEYTRUDA®, and SCH-900475, is an anti-PD-1 antibody described in WO2009/114335. CT-011, also known as hBAT or hBAT-1, is an anti-PD-1 antibody described in WO2009/101611. AMP-224, also known as B7-DCIg, is a PDL2-Fc fusion soluble receptor described in WO2010/027827 and WO2011/066342.

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to CD80 or CD86 on the surface of antigen-presenting cells. CTLA4 is a member of the immunoglobulin superfamily that is expressed on the surface of Helper T cells and transmits an inhibitory signal to T cells. CTLA4 is similar to the T-cell co-stimulatory protein, CD28, and both molecules bind to CD80 and CD86, also called B7-1 and B7-2 respectively, on antigen-presenting cells. CTLA4 transmits an inhibitory signal to T cells, whereas CD28 transmits a stimulatory signal. Intracellular CTLA4 is also found in regulatory T cells and may be important to their function. T cell activation through the T cell receptor and CD28 leads to increased expression of CTLA-4, an inhibitory receptor for B7 molecules.

In some embodiments, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or VH and/or VL domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129, WO 01/14424, WO 98/42752; WO 00/37504 (CP675,206, also known as tremelimumab; formerly ticilimumab), U.S. Pat. No. 6,207,156; Hurwitz et al. (1998) Proc Natl Acad Sci USA 95(17): 10067-10071; Camacho et al. (2004) J Clin Oncology 22(145); Abstract No. 2505 (antibody CP-675206); and Mokyr et al. (1998) Cancer Res 58:5301-5304 can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001014424, WO2000037504, and U.S. Pat. No. 8,017,114; all incorporated herein by reference.

An exemplary anti-CTLA-4 antibody is ipilimumab (also known as 10D1, MDX-010, MDX-101, and Yervoy®) or antigen binding fragments and variants thereof (see, e.g., WO 01/14424). In other embodiments, the antibody comprises the heavy and light chain CDRs or VRs of ipilimumab. Accordingly, in one embodiment, the antibody comprises the CDR1, CDR2, and CDR3 domains of the VH region of ipilimumab, and the CDR1, CDR2 and CDR3 domains of the VL region of ipilimumab. In another embodiment, the antibody competes for binding with and/or binds to the same epitope on CTLA-4 as the above-mentioned antibodies. In another embodiment, the antibody has at least about 90% variable region amino acid sequence identity with the above-mentioned antibodies (e.g., at least about 90%, 95%, or 99% variable region identity with ipilimumab).

Other molecules for modulating CTLA-4 include CTLA-4 ligands and receptors such as described in U.S. Pat. Nos. 5,844,905, 5,885,796 and International Patent Application Nos. WO1995001994 and WO1998042752; all incorporated herein by reference, and immunoadhesins such as described in U.S. Pat. No. 8,329,867, incorporated herein by reference.

5. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present embodiments, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

6. Other Agents

It is contemplated that other agents may be used in combination with certain aspects of the present embodiments to improve the therapeutic efficacy of treatment. These additional agents include agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions would increase the anti-hyperproliferative effects on the neighboring hyperproliferative cell population. In other embodiments, cytostatic or differentiation agents can be used in combination with certain aspects of the present embodiments to improve the anti-hyperproliferative efficacy of the treatments. Inhibitors of cell adhesion are contemplated to improve the efficacy of the present embodiments. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with certain aspects of the present embodiments to improve the treatment efficacy.

### VI. Articles of Manufacture or Kits

An article of manufacture or a kit is provided comprising antigen-specific immune cells, TCRs, or antigen peptides (e.g., EGFR peptide) is also provided herein. The article of manufacture or kit can further comprise a package insert comprising instructions for using the antigen-specific immune cells to treat or delay progression of cancer in an individual or to enhance immune function of an individual having cancer. Any of the antigen-specific immune cells described herein may be included in the article of manufacture or kits. Suitable containers include, for example, bottles, vials, bags and syringes. The container may be formed from a variety of materials such as glass, plastic (such as polyvinyl chloride or polyolefin), or metal alloy (such as stainless steel or hastelloy). In some embodiments, the container holds the formulation and the label on, or associated with, the container may indicate directions for use. The article of manufacture or kit may further include other materials desirable from a commercial and user standpoint, including other buffers, diluents, filters, needles, syringes, and package inserts with instructions for use. In some embodiments, the article of manufacture further includes one or more of another agent (e.g., a chemotherapeutic agent, and anti-neoplastic agent). Suitable containers for the one or more agent include, for example, bottles, vials, bags and syringes.

### VII. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—Results and Methods

### A. Results

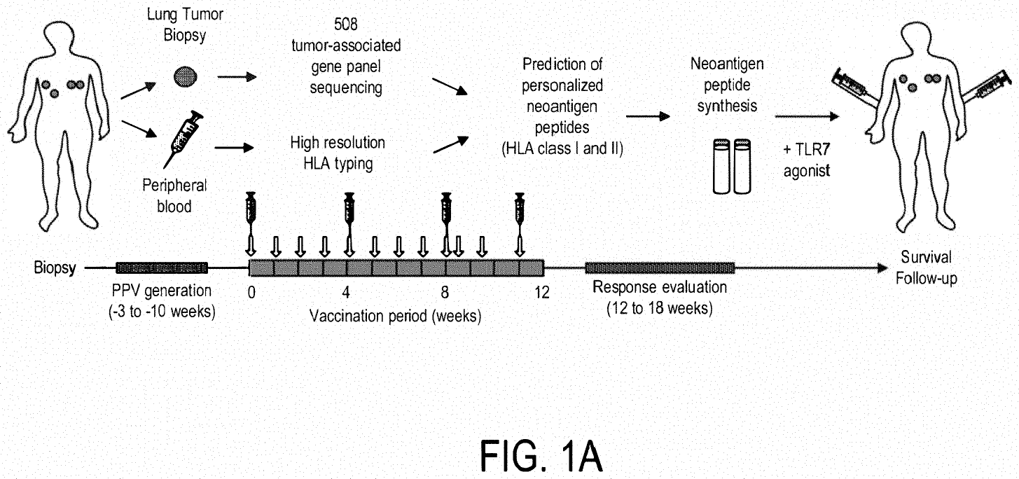

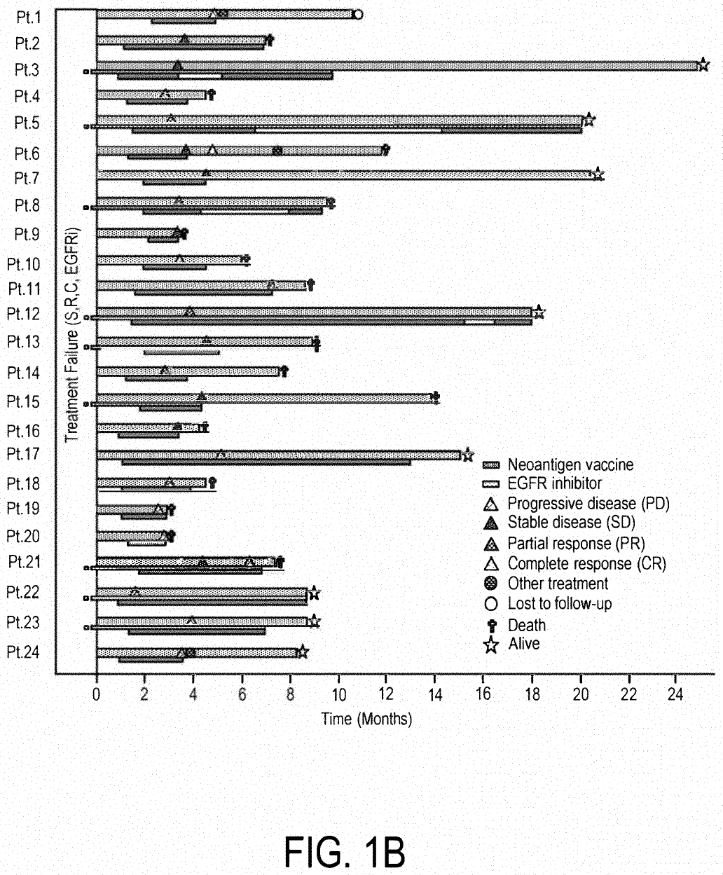

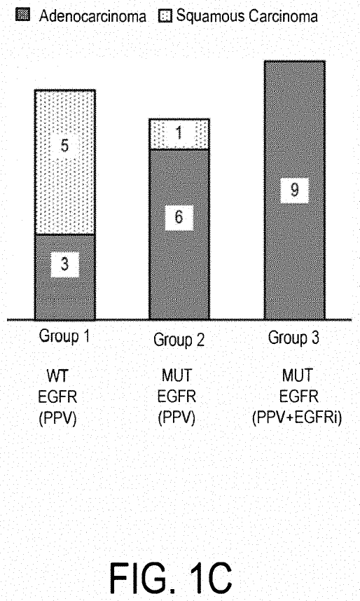

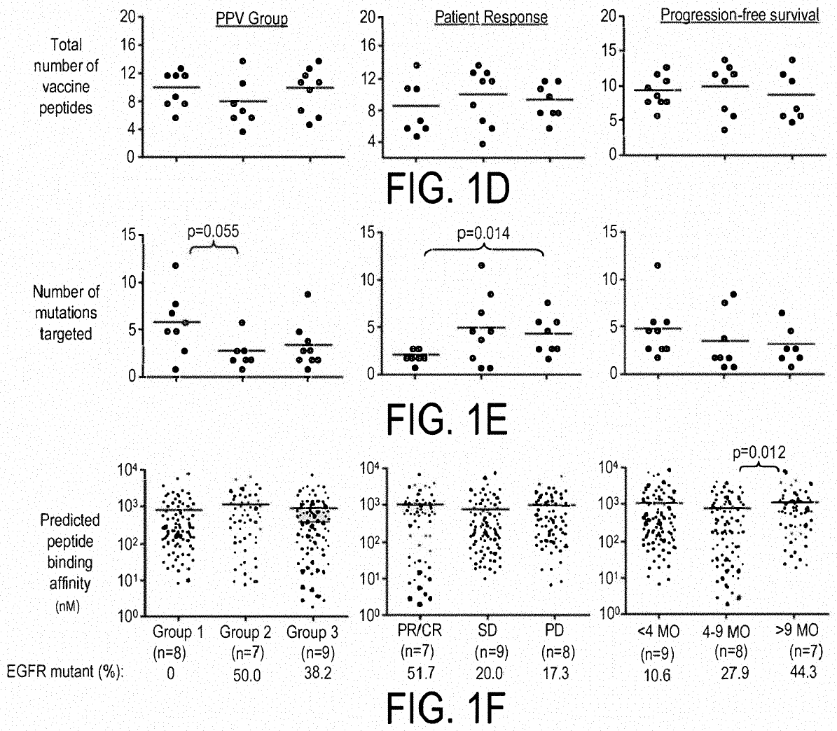

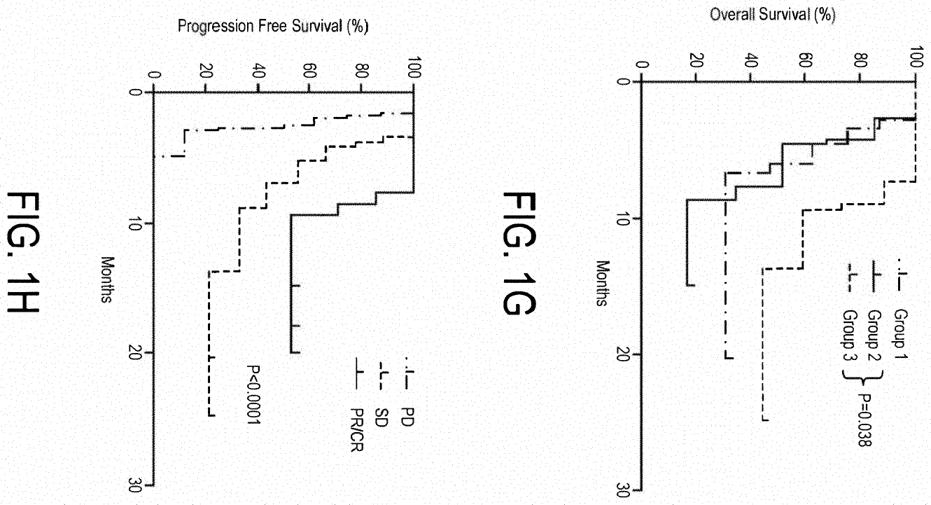

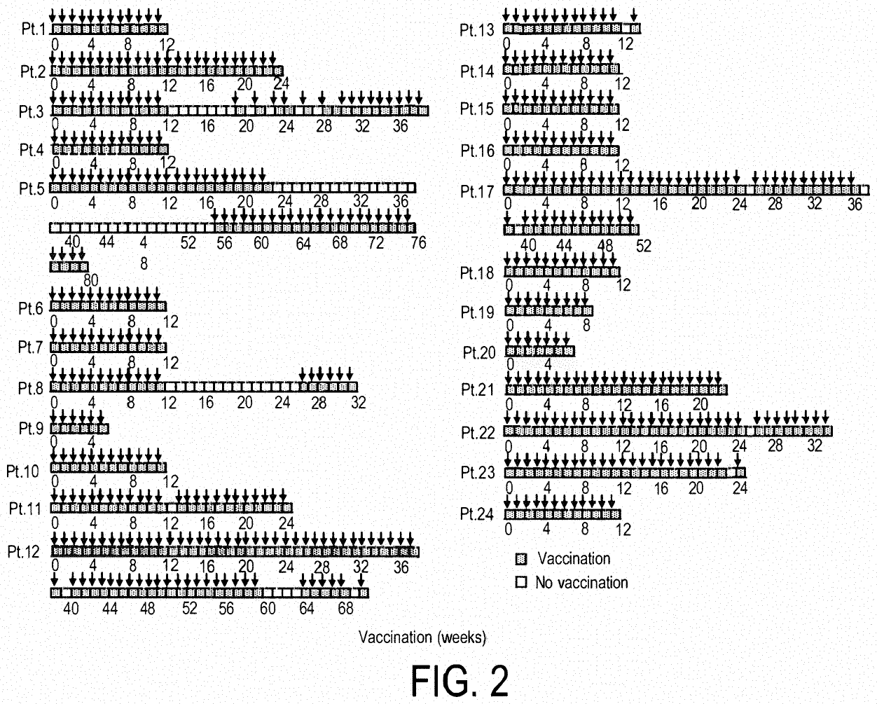

Patient characteristics, PPV administration, and response assessment. Previously reported was a stage IV NSCLC patient who experienced a remarkable regression of multiple lung tumors following PPV that was associated with CD8⁺ T-cell responses against the widely shared EGFR-L858R mutation (Li et al., 2016). Based on this case study, a phase Ib clinical trial of PPV for stage III/IV NSCLC patients was initiated to determine the safety and feasibility of PPV, with secondary endpoints being to assess potential clinical survival benefit in addition to the immunogenicity of personalized and shared NeoAgs (FIG. 1A). 24 patients (18 adenocarcinoma and 6 squamous cell carcinoma) were successfully enrolled and received 12 weekly immunizations with PPV followed by response evaluation by CT scans. Clinical responses were determined using RECIST criteria 3 to 4 months following the initiation of PPV. If desired, patients were given the option to continue PPV beyond 12 weeks; the immunization time courses of all 24 patients are shown in FIG. 2. Three of the patients (Pts. 1, 6, and 24) received alternative salvage therapy following disease progression after 12 weeks of immunization, and were taken off study. Five additional patients did not complete CT-based staging: three patients (Pts. 9, 19, and 20) expired from disease progression during the 12 weeks of vaccination, and two patients (Pts. 13 and 15) received follow-up at outside hospitals that were communicated to the authors. A complete summary of patient treatments and clinical outcomes is shown in FIG. 1B.

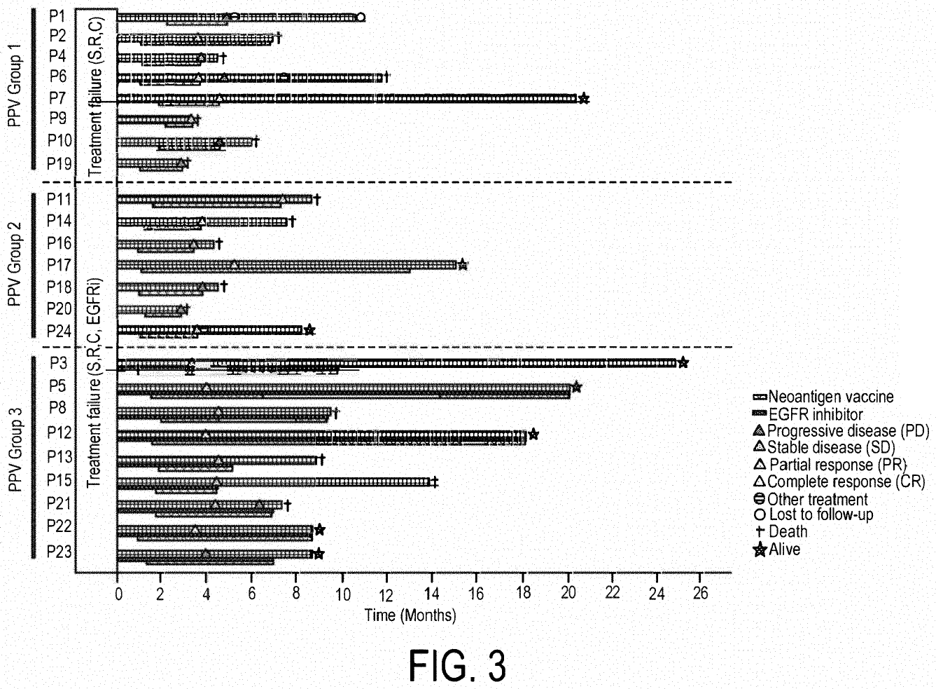

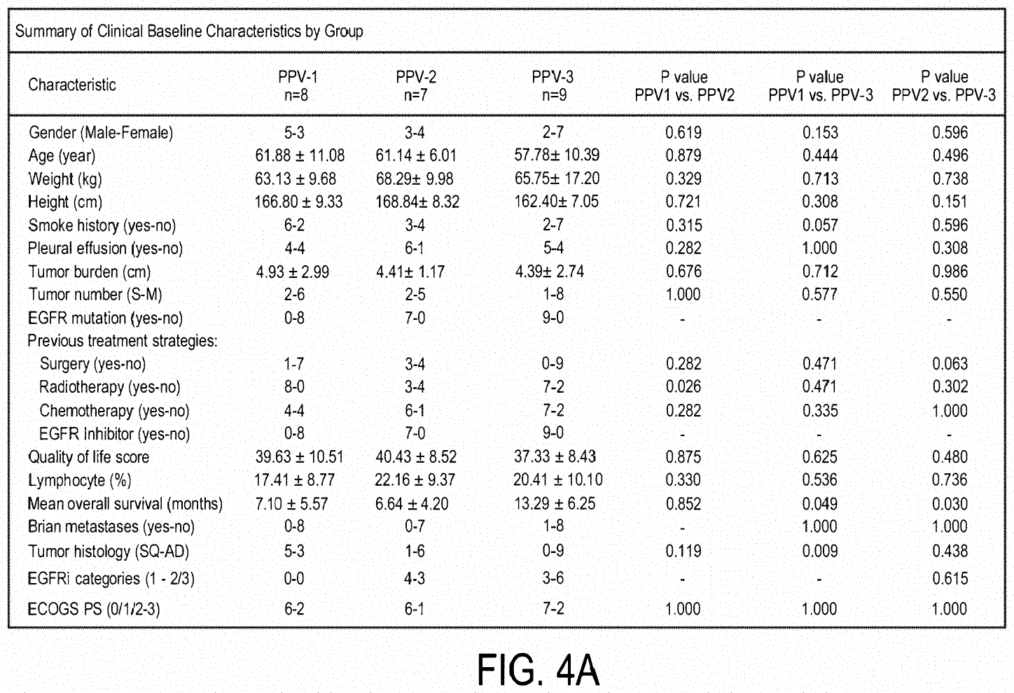

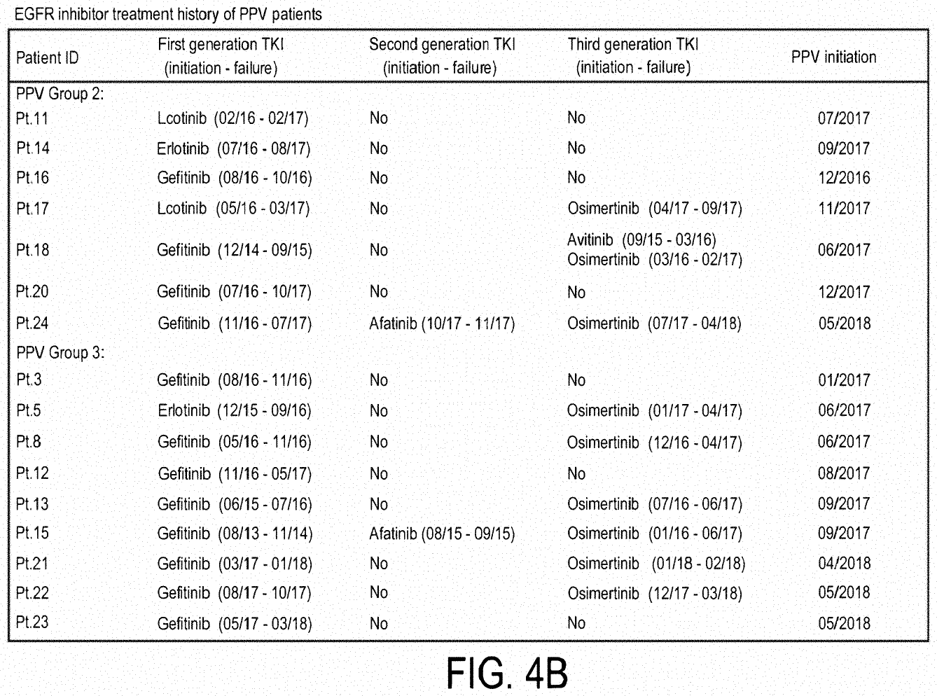

All 24 NSCLC patients had previously failed multiple lines of conventional therapy, including surgery, radiation therapy, and/or chemotherapy. Sixteen of the patients bearing EGFR-mutated tumors had also previously progressed on EGFRi therapy, with 9 of these patients failing 2 or more different EGFRi drugs. These 16 patients were given the option of either stopping or continuing EGFRi therapy concurrent with PPV immunization; 9 patients chose to continue receiving EGFRi and 7 patients opted to stop EGFRi therapy. Aside from these 9 patients, none of other 15 patients on the PPV trial received any other treatment during the follow-up period. Although patients were not initially randomized into separate cohorts, as shown below retrospective analyses revealed distinct response and survival profiles when the 24 patients were divided into the following 3 groups: EGFR wild-type (WT) patients receiving PPV only (Group 1), EGFR-mutated patients receiving PPV only (Group 2); and EGFR-mutated patients receiving PPV with concurrent EGFRi (Group 3) (FIG. 1C; FIG. 3). The clinical characteristics of the 3 patient groups did not differ significantly at baseline (FIG. 4A). Detailed EGFR inhibitor histories of Group 2 and 3 patients are shown in FIG. 4B.

Personalized neoantigen vaccine design. Large-scale whole exome sequencing studies have demonstrated that NSCLC has one of the highest mutational burdens of all cancer types, with individual patient tumors often expressing >100 or significantly more nonsynonymous somatic mutations (Rizva et al., 2015; Lawrence et al., 2013). Based on such profiles, current HLA class I peptide-binding prediction algorithms typically generate hundreds of potential NeoAg peptide candidates, many more than can be incorporated into a single multiepitope peptide vaccine.

However, most of these mutations are considered “branch” mutations expressed by tumor sub-clones but not by all tumor cells; these mutations are also less likely to constitute driver mutations essential for maintaining tumor survival (Hao et al., 2016). In order to focus on targeting shared driver mutations, we chose to perform mutational profiling on a panel of 508 known cancer-associated genes using DNA from needle-biopsied fresh tumor tissue (Table 4). Vaccine peptides were chosen primarily based on the highest predicted binding affinity of mutation-encoding NeoAgs to each patient's individual HLA class I and class II allotypes (Table 5, Methods). Each patient was immunized with a unique, personalized mixture of short and long NeoAg peptides dissolved in saline, divided into 2 pools and administered subcutaneously into opposite extremities. Imiquimod was applied topically after each immunization to provide co-stimulatory signals through Toll-like receptor (TLR)7 (FIG. 1A).

TABLE 4
508 cancer-associated gene panel.

| Pathways | Gene list |
|---|---|
| MAPK signal | KRAS NF NF2 MAP3K1 ARAF BRAF RAF1 NRAS HRAS MAP2K2 |
| | MAP2K4 MAPKSIP1 MAPK1 MAPK3 MAPK8 JUN ANGPT1 ANGPT2 |
| | DUSP6 FGF19 FGF23 IGF1R IGF1 IGF2 MAP3K13 NTRK2 MAP2K1 |
| | NTRK1 YES1 |
| PI3K signal | PIK3CA PIK3CB PIK3CG PTEN PIK3R1 PIK3C2A PIK3C2B PIK3C2G |
| | PIK3C3 TLR4 MTOR STK11 TSC1 TSC2 NAV3 AKT3 AKT1 AKT2 PDK1 |
| | PRKCA PRKCB PRKCG AXL FLCN HGF TMEM127 CREBBP ERCC1 |
| | RHOA NTRK2 MYB Rheb RPS6KB1 RPTOR PIK3R2 ITGA8 |
| STAT signal | JAK1 JAK2 JAK3 STAT4 STAT5B SOCS1 CBL IFNAR1 IFNAR2 CRLF2 |
| | ERCC1 CREBBP MPL PIK3R2 |
| TGF-β signal | SMAD4 TGFBR2 ACVR1B SMAD2 ACVR2A BMPR1A INHBA ITGB2 |
| | ELAC2 FNTA MED12 RICTOR CREBBP REL ROCK1 |
| Cell | CDKN2A RB1 CDK2 CDK4 CDK8 CDK6 CDK12 CCND1 CCND2 CCND3 |
| cycle/Apoptosis | CDKN1A CDKN1B CDKN2C CCNE1 CDKN2B MCL1 BCL2 BCL2L1 |
| | BCL2L2 AURKA AURKB CDC25C BAK1 BTG1 CASP8 CDC73 CFLAR |
| | CYLD EPCAM PMS1 PMS2 RAD50 RAD51 RAD51C RAD51D RAD52 |
| | RAD54L XIAP RPA1 NEK11 NPM1 PTP4A3 NOTCH1 POLE PLK1 |
| | PTPN13 CREBBP ERCC1 GSTP1 MEN1 NFKBIA NTRK1 REL RRM1 |
| | SLC19A1 TERT TYMS PIK3R2 |
| HH signal | SMO PTCH1 PTCH2 SPOP TACR1 AMER1 SUFU |
| APC signal | APC CTNNA1 CTNNB1 TBL1XR1 SOX17 SOX10 AXIN1 AXIN2 GSK3B |
| | APCDD1 |
| NOTCH signal | NOCTH1 NOCTH2 NOTCH3 NOTCH4 NR3C1 AMER1 CREBBP PAX5 |
| | REL SPEN |
| Transcription | VHL GATA1 GATA3 EP300 CTCF TAF1 TSHZ2 RUNX1 RUNX1T1 |
| regulators | MECOM TBX3 SIN3A EIF4A2 PHF6 CBFB SOX9 ELF3 VEZF1 CEBPA |
| | FOXA1 FOXA2 NKX2-1 ERG ETV1 ETV6 MYC NFE2L2 NFE2L3 MED12 |
| | SF3B1 U2AF1 SRSF2 PCBP1 BCL6 ARHGAP35 BCOR BCORL1 C11or30 |
| | DAXX DNMT1 EGR3 ESR1 EWSR1 FOXL2 FUBP1 HNF1A IKZF1 IRF4 |
| | MAX MEF2B CIC KLF4 NCOA1 NCOA2 NCOR1 TOP1 TOP2A TOP2B |
| Chromatin | HIST1H1C H3F3A H3F3C HIST1H2BD HIST1H3B MLL2 MLL3 MLL4 |
| modifications | ARID1A PBRM1 SETD2 NSD1 SETBP1 KDM5C KDM6A ARID5B |
| | ASXL1 EZH2 DNMT3A TET2 MLL SMARCA1 SMARCA4 SMARCB1 |
| | ARID2 ARID1B CHD1 CHD2 CHD4 HDAC1 HDAC2 HDAC3 HDAC4 |
| | HDAC6 HDAC8 EP300 DOT1L SPOP |
| DNA damage | TP53 ATM ATRX ATR STAG2 BAP1 BRCA1 BRCA2 SMC1A SMC3 |
| control | CHEK1 CHEK2 RAD21 ERCC2 MDM2 MSH2 MSH3 MSH4 MSH5 MSH6 |
| | MLH1 MLH3 BLM BRIP1 CUL4A CUL4B FANCA FANCC FANCD2 |
| | FANCG FANCI FANCM MRE11A MUTYH NBN PARP1 PARP2 PARP3 |
| | PARP4 XPC XRCC3 PALB2 DPYD ERCC1 PRDM1 REL RRM1 SLC19A1 |
| | SOD2 TMPRSS2 TYMS XRCC1 ZNF703 |
| RTK signal | EGFR FLT1 FLT4 FLT3 EPHA2 EPHA3 EPHA5 EPHB1 ERBB2 ERBB3 |
| | ERBB4 PDGFRA PDGFRB EPHB6 FGFR1 FGFR2 KIT FGFR3 FGFR4 |
| | MET ALK RET ROS1 CRKL VEGFA VEGFB ABL1 ABL2 AR DDR1 |
| | DDR2 FGF3 FGF4 FGF6 IRS2 KDR LCK LYN TEK CAMK2G ETV4 |
| | MAP2K1 NTRK1 NTRK3 PAK3 REL RICTOR SLC19A1 YES1 |
| Others | CSF1R WWP1 RNF43 GNA11 GNA13 GNAQ GNAS GNRHR GPR124 |
| | HRH2 TSHR LRRK2 PRKAA1 GRIN2A EML4 KIF5B TUBA1A TUBB |
| | TUBD1 TUBE1 TUBG1 FH RAC1 RAC2 RPL22 RPL5 TNFAIP3 |
| | TNFRSF14 TNFRSF8 TNFSF11 TNFSF13B IDH1 IDH2 CBR1 CYP17A1 |
| | FPGSALOX12B CROT SDHB SDHC SDHD PPP2R1A PTPN11 B4GALT3 |
| | EXT1 EXT2 BCR LIMK1 CRIPAK EPPK1 HSP90AA1 HSPA4 HSD3B2 |
| | PIGF PNRC1 POLQ PRKAR1A PSMB1 PSMB2 PSMB5 RARA RARB |
| | RARG ROBO1 ROBO2 SSTR2 NUP93 MALAT1 RNASEL SRC XPO1 |
| | AARS ABCB1 ABCC4 ALG10 ASPSCR1 ATF1 ATIC ATP1A1 ATP2B3 |
| | BCL9L C1QB C1QC C8orf34 CACNA1D NF1 CALR CBR3 CD22 CD274 |
| | CD33 CD3D CD3E CD3G CD52 CD80 CDA CHD8 CLTC CREB1 CTNND1 |
| | CYP19A1 CYP2C8 CYP2D6 CYP3A4 CYP3A5 DCK DOCK2 DOCK4 |
| | DROSHA EEF1A1 ELMO1 EPAS1 EPOR ESR2 FAT1 FAT3 FAT4 FBN2 |
| | FBXO11 FGD4 FUS GATA2 GNAI1 GSTA1 HLA-A NOTCH2 HSH2D |
| | IARS2 IL13RA2 IL2RA IL2RB IL2RG IL6ST JAZF1 KCNH2 PDE4DIP |
| | PRX CSMD3 HECTD4 PPP1R17 SH3PXD2A LRP1B MBTPS2 OTOA |
| | OR2T4 MITF OR4C6 OR5L2 AQP12A TUBGCP5 PDE1C BRD3 CDK13 |
| | SDK2 KMT2A KMT2B KMT2C KMT2D |

Among the 24 enrolled patients, a mean of 6.1 coding mutations were detected per tumor (range, 1 to 20). Of the 16 patients with EGFR-mutated tumors, 14 harbored common EGFR driver mutations (7 with L858R point mutations and 7 with Exon 19 deletions). 2 of which were accompanied by T790M mutations known to confer resistance to 1st generation EGFRi. The other 2 additional patients harbored the comparatively rare H773L mutation. The number of vaccine peptides administered ranged from 5 to 14 per patient (mean, 9.4), which was primarily determined by the number of NeoAgs predicted to bind to patient HLA class I (mean, 6.5 peptides) or HLA class II (mean=2.9 peptides). Patients in Groups 2 and 3 received a mean of 4 mutated EGFR peptides each (2.8 short and 1.2 long). The number of immunizing peptides or mean predicted peptide binding affinity did not differ significantly between groups; however, patients in Groups 2 and 3 received vaccines targeting less somatic mutations overall (FIGS. 1D-1F). This was due to the intentional targeting of individual EGFR mutations with multiple NeoAg peptides, an approach that had shown success for inducing the lung tumor regression in our initial case study patient (Li et al., 2016; Table 5).

TABLE 5
Personalized neoantigen peptides and predicted HLA binding affinity.
*Peptide binding affinities predicted by NetMHC4.0,
NetMHCpan4.0 (HLA class 1) and NetMHCII2.3 (HLA class II)
**Delta score = WT peptide affinity minus mutant peptide affinity

| | | | | | | | | Mutant | WT | |
|---|---|---|---|---|---|---|---|---|---|---|
| | | | | | | | | peptide | peptide | |
| | | | | | | | | binding | binding | Peptide |
| Pt | | | PFS | | | Neoantigen | HLA | affinity | affinity | Delta |
| ID | Cohort | Response | (months) | Gene | Mutation | peptide | allele | (nM)* | (nM)* | Score** |
| Pt.1 | 1 | PD | 4.9 | CSMD3 | S757R | 1. WTIISDP | DRB1* | 1935 | 1871 | −64 |
| | | | | | | GRRIHLS | 0901 | | | |
| | | | | | | END | | | | |
| | | | | | | (SEQ ID | | | | |
| | | | | | | NO: 794) | | | | |
| | | | | ROSI | 11530K | 2. STYMIQK | A*3201 | 1181 | 256 | −925 |
| | | | | | | AV | | | | |
| | | | | | | (SEQ ID | | | | |
| | | | | | | NO: 795) | | | | |
| | | | | | | 3. PFSTYMI | DRB1* | 317 | 337 | 29 |
| | | | | | | QKAVKNY | 0901 | | | |
| | | | | | | YSD | | | | |
| | | | | | | (SEQ ID | | | | |
| | | | | | | NO: 796) | | | | |
| | | | | TP53 | R248L | 4. MNLRPIL | B*5201 | 770 | 1514 | 744 |
| | | | | | | TI | | | | |
| | | | | | | (SEQ ID | | | | |
| | | | | | | NO: 797) | | | | |
| | | | | | | 5. MNLRPIL | B*3201 | 2313 | 5578 | 3265 |
| | | | | | | TII | | | | |
| | | | | | | (SEQ ID | | | | |
| | | | | | | NO: 798) | | | | |
| | | | | | | 6. SSCMGGM | DRB1* | 176 | 435 | 259 |
| | | | | | | NLRPILT | 0901 | | | |
| | | | | | | IIT | | | | |
| | | | | | | (SEQ ID | | | | |
| | | | | | | NO: 799) | | | | |
| | | | | CDKN2A | GHEV | 7. TLVVLHR | DRB1* | 174 | 180 | 6 |
| | | | | | | AVARLDV | 0901 | | | |
| | | | | | | RDA | | | | |
| | | | | | | (SEQ ID | | | | |
| | | | | | | NO: 800) | | | | |
| | | | | NF1 | T522A | 8. TQGSAAE | A*2402 | 2145 | 3036 | 881 |
| | | | | | | LI | | | | |
| | | | | | | (SEQ ID | | | | |
| | | | | | | NO: 801) | | | | |
| | | | | MLL3 | G292V | 9. RCAFCKH | DRB1* | 54 | 84 | 30 |
| | | | | | | LVATIKC | 0901 | | | |
| | | | | | | CEE | | | | |
| | | | | | | (SEQ ID | | | | |
| | | | | | | NO: 802) | | | | |
| | | | | GNAS | Q237H | 10. KWIHCFN | A*2402 | 1445 | 1129 | −316 |
| | | | | | | DV | | | | |
| | | | | | | (SEQ ID | | | | |
| | | | | | | NO: 803) | | | | |
| | | | | | | 11. HCFNDVT | A*3201 | 1321 | 1194 | −127 |
| | | | | | | AI | | | | |
| | | | | | | (SEQ ID | | | | |
| | | | | | | NO: 804) | | | | |
| | | | | NOTCH2 | L2224M | 12. TVLPSVS | DRBP1* | 184 | 269 | 85 |
| | | | | | | QMLSHHH | 0901 | | | |
| | | | | | | IVS | | | | |
| | | | | | | (SEQ ID | | | | |
| | | | | | | NO: 805) | | | | |
| Pt.2 | 1 | SD | 6.8 | IL6ST | D324Y | 1. EASGITY | A*0101 | 1163 | 30783 | 29620 |
| | | | | | | EY | | | | |
| | | | | | | (SEQ ID | | | | |
| | | | | | | NO: 806) | | | | |
| | | | | | | 2. ITYEYRP | A*3001 | 22 | 69 | 38 |
| | | | | | | SK | | | | |
| | | | | | | (SEQ ID | | | | |
| | | | | | | NO: 807) | | | | |
| | | | | | | 3. GITYEYR | A*3001 | 197 | 739 | 542 |
| | | | | | | PSK | | | | |
| | | | | | | (SEQ ID | | | | |
| | | | | | | NO: 808) | | | | |
| | | | | | | 4. ITYEYRP | A*3001 | 359 | 1182 | 823 |
| | | | | | | SKA | | | | |
| | | | | | | (SEQ ID | | | | |
| | | | | | | NO: 809) | | | | |
| | | | | | | 5. YEYRPSK | B*1301 | 899 | 4456 | 3557 |
| | | | | | | APSF | | | | |
| | | | | | | (SEQ ID | | | | |
| | | | | | | NO: 810) | | | | |
| | | | | | | 6. EASGITY | DRB1* | 292 | 7479 | 7187 |
| | | | | | | EYRPSKA | 0701 | | | |
| | | | | | | PSF | | | | |
| | | | | | | (SEQ ID | | | | |
| | | | | | | NO: 811) | | | | |
| Pt.3 | 3 | SD | 24.8 | MAPZK4 | 1367T | 1. LLKHPFT | A*3001 | 2262 | 513 | −1749 |
| | | | | | | LM | | | | |
| | | | | | | (SEQ ID | | | | |
| | | | | | | NO: 812) | | | | |
| | | | | | | 2. KELLKHP | A*0201 | 60 | 166 | 106 |
| | | | | | | FTL | | | | |
| | | | | | | (SEQ ID | | | | |
| | | | | | | NO: 813) | | | | |
| | | | | | | 3. FTLMYEE | A*0201 | 76 | 76 | 0 |
| | | | | | | RAV | | | | |
| | | | | | | (SEQ ID | | | | |
| | | | | | | NO: 814) | | | | |
| | | | | | | 4. KELLKHP | DRB1* | 390 | 630 | 240 |
| | | | | | | FTLMYEE | 0701 | | | |
| | | | | | | RAV | | | | |
| | | | | | | (SEQ ID | | | | |
| | | | | | | NO: 815) | | | | |
| | | | | | | 7. LKHPTLM | C*1202 | 785 | NA | NA |
| | | | | | | Y | | | | |
| | | | | | | (SEQ ID | | | | |
| | | | | | | NO: 816) | | | | |
| | | | | EGFR | 746_ | 5. AIKTSPK | A*3001 | 1047 | NA | NA |
| | | | | | 750del | ANK | | | | |
| | | | | | | (SEQ ID | | | | |
| | | | | | | NO: 20) | | | | |
| | | | | | | 6. VKIPVAI | DRB1* | 2587 | 785 | −1802 |
| | | | | | | KTSPKAN | 0701 | | | |
| | | | | | | KEI | | | | |
| | | | | | | (SEQ ID | | | | |
| | | | | | | NO: 817) | | | | |
| Pt.4 | 1 | PD | 2 | TP53 | L330H | 1. GEYFTHQ | B*4001 | 477 | 825 | 348 |
| | | | | | | IR | | | | |
| | | | | | | (SEQ ID | | | | |
| | | | | | | NO: 818) | | | | |
| | | | | | | 2. LDGEYFT | DRB1* | 612 | 632 | 20 |
| | | | | | | HQIRGRE | 0901 | | | |
| | | | | | | RFE | | | | |
| | | | | | | (SEQ ID | | | | |
| | | | | | | NO: 819) | | | | |
| | | | | SH3PXD2 | R119Gfs* | 3. DEVFGSS | B*4001 | 1098 | NA | NA |
| | | | | | 38 | RL | | | | |
| | | | | | | (SEQ ID | | | | |
| | | | | | | NO: 820) | | | | |
| | | | | A | | | | | | |
| | | | | | | 4. RLDPRMS | A*1101 | 656 | NA | NA |
| | | | | | | TLQK | | | | |
| | | | | | | (SEQ ID | | | | |
| | | | | | | NO: | | | | |
| | | | | | | 821 | | | | |
| | | | | | | 5. RGNQCGC | DQB1* | 2569 | NA | NA |
| | | | | | | PAGLSRP | 0301 | | | |
| | | | | | | RRT | | | | |
| | | | | | | (SEQ ID | | | | |
| | | | | | | NO: 822) | | | | |
| | | | | ATM | S2408L | 6. LEFENKQ | B*4001 | 9 | 9 | 0 |
| | | | | | | AL | | | | |
| | | | | | | (SEQ ID | | | | |
| | | | | | | NO: 823) | | | | |
| | | | | | | 7. YMKSLEF | A*1101 | 544 | 385 | −159 |
| | | | | | | ENK | | | | |
| | | | | | | (SEQ ID | | | | |
| | | | | | | NO: 824) | | | | |
| | | | | | | 8. SLEFENK | B*4001 | 80 | 82 | 2 |
| | | | | | | QAL | | | | |
| | | | | | | (SEQ ID | | | | |
| | | | | | | NO: 825) | | | | |
| Pt.5 | 3 | PR | 20 | EGFR | L858R | 1. KITDFGR | A*1101 | 163 | 20 | −143 |
| | | | | | | AK | | | | |
| | | | | | | (SEQ ID | | | | |
| | | | | | | NO: 4) | | | | |
| | | | | | | 2. TDFGRAK | C*0602 | 624 | 4139 | 3515 |
| | | | | | | LL | | | | |
| | | | | | | (SEQ ID | | | | |
| | | | | | | NO: 15) | | | | |
| | | | | | | 3. VKITDFG | A*1101 | 644 | 54 | −590 |
| | | | | | | RAK | | | | |
| | | | | | | (SEQ ID | | | | |
| | | | | | | NO: 16) | | | | |
| | | | | | | 4. ITDFGRA | C*0102 | J 826 | 2440 | 614 |
| | | | | | | KL | | | | |
| | | | | | | (SEQ ID | | | | |
| | | | | | | NO: 3) | | | | |
| | | | | | | 5. HVKITDF | DRB1* | 1591 | 945 | −646 |
| | | | | | | GRAKLLG | 0701 | | | |
| | | | | | | AEE | | | | |
| | | | | | | (SEQ ID | | | | |
| | | | | | | NO: 826) | | | | |
| Pt.6 | 1 | SD | 3.9 | TP53 | R248L/ | 1. SSCMGGM | A*1101 | 56 | 170 | 113 |
| | | | | | Q33IE | NLR | | | | |
| | | | | | | (SEQ ID | | | | |
| | | | | | | NO: 827) | | | | |
| | | | | | | 2. SSCMGGM | DRB1* | 301 | 1714 | 1413 |
| | | | | | | NLRPILT | 0101 | | | |
| | | | | | | IIT | | | | |
| | | | | | | (SEQ ID | | | | |
| | | | | | | NO: 799) | | | | |
| | | | | | | 3. LDGEYFT | DRB1* | 1491 | 785 | −706 |
| | | | | | | LEIRGRE | 0101 | | | |
| | | | | | | RFE | | | | |
| | | | | | | (SEQ ID | | | | |
| | | | | | | NO: 828) | | | | |
| | | | | FLT3 | A425D | 4. IFHAEND | DQB1* | 3178 | 7742 | 4564 |
| | | | | | | DDQFTKM | 0501 | | | |
| | | | | | | FTL | | | | |
| | | | | | | (SEQ ID | | | | |
| | | | | | | NO: 829) | | | | |
| | | | | EPHA3 | | 5. FEIRART | B*4002 | 144 | 9273 | 9129 |
| | | | | | | AA | | | | |
| | | | | | | (SEQ ID | | | | |
| | | | | | | NO: 830) | | | | |
| | | | | | | 6. TIYVFEI | A*1101 | 373 | 206 | −167 |
| | | | | | | RAR | | | | |
| | | | | | | (SEQ ID | | | | |
| | | | | | | NO: 831) | | | | |
| | | | | | | 7. KPDTIYV | DRB1* | 175 | 95 | −80 |
| | | | | | | FEIRART | 0101 | | | |
| | | | | | | AAG | | | | |
| | | | | | | (SEQ ID | | | | |
| | | | | | | NO: 832) | | | | |
| | | | | JAK2 | GI80W | 9. QEECLWM | B*4002 | 172 | 355 | 183 |
| | | | | | | AV | | | | |
| | | | | | | (SEQ ID | | | | |
| | | | | | | NO: 833) | | | | |
| | | | | | | 10. EECLWMA | B*4002 | 215 | 120 | −95 |
| | | | | | | VL | | | | |
| | | | | | | (SEQ ID | | | | |
| | | | | | | NO: 834) | | | | |
| Pt.7 | 1 | SD | 20.3 | TP53 | R267G | 1. LLGGNSF | A*0206 | 26 | 70 | 44 |
| | | | | | | EV | | | | |
| | | | | | | (SEQ ID | | | | |
| | | | | | | NO: 835) | | | | |
| | | | | | | 2. NLLGGNS | A*3301 | 308 | 121 | −187 |
| | | | | | | FEVR | | | | |
| | | | | | | (SEQ ID | | | | |
| | | | | | | NO: 836) | | | | |
| | | | | | | 3. TLEDSSG | DRB1* | 586 | 1688 | 1103 |
| | | | | | | NLLGGNS | 0101 | | | |
| | | | | | | FEVRVC | | | | |
| | | | | | | (SEQ ID | | | | |
| | | | | | | NO: 837) | | | | |
| | | | | ROCK1 | N163K | 4. YMPGGDL | A*0206 | 93 | 50 | −43 |
| | | | | | | VKL | | | | |
| | | | | | | (SEQ ID | | | | |
| | | | | | | NO: 838) | | | | |
| | | | | | | 5. EYMPGGD | DRB1* | 20 | 37 | 17 |
| | | | | | | LVKLMSN | 0101 | | | |
| | | | | | | YDVPKK | | | | |
| | | | | | | (SEQ ID | | | | |
| | | | | | | NO: 839) | | | | |
| | | | | MLL3 | R45440 | 6. GEQDHTF | 13*4403 | 1829 | 3676 | 1847 |
| | | | | | | RV | | | | |
| | | | | | | (SEQ ID | | | | |
| | | | | | | NO: 840) | | | | |
| | | | | IDH2 | 1269M | 7. RFKDIFQ | CM 402 | 130 | 1364 | 1234 |
| | | | | | | EM | | | | |
| | | | | | | (SEQ ID | | | | |
| | | | | | | NO: 841 | | | | |
| | | | | | | 8. IFQEMFD | A*3301 | 2106 | 1306 | −800 |
| | | | | | | KHYK | | | | |
| | | | | | | (SEQ ID | | | | |
| | | | | | | NO: 842) | | | | |
| | | | | PIK3CA | E545K | 9. SEITKQE | B*4403 | 503 | 310 | −193 |
| | | | | | | KDF | | | | |
| | | | | | | (SEQ ID | | | | |
| | | | | | | NO: 843) | | | | |
| | | | | NOTCH4 | E1532Q | 10. AEVDQDG | B*4403 | 681 | 843 | 162 |
| | | | | | | VVM | | | | |
| | | | | | | (SEQ ID | | | | |
| | | | | | | NO: 844) | | | | |
| | | | | | | 11. ALKPKAE | DQB1* | 227 | 244 | 17 |
| | | | | | | VDQDGVV | 0602 | | | |
| | | | | | | MCSGPE | | | | |
| | | | | | | (SEQ ID | | | | |
| | | | | | | NO: 845) | | | | |
| | | | | PTEN | R130G | 12. AAIHCKA | DQB1*0602 | 39 | 90 | 51 |
| | | | | | | GKGGTGV | | | | |
| | | | | | | MICAYL | | | | |
| | | | | | | (SEQ ID | | | | |
| | | | | | | NO: 846) | | | | |
| Pt.8 | 3 | PR | 9.5 | EGFR | L8SSR | 1. KITDPGR | A*1101 | 163 | 20 | −144 |
| | | | | | | AK | | | | |
| | | | | | | (SEQ ID | | | | |
| | | | | | | NO: 4) | | | | |
| | | | | | | 2. FGRAKLL | B*5401 | 3471 | 2163 | −1308 |
| | | | | | | GA | | | | |
| | | | | | | (SEQ ID | | | | |
| | | | | | | NO: 6) | | | | |
| | | | | | | 3. VKITDFG | AO 101 | 644 | 53 | −591 |
| | | | | | | RAK | | | | |
| | | | | | | (SEQ ID | | | | |
| | | | | | | NO: 16) | | | | |
| | | | | | | 4. HVKITDF | A*1101 | 4197 | 412 | −3785 |
| | | | | | | GRAK | | | | |
| | | | | | | (SEQ ID | | | | |
| | | | | | | NO: 9) | | | | |
| | | | | | | 5. HVKITDF | DQB1* | 1291 | 926 | −366 |
| | | | | | | GRAKLLG | 0301 | | | |
| | | | | | | AEE | | | | |
| | | | | | | (SEQ ID | | | | |
| | | | | | | NO: 826) | | | | |
| | | | | TP33 | P152L/ | 6. QLWVDST | B*3901 | 3724 | 26389 | 22665 |
| | | | | | M2371 | PL | | | | |
| | | | | | | (SEQ ID | | | | |
| | | | | | | NO: 847) | | | | |
| | | | | | | 7. STPLPGT | A*1101 | 1192 | 2375 | 183 |
| | | | | | | RVR | | | | |
| | | | | | | (SEQ ID | | | | |
| | | | | | | NO: 848) | | | | |
| | | | | | | 8. TPLPGTR | B*5401 | 598 | 4908 | 4310 |
| | | | | | | VRA | | | | |
| | | | | | | (SEQ ID | | | | |
| | | | | | | NO: 849) | | | | |
| | | | | | | 9. QLWVDST | DQB1* | 819 | 2271 | 1452 |
| | | | | | | PLPGTRV | 0301 | | | |
| | | | | | | RAM | | | | |
| | | | | | | (SEQ ID | | | | |
| | | | | | | NO: 850) | | | | |
| | | | | | | 10. NYICNSS | C*0702 | 1242 | 381 | −861 |
| | | | | | | CM | | | | |
| | | | | | | (SEQ ID | | | | |
| | | | | | | NO: 851) | | | | |
| | | | | | | 11. CTTIHYN | DRB1* | 1409 | 1719 | 310 |
| | | | | | | YICNSSC | 1501 | | | |
| | | | | | | MGG | | | | |
| | | | | | | (SEQ ID | | | | |
| | | | | | | NO: 852) | | | | |
| Pt.9 | 1 | SD | 3.4 | NTRK1 | Q500P | 1. LPEPDKM | B*5101 | 8862 | 12804 | 3942 |
| | | | | | | LVAV | | | | |
| | | | | | | (SEQ ID | | | | |
| | | | | | | NO: 853) | | | | |
| | | | | MBTPS2 | R18IS | 2. IAAISEQ | C*0303 | 22 | 38 | 16 |
| | | | | | | VRF | | | | |
| | | | | | | (SEQ ID | | | | |
| | | | | | | NO: 854) | | | | |
| | | | | SMARCA4 | R1135L | 3. KAEDLGM | A*1101 | 151 | 177 | 26 |
| | | | | | | LLK | | | | |
| | | | | | | (SEQ ID | | | | |
| | | | | | | NO: 855) | | | | |
| | | | | OTOA | D502Y | 4. YTAPGIV | C*0303 | 11 | 4619 | 4599 |
| | | | | | | EI | | | | |
| | | | | | | (SEQ ID | | | | |
| | | | | | | NO: 856) | | | | |
| | | | | IRS2 | D345Y | 5. LVRRSRT | B*0702 | 54 | 54 | 0 |
| | | | | | | YSL | | | | |
| | | | | | | (SEQ ID | | | | |
| | | | | | | NO: 857) | | | | |
| | | | | | | 6. SQTGLVR | DRB1* | 188 | 2874 | 2686 |
| | | | | | | RSRTYSL | 1501 | | | |
| | | | | | | AATP | | | | |
| | | | | | | (SEQ ID | | | | |
| | | | | | | NO: 858) | | | | |
| | | | | TP53 | V2161 | 7. FRHSLVV | C*0702 | 40 | 31 | −9 |
| | | | | | | PY | | | | |
| | | | | | | (SEQ ID | | | | |
| | | | | | | NO: 859) | | | | |
| | | | | MAP2K4 | Q327L | 8. QVVKGDP | C*0303 | 129 | 16462 | 16333 |
| | | | | | | PL | | | | |
| | | | | | | (SEQ ID | | | | |
| | | | | | | NO: 860) | | | | |
| | | | | PIKSR2 | A312S | 9. KPPKAKP | B*0702 | 6512 | 7562 | 1050 |
| | | | | | | SPTY | | | | |
| | | | | | | (SEQ ID | | | | |
| | | | | | | NO: 861) | | | | |
| | | | | OR2T4 | F198L | 10. TPITMTL | B*0702 | 34 | 67 | 33 |
| | | | | | | PF | | | | |
| | | | | | | (SEQ ID | | | | |
| | | | | | | NO: 862) | | | | |
| | | | | JAK2 | R588Kfs* | 11. EVLLKVL | DRB1* | 163 | NA | NA |
| | | | | | 8 | DKAHKLF | 1501 | | | |
| | | | | | | RVFL | | | | |
| | | | | | | (SEQ ID | | | | |
| | | | | | | NO: 863) | | | | |
| | | | | MITF | E38D | 12. SSADHPG | A*1101 | 49 | 74 | 25 |
| | | | | | | ASK | | | | |
| | | | | | | (SEQ ID | | | | |
| | | | | | | NO: 864) | | | | |
| | | | | EPHA5 | P734S | 13. SIMGQFD | DQB1* | 91 | 512 | 421 |
| | | | | | | HSNIIHL | 0602 | | | |
| | | | | | | EGVV | | | | |
| | | | | | | (SEQ ID | | | | |
| | | | | | | NO: 865) | | | | |
| Pt.10 | 1 | PD | 2.7 | MED12 | L1417H | 1. KTKPVHI | B*1527 | 1560 | 1539 | −21 |
| | | | | | | SSL | | | | |
| | | | | | | (SEQ ID | | | | |
| | | | | | | NO: 866) | | | | |
| | | | | AR | G292D | 2. LAECKDS | C*0801 | 2633 | 1460 | −1173 |
| | | | | | | LL | | | | |
| | | | | | | (SEQ ID | | | | |
| | | | | | | NO: 867) | | | | |
| | | | | OR5L2 | G108V | 3. CVVTEVF | A*0206 | 113 | 5884 | 5771 |
| | | | | | | LL | | | | |
| | | | | | | (SEQ ID | | | | |
| | | | | | | NO: 868) | | | | |
| | | | | | | 4. VVTEVFL | A*0206 | 30 | 25 | −5 |
| | | | | | | LA | | | | |
| | | | | | | (SEQ ID | | | | |
| | | | | | | NO: 869) | | | | |
| | | | | ITGA8 | Q786H | 5. QINITAV | A*0206 | 244 | 469 | 225 |
| | | | | | | AHV | | | | |
| | | | | | | (SEQ ID | | | | |
| | | | | | | NO: 870) | | | | |
| | | | | | | 6. QINITAV | DQB1* | 2821 | 1547 | −1274 |
| | | | | | | AHVEIRG | 0302 | | | |
| | | | | | | LEP | | | | |
| | | | | | | (SEQ ID | | | | |
| | | | | | | NO: 871) | | | | |
| | | | | NTRK3 | R130H | 7. FAKNPHL | 13*1527 | 87 | 210 | 123 |
| | | | | | | HY | | | | |
| | | | | | | (SEQ ID | | | | |
| | | | | | | NO: 872) | | | | |
| | | | | | | 8. FAKNPHL | A*0206 | 92 | 1137 | 1045 |
| | | | | | | HYI | | | | |
| | | | | | | (SEQ ID | | | | |
| | | | | | | NO: 873) | | | | |
| Pt.11 | 2 | PR | 8.6 | AQP12A | L28R | L. RLPVGAY | ANDIE | 33 | 22 | −11 |
| | | | | | | EV | | | | |
| | | | | | | (SEQ ID | | | | |
| | | | | | | NO: 874) | | | | |
| | | | | | | 2. KARLPVG | *030 | 2253 | 2418 | 165 |
| | | | | | | AY | | | | |
| | | | | | | (SEQ ID | | | | |
| | | | | | | NO: 875 ) | | | | |
| | | | | | | 3. RASKARL | B*0801 | 2949 | 6018 | 3069 |
| | | | | | | PV | | | | |
| | | | | | | (SEQ ID | | | | |
| | | | | | | NO: 876) | | | | |
| | | | | | | 4. RRASKAR | C*0602 | 991 | 4154 | 3163 |
| | | | | | | LP | | | | |
| | | | | | | (SEQ ID | | | | |
| | | | | | | NO: 877) | | | | |
| | | | | | | 5. ARRASKA | C*0702 | 2315 | 2688 | 373 |
| | | | | | | RL | | | | |
| | | | | | | (SEQ ID | | | | |
| | | | | | | NO: 878) | | | | |
| | | | | | | 6. ARLPVGA | A*0201 | 308 | 14 | −294 |
| | | | | | | YEV | | | | |
| | | | | | | (SEQ ID | | | | |
| | | | | | | NO: 879) | | | | |
| | | | | EGFR | H773L | 7. VMASVDN | A*0201 | 39 | 21040 | 21010 |
| | | | | | | PL | | | | |
| | | | | | | (SEQ ID | | | | |
| | | | | | | NO: 880) | | | | |
| Pt.12 | 3 | PR | 18 | EGFR | L858R | 1. TDFGRAK | B*3701 | 766 | 1 144 | 378 |
| | | | | | | LL | | | | |
| | | | | | | (SEQ ID | | | | |
| | | | | | | NO: 15) | | | | |
| | | | | | | 2. HVKITDF | DRB1* | 276 | 152 | −124 |
| | | | | | | GRAKLLG | 0901 | | | |
| | | | | | | AEE | | | | |
| | | | | | | (SEQ ID | | | | |
| | | | | | | NO: 826) | | | | |
| | | | | TUBGCP | V784L | 3. RLSISFE | A*0201 | 742 | 194 | −548 |
| | | | | 5 | | NL | | | | |
| | | | | | | (SEQ ID | | | | |
| | | | | | | NO: 881) | | | | |
| | | | | | | 4. ISFENLD | C*0602 | 7987 | 4390 | −3597 |
| | | | | | | TA | | | | |
| | | | | | | (SEQ ID | | | | |
| | | | | | | NO: 882) | | | | |
| | | | | | | 5. SISFENL | A*0201 | 1547 | 2694 | 1147 |
| | | | | | | DTA | | | | |
| | | | | | | (SEQ ID | | | | |
| | | | | | | NO: 883) | | | | |
| | | | | | | 6. RLSISFE | DRB1* | 1099 | 1556 | 457 |
| | | | | | | NLDTAKK | 0901 | | | |
| | | | | | | KLP | | | | |
| | | | | | | (SEQ ID | | | | |
| | | | | | | NO: 884) | | | | |
| Pt.13 | 3 | SD | 8.9 | ALK | R48U/ | L. EPLSYSL | A*3301 | 440 | 469 | 29 |
| | | | | | M1615L | LQR | | | | |
| | | | | | | (SEQ ID | | | | |
| | | | | | | NO: 885) | | | | |
| | | | | | | 2. GHYEDTI | DRB1* | 344 | 568 | 224 |
| | | | | | | LKSKNSL | 1302 | | | |
| | | | | | | NOPG | | | | |
| | | | | | | (SEQ ID | | | | |
| | | | | | | NO: 886) | | | | |
| | | | | EGFR | 745_750 | 3. HPLCGYH | B*0702 | 1299 | NA | NA |
| | | | | | del | EQV | | | | |
| | | | | | | (SEQ ID | | | | |
| | | | | | | NO: 887) | | | | |
| | | | | MTOR | Cl4835/ | 4. GRMRSLE | B*3901 | 163 | 377 | 214 |
| | | | | | 1218 | AL | | | | |
| | | | | | 5H | (SEQ ID | | | | |
| | | | | | | NO: 888) | | | | |
| | | | | | | 5. ELMLGRM | DQB1* | 192 | 150 | −42 |
| | | | | | | RSLEALG | 0602 | | | |
| | | | | | | EWGQ | | | | |
| | | | | | | (SEQ ID | | | | |
| | | | | | | NO: 889) | | | | |
| | | | | | | 6. FHLKGHE | B*3901 | 75 | 158 | 83 |
| | | | | | | DL | | | | |
| | | | | | | (SEQ ID | | | | |
| | | | | | | NO: 890) | | | | |
| | | PR | | RET | E805fs*64 | 7. HRGCSIW | A*3301 | 117 | NA | NA |
| | | | | | | PR | | | | |
| | | | | | | (SEQ ID | | | | |
| | | | | | | NO: 891) | | | | |
| | | | | | | 8. SPWATSS | B*0702 | 56 | NA | NA |
| | | | | | | HL | | | | |
| | | | | | | (SEQ ID | | | | |
| | | | | | | NO: 892) | | | | |
| | | | | | | 9. DGPLLLI | DRB1* | 33 | NA | NA |
| | | | | | | VSTPNTA | 1302 | | | |
| | | | | | | PCGA | | | | |
| | | | | | | (SEQ ID | | | | |
| | | | | | | NO: 893) | | | | |
| | | | | PTEN | Q2701/ | 10. VAQYPFE | DRB1* | 648 | 2210 | 1562 |
| | | | | | H549L | DHNPPLL | 1302 | | | |
| | | | | | | ELIK | | | | |
| | | | | | | (SEQ ID | | | | |
| | | | | | | NO: 894) | | | | |
| | | | | | | 11. DVSDNEP | A*3301 | 783 | 460 | −323 |
| | | | | | | DLYR | | | | |
| | | | | | | (SEQ ID | | | | |
| | | | | | | NO: 895) | | | | |
| | | | | ROSI | E1787fs*8 | 12. YYILDKK | A*3301 | 1634 | NA | NA |
| | | | | | | EHFK | | | | |
| | | | | | | (SEQ ID | | | | |
| | | | | | | NO: 896) | | | | |
| Pt.14 | 2 | | 7.6 | EGER | L858R/ | 1. KITDFGR | A*1101 | 163 | 20 | −143 |
| | | | | | E709V | AK | | | | |
| | | | | | | (SEQ ID | | | | |
| | | | | | | NO: 4) | | | | |
| | | | | | | 2. HVKITDF | A*3101 | 10 | 5090 | 5080 |
| | | | | | | GR | | | | |
| | | | | | | (SEQ ID | | | | |
| | | | | | | NO: 1 | | | | |
| | | | | | | 3. VKITDFG | AG 101 | 644 | 54 | −590 |
| | | | | | | RAK | | | | |
| | | | | | | (SEQ ID | | | | |
| | | | | | | NO: 16) | | | | |
| | | | | | | 4. HVKITDF | DQB1*0301 | 1215 | 925 | −290 |
| | | | | | | GRAKLLG | | | | |
| | | | | | | AEE | | | | |
| | | | | | | (SEQ ID | | | | |
| | | | | | | NO: 826) | | | | |
| | | | | | | S. RILKVTE | A*1101 | 12 | 24 | 12 |
| | | | | | | FK | | | | |
| | | | | | | (SEQ ID | | | | |
| | | | | | | NO: 897) | | | | |
| | | | | | | 6. RILKVTE | A*1101 | 24 | 54 | 30 |
| | | | | | | FKK | | | | |
| | | | | | | (SEQ ID | | | | |
| | | | | | | NO: 898) | | | | |
| | | | | | | 7. LRILKVT | A*1101 | 62 | 155 | 93 |
| | | | | | | EFK | | | | |
| | | | | | | (SEQ ID | | | | |
| | | | | | | NO: 899) | | | | |
| | | | | | | 8. QALLRIL | DRB1* | 740 | 1839 | 1099 |
| | | | | | | KVTEFKK | 0901 | | | |
| | | | | | | IKV | | | | |
| | | | | | | (SEQ ID | | | | |
| | | | | | | NO: 900) | | | | |
| | | | | BRCA2 | 12840V | 9. TSSGLYV | A*3101 | 10 | 12 | 2 |
| | | | | | | FR | | | | |
| | | | | | | (SEQ ID | | | | |
| | | | | | | NO: 901) | | | | |
| | | | | | | 10. GLYVFRN | A*3101 | 16 | 20 | 4 |
| | | | | | | ER | | | | |
| | | | | | | (SEQ ID | | | | |
| | | | | | | NO: 902) | | | | |
| | | | | | | 11. LKTSSGL | A*3101 | 8 | 9 | 1 |
| | | | | | | YVFR | | | | |
| | | | | | | (SEQ ID | | | | |
| | | | | | | NO: 903) | | | | |
| | | | | | | 12. SGLYVFR | A *3101 | 42 | 48 | 6 |
| | | | | | | NER | | | | |
| | | | | | | (SEQ ID | | | | |
| | | | | | | NO: 904) | | | | |
| | | | | | | 13. EKTSSGL | A*3101 | 21 | 27 | 6 |
| | | | | | | YVFR | | | | |
| | | | | | | (SEQ ID | | | | |
| | | | | | | NO: | | | | |
| | | | | | | 905) | | | | |
| | | | | | | 14. EKTSSGL | DQB1* | 421 | 745 | 324 |
| | | | | | | YVERNER | 0301 | | | |
| | | | | | | EEE | | | | |
| | | | | | | (SEQ ID | | | | |
| | | | | | | NO: 906) | | | | |
| Pt.15 | 3 | SD | 13.8 | EGER | T790M/ | 1. QLIMQLM | A*0206 | 116 | 62 | −54 |
| | | | | | E746_ | PF | | | | |
| | | | | | A750del | (SEQ ID | | | | |
| | | | | | | NO: 62) | | | | |
| | | | | | | 2. LTSTVQL | B*3501 | 1274 | 18568 | 17294 |
| | | | | | | IM | | | | |
| | | | | | | (SEQ ID | | | | |
| | | | | | | NO: 65) | | | | |
| | | | | | | 3. MQLMPFG | A*0206 | 23 | 79 | 56 |
| | | | | | | CL | | | | |
| | | | | | | (SEQ ID | | | | |
| | | | | | | NO: 61) | | | | |
| | | | | | | 4. VQLIMQL | A*0206 | 244 | 134 | −no |
| | | | | | | MPF | | | | |
| | | | | | | (SEQ ID | | | | |
| | | | | | | NO: 591 | | | | |
| | | | | | | 5. LTSTVQL | DQB1* | 288 | 299 | 11 |
| | | | | | | IMQLMPF | 0602 | | | |
| | | | | | | GCL | | | | |
| | | | | | | (SEQ ID | | | | |
| | | | | | | NO: 907) | | | | |
| | | | | | | 6. IPVAIKT | B*3501 | 2442 | NA | NA |
| | | | | | | SP | | | | |
| | | | | | | (SEQ ID | | | | |
| | | | | | | NO: 27) | | | | |
| | | | | | | 7. IPVAIKT | A*1101 | 130 | NA | NA |
| | | | | | | SPK | | | | |
| | | | | | | (SEQ ID | | | | |
| | | | | | | NO: 19) | | | | |
| | | | | | | 8. VKIPVAI | DRB1* | 678 | NA | NA |
| | | | | | | KTSPKAN | 1501 | | | |
| | | | | | | KEI | | | | |
| | | | | | | (SEQ ID | | | | |
| | | | | | | NO: 817) | | | | |
| | | | | BRD3 | E709K | 9. RLSSSSS | A*101 | 52 | 17001 | 16949 |
| | | | | | | SK | | | | |
| | | | | | | (SEQ ID | | | | |
| | | | | | | NO: 908) | | | | |
| | | | | CDK13 | A1177P | 10. AQPAVQS | A*0206 | 579 | 236 | −343 |
| | | | | | | AF | | | | |
| | | | | | | (SEQ ID | | | | |
| | | | | | | NO: 909) | | | | |
| | | | | | | 11. LVETDAA | B*4001 | 267 | 361 | 94 |
| | | | | | | QPAV | | | | |
| | | | | | | (SEQ ID | | | | |
| | | | | | | NO: 910) | | | | |
| | | | | | | 12. KVETDAA | DQB1* | 248 | 35 | −213 |
| | | | | | | QPAVQSA | 0602 | | | |
| | | | | | | FAV | | | | |
| | | | | | | (SEQ ID | | | | |
| | | | | | | NO: 911) | | | | |
| | | | | SDK2 | E1596K | 13. NLNKHRR | A*1101 | 1328 | 32996 | 31668 |
| | | | | | | YK | | | | |
| | | | | | | (SEQ ID | | | | |
| | | | | | | NO: 912) | | | | |
| | | | | | | 14. NLNKHRR | DRB1* | 32 | 130 | 98 |
| | | | | | | YKIRMSV | 1501 | | | |
| | | | | | | YNA | | | | |
| | | | | | | (SEQ ID | | | | |
| | | | | | | NO: 913) | | | | |
| Pt.16 | 2 | SD | 4.2 | EGFR | L8S8R | L. KITDFGR | A*1101 | 163 | 20 | −143 |
| | | | | | | AK | | | | |
| | | | | | | (SEQ ID | | | | |
| | | | | | | NO: 4) | | | | |
| | | | | | | 2. VKITDFG | A*1101 | 644 | 54 | −590 |
| | | | | | | RAK | | | | |
| | | | | | | (SEQ ID | | | | |
| | | | | | | NO: 161 | | | | |
| | | | | | | 3. HVKITDF | A*1101 | 4197 | 412 | −3785 |
| | | | | | | GRAK | | | | |
| | | | | | | (SEQ ID | | | | |
| | | | | | | NO: 9) | | | | |
| | | | | | | 4. HVKITDF | DRB1* | 276 | 152 | −124 |
| | | | | | | GRAKLLG | 0901 | | | |
| | | | | | | AEE | | | | |
| | | | | | | (SEQ ID | | | | |
| | | | | | | NO: 826) | | | | |
| Pt.17 | 2 | CR | 14.9 | EGFR | 747_751 | L. AIKESPK | A*1101 | 479 | NA | NA |
| | | | | | del/ | ANK | | | | |
| | | | | | | (SEQ ID | | | | |
| | | | | | | NO: 32) | | | | |
| | | | | | T790M | | | | | |
| | | | | | | 2. KIPVAIK | DRB1* | 2970 | NA | NA |
| | | | | | | ESPKANK | 0901 | | | |
| | | | | | | EIL | | | | |
| | | | | | | (SEQ ID | | | | |
| | | | | | | NO: 914) | | | | |
| | | | | | | 3. LTSTVQL | C*1502 | 584 | 8852 | 8268 |
| | | | | | | IM | | | | |
| | | | | | | (SEQ ID | | | | |
| | | | | | | NO: 65) | | | | |
| | | | | | | 4. STVQLIM | C*1502 | 658 | 1615 | 957 |
| | | | | | | QL | | | | |
| | | | | | | (SEQ ID | | | | |
| | | | | | | NO: 68) | | | | |
| | | | | | | 5. LTSTVQL | DRB1* | 918 | 1127 | 209 |
| | | | | | | IMQLMPF | 0901 | | | |
| | | | | | | GCL | | | | |
| | | | | | | (SEQ ID | | | | |
| | | | | | | NO: 907) | | | | |
| | | | | TP53 | V2721 | 6. LLGRNSF | DRB1* | 1201 | 1085 | −116 |
| | | | | | | ELRVCAC | 0901 | | | |
| | | | | | | PGR | | | | |
| | | | | | | (SEQ ID | | | | |
| | | | | | | NO: 915) | | | | |
| Pt.18 | 2 | PD | 1.9 | EGFR | L858R | 1. ITDFGRA | C*0401 | 7351 | 9928 | 2577 |
| | | | | | | KL | | | | |
| | | | | | | (SEQ ID | | | | |
| | | | | | | NO: 3) | | | | |
| | | | | | | 2. TDFGRAK | B*1302 | 6116 | 2192 | −3924 |
| | | | | | | LL | | | | |
| | | | | | | (SEQ ID | | | | |
| | | | | | | NO: 15) | | | | |
| | | | | | | 3. HVKIFGR | DRB1* | 1591 | 945 | −646 |
| | | | | | | AKLLGAE | 0701 | | | |
| | | | | | | E | | | | |
| | | | | | | (SEQ ID | | | | |
| | | | | | | NO: 826) | | | | |
| | | | | PMS1 | 18281 | 4. IKLIPDV | DRB1* | 3422 | 490 | −2932 |
| | | | | | | STTENYL | 0701 | | | |
| | | | | | | EIE | | | | |
| | | | | | | (SEQ ID | | | | |
| | | | | | | NO: 916) | | | | |
| | | | | | | 5. KLIPGVS | A*0201 | 119 | 409 | 290 |
| | | | | | | TT | | | | |
| | | | | | | (SEQ ID | | | | |
| | | | | | | NO: 917) | | | | |
| | | | | | | 6. IKLIPGV | A*0201 | 1802 | 4470 | 2668 |
| | | | | | | STT | | | | |
| | | | | | | (SEQ ID | | | | |
| | | | | | | NO: 918) | | | | |
| Pt.19 | 1 | PD | 1.7 | TP53 | R175H | 1. EVVRHCP | A*3303 | 217 | 168 | −49 |
| | | | | | | HHER | | | | |
| | | | | | | (SEQ ID | | | | |
| | | | | | | NO: 919) | | | | |
| | | | | | | 2. VVRHCPH | A*3303 | 107 | 145 | 38 |
| | | | | | | HER | | | | |
| | | | | | | (SEQ ID | | | | |
| | | | | | | NO: 920) | | | | |
| | | | | | | 3. AIYKQSQ | DRB1* | 238 | 242 | 4 |
| | | | | | | HMTEVVR | 0901 | | | |
| | | | | | | IICP | | | | |
| | | | | | | (SEQ ID | | | | |
| | | | | | | NO: 920 | | | | |
| | | | | BRAF | G466V | 4. SVSFGTV | A*3303 | 521 | 4016 | 3495 |
| | | | | | | YK | | | | |
| | | | | | | (SEQ ID | | | | |
| | | | | | | NO: 922) | | | | |
| | | | | | | 5. GSVSFGT | C*1403 | 1380 | 2873 | 1493 |
| | | | | | | VY | | | | |
| | | | | | | (SEQ ID | | | | |
| | | | | | | NO: 923) | | | | |
| | | | | | | 6. PDGQITV | DRB1* | 839 | 4998 | 4159 |
| | | | | | | GRIGSVS | 1302 | | | |
| | | | | | | FG | | | | |
| | | | | | | (SEQ ID | | | | |
| | | | | | | NO: 924) | | | | |
| | | | | KRAS | G12C | 7. KLVVVGA | A*0201 | 373 | 506 | 133 |
| | | | | | | CGV | | | | |
| | | | | | | (SEQ ID | | | | |
| | | | | | | NO: 925) | | | | |
| | | | | | | 8. EYKLVVV | DRB1* | 294 | 190 | −104 |
| | | | | | | GACGVGK | 0901 | | | |
| | | | | | | SAL | | | | |
| | | | | | | (SEQ ID | | | | |
| | | | | | | NO: 926) | | | | |
| | | | | BCL2L2 | T715M | 9. VLMGAVA | A*0201 | 63 | 1053 | 990 |
| | | | | | | LGA | | | | |
| | | | | | | (SEQ ID | | | | |
| | | | | | | NO: 927) | | | | |
| | | | | | | 10. VLMGAVA | A*0201 | 312 | 3591 | 3279 |
| | | | | | | LGAL | | | | |
| | | | | | | (SEQ ID | | | | |
| | | | | | | NO: 928) | | | | |
| | | | | TNFSF11 | P33L | 11. LPHEGPL | DRB1* | 4217 | 15098 | 10881 |
| | | | | | | HALPPPA | 0901 | | | |
| | | | | | | PHOP | | | | |
| | | | | | | (SEQ ID | | | | |
| | | | | | | NO: 929) | | | | |
| | | | | DOCK2 | R1200C | 12. CMSCTVN | A*0207 | 179 | 79 | −3674 |
| | | | | | | LL | | | | |
| | | | | | | (SEQ ID | | | | |
| | | | | | | NO: 930) | | | | |
| Pt.20 | 2 | PD | 2.8 | EGFR | L8S8R/ | 1. AILLGAE | A*1101 | 118 | 12076 | 1 1958 |
| | | | | | K8601 | EK | | | | |
| | | | | | | (SEQ ID | | | | |
| | | | | | | NO: 931) | | | | |
| | | | | | | 2. RAILLGA | A*1101 | 280 | 2452 | 2172 |
| | | | | | | EEK | | | | |
| | | | | | | (SEQ ID | | | | |
| | | | | | | NO: 932) | | | | |
| | | | | | | 3. TDFGRAI | B*5001 | 1386 | 1695 | 309 |
| | | | | | | LL | | | | |
| | | | | | | (SEQ ID | | | | |
| | | | | | | NO: 933) | | | | |
| | | | | | | 4. HVKITDF | DRB1* | 1462 | 945 | −517 |
| | | | | | | GRAILLG | 0701 | | | |
| | | | | | | AEE | | | | |
| | | | | | | (SEQ ID | | | | |
| | | | | | | NO: 934) | | | | |
| | | | | | | 5. VKITDFG | DQB1* | 1027 | 1168 | 141 |
| | | | | | | RAILLGA | 0301 | | | |
| | | | | | | EEK | | | | |
| | | | | | | (SEQ ID | | | | |
| | | | | | | NO: 935) | | | | |
| | | | | TP53 | F113S | 6. GSLHSGT | A*1101 | 68 | 26 | −42 |
| | | | | | | AK | | | | |
| | | | | | | (SEQ ID | | | | |
| | | | | | | NO: 936) | | | | |
| | | | | | | 7. LGSLHSG | A*1101 | 418 | 148 | −270 |
| | | | | | | TAK | | | | |
| | | | | | | (SEQ ID | | | | |
| | | | | | | NO: 937) | | | | |
| | | | | | | 8. GSYGFRL | DQB1* | 198 | 840 | 642 |
| | | | | | | GSLHSGT | 0301 | | | |
| | | | | | | AKS | | | | |
| | | | | | | (SEQ ID | | | | |
| | | | | | | NO: 938) | | | | |
| | | | | BRAE | V600E | 9. GDFGLAT | A*1101 | 2008 | 2401 | 393 |
| | | | | | | EK | | | | |
| | | | | | | (SEQ ID | | | | |
| | | | | | | NO: 939) | | | | |
| | | | | | | 10. IGDFGLA | A*1101 | 2352 | 2884 | 532 |
| | | | | | | TEK | | | | |
| | | | | | | (SEQ ID | | | | |
| | | | | | | NO: 940) | | | | |
| | | | | | | 11. LGDFGLA | DQB1* | 2155 | 240 | −1915 |
| | | | | | | TEKSRWS | 0301 | | | |
| | | | | | | GSH | | | | |
| | | | | | | (SEQ ID | | | | |
| | | | | | | NO: 941) | | | | |
| Pt.21 | 3 | SD | 5.2 | EGER | 745_ | 1. AITSPKA | A*1102 | 83 | NA | NA |
| | | | | | 750del | NK | | | | |
| | | | | | | (SEQ ID | | | | |
| | | | | | | NO: 942) | | | | |
| | | | | | | 2. KIPVAIT | A*1102 | 39 | NA | NA |
| | | | | | | SPK | | | | |
| | | | | | | (SEQ ID | | | | |
| | | | | | | NO: 943) | | | | |
| | | | | | | 3. KVKIPVA | DQB1* | 479 | NA | NA |
| | | | | | | ITSPKAN | 0301 | | | |
| | | | | | | KEI | | | | |
| | | | | | | (SEQ ID | | | | |
| | | | | | | NO: 944) | | | | |
| | | | | KMT2A | 03131S | 4. LGPMGGS | B*5101 | 2991 | 3661 | 670 |
| | | | | | | LTL | | | | |
| | | | | | | (SEQ ID | | | | |
| | | | | | | NO: 945) | | | | |
| | | | | | | 5. SVLGPMG | DRB1* | 2022 | 3858 | 1637 |
| | | | | | | GSLTLTT | 1501 | | | |
| | | | | | | GLN | | | | |
| | | | | | | (SEQ ID | | | | |
| | | | | | | NO: 946) | | | | |
| | | | | GATA2 | S429T | 6. KSSPFTA | C*1502 | 878 | 339 | −539 |
| | | | | | | AA | | | | |
| | | | | | | (SEQ ID | | | | |
| | | | | | | NO: 947) | | | | |
| | | | | | | 7. FTAAALA | A*1102 | 3104 | 5471 | 2367 |
| | | | | | | GH | | | | |
| | | | | | | (SEQ ID | | | | |
| | | | | | | NO: 948) | | | | |
| | | | | | | 8. MQEKSSP | DRB1* | 1336 | 1924 | 588 |
| | | | | | | FTAAALA | 1501 | | | |
| | | | | | | GHM | | | | |
| | | | | | | (SEQ | | | | |
| | | | | | | ID | | | | |
| | | | | | | NO: 949) | | | | |
| | | | | JAKS | R8700 | 9. QEIQILK | B*4001 | 20 | 9 | −11 |
| | | | | | | AL | | | | |
| | | | | | | (SEQ ID | | | | |
| | | | | | | NO: 950) | | | | |
| | | | | | | 10. RDFQQEI | A*2402 | 1608 | 1078 | −539 |
| | | | | | | QIL | | | | |
| | | | | | | (SEQ ID | | | | |
| | | | | | | NO: 951) | | | | |
| | | | | | | 11. QQEIQIL | B*4001 | 182 | 107 | −75 |
| | | | | | | KAL | | | | |
| | | | | | | (SEQ ID | | | | |
| | | | | | | NO: 952) | | | | |
| | | | | | | 12. FQQEIQI | A*1102 | 193 | 540 | 347 |
| | | | | | | LK | | | | |
| | | | | | | (SEQ ID | | | | |
| | | | | | | NO: 953) | | | | |
| | | | | | | 13. QQRDFQQ | DRB1* | 75 | 55 | −20 |
| | | | | | | EIQILKA | 1501 | | | |
| | | | | | | LHS | | | | |
| | | | | | | (SEQ ID | | | | |
| | | | | | | NO: 954) | | | | |
| Pt.22 | 3 | PR | 8.7 | EGFR | H773L/ | 1. VMASVDN | A*1201 | 30 | 21040 | 21010 |
| | | | | | V774M | PL | | | | |
| | | | | | | (SEQ ID | | | | |
| | | | | | | NO: 880) | | | | |
| | | | | | | 2. MASVDNP | B*1511 | 1019 | 8386 | 7367 |
| | | | | | | LM | | | | |
| | | | | | | (SEQ ID | | | | |
| | | | | | | NO: 955) | | | | |
| | | | | | | 3. NPLMCRL | A*0201 | 94 | 23140 | 23046 |
| | | | | | | LGI | | | | |
| | | | | | | (SEQ ID | | | | |
| | | | | | | NO; 956) | | | | |
| | | | | | | 4. VDNPLMC | B*3704 | 4030 | 2935 | −1095 |
| | | | | | | RL | | | | |
| | | | | | | (SEQ ID | | | | |
| | | | | | | NO: 957) | | | | |
| | | | | | | 5. MASVDNP | DRB1* | 864 | 1365 | 501 |
| | | | | | | LMCRLLG | 0901 | | | |
| | | | | | | ICL | | | | |
| | | | | | | (SEQ ID | | | | |
| | | | | | | NO: 958) | | | | |
| | | | | FGFR1 | R734W | 6. TNELYMM | DRB1* | 396 | 494 | 98 |
| | | | | | | MWDCWHA | 0901 | | | |
| | | | | | | VPS | | | | |
| | | | | | | (SEQ ID | | | | |
| | | | | | | NO: 959) | | | | |
| | | | | | | 7. MMMWDCW | A*0201 | 3 | 5 | 2 |
| | | | | | | HA | | | | |
| | | | | | | (SEQ ID | | | | |
| | | | | | | NO: | | | | |
| | | | | | | 960) | | | | |
| | | | | | | 8. MMWDCWH | A*0201 | 2 | 14 | 12 |
| | | | | | | AV | | | | |
| | | | | | | (SEQ ID | | | | |
| | | | | | | NO: 961) | | | | |
| | | | | | | 9. YMMMWDC | A*0201 | 4 | 8 | 4 |
| | | | | | | WHA | | | | |
| | | | | | | (SEQ ID | | | | |
| | | | | | | NO: | | | | |
| | | | | | | 962) | | | | |
| | | | | | | 10. MMMWDCW | A*0201 | 3 | 5 | 2 |
| | | | | | | HAV | | | | |
| | | | | | | (SEQ ID | | | | |
| | | | | | | NO: | | | | |
| | | | | | | 963) | | | | |
| | | | | | | 11. YMMMWDC | A*0201 | 6 | 9 | 3 |
| | | | | | | WHAV | | | | |
| | | | | | | (SEQ ID | | | | |
| | | | | | | NO: 964) | | | | |
| Pt.23 | 3 | PD | 2.9 | EGFR | 746_ | L. IPVAIKT | A*0301 | 82 | NA | NA |
| | | | | | 750del | SPK | | | | |
| | | | | | | (SEQ ID | | | | |
| | | | | | | NO: 19) | | | | |
| | | | | | | 2. AIKTSPK | A*0301 | 138 | NA | NA |
| | | | | | | ANK | | | | |
| | | | | | | (SEQ ID | | | | |
| | | | | | | NO: 20) | | | | |
| | | | | | | 3. VKIPVAI | DRB1* | 751 | NA | NA |
| | | | | | | KTSPKAN | 1301 | | | |
| | | | | | | KEI | | | | |
| | | | | | | (SEQ ID | | | | |
| | | | | | | NO: 817) | | | | |
| | | | | TP53 | G105V | 4. TYQVSYG | A*3101 | 19 | 20 | 1 |
| | | | | | | FR | | | | |
| | | | | | | (SEQ ID | | | | |
| | | | | | | NO: 965) | | | | |
| | | | | | | 5. YQVSYGF | B*3701 | 441 | 971 | 530 |
| | | | | | | RL | | | | |
| | | | | | | (SEQ ID | | | | |
| | | | | | | NO: 966) | | | | |
| | | | | | | 6. KTYQVSY | A*3101 | 7 | 8 | 1 |
| | | | | | | GFR | | | | |
| | | | | | | (SEQ ID | | | | |
| | | | | | | NO: 967) | | | | |
| | | | | | | 7. QKTYQVS | A*3101 | 19 | 27 | 8 |
| | | | | | | YGFR | | | | |
| | | | | | | (SEQ ID | | | | |
| | | | | | | NO: 968) | | | | |
| | | | | | | 8. VPSQKTY | DQB1* | 1338 | 5553 | 4215 |
| | | | | | | QVSYGFR | 0603 | | | |
| | | | | | | LGP | | | | |
| | | | | | | (SEQ ID | | | | |
| | | | | | | NO: 969) | | | | |
| | | | | EXT1 | R595H | 9. HSHFWDN | A*3101 | 326 | 50 | −276 |
| | | | | | | SK | | | | |
| | | | | | | (SEQ ID | | | | |
| | | | | | | NO: 970) | | | | |
| | | | | | | 10. ERIVGYP | DRB1* | 1557 | 1433 | −12 |
| | | | | | | AHSHFWD | 0403 | | | |
| | | | | | | NSK | | | | |
| | | | | | | (SEQ ID | | | | |
| | | | | | | NO: 971) | | | | |
| Pt.24 | 2 | PD | 2.6 | EGFR | 746_ | 1. KTSPKAN | B*5701 | 1053 | NA | NA |
| | | | | | 750del | KEI | | | | |
| | | | | | | (SEQ ID | | | | |
| | | | | | | NO: 29) | | | | |
| | | | | | | 2. VKIPVAI | DRB1* | 409 | NA | NA |
| | | | | | | KTSPKAN | 1602 | | | |
| | | | | | | KEI | | | | |
| | | | | | | (SEQ ID | | | | |
| | | | | | | NO: 817) | | | | |
| | | | | DAXX | 440N | 3. RAETDDE | DQB1* | 3440 | 2393 | −1047 |
| | | | | | | DNEESDE | 0502 | | | |
| | | | | | | EEE | | | | |
| | | | | | | (SEQ ID | | | | |
| | | | | | | NO: 972) | | | | |
| | | | | TUBDI | V396A | 4. KSAVLAS | B*5701 | 530 | 998 | 468 |
| | | | | | | NSQF | | | | |
| | | | | | | (SEQ ID | | | | |
| | | | | | | NO: 973) | | | | |
| | | | | | | 5. KYEKSAV | DRB* | 115 | 204 | 89 |
| | | | | | | LASNSQF | 1602 | | | |
| | | | | | | LVK | | | | |
| | | | | | | (SEQ ID | | | | |
| | | | | | | NO: 974) | | | | |
| | | | | ATR | H891R | 6. DLVPFAL | DRB1*1602 | 154 | 231 | 77 |
| | | | | | | LRLLHCL | | | | |
| | | | | | | LSK | | | | |
| | | | | | | (SEQ ID | | | | |
| | | | | | | NO: 975) | | | | |
| | | | | ETVI | D38G | 7. RKRKFIN | DRB1* | 447 | 1143 | 696 |
| | | | | | | RGLAHDS | 1301 | | | |
| | | | | | | EEL | | | | |
| | | | | | | (SEQ ID | | | | |
| | | | | | | NO: 976) | | | | |
| | | | | PIK3CA | N145S | 8. QDFRRNI | DRB1* | 479 | 1028 | 549 |
| | | | | | | LSVCKEA | 1602 | | | |
| | | | | | | VDL | | | | |
| | | | | | | (SEQ ID | | | | |
| | | | | | | NO: 977) | | | | |

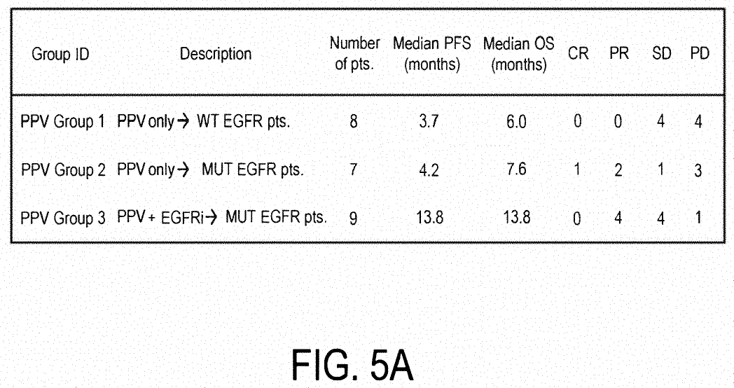

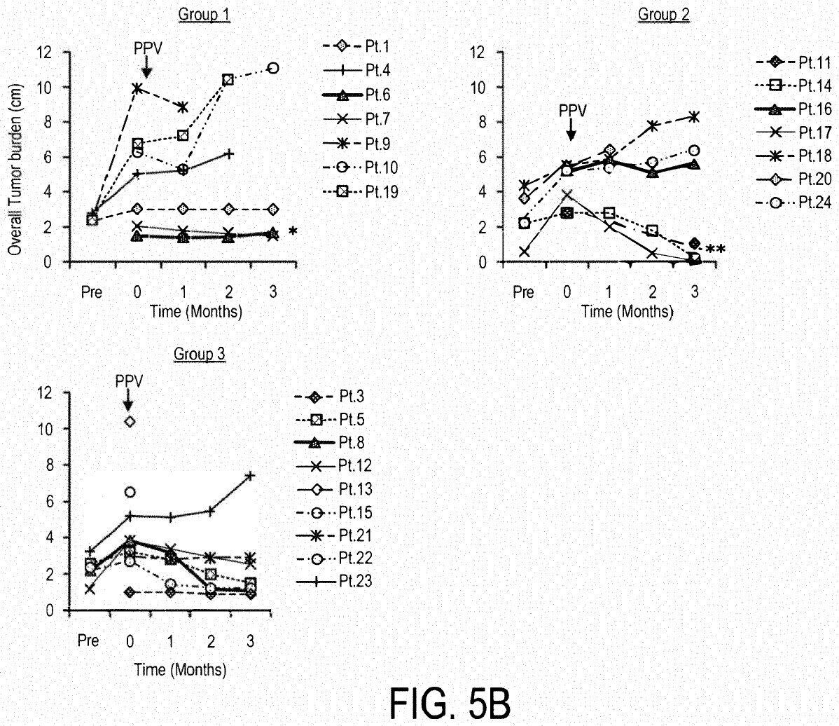

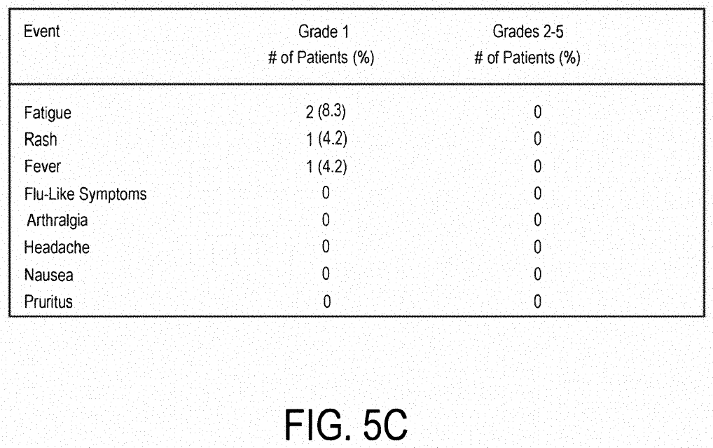

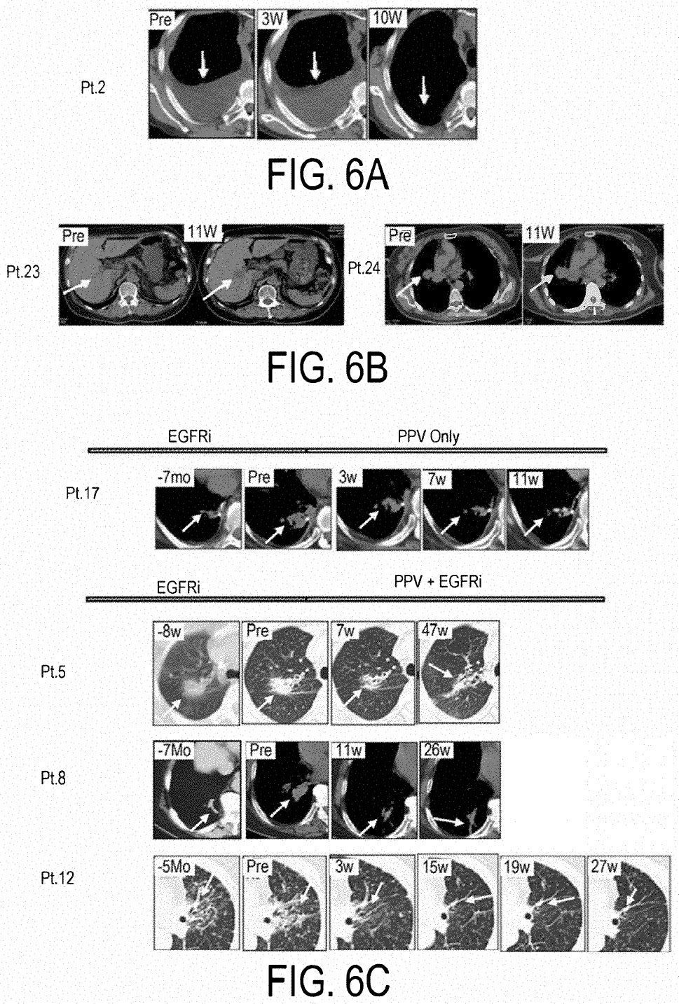

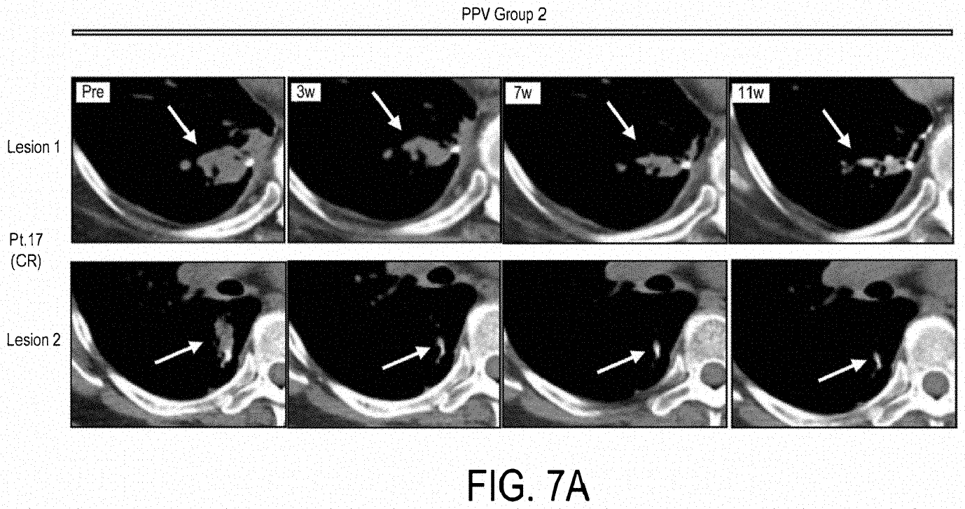

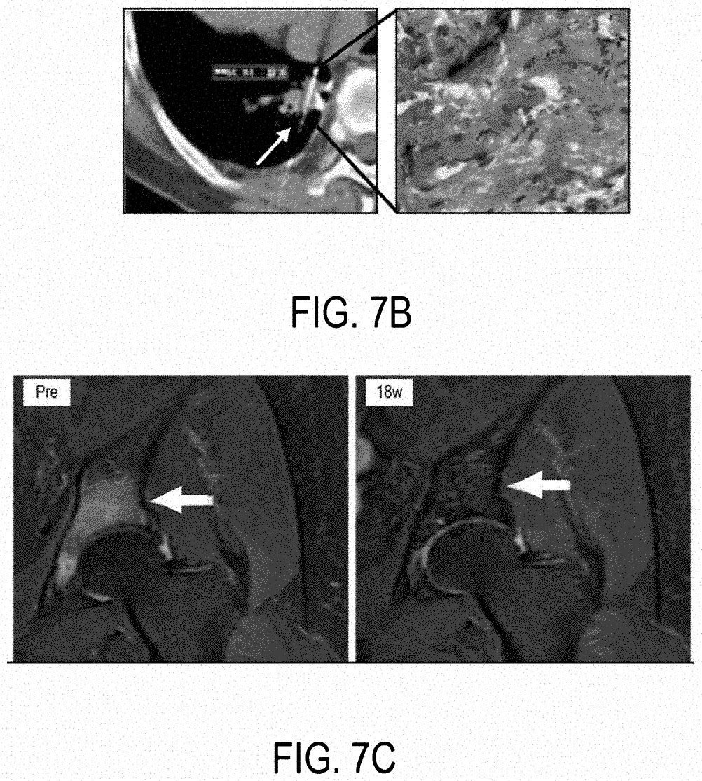

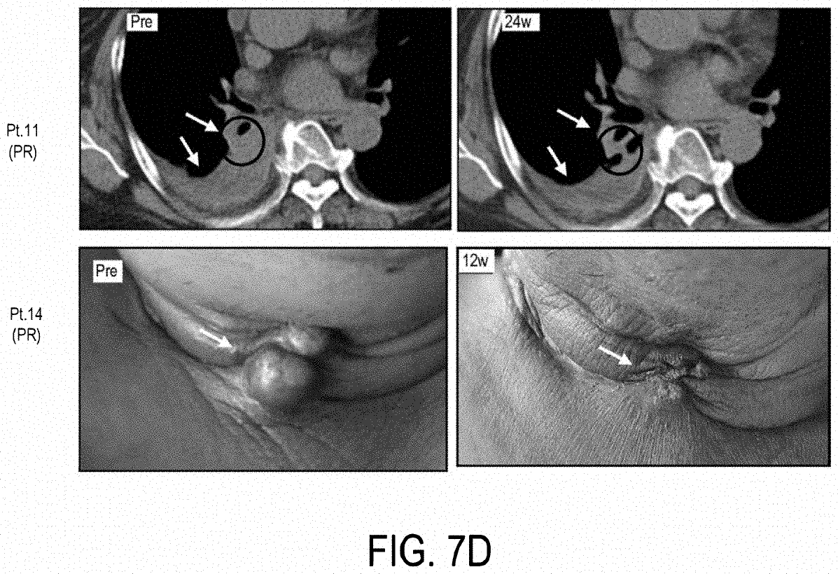

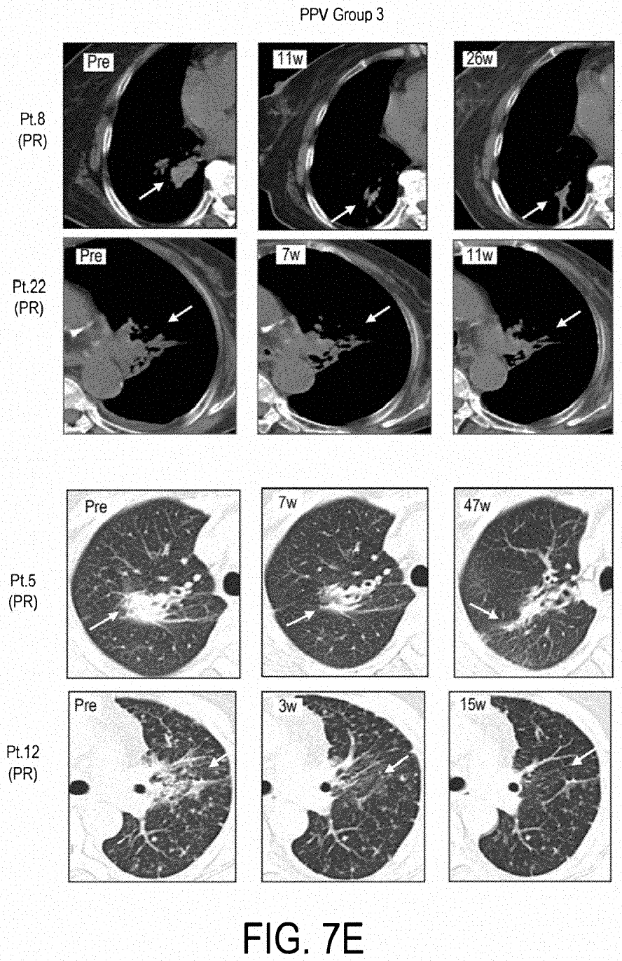

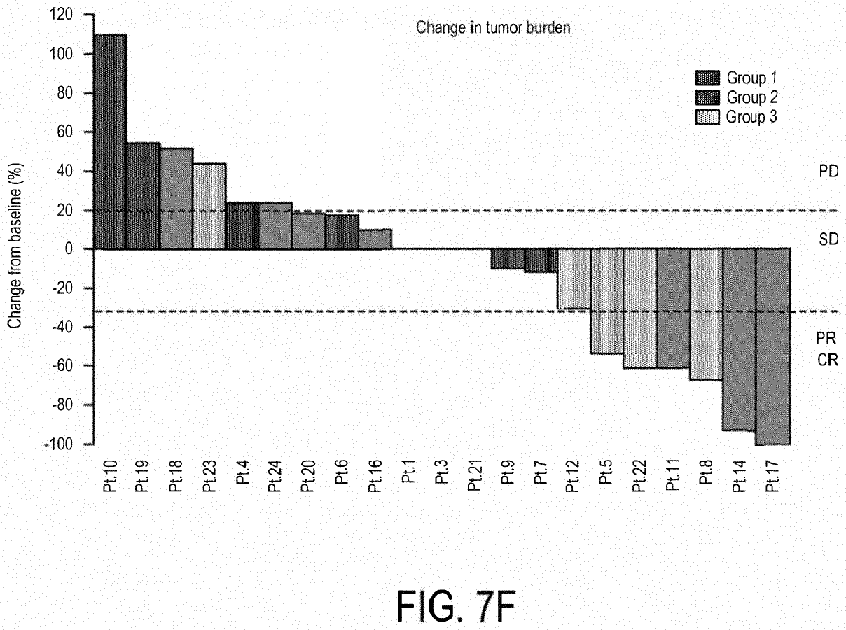

PPV induced clinical responses in multiple patients with EGFR-mutated tumors. Clinical outcomes for all 24 patients are shown in Table 6, with Group outcomes summarized in FIGS. 5A & 5B and Table 7. Aside from Grade 1 transient rashes, fatigue and/or fever experienced by 3 patients, no treatment-related adverse events were observed (FIG. 5C). Of the EGFR-W7T patients in Group 1, 4 showed stable disease (SD) and 4 had progressive disease (PD) after 12 weeks of PPV. Although Patient 2 experienced clearance of pleural fluid, no tumor regressions was observed (FIGS. 6A & 6B). By contrast, objective clinical responses were observed in 3 of the 7 patients in Group 2, including 2 PRs (Pts. 11 and 14) and one CR (Pt. 17) that was confirmed by a post-treatment biopsy showing no viable tumor cells (FIGS. 7 A-7D). Furthermore, 4 of the 9 patients in Group 3 experienced partial responses (PR), with 4 additional patients deemed SD (FIGS. 7E & 7F; FIG. 5A). Group 3 patients responded in spite of previous disease progression on EGFRi therapy (FIG. 6C).

TABLE 6
Clinical outcomes of 24 personalized neoantigen vaccine patients. F, female; M, male; SR, surgical removal; RT, radiotherapy;
CM, chemotherapy; EGFRi, EGFR inhibitor; PFS, progression-free survival; CR, complete response; PR, partial response;
PD, progressive disease; SD, stable disease.

| | | | | | Number | Period of | | | | Treatments |
|---|---|---|---|---|---|---|---|---|---|---|
| | | | Previous | | of | vaccination | Side | Patient | PFS | after |
| Pt. ID | Stage | Sex | treatment | Cohort | vaccines | (weeks) | effects | response | (months) | vaccination |
| Pt. 1 | IV | F | SR, RT | 1 | 12 | 11 | No | PD | 4.9 | CM |
| Pt. 2 | IV | F | RT, CM | 1 | 24 | 23 | No | SD | 6.8 | No |
| Pt. 3 | IV | M | CM, EGFRi | 3 | 28 | 37.9 | Fatigue | SD | 24.8 | No |
| Pt. 4 | IIIB | M | RT, CM | 1 | 12 | 10.4 | No | PD | 2 | No |
| Pt. 5 | IV | F | RT, CM, EGFRi | 3 | 49 | 79.6 | No | PR | 20 | No |
| Pt. 6 | IIIB | M | RT, CM | 1 | 12 | 11 | No | SD | 3.9 | RT |
| Pt. 7 | IV | M | RT | 1 | 12 | 11 | No | SD | 20.3 | No |
| Pt. 8 | IV | F | RT, EGFRi | 3 | 18 | 30.9 | No | PR | 9.5 | No |
| Pt. 9 | IV | M | RT | 1 | 6 | 5.1 | No | SD | 3.4 | No |
| Pt. 10 | IIIA | M | RT | 1 | 12 | 11 | No | PD | 2.7 | No |
| Pt. 11 | IV | F | RT, CM, EGFRi | 2 | 24 | 24 | No | PR | 8.6 | No |
| Pt. 12 | IV | F | RT, CM, EGFRi | 3 | 65 | 70.6 | No | PR | 18 | No |
| Pt. 13 | IV | F | CM, EGFRi | 3 | 13 | 13.4 | No | SD | 8.9 | No |
| Pt. 14 | IIIB | M | SR, RT, CM, | 2 | 12 | 11 | No | PR | 7.6 | No |
| | | | EGFRi | | | | | | | |
| Pt. 15 | IV | F | RT, CM, EGFRi | 3 | 12 | 11 | No | SD | 13.8 | No |
| Pt. 16 | IV | M | RT, CM, EGFRi | 2 | 12 | 11 | No | SD | 4.2 | No |
| Pt. 17 | IV | F | SR, EGFRi | 2 | 49 | 51 | No | CR | 14.9 | No |
| Pt. 18 | IIIB | F | CM, EGFRi | 2 | 12 | 11 | Rash, | PD | 1.9 | No |
| | | | | | | | Fatigue | | | |
| Pt. 19 | IV | F | RT, CM | 1 | 9 | 8 | Fever | PD | 1.7 | No |
| Pt. 20 | IV | M | CM, EGFRi | 2 | 7 | 5.9 | No | PD | 2.8 | No |
| Pt. 21 | IV | M | RT, CM, EGFRi | 3 | 23 | 22.3 | No | SD | 5.2 | No |
| Pt. 22 | IV | F | RT, EGFRi | 3 | 33 | 33 | No | PR | 8.7 | No |
| Pt. 23 | IV | F | RT, CM, EGFRi | 3 | 24 | 24 | No | PD | 2.9 | No |
| Pt. 24 | IV | F | SR, CM, EGFRi | 2 | 12 | 11 | No | PD | 2.6 | EGFRi |

TABLE 7
part 1. EGFR neoantigen vaccine peptides associated
with patient clinical responses.
*Peptides included in vaccine of PR
patient from Li et at., 2016.
⁺Pt. 17 is a complete responder.

| | Vaccine | | | # CR/ | | | |
|---|---|---|---|---|---|---|---|
| HLA | peptide | # Patients | Patient | PR | #SD | # PD | PFS |
| class | sequence | immunized | IDs | patients | patients | patients | (months) |
| I | KITDFGRAK* | 4 | 5, 8, | 3 | 1 | 0 | 20.0, 9.5, |
| | (SEQ ID | | 14, 16 | | | | 7.6, 4.2 |
| | NO: 4) | | | | | | |
| I | VKITDFGRAK | 4 | 5, 8, | 3 | 1 | 0 | 20.0. 9.5, |
| | (SEQ ID | | 14, 16 | | | | 7.6,4.2 |
| | NO: 16) | | | | | | |
| I | TDFGRAKLL | 3 | 5, 12, | 2 | 0 | 1 | 20.0, 18.0, |
| | (SEQ ID | | 18 | | | | 1.9 |
| | NO: 15) | | | | | | |
| I | VMASVDNPL | 2 | 11, 22 | 2 | 0 | 0 | 8.6, 8.7 |
| | (SEQ ID | | | | | | |
| | NO: 880) | | | | | | |
| I | LTSTVQLIM | 2 | 15, 17⁺ | 1 | 1 | 0 | 13.8, 14.9 |
| | (SEQ ID | | | | | | |
| | NO: 65) | | | | | | |
| I | ITDFGRAKL | 2 | 5, 18 | 1 | 0 | 1 | 20.0, 1.9 |
| | (SEQ ID | | | | | | |
| | NO: 3) | | | | | | |
| I | HVKITDFGRAK* | 2 | 8, 16 | 1 | 1 | 0 | 9.5, 4.2 |
| | (SEQ | | | | | | |
| | ID NO: 9) | | | | | | |
| I | FGRAKLLGA | 1 | 8 | 1 | 0 | 0 | 9.5 |
| | (SEQ ID | | | | | | |
| | NO: 6) | | | | | | |
| I | HVKITDFGR* | 1 | 14 | 1 | 0 | 0 | 7.6 |
| | (SEQ ID | | | | | | |
| | NO: 1) | | | | | | |
| I | RILKVTEFK | | | | | | |
| | (SEQ ID NO: | | | | | | |
| | 897) | | | | | | |
| I | RILKVTEFKK | | | | | | |
| | (SEQ ID | | | | | | |
| | NO: 898) | | | | | | |
| I | LRILKVTEFK | | | | | | |
| | (SEQ ID | | | | | | |
| | NO: 899) | | | | | | |
| I | STVQLIMQL | 1 | 17⁺ | 1 | 0 | 0 | 14.9 |
| | (SEQ ID | | | | | | |
| | NO: 68) | | | | | | |
| I | AIKESPKANK | | | | | | |
| | (SEQ ID | | | | | | |
| | NO: 32) | | | | | | |
| I | MASVDNPLM | 1 | 22 | 1 | 0 | 0 | 8.7 |
| | (SEQ ID | | | | | | |
| | NO: 955) | | | | | | |
| I | NPLMCRLLGI | | | | | | |
| | (SEQ ID | | | | | | |
| | NO: 956) | | | | | | |
| I | VDNPLMCRL | | | | | | |
| | (SEP ID | | | | | | |
| | NO: 957) | | | | | | |
| II | HVKITDFGRA | 6 | 5, 8, | 4 | 1 | 1 | 20.0, 9.5, |
| | KLLGAEE | | 12, 14, | | | | 18.0, 7.6, |
| | (SEQ ID | | 16, 18 | | | | 4.2, 1.9 |
| | NO: 826) | | | | | | |
| II | QALLRILKVT | 1 | 14 | 1 | 0 | 0 | 7.6 |
| | EFKKIKV | | | | | | |
| | (SEQ ID | | | | | | |
| | NO: 900) | | | | | | |
| II | KIPVAIKES | 1 | 17⁺ | 1 | 0 | 0 | 14.9 |
| | PKANKEIL | | | | | | |
| | (SEQ ID | | | | | | |
| | NO: 914) | | | | | | |
| II | LTSTVQLIM | 1 | 17⁺ | 1 | 0 | 0 | 14.9 |
| | QLMPFGCL | | | | | | |
| | (SEQ ID | | | | | | |
| | NO: 907) | | | | | | |
| II | MASVDNPLM | 1 | 22 | 1 | 0 | 0 | 8.7 |
| | CRLLGICL | | | | | | |
| | (SEQ ID | | | | | | |
| | NO: 958) | | | | | | |

part 2. EGFR neoantigen vaccine peptides associated with
patient clinical responses. *Peptides included in vaccine
of PR patient from Li et al., 2016.
⁺⁺patients expressed each listed HLA class II allotype.

| | | | HLA | |
|---|---|---|---|---|
| | Predicted | | population | HLA |
| | HLA | | prevalence | population |
| Vaccine | binding | | in Europe/ | prevalence |
| peptide | affinity | HLA | N. America | in Asia |
| sequence | (nM) | restriction | (%) | (%) |
| KITDFGRAK* | 163 | A*1101 | 13.2 | 34.3 |
| (SEQ ID NO: 4) | | | | |
| VKITDFGRAK | 644 | | | |
| (SEQ ID NO: 16) | | | | |
| TDFGRAKLL | 624 | C*0602 | 18.4 | 11.9 |
| (SEQ ID NO: 15) | 766 | B*3701 | 2.7 | 4.2 |
| | 6116 | B*1302 | 4.8 | 18.4 |
| VMASVDNPL | 30 | A*0201 | 43.1 | 32.3 |
| (SEQ ID NO: 880) | | | | |
| LTSTVQLIM | 1274 | B*3501 | 12.6 | 7.4 |
| (SEQ ID NO: 65) | 584 | C*1502 | 6.4 | 8.1 |
| ITDFGRAKL | 1826 | C*0102 | 8.6 | 33.7 |
| (SEQ ID NO: 3) | 7351 | C*0401 | 23.1 | 12.0 |
| HVKITDFGRAK* | 4197 | A*1101 | 13.2 | 34.3 |
| (SEQ ID NO: 9) | | | | |
| FGRAKLLGA | 3471 | B*5401 | 0.3 | 5.8 |
| (SEQ ID NO: 6) | | | | |
| HVKITDFGR* | 10 | A*3101 | 8.5 | 5.7 |
| (SEQ ID NO: 1) | | | | |
| RILKVTEFK | 12 | A*1101 | 13.2 | 34.3 |
| (SEQ ID NO: 897) | | | | |
| RILKVTEFKK | 24 | | | |
| (SEQ ID NO: 898) | | | | |
| LRILKVTEFK | 62 | | | |
| (SEQ ID NO: 899) | | | | |
| STVQLIMQL | 658 | C*1502 | 6.4 | 8.1 |
| (SEQ ID NO: 68) | | | | |
| AIKESPKANK | 479 | A* 1101 | 13.2 | 34.3 |
| (SEQ ID NO: 32) | | | | |
| MASVDNPLM | 1019 | B*1511 | <0.1 | 5.4 |
| (SEQ ID NO: 955) | | | | |
| NPLMCRLLGI | 94 | A*0201 | 43.1 | 32.3 |
| (SEQ ID NO: 956) | | | | |
| VDNPLMCL | 4030 | B*3704 | <0.1 | <0.1 |
| (SEQ ID NO: 957) | | | | |
| HVKITDFGRAKLLGAEE | 275 | DRB1*0701⁺⁺ | 22.2 | 15.9 |
| (SEQ ID NO: 826) | 102 | DRB1*0901⁺⁺ | 2.7 | 29.6 |
| | 22 | DQB1*0301⁺⁺ | 32.4 | 41.5 |
| QALLRILKVTEFKKIKV | 241 | DRB1*0901 | 2.7 | 29.6 |
| (SEQ ID NO: 900) | | | | |
| KIPVAIKESPKANKEIL | 2970 | | | |
| (SEQ ID NO: 914) | | | | |
| LTSTVQLIMQLMPFGCL | 484 | | | |
| (SEQ ID NO: 907) | | | | |
| MASVPNPLMCRLLGICL | 864 | | | |
| (SEQ ID NO: | | | | |

958)

part 3. EGFR neoantigen vaccine peptides associated with
patient clinical responses.
*Peplides included in vaccine of PR
patient front Li et al., 2016.
**T790M prevalence is rare in untreated patients (<2%),
but is acquired in ~50% in FGFRi-treated patients that
develop resistance.

| | | | | Mutation | |
|---|---|---|---|---|---|
| | | EGFR | | prevalence | |
| | | mutation | | in | Mutation |
| | | frequency | EGFR | EGFR- | prevalence |
| | EGFR | in | mutation | mutated | in |
| | Mutation | lung | frequency | lung | EGFR- |
| | type | cancer, | in | cancer, | mutated |
| Vaccine | EGFR | Europe/ | lung | Europe/ | lung cancer, |
| peptide | Mutation | N. America | cancer, | N. America | Asia |
| sequence | type | (%) | Asia (%) | (%) | (%) |
| KITDFGRAK* | L858R | 8.8 | 24.7 | 37.1 | 44.3 |
| (SEQ ID NO: 4) | | | | | |
| VKITDFGRAK | | | | | |
| (SEQ ID NO: 16) | | | | | |
| TDFGRAKLL | | | | | |
| (SEQ ID NO: 15) | | | | | |
| VMASVDNPL | H773L | <0.1 | <0.1 | <0.5 | <0.5 |
| (SEQ ID NO: | | | | | |
| 880) | | | | | |
| LTSTVQLIM | T790M** | <1.0 | <2.0 | <5.0 | <5.0 |
| (SEQ ID NO: 65) | | | | (~50)** | (~50)** |
| ITDFGRAKL | L858R | 8.8 | 24.7 | 37.1 | 44.3 |
| (SEQ ID NO: 3) | | | | | |
| HVKITDFGRAK* | | | | | |
| (SEQ ID NO: | | | | | |
| 9) | | | | | |
| FGRAKLLGA | | | | | |
| (SEQ ID NO: 6) | | | | | |
| HVKITDFGR* | | | | | |
| (SEQ ID NO: 1) | | | | | |
| RILKVTEFK | E709V | <0.1 | <0.1 | <0.1 | <0.1 |
| (SEQ ID NO: 897) | | | | | |
| RILKVTEFKK | | | | | |
| (SEQ ID NO: 898) | | | | | |
| LRILKVTEFK | | | | | |
| (SEQ ID NO: | | | | | |
| 899) | | | | | |
| STVQLIMQL | T790M** | <1.0 | <2.0 | <5.0 | <5.0 |
| (SEQ ID NO: 68) | | | | (~50)** | (~50)** |
| AIKESPKANK | 747_751 | <1.0 | <2.0 | <2.0 | <3.0 |
| (SEQ ID NO: 32) | Del | | | | |
| MASVDNPLM | H773L/ | <0.1 | <0.1 | <0.1 | <0.1 |
| (SEQ ID NO: | V774M | | | | |
| 955) | | | | | |
| NPLMCRLLGI | | | | | |
| (SEQ ID NO: | | | | | |
| 956) | | | | | |
| VDNPLMCRL | | | | | |
| (SEQ ID NO: | | | | | |
| 957) | | | | | |
| HVKITDFGRAKL | L858R | 8.8 | 24.7 | 37.1 | 44.3 |
| LGAEE (SEQ | | | | | |
| ID NO: 826) | | | | | |
| QALLRILKVTEF | E709V | <0.1 | <0.1 | <0.1 | <0.1 |
| KKIKV (SEQ | | | | | |
| ID NO: 900) | | | | | |
| K1PVAIKESPKA | 747_751 | <1.0 | <2.0 | <2.0 | <3.0 |
| NKEIL (SEQ | Del | | | | |
| ID NO: 914) | | | | | |
| LTSTVQLIMQEM | T790M** | <1.0 | <2.0 | <5.0 | <5.0 |
| PFGCL (SEQ | | | | (~50)** | (~50)** |
| ID NO: 907) | | | | | |
| MASVDNPLMCRL | II773L/ | <0.1 | <0.1 | <0.1 | <0.1 |

| | |
|---|---|
| LGICL (SEP | V774M |
| ID NO: 958) | |

part 4. EGFR neoantigen vaccine peptides associated with
patient clinical responses.
*Peptides included in vaccine of PR
patient from Li et al., 2016.

| | | | Potential | |
|---|---|---|---|---|
| | Potential | | target | Potential |
| | target | Potential | prevalence in | target |
| | prevalence | target | European/ | prevalence |
| | in | prevalence | N. American | in Asian |
| | European/ | in | EGFR- | EGER- |
| | N. American | Asian | mutated | mutated |
| | lung | lung | lung | lung |
| Vaccine | cancer | cancer | cancer | cancer |
| peptide | patients | patients | patients | patients |
| sequence | (%) | (%) | (%) | (%) |
| KITDFGRAK* | 1.2 | 8.4 | 4.9 | 15.2 |
| (SEQ ID NO: 4) | | | | |
| VKITDFGRAK | 1.2 | 8.4 | 4.9 | 15.2 |
| (SEQ ID NO: 16) | | | | |
| TDFGRAKLL | 1.6 | 2.9 | 6.8 | 5.3 |
| (SEQ ID NO; 15) | | | | |
| | 0.2 | 1.0 | 1.0 | 1.9 |
| | 0.4 | 4.5 | 1.8 | 8.2 |
| VMASVDNPL | <0.1 | <0.1 | <0.3 | <0.3 |
| (SEQ ID NO: 880) | | | | |
| LTSTVQLIM | <0.2 | <0.2 | <1.0 | <0.5 |
| (SEQ ID NO: 65) | | | (6.3) | (3.7) |
| | <0.2 | <0.2 | <0.5 | <0.5 |
| | | | (3.2) | (4.0) |
| ITDFGRAKL | 0.8 | 8.8 | 3.2 | 14.0 |
| (SEQ ID NO: 3) | | | | |
| | 2 | 2.9 | 8.6 | 5.3 |
| HVKITDFGRAK* | 1.2 | 8.3 | 4.9 | 14.9 |
| (SEQ ID NO: 9) | | | | |
| FGRAKLLGA | <0.1 | 1.4 | <0.3 | 2.6 |
| (SEQ ID NO: 6) | | | | |
| HVKITDFGR* | 0.7 | 1.4 | 3.1 | 2.5 |
| (SEQ ID NO: 1) | | | | |
| RILKVTEFK | <0.1 | <0.1 | <0.1 | <0.1 |
| (SEQ ID NO: 897) | | | | |
| RILKVTEFKK | <0.1 | <0.1 | <0.1 | <0.1 |
| (SEQ ID NO: 898) | | | | |
| LRILKVTEFK | <0.1 | <0.1 | <0.1 | <0.1 |
| (SEQ ID NO: 899) | | | | |
| STVOLIMQL | <0.2 | <0.2 | <0.5 | <0.5 |
| (SEQ ID NO: 68) | | | (3.2) | (4.0) |
| AIKESPKANK | <0.2 | <0.5 | <0.4 | <1.0 |
| (SEQ ID NO: 32) | | | | |
| MASVDNPLM | <0.1 | <0.1 | <0.1 | <0.1 |
| (SEQ ID NO: 955) | | | | |
| NPLMCRLLGI | <0.1 | <0.1 | <0.1 | <0.1 |
| (SEQ ID NO: 956) | | | | |
| VDNPLMCRL | <0.1 | <0.1 | <0.1 | <0.1 |
| (SEQ ID NO: 957) | | | | |
| HVKITDFGRAKLLGAEE | 2.0 | 3.9 | 8.2 | 7.0 |
| (SEQ ID NO: 826) | 0.2 | 7.3 | 1.0 | 13.1 |
| | 2.9 | 10.3 | 12.0 | 18.4 |
| QALLRILKVTEFKKIKV | <0.1 | <0.1 | <0.1 | <0.1 |
| (SEQ ID NO: 900) | | | | |
| KIPVAIKESPKANKEIL | <0.1 | <1.0 | <0.5 | <1.0 |
| (SEQ ID NO: 914) | | | | |
| LTSTVQLIMQLMPFGCL | <0.1 | <1.0 | <0.5 | <2.0 |
| (SEQ ID NO: 907) | | | (1.4) | (14.8) |
| MASVDNPLMCRLLGICL | <0.1 | <0.1 | <0.1 | <0.1 |
| (SEQ ID NO: 958) | | | | |

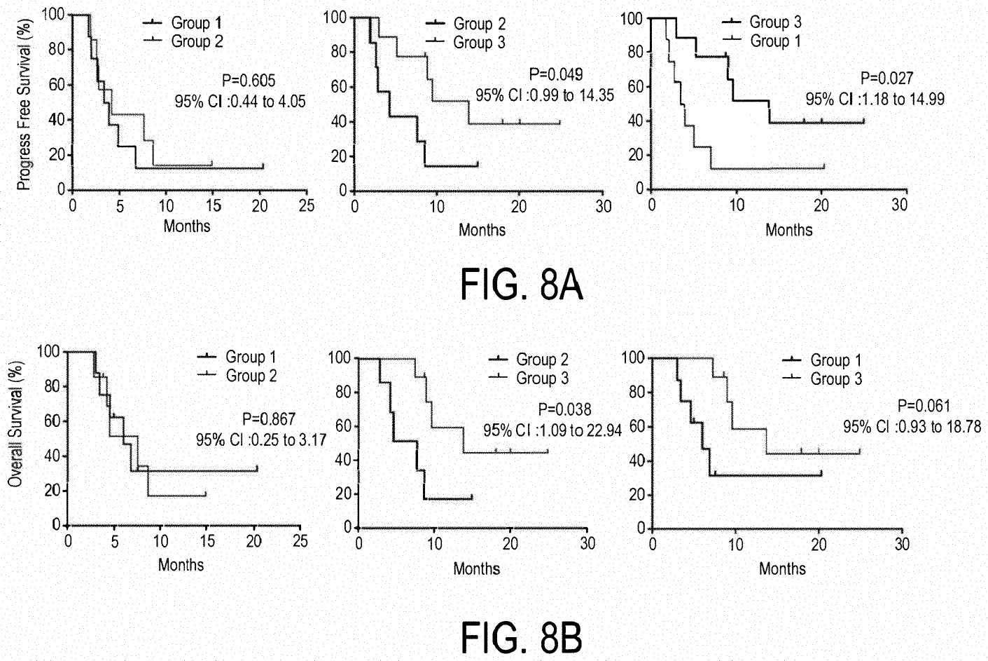

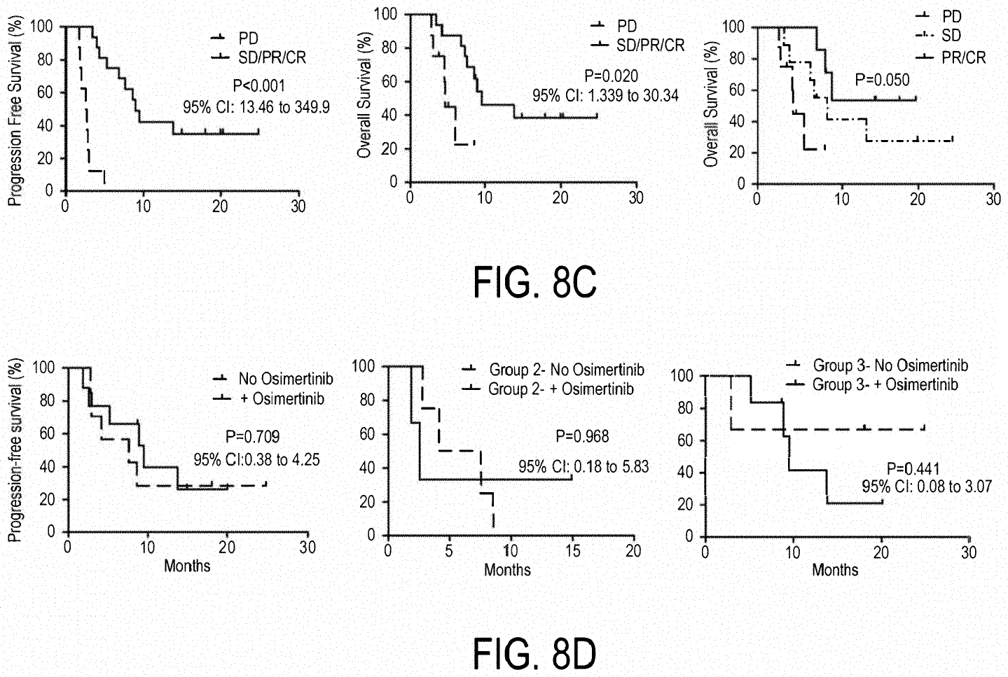

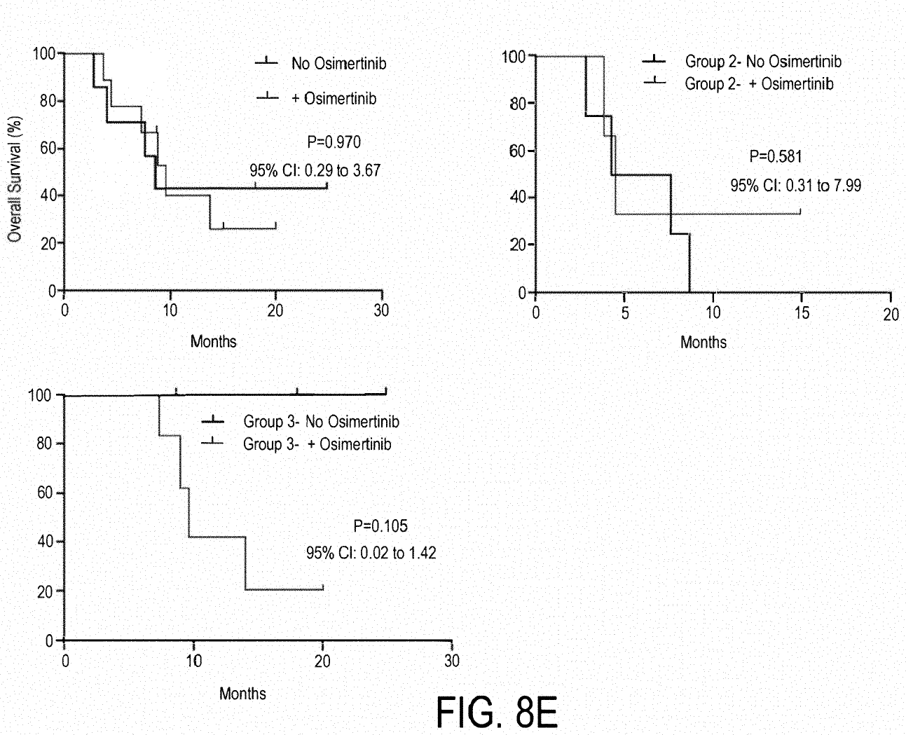

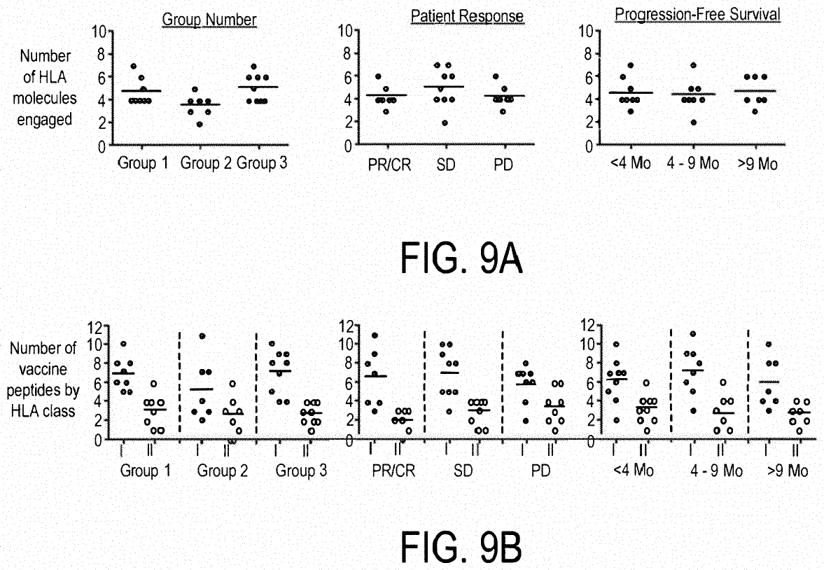

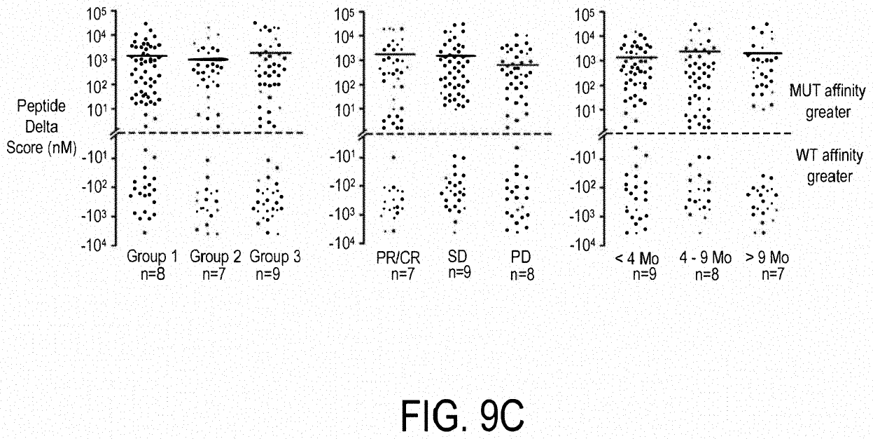

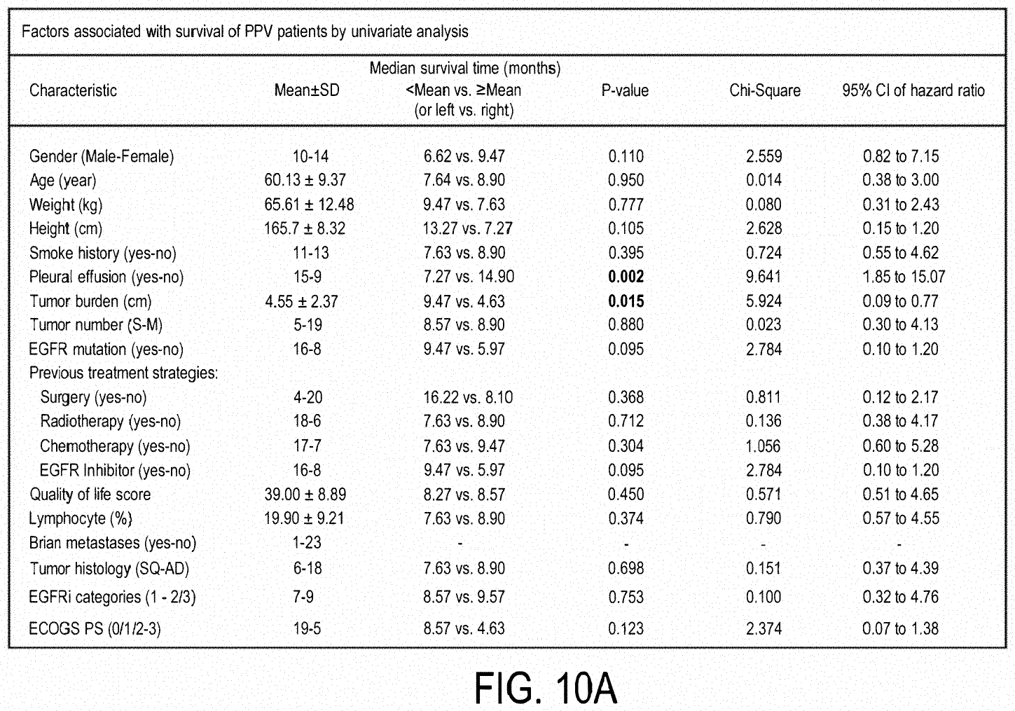

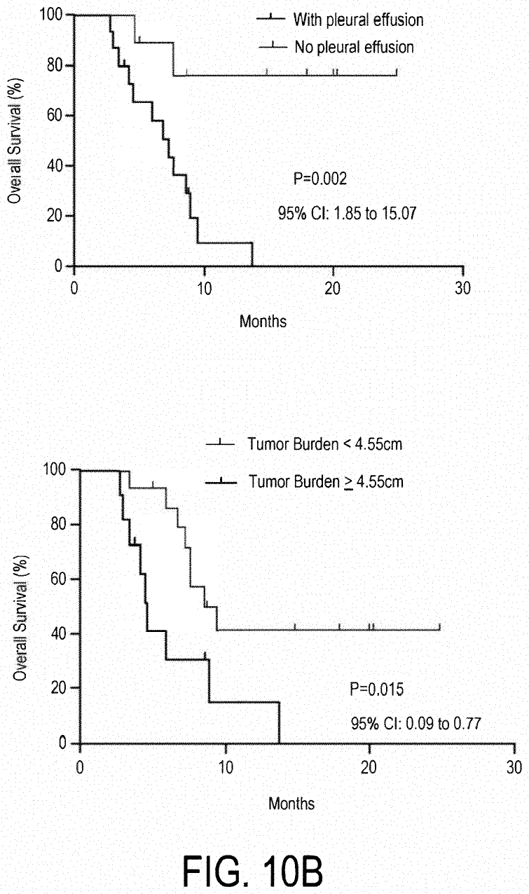

Notably, none of the patients in Group 3 had a break or “drug holiday” from EGFRi treatment prior to treatment on the trial, supporting that the NeoAg vaccination played a key role in these clinical responses. Notably, Group 3 patients experienced longer median OS compared with Group 2 patients (13.8 vs. 7.6 months, P=0.038, FIG. 1G; FIGS. 8A & 8B). Patients that experienced clinical benefit (PR/CR or SD) also demonstrated significantly extended OS and PFS (FIG. 1H; FIG. 8C). However, neither clinical response nor progression-free survival (PFS) were associated with the number of immunizing peptides, peptide length, predicted HLA binding affinity, the number of HLA molecules engaged, peptide delta score, or EGFRi treatment history (FIGS. 1D-1F; FIGS. 8D & 8D; FIGS. 9A-9C). PR/CR patients received vaccines targeting significantly fewer somatic mutations compared to the vaccines of PD patients (P=0.014). This was a consequence of the 7 responding patients receiving a significantly higher proportion of EGFR NeoAg peptides in their vaccines (P<0.001, FIG. 1F), supporting the notion that EGFR NeoAg vaccination was linked to the clinical responses observed. Univariate analysis showed that presence of pleural effusion and elevated tumor burden were two risk factors negatively impacting patient survival outcomes (FIGS. 10A & 10B); both are well-known risk factors for NSCLC (Morgensztern et al., 2012a; Morgensztern et al., 2012b).

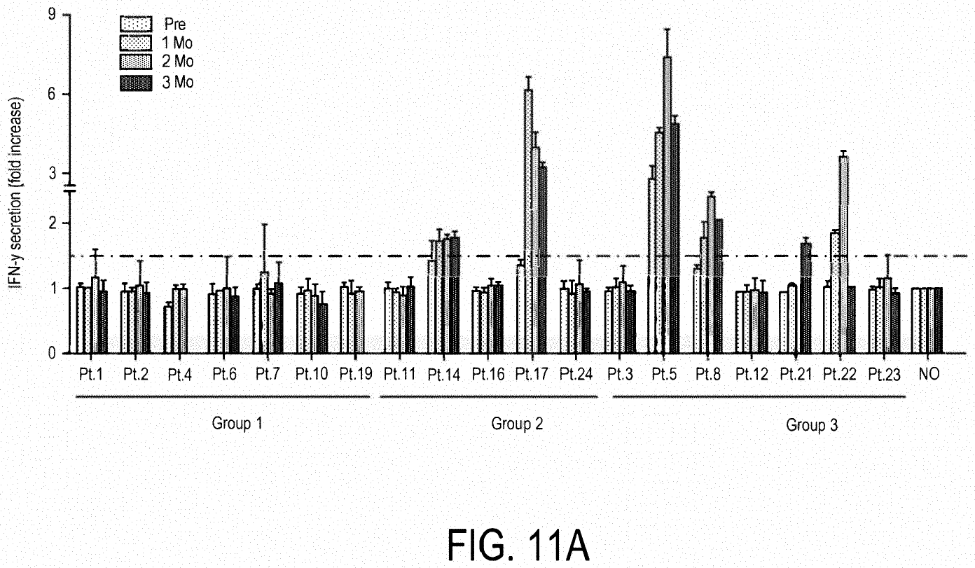

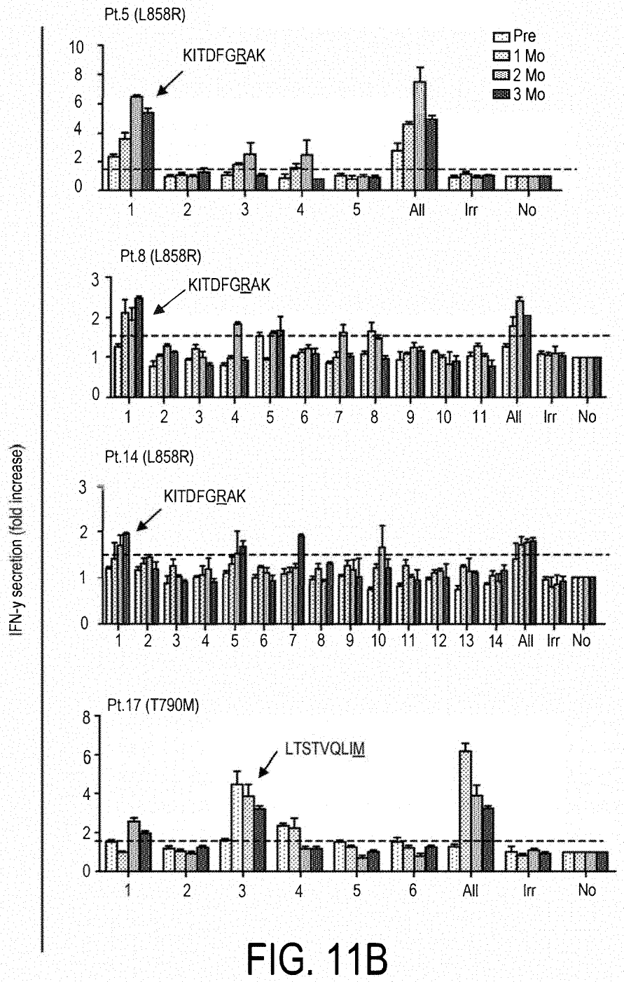

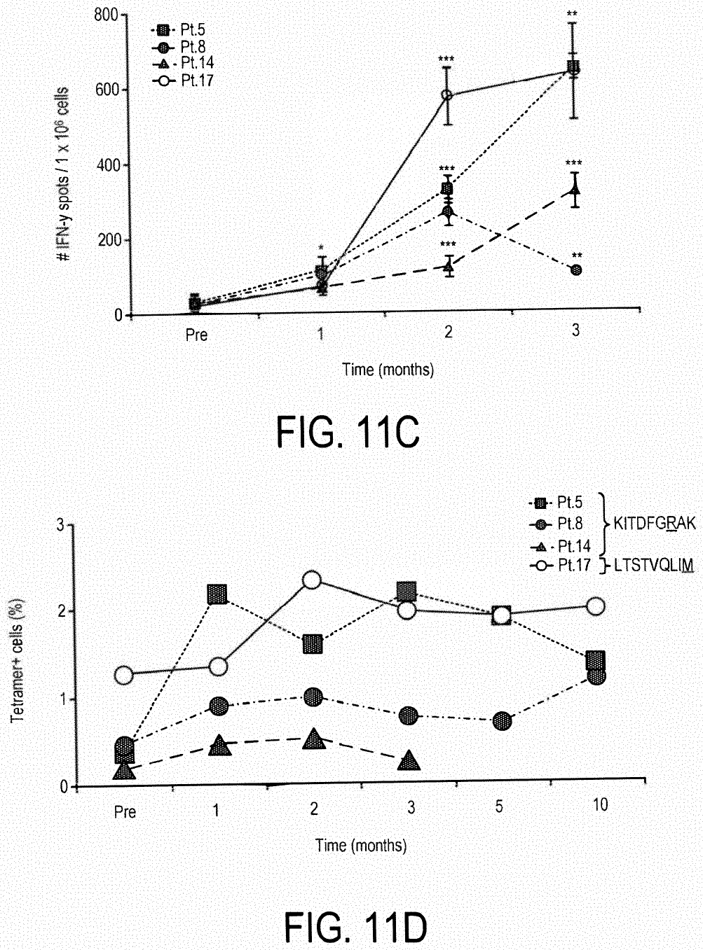

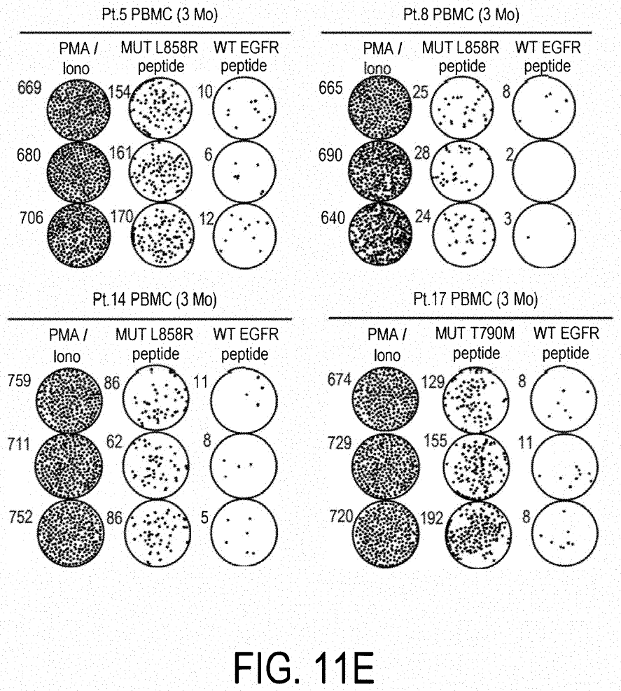

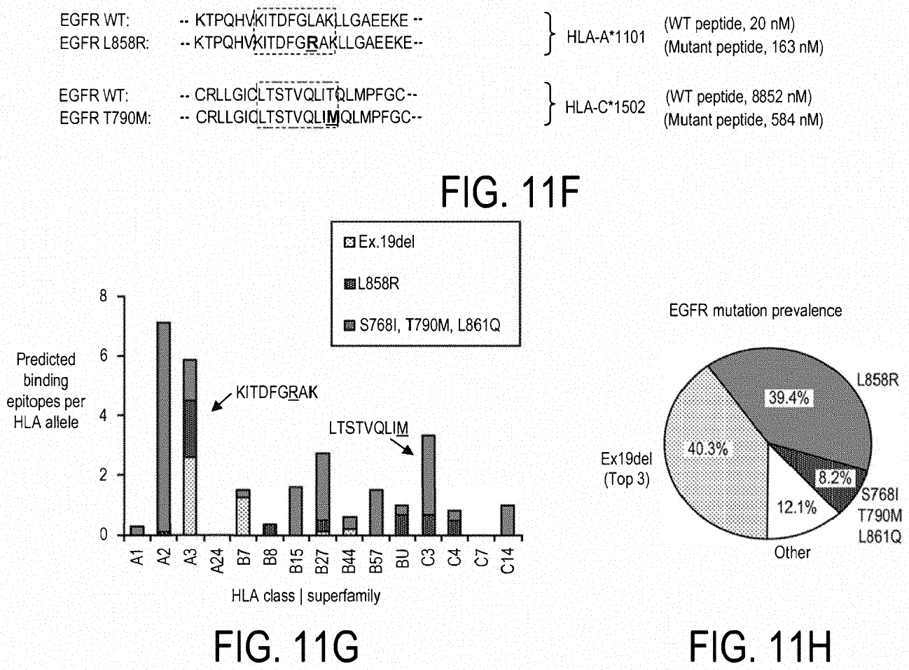

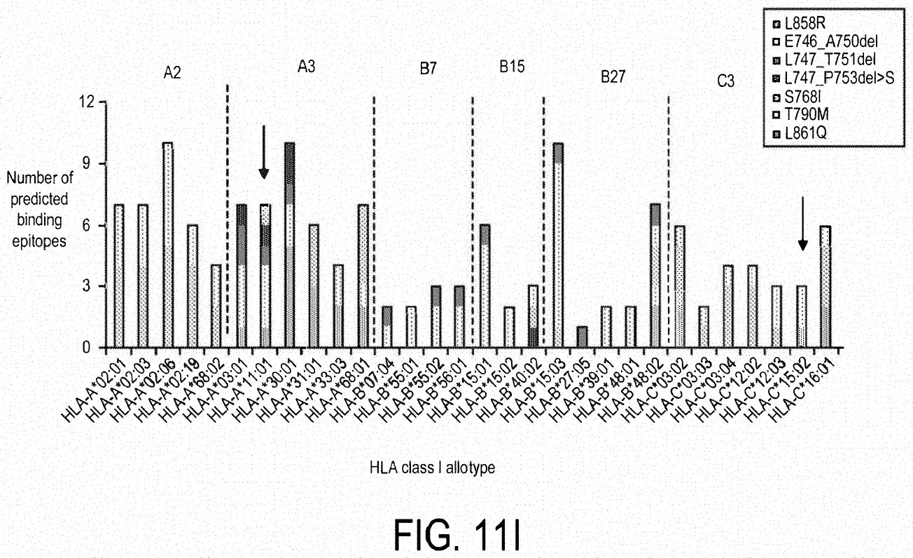

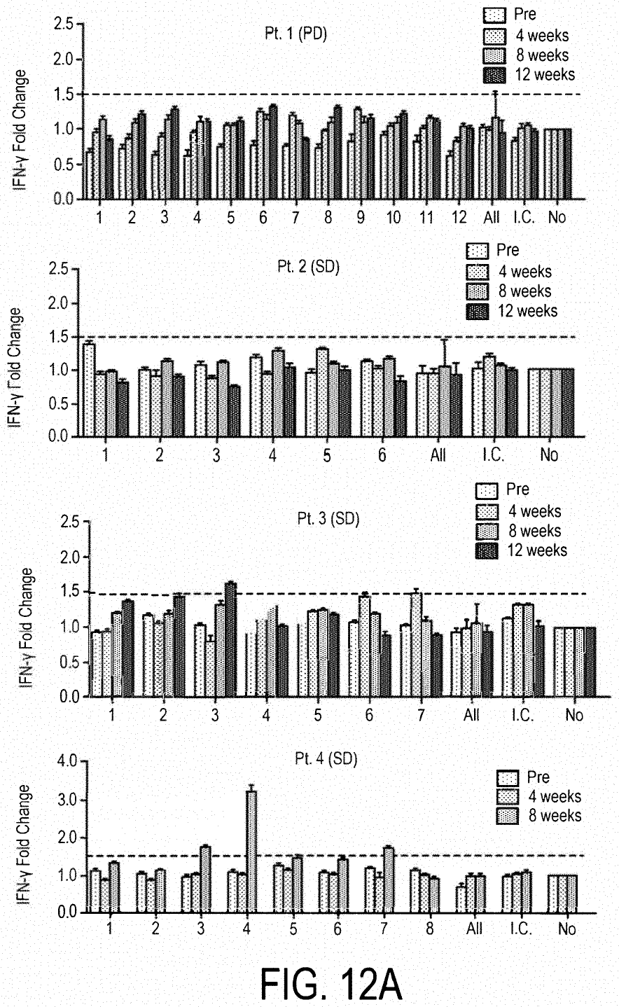

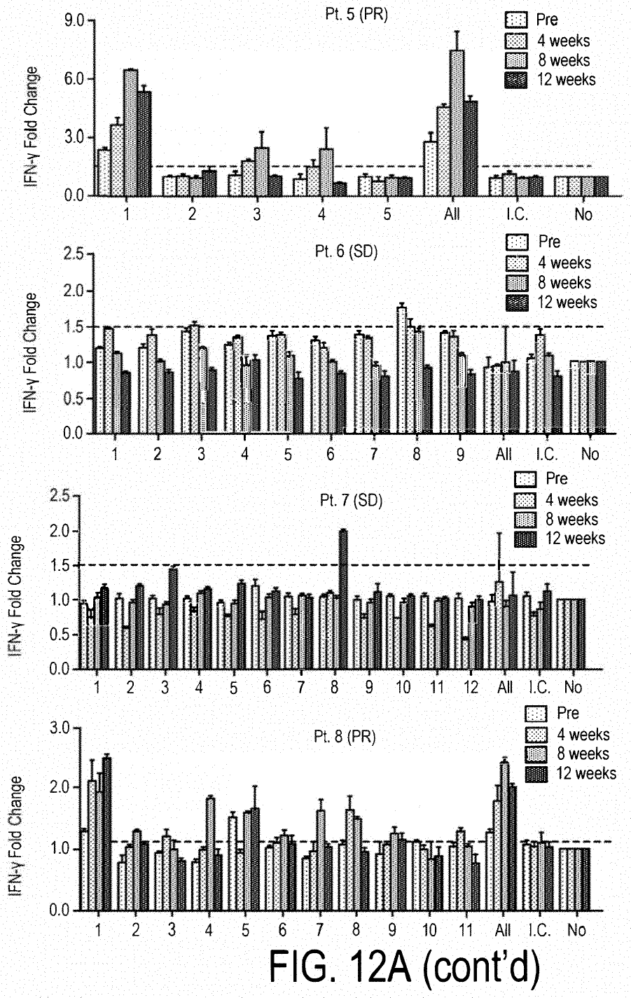

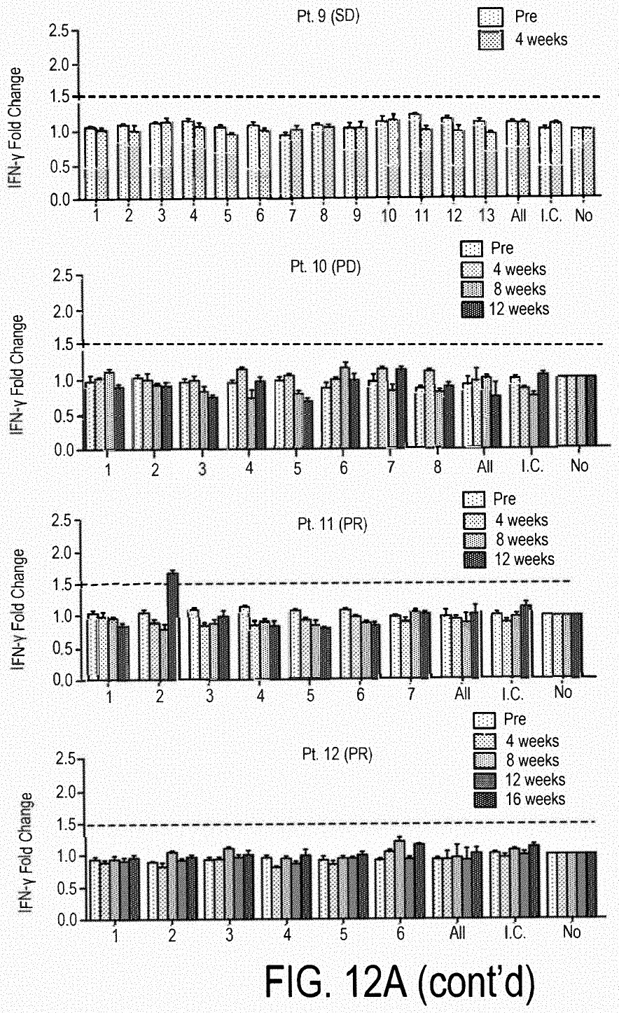

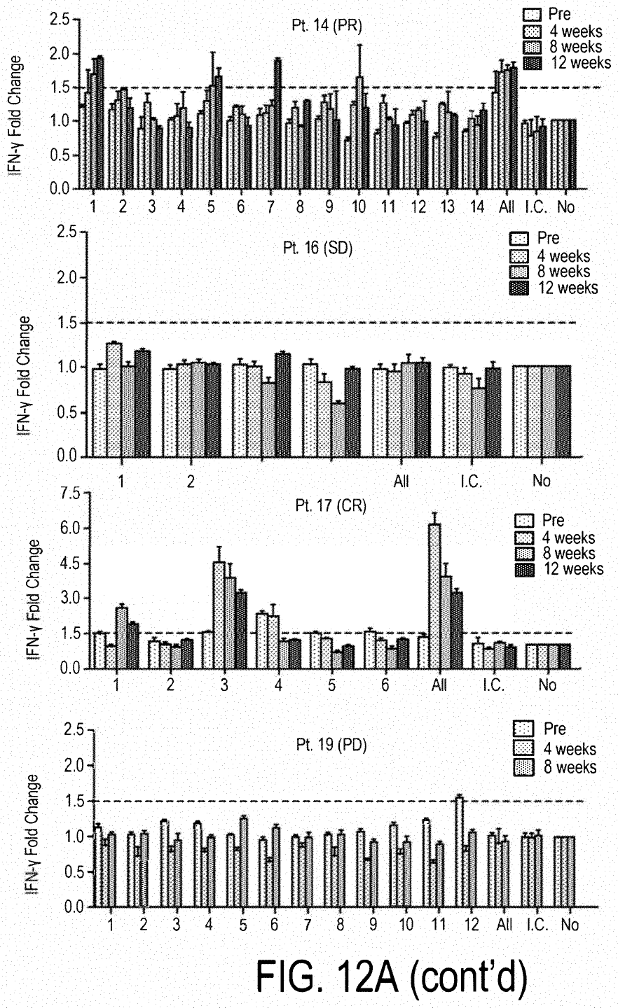

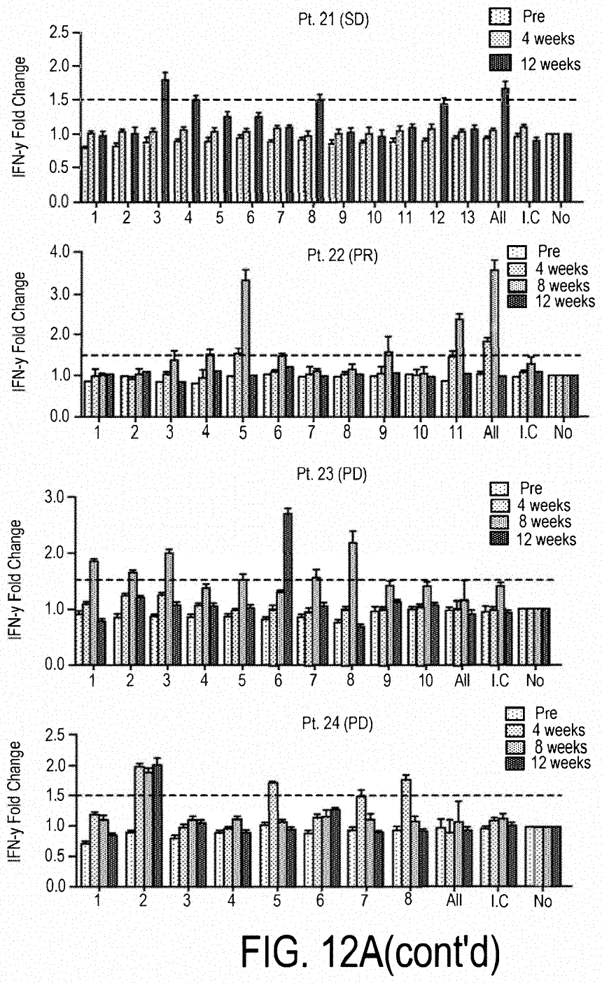

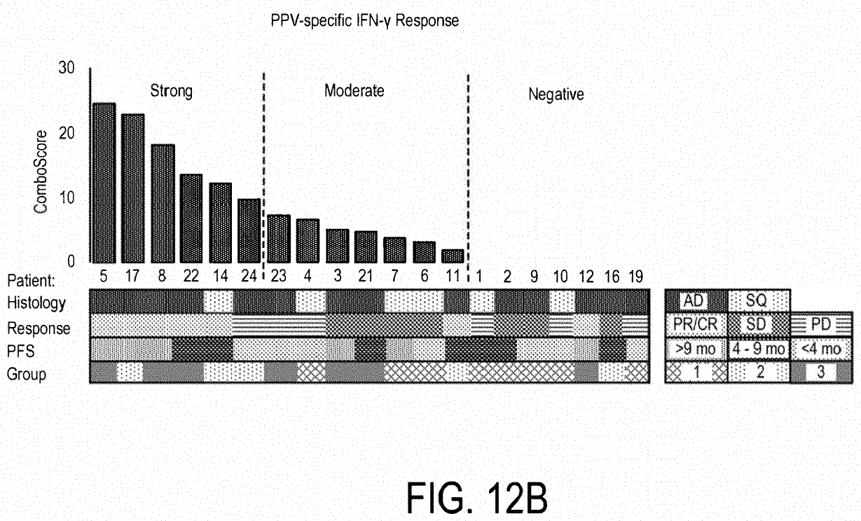

PPV induced NeoAg-specific T-cell responses against shared EGFR mutations. To better understand the nature of the antitumor responses, sequential immune monitoring was performed on samples of patient peripheral blood mononuclear cells (PBMC) collected pre- or post-PPV (see Methods). Vaccine-induced immune responses were initially screened by stimulating individual patient PBMC with pools of their immunizing peptides and measuring specific interferon-gamma (IFN-γ) secretion by ELISA (FIG. 11A). In this assay, peptide pool-specific reactivity was detected in 6 of the 20 patients assessed, all from Group 2 (2 of 5 pts.) or Group 3 (4 of 7 pts.). Peptide deconvolution revealed individual NeoAg peptide-specific IFN-γ responses in 7 additional PPV patients (FIG. 12A; Table 5). Based on these results, we calculated an Immune Response ComboScore (IRC) that accounted for the breadth, intensity, and persistence of PPV-specific IFN-γ responses (see Methods; FIG. 12B). Of the 7 patients with the lowest ComboScores (IRC=zero), 5 patients were from Group 1 and only one (Pt. 12 from Group 3) had experienced a clinical objective response. By contrast, the 6 patients with the highest IRCs were all from Group 2 or Group 3, and 5 of these patients were clinical responders that had experienced PFS longer than the median PFS time (FIG. 12B).

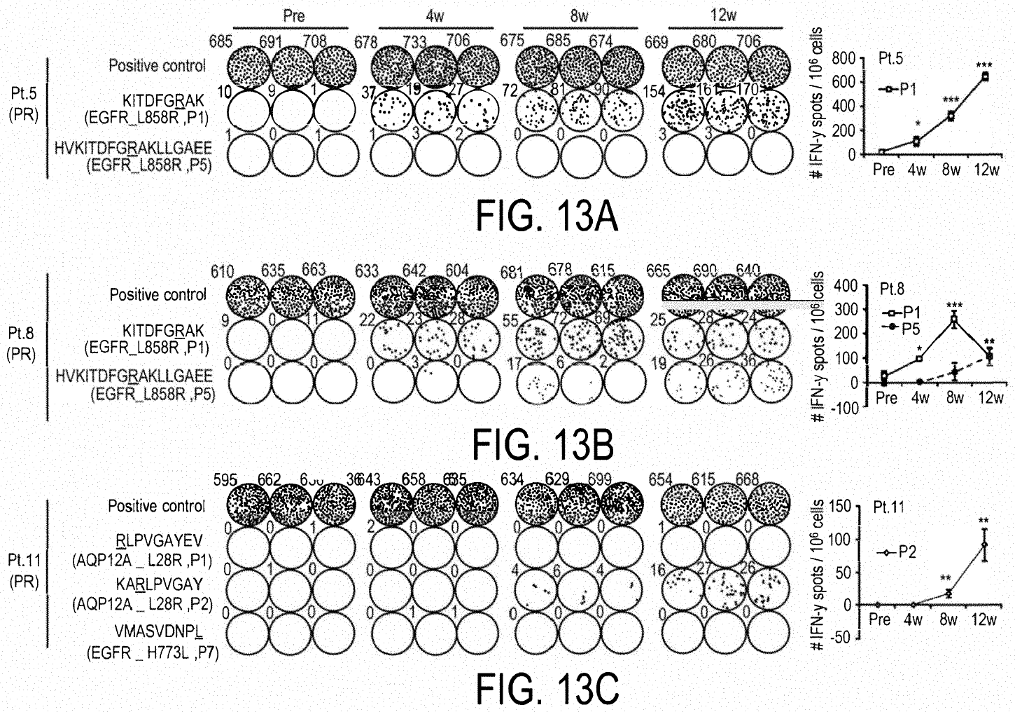

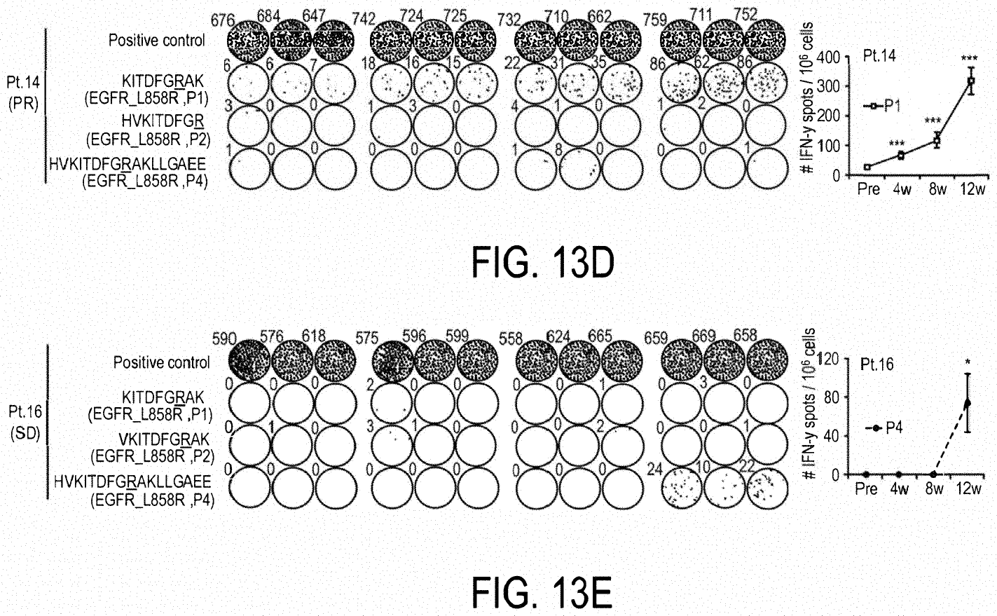

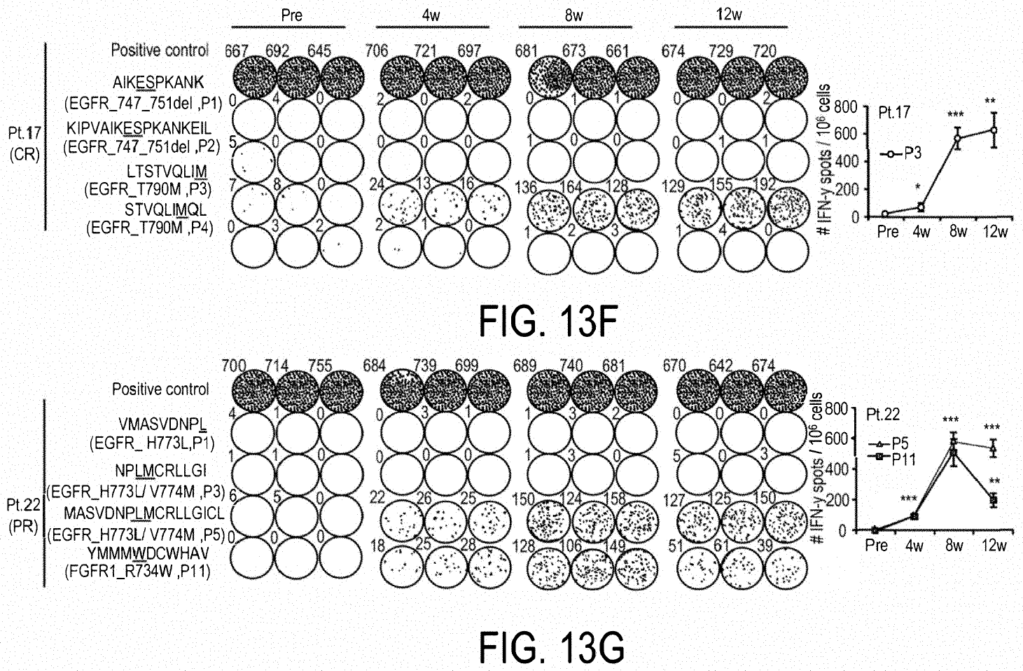

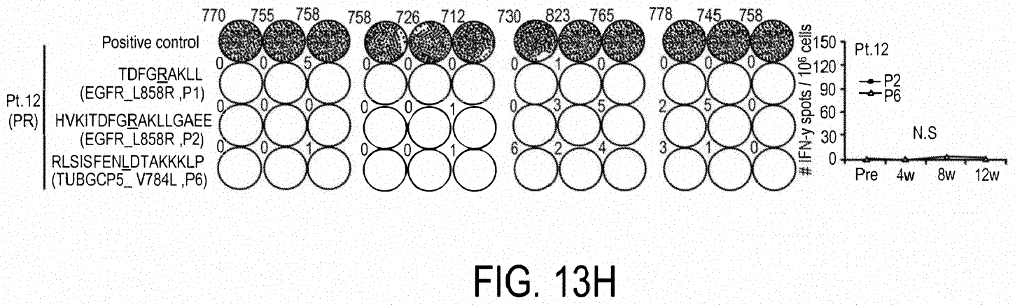

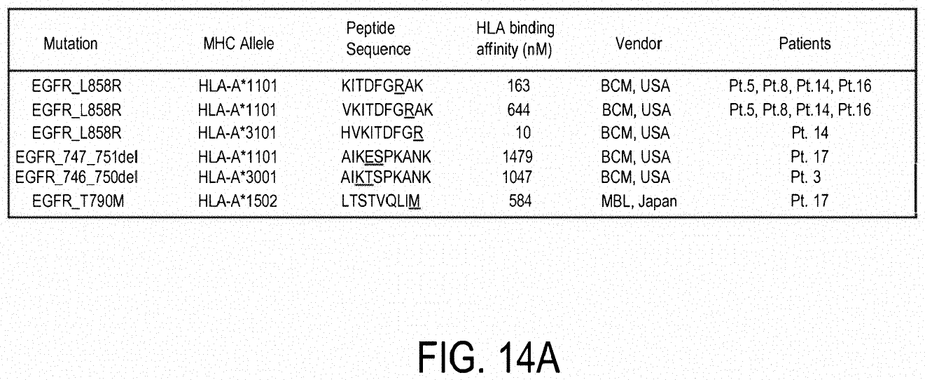

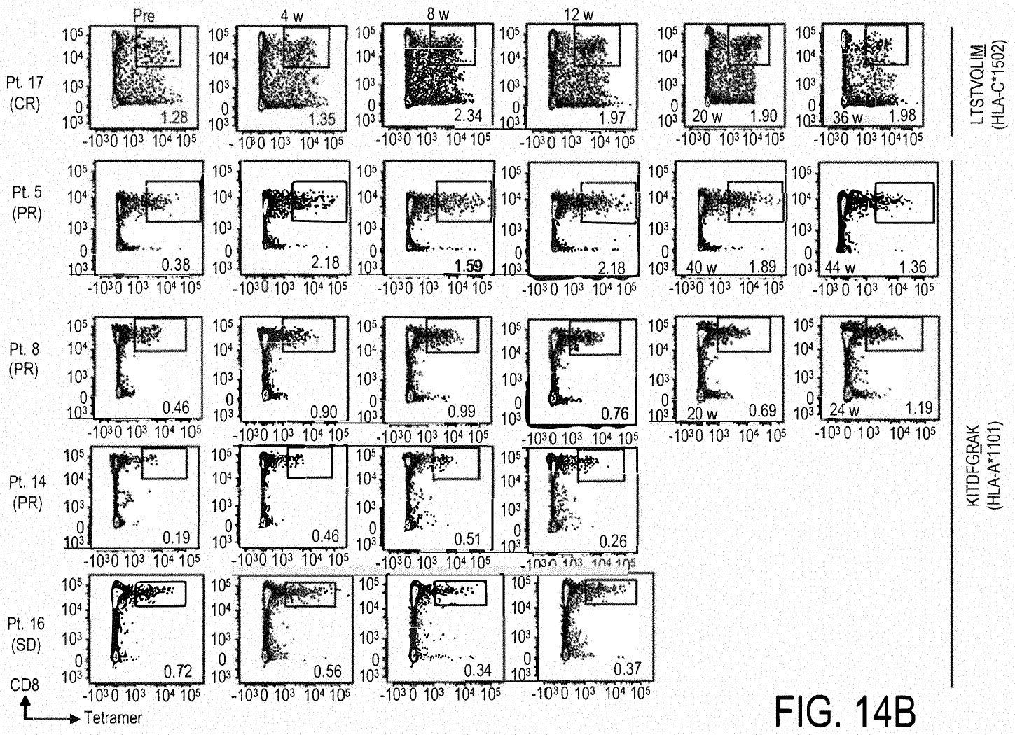

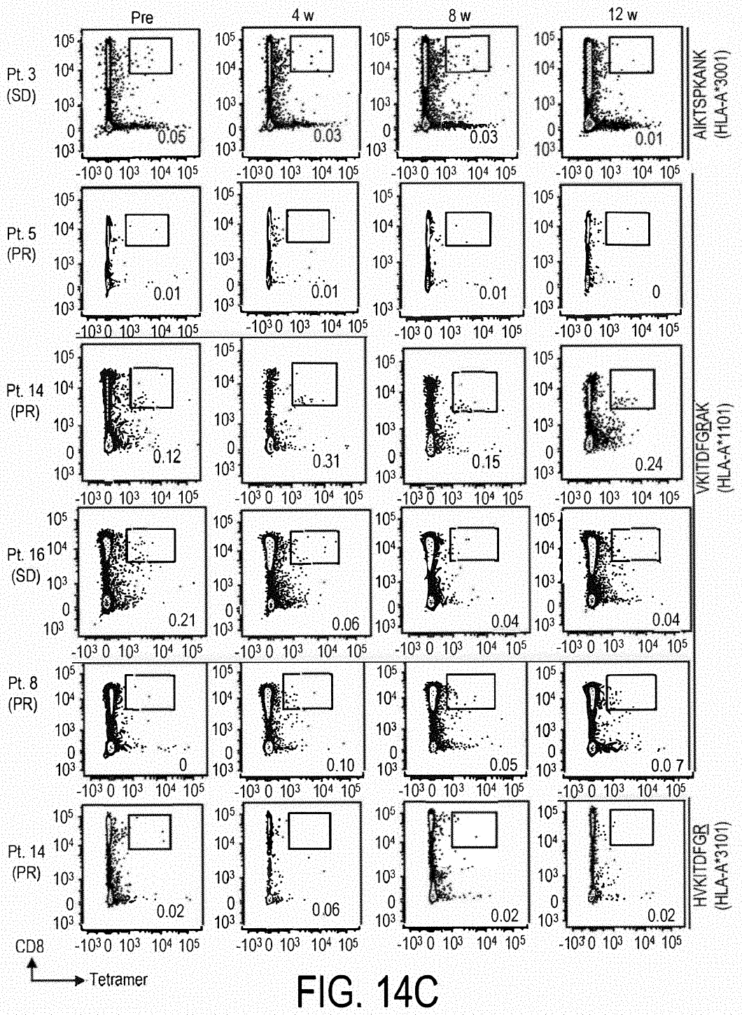

ELISPOT-based immune monitoring revealed that EGFR mutations constituted the dominant targets of NeoAg-specific T-cell responses in 5 out of the 6 responding patients for which vaccine-induced responses were observed. While Pt. 11 generated a moderate IFN-γ response to a mutated AQP12A(L28R) NeoAg peptide restricted to HLA-A *0301, they did not generate any detectable response against an HLA-A*0201-restricted EGFR(H773L) NeoAg vaccine peptide (FIG. 13). By contrast, 3 different responding HLA-A *1101 patients (Pts. 5, 8, and 14) generated dominant immune reactivity against the A*1101-restricted peptide KITDFGRAK (SEQ ID NO: 4), encompassing the highly shared EGFR-L858R mutation (FIG. 11B). Likewise, CR Pt. 17 demonstrated a strong response against the HLA-C*1502-restricted, T790M-containing peptide LTSTVQLIM (SEQ ID NO: 65). ELISPOT and HLA/peptide tetramer staining assays confirmed specific CD8 T cell responses against both of these NeoAg peptides, with both assays showing incremental increases in T-cell frequencies for up to 3 months during immunization (FIGS. 11C & 11D; FIG. 13; FIG. 14). Importantly, vaccine-induced T cells from 4 of the patients were functionally capable of distinguishing between wild-type and mutant EGFR peptides (FIG. 11E).

Patient 22 (PR), in addition to generating a robust response against an A*0201-restricted FGFR1(R734W) NeoAg peptide, also generated vaccine-induced reactivity against a long DRB1 *0901-restricted NeoAg peptide (MASVDNPLMCRLLGICL) (SEQ ID NO: 958) containing the compound EGFR mutation H773L/V774M. Pts. 8 and 16 both generated immune responses against a long HLA class II-restricted EGFR NeoAg peptide (HVKITDFGRAKLLGAEE) (SEQ ID NO: 826) containing the L858R mutation (FIG. 13). However, three other patients (5, 12, and 14) vaccinated with the same long L858R peptide did not generate detectable antigen-specific immune responses, despite sharing relevant HILA class II allotypes with Pts. 8 and 16. Of the 4 HLA-A* 1101 patients immunized with the KITDFGRAK peptide (SEQ ID NO: 4), Pt. 16 was the lone patient who failed to generate a detectable CD8⁺ T cell response against this NeoAg, and was also the only one of the 4 patients to not experience a clinical response (SD). Collectively, these results provide evidence that multiple distinct EGFR mutations can be immunogenic targets of NeoAg vaccine-specific CD4⁺ and CD8⁺ T-cell responses, which are associated with clinical objective responses in NSCLC patients.

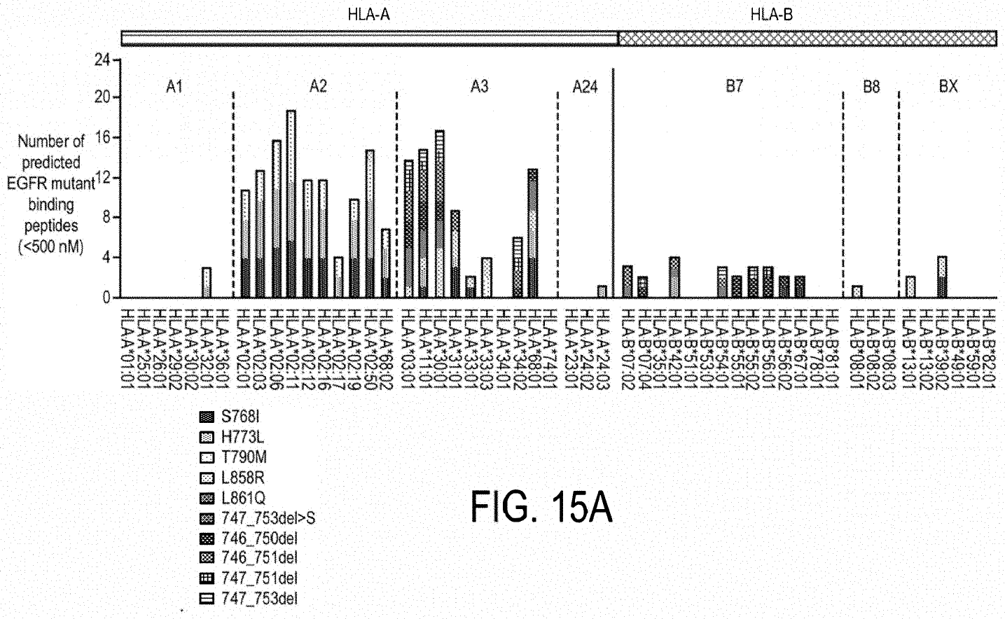

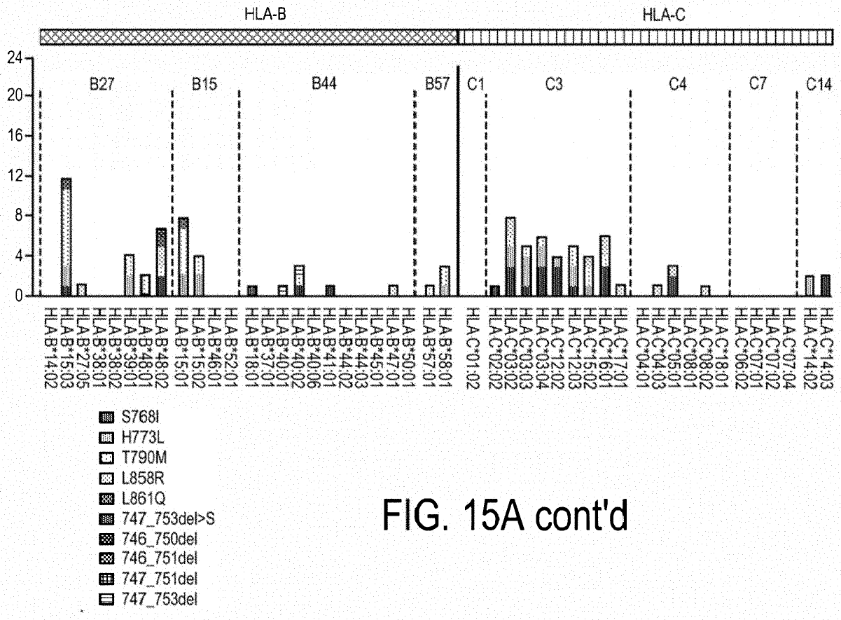

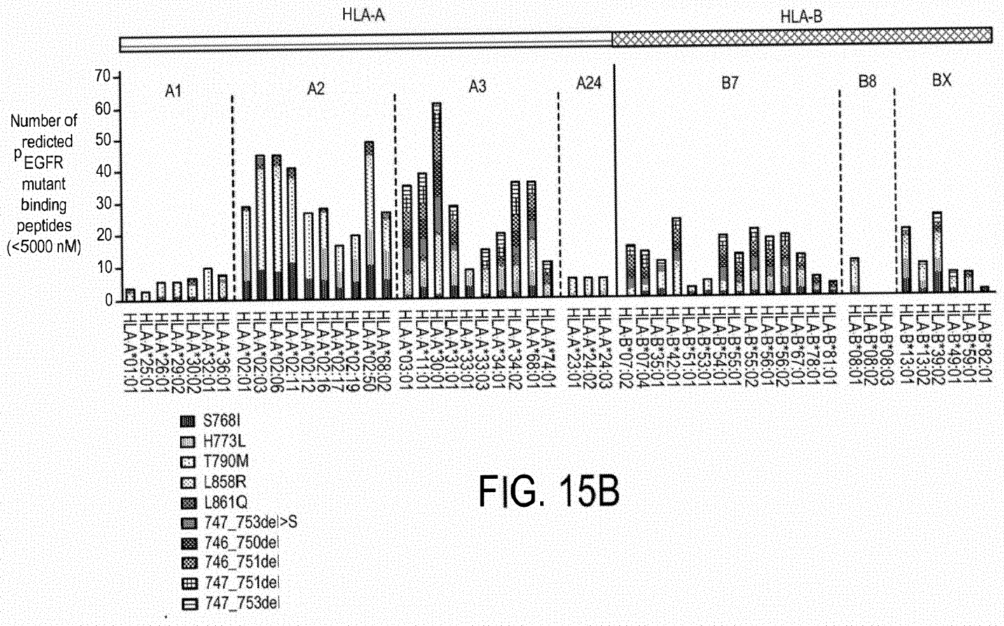

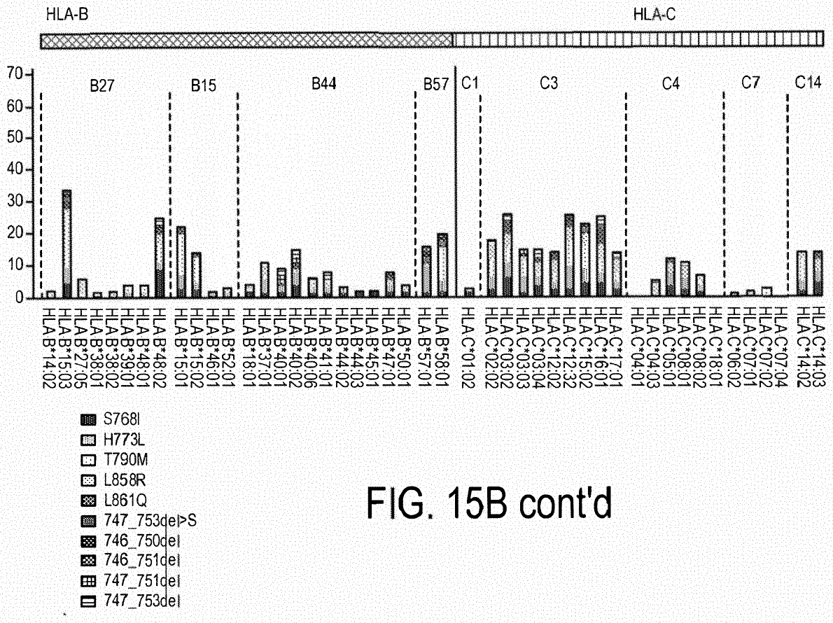

Surprisingly, the KITDFGRAK NeoAg peptide (SEQ ID NO: 4) is predicted to bind HLA-A*1101 with lower affinity (163 nM) than the corresponding WT peptide (20 nM), though it still falls well within typical range for moderate-affinity HLA binders (FIG. 11F). By contrast, since the EGFR-T790M mutation converts the peptide C-terminal anchor from a polar threonine residue to a hydrophobic methionine residue, binding of the NeoAg LTSTVQLIM peptide (SEQ ID NO: 65) to HLA-C*1502 is strongly favored over the WT peptide (FIG. 11F). Global analysis of HLA peptide-binding preferences revealed a striking skewing of EGFR NeoAg presentation at the HLA superfamily level: While L858R and Exon 19 deletion mutations, which together comprise >80% of all EGFR mutations, produce NeoAgs with elevated basic amino acid content favoring binding primarily to A3 superfamily members including HLA-A* 101, NeoAgs containing shared 57681, T790M, and L861Q mutations are more hydrophobic and are thus favored to bind members of the A2, B15, B27 and C3 superfamilies, which includes HLA-C* 1502 (FIG. 11G-11I; Kobayashi et al., 2016). By contrast, HLA class I allotypes within the A1, A24, B8, and C7 superfamilies are not expected to bind and present most shared EGFR NeoAgs (FIGS. 15A & 15B). HLA class II molecules are predicted to bind peptides containing a wide array of EGFR mutations, and class II superfamilies are not predicted to show skewed binding preferences, with the potential exception of the DP1 and DP3 allotypes (FIG. 15C; Sidney et al., 2008; Harjanto et al., 2014; Jensen et al., 2018).

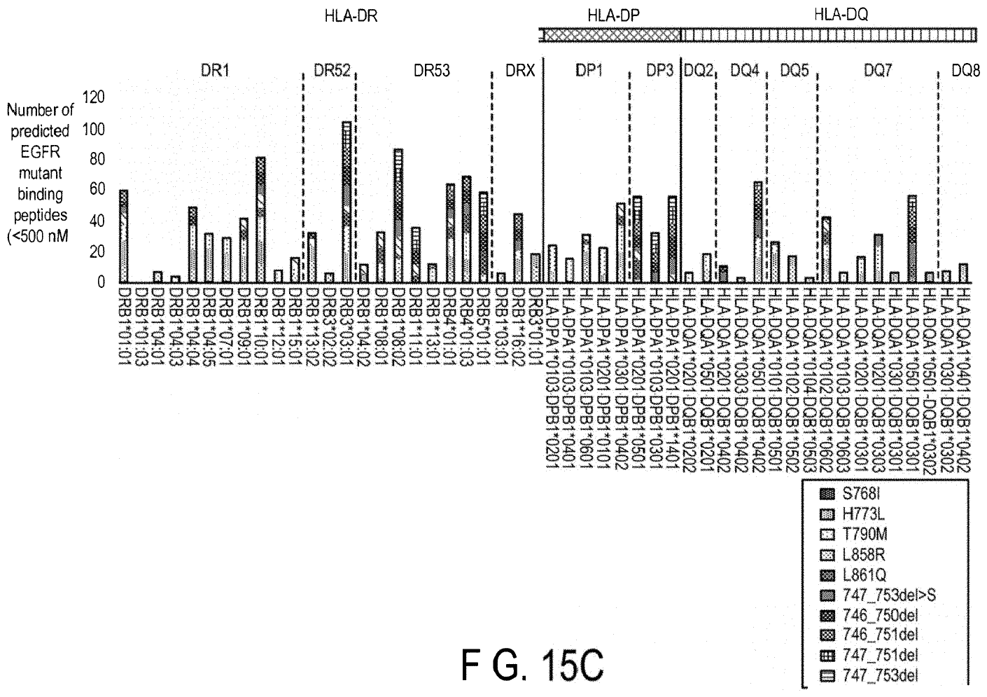

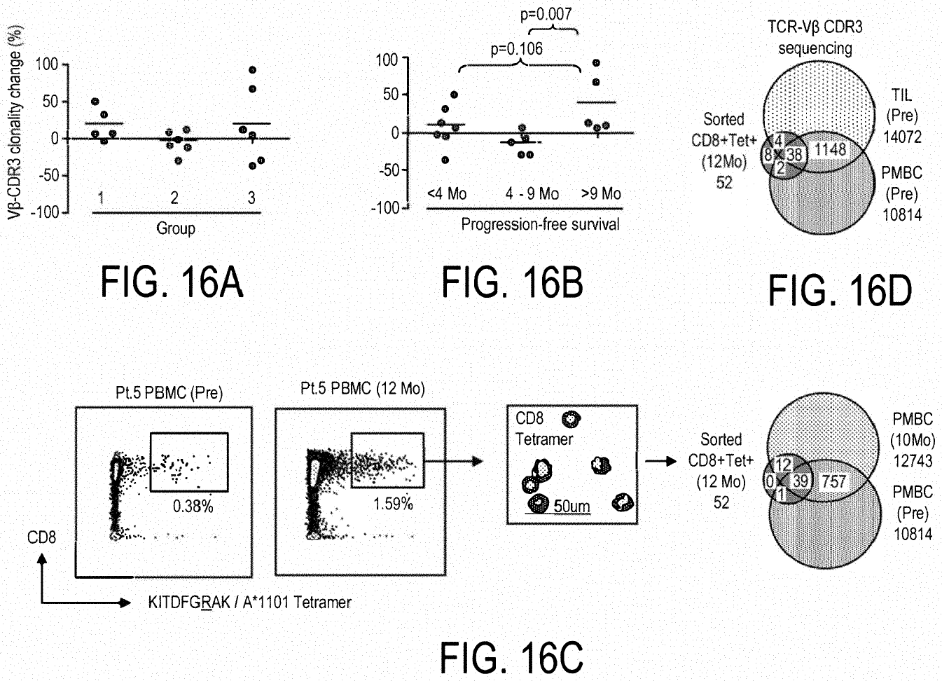

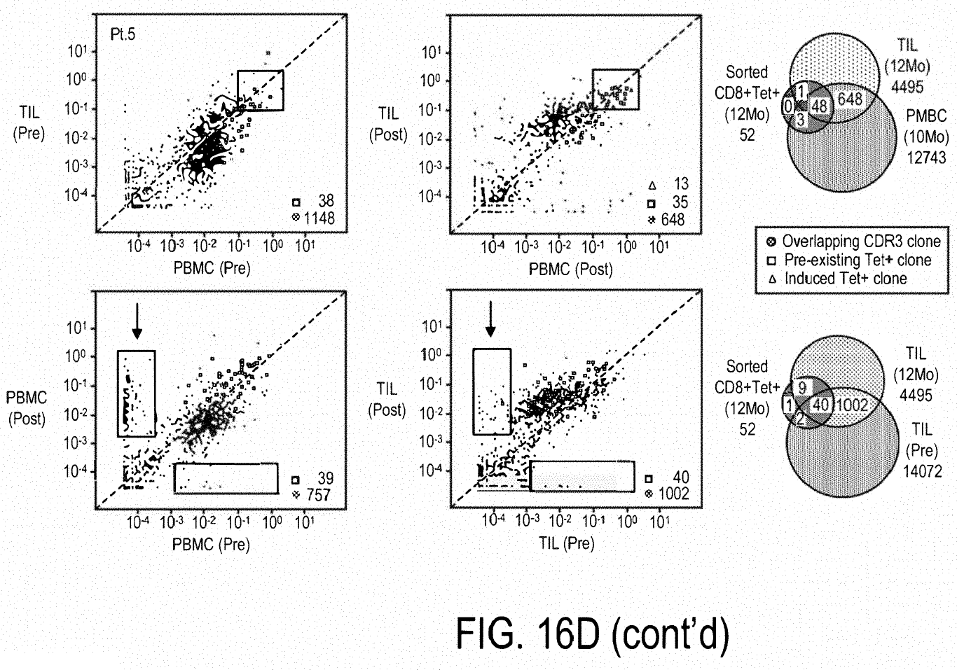

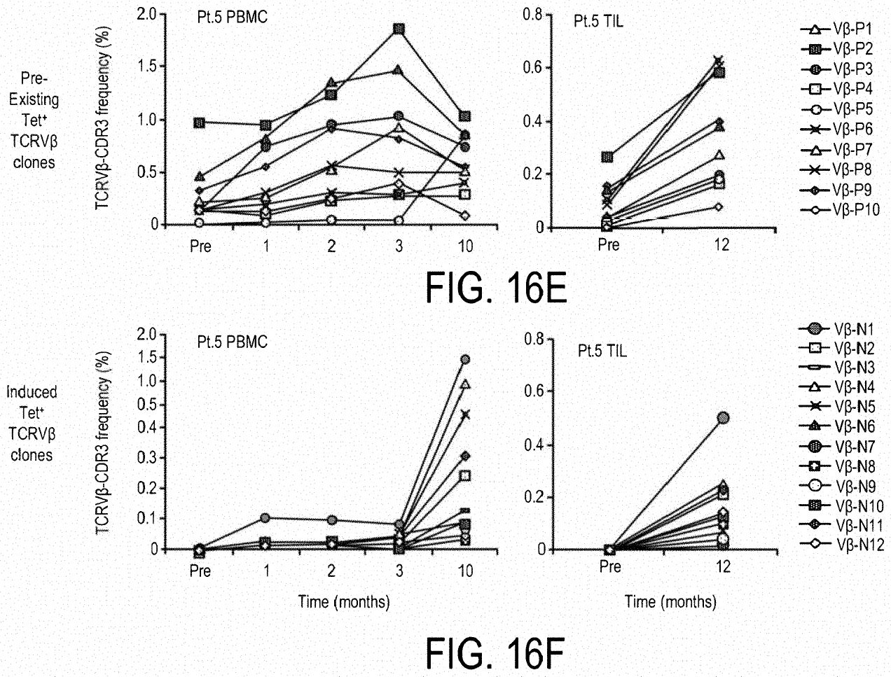

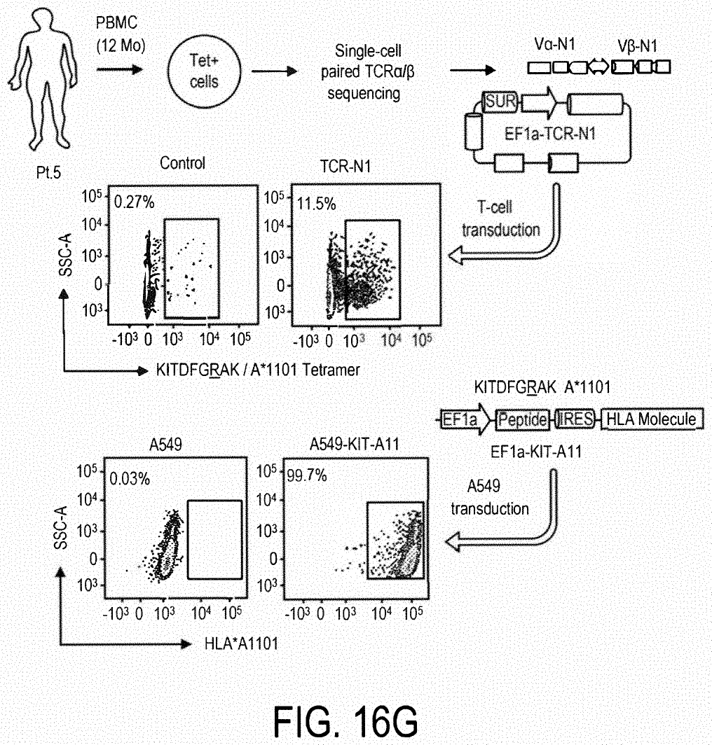

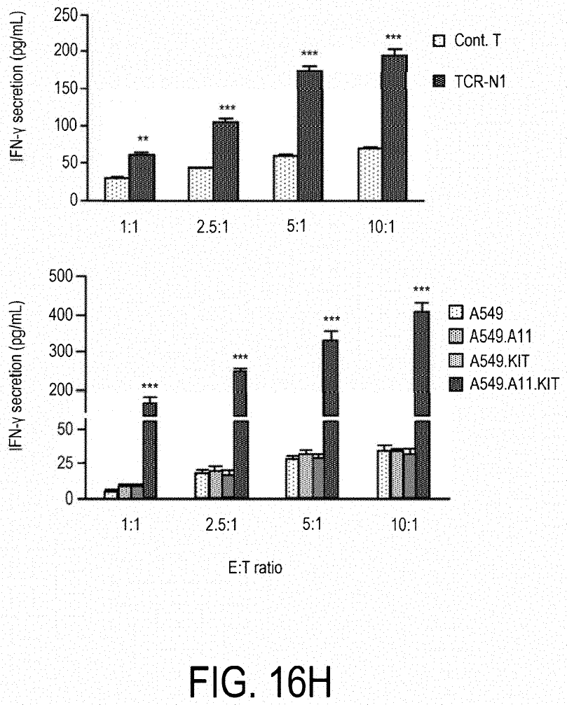

PPV drove proliferation and tumor infiltration of EGFR NeoAg-specific T cells. TCRVβ-CDR3 sequencing was performed on DNA isolated from pre- and post-vaccine PBMC collected from 17 PPV patients. Clonality scores were calculated for each time point, demonstrating increased T-cell clonality in 10 patients after PPV, with 5 patients showing decreases in clonality and 2 patients remaining unchanged (mean change, +13.2%, FIG. 16A). The five patients with the longest PFS (>9 months) all showed increased T-cell clonality post-PPV, particularly Patients 5 and 8 (69.8% and 95.5%, respectively, FIG. 16B). These results were consistent with the expansion of NeoAg-specific T-cells detected by immune monitoring in these two patients post-PPV (FIG. 12; FIG. 13; FIG. 14).

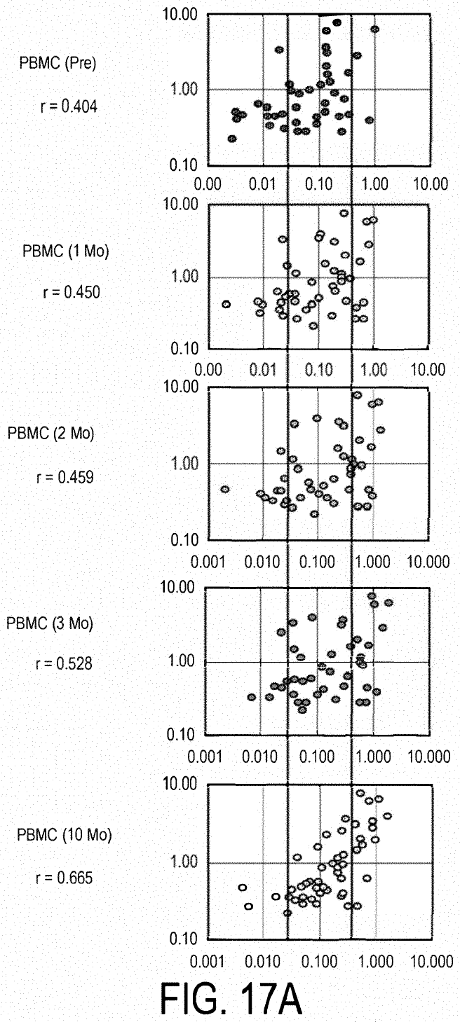

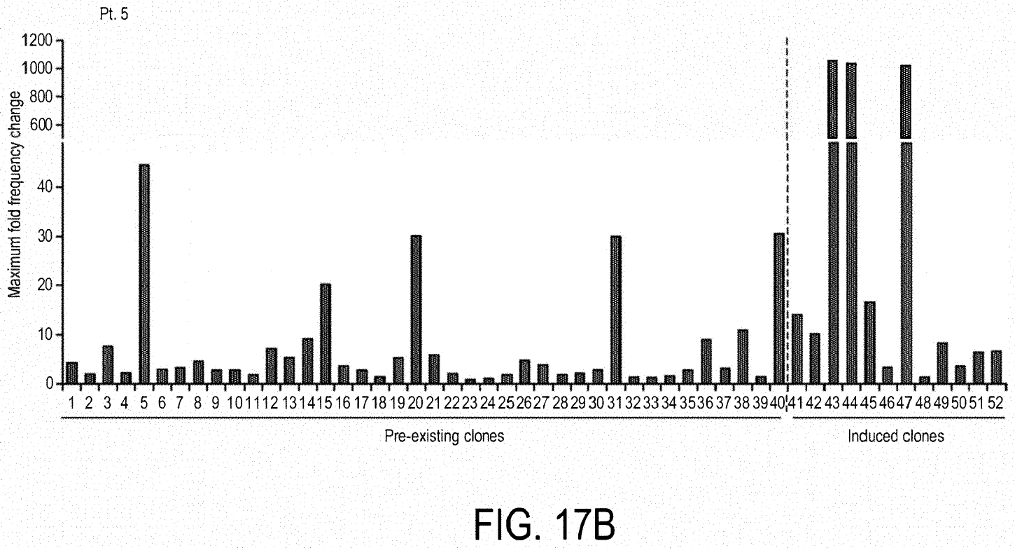

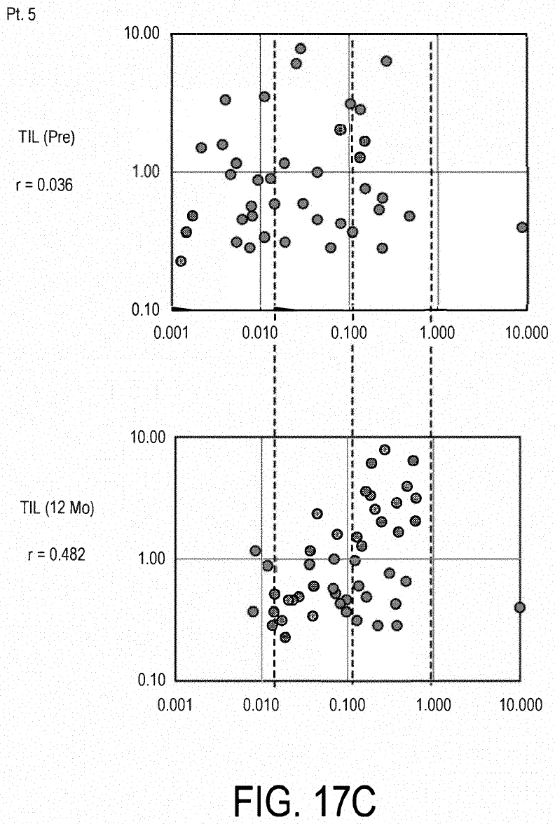

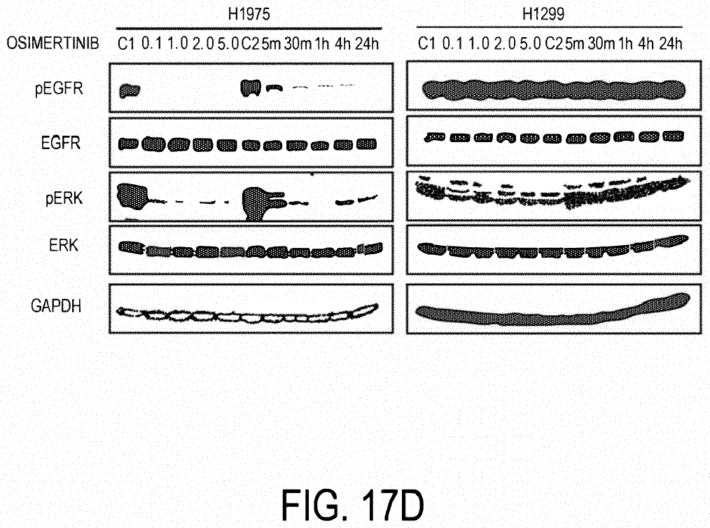

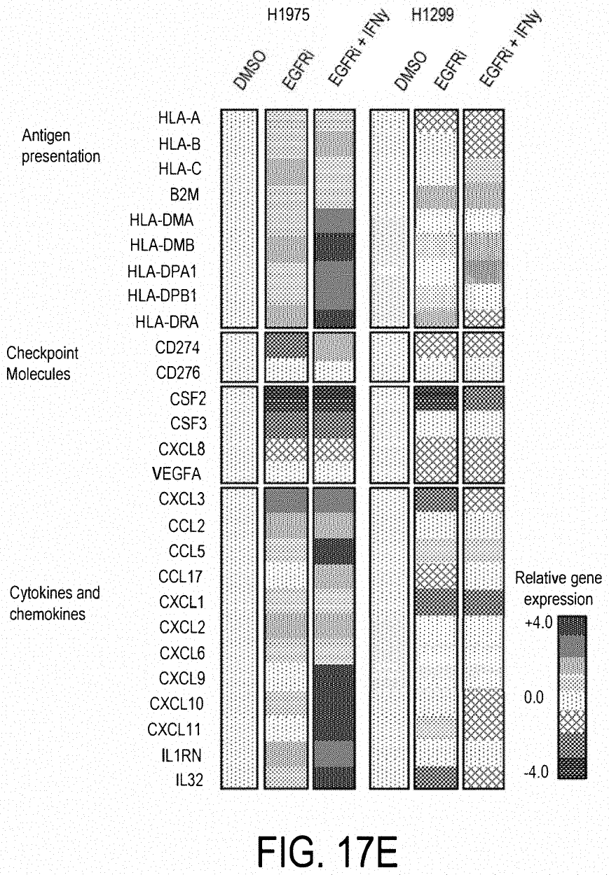

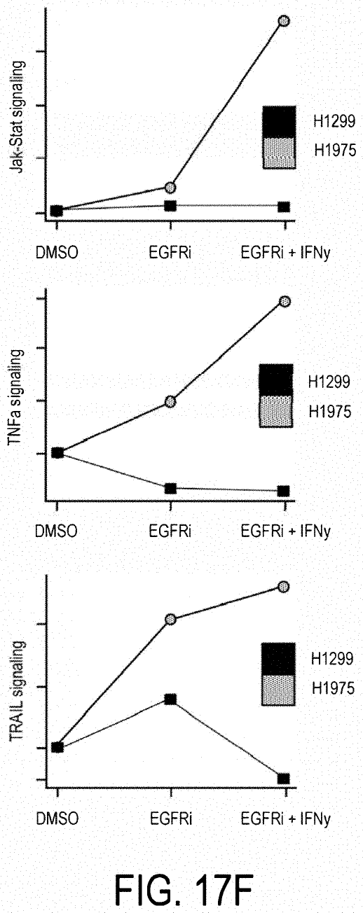

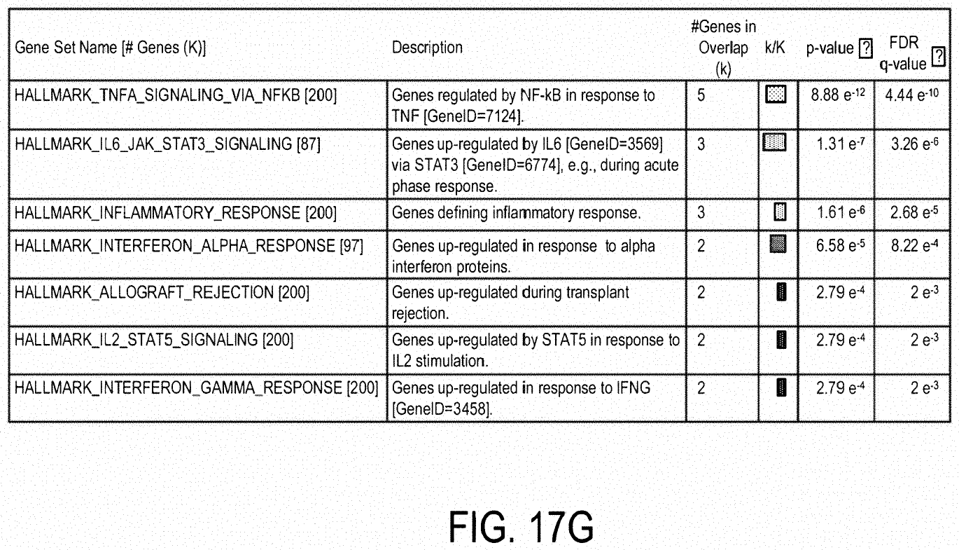

Patient 5 experienced a post-PPV objective clinical response with no disease progression at >20 months, which provided a unique opportunity to analyze the longer-term dynamics of the NeoAg-specific immune response in this patient. An A*1101/KITDFGRAK (SEQ ID NO: 4) tetramer was used to sort EGFR(L858R)-specific CD8⁺ T cells from PBMC drawn 12 months following the initiation of PPV (FIG. 16C; FIG. 14). Sorted NeoAg-specific cells then underwent single-cell TCRα/β sequencing, from which 52 high-confidence TCR clones were identified (Tet⁺, see Methods). Tumor biopsies taken pre-treatment or at 12 months post-PPV also underwent TCRVβ-CDR3 sequencing of tumor-infiltrating lymphocytes (TIL), allowing for a detailed comparison of CDR3 frequencies in the blood and tumor compartments prior to and post-immunization (Table 7). As shown in FIG. 16D, CDR3 clones that overlapped in both PBMC and TIL pre-PPV were present at higher frequencies within the blood, including the NeoAg-specific Tet⁺ clones (FIGS. 17A & 17B). By contrast, post-vaccine samples showed only half the number of CDR3 clones overlapping between blood and TIL, but these clones were present at significantly higher frequencies within the TIL compartment. Importantly, NeoAg-specific Tet⁺ clones demonstrated significant frequency increases in both the PBMC and TIL compartments post-immunization, including 13 new T-cell clones not detected in pretreatment samples (FIG. 16D; FIG. 17C). Comparison of PBMC CDR3 sequences pre- and post-PPV showed that while a subset of T-cells (including Tet* clones) increased after immunization, most other T-cell clones decreased in frequency. By contrast, at the tumor site nearly all CDR3 clones showed increased frequencies after immunization, including 35 of 40 Tet⁺ clones (FIG. 16D).

One striking feature revealed by this analysis was that 40 of the 52 Tetr clones sorted post-PPV were already present at elevated frequencies in pre-PPV PBMC and TIL samples, suggesting that spontaneous priming of EGFR NeoAg-specific cells had occurred prior to immunization. However, most of these Tet⁺ CDR3 clones showed significant increases in PBMC frequency over the 12-week PPV course that were associated with the patients clinical response, consistent with the IFN-γ immune monitoring results (FIG. 16E; FIG. 7E; FIGS. 11A-11D). Interestingly, PPV also appeared to stimulate the expansion of approximately a dozen Tet⁺ clones that were not detectable in pre-treatment PBMC but showed significantly elevated frequencies several months later in both PBMC and at the tumor site (FIGS. 16D & 16F; FIG. 17B). One such induced Tet⁺ clone, Vβ-N1, increased >500-fold in frequency at the tumor site. TCRα/β chains from this Tet⁺ clone were subcloned into a lentiviral vector and used to transduce PBMC-derived T-cells to express TCR-N1 (FIG. 16G). EGFR NeoAg-specific recognition was confirmed by co-culturing TCR-N1 transduced T cells with A549 tumor target cells engineered to express HLA-A*1101 and/or the KITDFGRAK (SEQ ID NO: 4) minigene (FIGS. 16G & 16H). Collectively, this data provides evidence that peptide vaccination of Pt. 5 stimulated a significant expansion of NeoAg-specific CD8⁺ T cells in peripheral blood, ultimately leading to increased frequencies of these Tet⁺ T cells at the tumor site. It remains unknown whether the increased tumor infiltration by Tet⁺ cells was driven by enhanced T-cell trafficking to tumor, T-cell proliferation resulting from NeoAg recognition at the tumor site, or both processes.

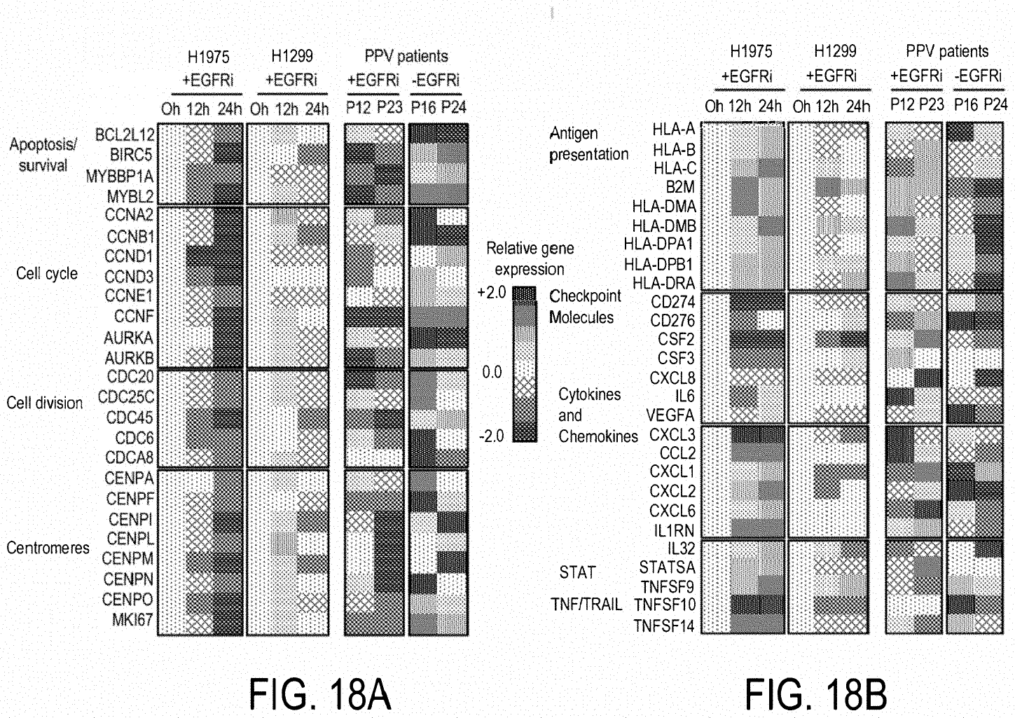

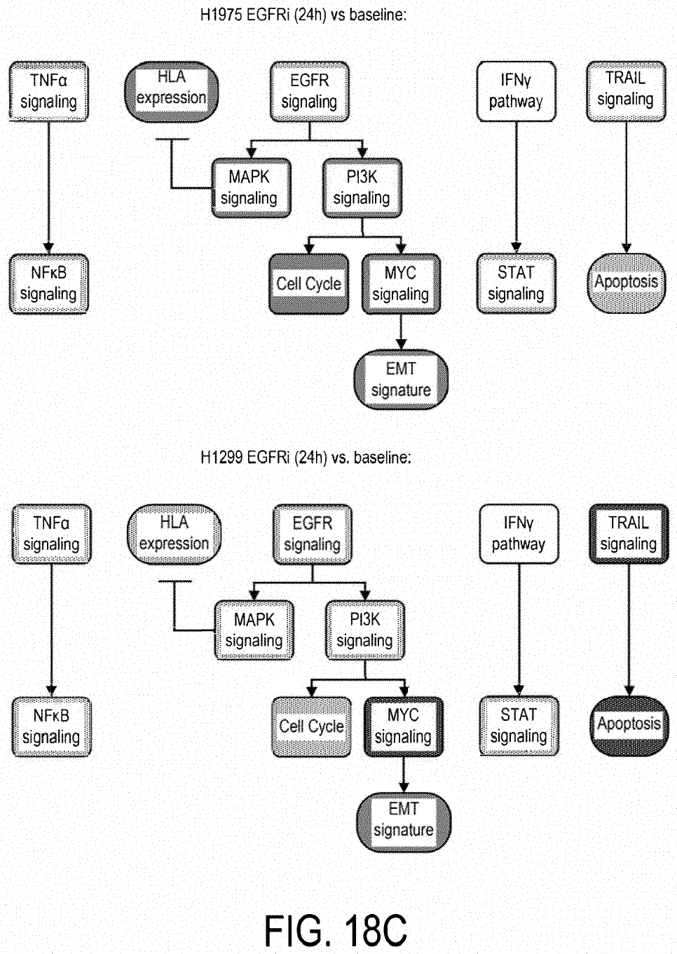

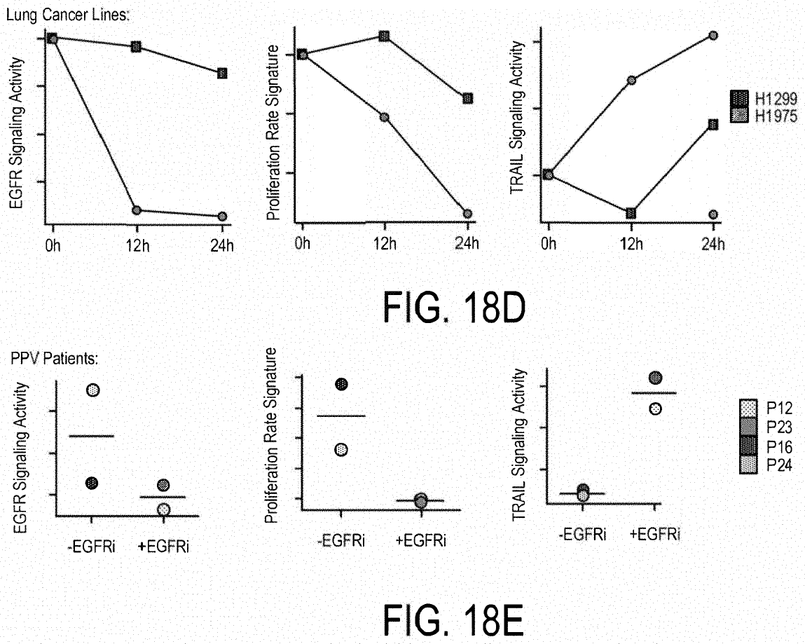

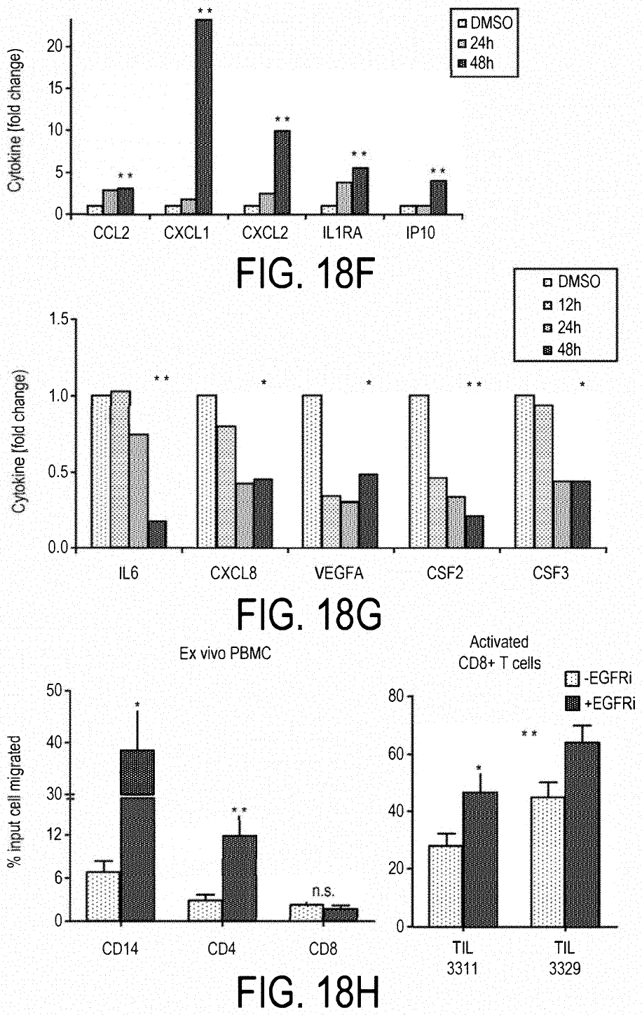

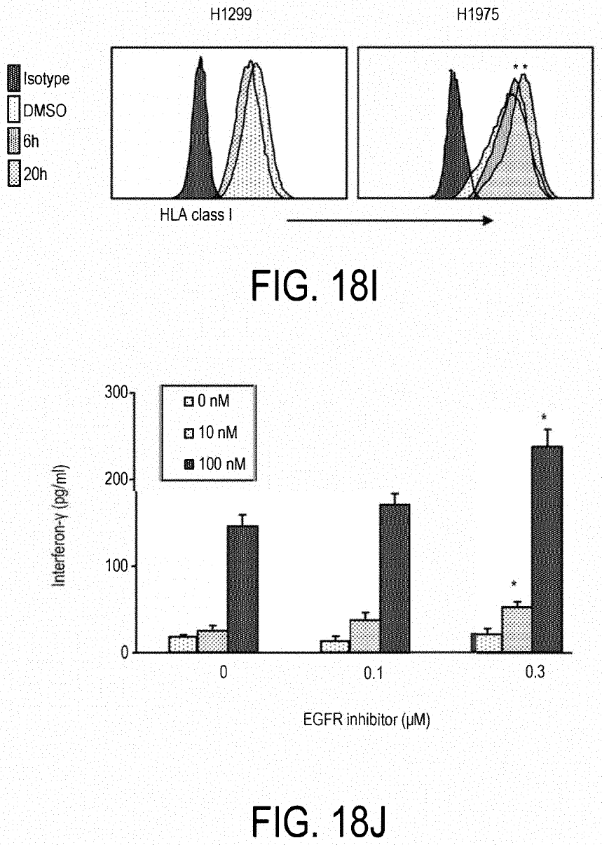

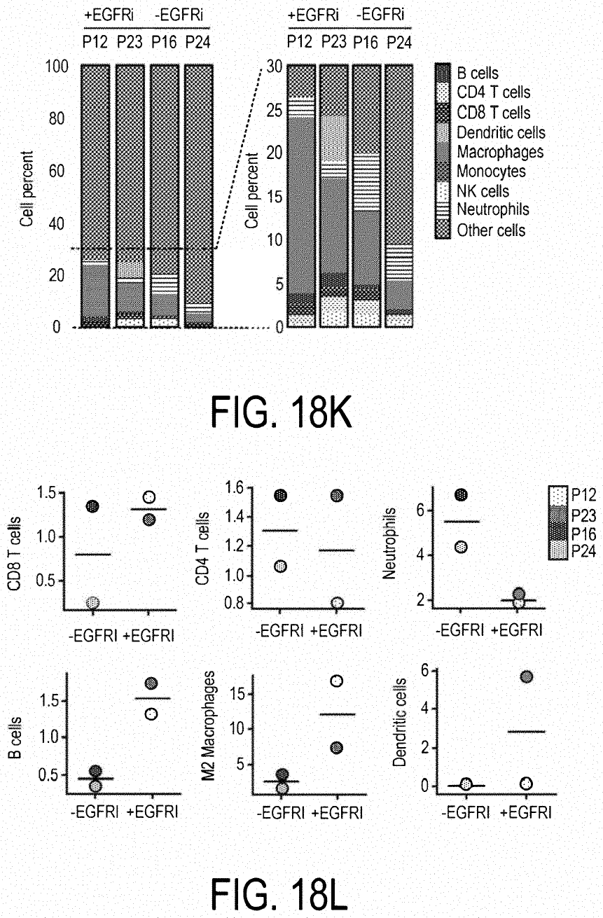

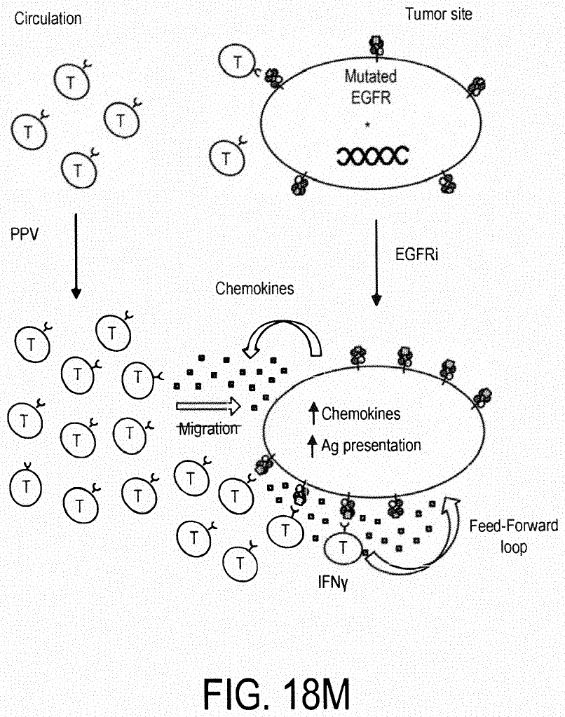

EGFRi promotes immune cell infiltration, antigen presentation, and T-cell activation. Although Group 2 and Group 3 patients showed similar clinical objective response rates following PPV, Group 3 patients showed significantly extended OS and PFS (FIG. 8). In order to better understand why Group 3 patients experienced better survival outcomes, we next assessed potential mechanisms by which EGFRi therapy may synergize with vaccination. Two lung cancer cell lines, H1975 (EGFR-mutated: L858R⁺/T790M⁺) and H1299 (EGFR-WT), were treated with EGFRi or DMSO and cell supernatants and total RNA were collected at multiple time points following treatment. As expected, EGFRi-treated H1975 cells showed decreased EGFR signaling that was confirmed by both RNAseq and Western blot analysis, in addition to decreased expression of genes associated with MYC signaling, proliferation, cell cycle, and apoptosis and survival (FIGS. 18A, 18C, and 18D; FIG. 17D). Examination of immune-related genes showed that EGFRi treatment increased the transcription of genes associated with TRAIL signaling and HLA class I and II antigen presentation, along with a concurrent decrease in checkpoint genes (FIGS. 18B & 18D; FIGS. 17E & 17F). Transcripts encoding for several chemokines and cytokines increased or decreased following EGFRi treatment, and Luminex analysis confirmed changes to 10 of them at the protein level in cell supernatants (FIGS. 18B, 17E, and 17F). Since EGFRi treatment upregulated CXCL1, CXCL2, and CCL2, chemokines well-known to promote immune cell migration, we next examined how peripheral blood leukocytes migrated in response to EGFRi- or DMSO-treated H1975 cell supernatants (FIGS. 17E & 17F). Both CD4 T cells and CD14⁺ monocytes from ex vii PBMC demonstrated increased migration towards EGFRi-treated cell supernatants. As expected, though they required prior activation to upregulate their migration capacity, CD8⁺ T cells also showed significantly increased migration in response to the same cell supernatants (FIG. 18H). Surface HLA class I surface expression was also increased in H1975 but not H1299 cells following EGFRi treatment, resulting in antigen-specific CD8⁺ T cells producing more IFN-7 following recognition of EGFRi-treated H1975 tumor cells (FIGS. 18I & 18J; FIG. 17G). These results support the notion that EGFRi may promote immune cell infiltration and antigen presentation at the tumor site, thus augmenting antitumor immune responses (Wantanabe et al., 2019; Im et al., 2016).

To determine if tumor samples from Group 3 patients demonstrated similar gene signatures, RNAseq analysis was performed on tumor biopsies from a small subset of PPV patients. Two tumor specimens analyzed were from patients not on EGFRi therapy (Group 2 Pts. 16 and 24), and two specimens were from patients on EGFRi therapy (Group 3 Pts. 12 and 23). Biopsies were taken during PPV immunization with the exception of the Pt. 24 specimen, which was obtained pre-PPV. Gene expression signatures for cell cycle, cell division, and cell survival in EGFRi-treated Pts. 12 and 23 correlated well with those of EGFRi-treated H1975 cells, as did gene signatures for EGFR signaling and proliferation rate (FIGS. 18A, 18D, and 18E). Immune-related gene signatures from PPV patient tumors showed partial overlap with H1975 signatures, including some concordance with upregulated antigen presentation, most strikingly in PR Patient 12. With the exception of CCL2 and IL1RN, most chemokines and cytokine signatures showed little concordance, a likely consequence of tumor specimens containing RNA derived from infiltrating normal immune and stromal cells. To impute the immune cell content of the tumor specimens individually, tumor RNAseq data was analyzed with reference to immune cell-type specific genes. As shown in FIG. 18K, tumors from the 2 EGFRi-treated patients contained elevated levels of M2 macrophages and B cells and decreased neutrophil infiltration compared to the 2 patients not taking EGFRi. The on-EGFRi tumor biopsy from Pt. 23 was the only sample to show elevated levels of dendritic cells, and was also associated with increased CD4⁺ T cell and NK cell infiltration (FIG. 18L). Notably, tumor-infiltrating CD8+ T cells were found at elevated levels in all 3 patients on-PPV compared to the pre-PPV tumor sample from Pt. 24 (FIG. 18K). Although the number of patient samples analyzed is too small to make definitive conclusions, taken together these results suggest that EGFRi has the capacity to alter gene expression within the tumor microenvironment to promote immune cell infiltration and antigen presentation.

Collectively, our study supports the following model to explain the therapeutic synergy of peptide vaccination and EGFRi therapy (FIG. 18M): PPV administration stimulates the expansion of NeoAg-specific T cells in the circulation, while EGFRi promotes enhanced antigen presentation and chemokine secretion at the tumor site. Increased chemokines in turn augment the trafficking of immune cells including activated T cells to the tumor, where recognition of cognate tumor antigen by T-cells stimulates tumor cell destruction and the production of IFN-γ (Venugopalan et al., 2016). Since IFN-γ is known to strongly upregulate antigen presentation and chemokine production (Schoebom et al., 2007; Schroder et al., 2004; FIGS. 17E & 17F), the combination of PPV and EGFRi may stimulate an initial antitumor T-cell response, which subsequently initiates a ‘feed-forward’ loop at the tumor site to sustain the antitumor immune response (FIG. 18M). Such sustained immune responses would provide an explanation for the extended PFS that we observed in Group 3 PPV patients, in addition to the long-term expansion of induced NeoAg-specific T-cell clones observed in Patient 5.

Discussion. This may be the first report demonstrating that peptide vaccination against shared NeoAgs can induce objective clinical regressions in multiple cancer patients. Owing to HLA diversity and the overwhelming prevalence of private mutations, no previous vaccine study has reported immunizing more than a single patient with potentially shared NeoAg peptides. However, the relatively high prevalence of both HLA-A* 1101 and EGFR(L858R) mutations in Asian NSCLC patients predicted that −15% of our EGFR-mutated patients would share this combined phenotype (Kobayashi et al., 2016; Gonzalez-Galarza et al., 2015). This provided a unique opportunity to immunize 5 such patients (4 in this study) with vaccines containing EGFR NeoAg peptides in common, including the A*1101-restricted KITDFGRAK peptide (SEQ ID NO: 4) (Li et al., 2016). Remarkably, 4 of the 5 patients experienced tumor regressions within 12 weeks of NeoAg vaccination, with all 4 patients demonstrating significantly increased or dominant KITDFGRAK (SEQ ID NO: 4)-specific CD8⁺ T-cell reactivity during the time of the clinical responses (Li et al., 2016). By showing that multiple A*101/EGFR(L858R) patients responded clinically to immunization with shared NeoAg peptides, this study provides an important first proof-of-concept in cancer vaccine studies.

Our study also found evidence that at least 2 other EGFR mutations can be immunogenic in NSCLC patients, with 2 additional clinical responses being associated with dominant vaccine-induced co4+ or cos+ T-cell responses against the H773L/V774M or T790M mutations, respectively (FIG. 12A). Although T790M has a low prevalence in primary NSCLC, it develops frequently as a resistance mechanism to first-line EGFRi therapy (Kobayashi et al., 2005; Xu et al., 2017; El Nadi et al., 2018). The T790M-containing LTSTVQLIM peptide (SEQ ID NO: 65) was the dominant NeoAg target of CD8⁺ T-cells in the only complete responder in our study (Pt. 17), and thus constitutes another promising potential shared target for the −7% of patients worldwide that express HLA-C*1502. Non-EGFR NeoAg-specific immune responses were also detected in responding patients, most notably CD8⁺ T-cell responses against AQP12A(L28R) in Pt. 11 and FGFR1(R734) in Pt. 22. Although our study was not designed to directly compare the immunogenicity of EGFR NeoAgs to those derived from other mutated genes, we did observe that the preponderance of immune reactivity was focused on mutated EGFR targets, as evidenced by the discrepancy in IRC scores between patients in Group 1 and Pts. in Groups 2 and 3 (FIG. 12B). Since there is no reason to suspect that EGFR-derived NeoAgs would be inherently more immunogenic than those derived from other mutated proteins, the explanation for this finding may be related to the use of EGFRi by Group 2/3 patients, as discussed further below. Several non-responding patients also generated PPV-induced T-cell reactivity, including against mutated NeoAgs derived from IDH2, MAP2K4, PIK3CA, TPS3, and EGFR(746_750del). We hypothesize that the lack of clinical responses in these patients may reflect lack of NeoAg presentation by patient tumors for any number of potential reasons, including dysfunctional antigen processing, immune editing, HLA loss, or neoantigen promoter hypermethylation (McGranahan et al., 2017; Bedognetti et al., 2019; Rosenthal et al., 2019).

Since peptide-based cancer vaccine studies have only rarely reported clinical responses following immunization, it is worth discussing the unique features of our vaccination approach. To activate both CD4⁺ and CD8⁺ T cell-mediated immunity, we chose to immunize patients with mixtures of long and short NeoAg peptides, often including multiple peptides (up to six) against the same targeted NeoAg (Table 5). Peptides were solubilized and administered in saline to avoid any inhibitory long-term antigen depot effects; in order to compensate for the typically short half-life of saline-solubilized peptides, vaccinations were administered weekly (Li et al., 2016; Overwijk et al., 2015). Focusing the mutation calling on a panel of 508 cancer-associated, potential driver genes greatly simplified PPV design, but also restricted the number of potential NeoAg targets identified per patient, a significant limitation of our study. However, this more focused approach did allow for EGFR mutations to be identified as shared NeoAgs with promising therapeutic potential. For a vaccine adjuvant, we employed topically-applied Imiquimod cream, a TLR7 agonist known to be moderately effective at activating local antigen-presenting cells. Based on the breadth, magnitude, and timing of the NeoAg-specific T-cell responses observed, we interpret that our immunization approach was likely most effective at boosting T-cell responses that had previously been spontaneously primed in patients, while demonstrating limited efficacy for priming new T-cell responses. Incorporating more potent and promising vaccine adjuvants such as polyl:C, anti-CD40, or STING agonists into future peptide vaccine formulations may help to address lack of de novo T-cell priming. Nevertheless, PPV-mediated activation of pre-existing immune responses was effective at inducing multiple clinical responses in our study, a finding that strongly supports NeoAg immunoreactivity pre-screening to guide future vaccine design for NSCLC patients (Malekzadeh et al., 2019).

EGFRi therapy, while not impacting the clinical response rate to PPV nor the magnitude of PPV-induced immune responses, did have a surprisingly positive impact on patient OS and PFS in spite of prior failure as a monotherapy (FIG. 1G; FIG. 5B; FIG. 6C; FIGS. 8A & 8B). Our mechanistic studies suggest that the immunomodulatory effects of EGFRi has the potential to not only augment the efficacy of cancer vaccines, but also improve other T-cell based immunotherapies such as checkpoint blockade or engineered T-cell therapies. However, these concepts will need to be rigorously tested in future studies to confirm their validity and utility. One of the more notable findings of our study was that all 7 PPV responders had tumors containing EGFR mutations, whereas none of the 8 patients with EGFR-WT tumors responded to PPV. In light of the pre-existing immune responses discussed above, we speculate that first-line EGFRi treatment may initially induce ‘immunogenic’ tumor cell death leading to spontaneous cross-priming of NeoAg-specific T-cells (Pol et al., 2015; Goodridge et al., 2013), which are subsequently boosted with PPV immunization. It remains to be determined if EGFR NeoAg-specific T-cell priming is favored over other tumor-associated NeoAgs; however, EGFRi drugs are known to bind irreversibly to mutated EGFR target proteins, which could conceivably impact their processing and subsequent NeoAg presentation by both APCs and tumor cells (Yamaoka et al., 2017). Future studies will be required to delineate the precise role of EGFRi therapy in the priming NeoAg-specific immune responses. It is important to note that several responding patients also generated specific immune responses against private NeoAgs (FIG. 12) that may have contributed to the clinical responses observed.

Although many significant challenges to personalized NeoAg identification remain to be addressed, these results provide the first evidence that shared NeoAgs can constitute therapeutic vaccine targets capable of inducing immune and clinical responses in multiple cancer patients. Based on the prevalence of HLA-A*1101 and L858R mutations, the KITDFGRAK NeoAg peptide (SEQ ID NO: 4) is estimated to be presented by up to 8.4% of Asian and 1.2% of North American NSCLC patients, making it one of the most widely shared NeoAgs in cancer (Kobayashi et al., 2016; Gonzalez-Galarza et al., 2015; Wirth and Kuhnel, 2017; Lu and Robbins, 2016). It is demonstrated that multiple other EGFR mutations can also be immunogenic for patients expressing specific HLA haplotypes, and that there is a natural antigen presentation ‘synergy’ between the most prevalent EGFR mutations and the HLA-A3 superfamily of class I allotypes, findings that have important practical implications for future vaccine development and patient selection. It is also important to note that since tumor burden and pleural effusion were both associated with worse overall survival of PPV patients (FIGS. 10A & 10B), immunization of earlier stage NSCLC patients, perhaps even prior to the development of EGFRi resistance, may be associated with better clinical outcomes. The results from this Phase 1 b trial though highly encouraging will need to be replicated and validated in the context of larger, randomized vaccine trials; however, the data presented provides a compelling rationale to initiate these studies.

### B. Methods

Data reporting. No statistical methods were employed to predetermine patient sample numbers. The study was not randomized and some investigators were not blinded during experiments and outcome assessments.

Clinical Trial Design and Treatment.

Between November 2016 and December 2018, a single-arm trial was conducted at Tianjin Beichen Hospital in China. The study protocol conformed to the ethical guidelines of the 1975 Declaration of Helsinki and was approved by the internationally-recognized ChiCTR and the Ethics Committee of Tianjin Beichen Hospital (Chinese Clinical Trial Registry (ChiCTR) No.: ChiCTR-IIR-16009867. Primary endpoints of the trial were safety and tolerability, feasibility of the personalized approach, and secondary endpoints were anti-tumor immune reactivity, clinical response and potential survival benefit. All patients provided written informed consent before enrolling in the study.

Patient eligibility. Twenty-four patients with stage III-IV NSCLC (18 adenocarcinoma and 6 squamous cell carcinoma) were enrolled in this clinical study of personalized neoantigen peptide vaccine (PPV) and were successfully immunized. The 24 study patients were selected according to the following inclusion criteria: adult patients aged 18 or more; clinical assessments classified all patients with NSCLC stage III/IV according to NCCN Clinical Practice Guidelines in Oncology, Version 3.2016, Non-Small Cell Lung Cancer and the Eighth Edition Lung Cancer Stage Classification; NSCLC diagnosis was confirmed by biopsy and pathological assessment; patients experienced disease recurrence after failing conventional treatments including surgery, chemotherapy, radiotherapy and/or EGFR inhibitor (EGFRi) therapy, and had no active treatments; patients showed good or moderate Eastern Cooperative Oncology Group (ECOG) performance status (PS≤3); patients were undergoing no other concurrent immunotherapies; pretreatment biopsy samples were available, and showed at least one genetic mutation; patients had a life expectancy of >3 months. Patients were excluded if they: were pregnant or lactating; had known or suspected autoimmune disease, or other immune system disease; had systemic cytotoxic chemotherapy or experimental drugs for treatment of metastatic NSCLC within 4 weeks prior to first dose of personalized vaccine (not including EGFRi); had participated in any other clinical trial involving another investigational agent within 4 weeks prior to first dose of personalized vaccine; had liver or kidney dysfunction, severe heart disease, coagulation dysfunction or hematopoietic impairment; had any active infection requiring systemic treatment; suffered from other current malignancies either in progress or treated within the past five years. Pre-treatment tumor biopsies were required for the trial, and post-treatment biopsies were optional and required additional patient consent. The clinical characteristics of study patients are shown in FIG. 4A and the EGFRi treatment history of the 16 EGFR-mutated patients are shown in FIG. 4B.

Generation of Personalised Neoantigen Vaccines.

Somatic mutation analysis. Patient tumor specimens were obtained by fine-needle biopsy of tumor sites in the lung or lymph node (Table 7). Tumor biopsies from individual patients underwent DNA sequencing using a 508 gene panel, in conjunction with standard clinical and pathology laboratory test procedures at Tianjin Beichen Hospital (Tianjin. China). This genotyping panel was designed to detect shared, tumor-associated driver mutations within 508 cancer-associated genes (Table 4). Tumor DNA was extracted from biopsy samples according to the instructions of the TIANamp Genomic DNA Kit (Tiangen, China), and detected by Hiseq X-10 (Illumina, USA) which profiled using exon capture by hybridization followed by next-generation DNA sequencing (HengJia Medical Laboratory, Tianjin, China). For somatic mutation calling, analyses of next generation sequencing data of tumor and matched PBMCs (as source of normal germline DNA) from the patients were used to identify the specific coding-sequence mutations, including single-, di- or tri-nucleotide variants that lead to single amino acid missense mutations and small insertions/deletions (indels). Output from Illumina software was processed by the Broad Picard Pipeline to yield BAM files, which contained aligned reads (bwa version 0.7.8, aligned to the NCBI Human Reference Genome Build hg19) with well-calibrated quality scores. Somatic single nucleotide variations (sSNVs), somatic small insertions and deletions were all detected using Varscan2 (version 2.4.3). All indels were manually reviewed using the Integrative Genomics Viewer (version 2.4.1). All somatic mutations, insertions and deletions were annotated using Annovar (version 2013-07-28 11:32:41). Neoantigen peptides were chosen based on nonsynonymous somatic mutations detected at a mutated variant allele frequency of 0.04 or higher.

HLA typing. Peripheral blood was drawn for high resolution HLA typing at the time of enrollment. Human leukocyte antigen (HLA) loci were typed via polymerase chain reaction-sequence-based typing (PCR-SBT) method employing a DNA amplification step (CapitalBio, China). Briefly, DNA was extracted from peripheral blood of patients according to the instructions of the Magic Beads DNA Extraction Kit (TANBead, China). Exons 2 and 3 of HLA class I genes (HLA-A, B, and C) and exon 2 of HLA class II a and P genes (HLA-DQ and DR) were amplified and purified, and PCR products were sequenced on ABI 3730XL DNA Analyzer (Applied Biosystems, USA). Sequence chromatograms were analyzed using ATF1.5 software (Conexio Genomics, Australia).

Vaccine peptide selection. Due to the high number of somatic mutations typically found in lung cancers and the fact that the majority constitute private ‘passenger’ mutations, we chose to target somatic mutations detected from a focused panel of 508 tumor-associated genes. The rationale for this approach was two-fold: (1) It would enable targeting of mutations more likely to be essential for the tumor phenotype and thus less likely to be lost through immune editing, and (2) It would increase the chances of identifying shared neoantigen targets that could potentially be beneficial to multiple NSCLC patients. Non-synonymous coding mutations detected by the 508-gene panel were translated in silico and the resulting neoantigen sequences were assessed for predicted binding affinity to patient HLA class I and class II molecules according to the HLA-peptide prediction algorithms NetMHC4.0, NetMHCpan3.0, NetMHCpan4.0, NetMHCII2.2 and NetMHCII2.3 (Andretta et al., 2015; Jensen et al., 2018). Immunizing neoantigen peptides were chosen primarily based on highest predicted binding affinity to the patient's HLA class I and class II molecules. However, vaccines were also designed to maximize the number of different HLA molecules engaged and minimize intra-HLA peptide competition when possible. Certain biochemical properties (such as elevated hydrophobicity or the presence of multiple cysteines) which can negatively impact the synthesizability or solubility of the immunizing peptides were also considered. In addition, we aimed to design individual patient vaccines to contain an approximately 2:1 ratio of short to long vaccine peptides, or as close as the somatic mutation profiling and HLA/peptide binding predictions would allow. For each patient, up to 14 peptides of 9 to 17 amino acids in length arising from up to 12 independent mutations were selected and prioritized. A mean of 9.4 neoantigen peptides per patient were chosen for peptide synthesis, which included on average 6.5 short, HLA class I-restricted peptides and 2.9 long, HLA class II-restricted peptides (Table 5).

Patient immunizations. Immunizing peptides were synthesized using standard solid-phase synthetic peptide chemistry, purified to >98% using reverse phase high performance liquid chromatography and tested for sterility and the presence of endotoxin to ensure safety and tolerability using methodologies consistent with Good Manufacturing Practice (HengJia Neoantigen Biotechnology (Tianjin) Co., Ltd.). As shown in Table 5, 5 to 14 peptides per patient were synthesized, solubilized individually in sterile phosphate-buffered saline (PBS), and mixed into 2 separate peptide cocktails, each with 2-7 short peptides and 1-3 long peptides in 1 ml total volume. Peptides binding to the same HLA allotypes were separated into different cocktails to reduce potential antigen competition. Patients received 200 μg of each peptide per immunization, injected subcutaneously into the left and right extremities, and administered weekly for a minimum of 12 weeks. In order to provide concurrent Toll-like receptor (TLR)-7 stimulation of pAPCs, Aldara cream with 5% imiquimod was applied topically as a vaccine adjuvant over the vaccine site immediately after peptide cocktail administration. Patients were permitted to continue immunizations after 12 weeks if desired and in the patient's best interest. 11 of the 24 patients continued to receive vaccinations beyond 12 weeks, as shown in FIG. 2 and Table 5.

Sample collection. Serial peripheral blood mononuclear cells (PBMC) samples were collected at pre-treatment, as well as at 4, 8, and 12 weeks post-vaccination. A maximum of 15 ml of blood was drawn per month, according to Tianjin Beichen Hospital regulations for advanced-stage cancer patients. Collection of additional blood samples beyond the 12 weeks of the trial period were optional and required additional patient consent. Extra blood samples were collected from Patients 5, 8, and 17, who had all experienced clinical objective responses. Viable PBMCs were collected and stored at −80° C. Collection of post-vaccine tumor biopsies was optional but not required in this trial due to the invasive nature of the procedure, uncertain feasibility, and the general reluctance of most patients. Tumor tissues obtained for further RNA sequencing and/or bulk T cell receptor VB CDR3 sequencing analysis were collected from PPV trial Patients 5, 12, 16, 23 and 24 after providing written informed consent.

Tumor response evaluation criteria. Objective tumor response assessments were made according to the Response Evaluation Criteria in Solid Tumors (RECIST version 1.1) guidelines. We utilized computerized tomography (CT) and/or magnetic resonance imaging (MRI) scans to measure selected target lesions. Patients were required to perform at least one pre-treatment scan for baseline measurements and another scan at 3 to 4 months post-vaccine for response assessment. Additional patient scans were taken monthly during the first 12 weeks of vaccination if feasible. Target lesions with a minimum size of 10 mm (15 mm for malignant lymph nodes) were measured in the longest diameter by three different radiologists, with the mean of the three independent measurements used for clinical assessments. A maximum of two target lesions per organ were measured, with the two largest lesions selected, up to a maximum of five lesions in total. Tumor burden was calculated as the sum of the diameters of all target lesions (FIG. 5B). Clinical responses were evaluated as follows: CR, complete disappearance of all target lesions; PR, partial response, defined as a 30% decrease in the sum of diameters of target lesions; PD, progressive disease, defined as a minimum 20% increase in the sum of diameters of target lesions or the appearance of new lesions; and SD, stable disease, defined as a change in tumor burden insufficient to qualify for PR, or PD. Clinical responses were assessed 3 to 4 months following the date of the first immunization.

Clinical trial statistical plan. Statistical analysis was primarily descriptive, including enumeration of patients who experienced any adverse events. All statistical tests were 2-sided with an alpha level of 0.05. Confidence intervals to be evaluated were constructed with a significance level of 0.05. Additional exploratory analyses of the data were conducted as deemed appropriate.

Analysis of Primary Endpoints. Treatment-associated adverse events were analyzed based on those categorized and graded according to the National Cancer Institute Common Terminology Criteria for Adverse Events (version 4.0). During the first 12 weeks of vaccine treatment and continued vaccination beyond 12 weeks, safety assessments were performed starting on the day of each vaccination for up to 48 hours afterwards. Safety assessments were performed every 2 months for patients with extended follow-up time. Disease-associated symptoms that were present at baseline (pre-treatment) were not reported unless they worsened after vaccination.

Analysis of Secondary Endpoints. Secondary endpoints including progression-free survival (PFS) and overall survival (OS) were summarized using the Kaplan-Meier method. Measurements of the immune responses via ELISA, ELISPOT and tetramer analysis prior to the first vaccination and every 4 weeks after each vaccination were summarized descriptively. Determinations of PFS and OS for enrolled subjects were calculated from the date of enrollment to disease progression and/or death, or 31Dec. 2018, respectively. Sub-Group Analyses. Based on differing response and survival profiles, we analyzed the enrolled patients according to subgroup based on wild-type (WT) or mutated (Mut) EGFR mutation status, and if EGFR inhibitor use was continued or stopped prior to the start of immunization. These groups were defined as EGER WT-PPV only (Group 1), EGFR Mut-PPV only (Group 2) and EGFR Mut-PPV+EGFRi (Group 3).

Enzyme-linked immunosorbent (ELISA) and Enzyme-linked Immunospot (ELISPOT) assays. Peripheral blood mononuclear cells (PBMCs) collected prior to the start of immunization and at different time points following PPV were isolated by Ficoll density gradient centrifugation and counted in the presence of trypan blue dye to evaluate viability prior to cryopreservation. For ELISA analysis, 5×10⁵ PBMCs in RPMI-1640 containing 10% FBS were added to each well of 96-well plate with a total volume of 250 ul. PBMC were cultured in the presence of vaccine peptide pools, individual vaccine peptides, or irrelevant control peptides (7.5 ug/ml) along with 300 IU/ml interleukin (IL-2) in a 37° C. humidified incubator with 5% CO₂ for 5 days. Following 24 hours of peptide re-stimulation, the IFN-γ concentration of cell supernatants was measured using a human IFN-γ ELISA kit (Dakewe, China), according to the manufacturer's instructions. A level of IFN-γ secretion 1.5-fold or greater over background signal with no peptide control was considered to be a positive immune response. Immune Response ComboScores (IRC) that considered breadth, intensity, and persistence of vaccine-induced immune responses were calculated for each patient as follows: ComboScore=sum of fold changes >1.5 for all vaccine peptides at all time points (pre-treatment, 4, 8, and 12 weeks). For ELISPOT assays, 2.5×10 PBMCs were prepared in RPMI-1640 containing 0.5% FBS with a total volume of 150 μL for each well in a 96-well plate. Ex vivo PBMC were stimulated in triplicate with individual vaccine peptides at a final concentration of 10 ug/ml and plates were incubated at 37° C. in humidified incubator with 5% CO₂ for 36 hours. Spot detection was performed using a Human IFN-γ ELISpotPRO kit (MABTECH Inc., USA), and normalized to the number of IFN-γ spots detected per 10⁶ PBMC.

Tetramer staining and flow cytometric analyses. Selected custom phycoerythrin (PE) or APC-conjugated HLA-peptide tetramers (Baylor College of Medicine, USA; MBL, Japan) were successfully generated (FIG. 13A). PBMCs were thawed and resuspended in RPMI-1640 containing 0.5% fetal bovine serum (FBS). 100 μL PBS-1% BSA containing fluorophore-conjugated HLA/peptide tetramer (1:50 dilution) was added to 5×10⁵ PBMCs and incubated at room temperature for 20 min in the dark. Cells were washed with PBS-1% BSA, stained with FITC- or PE-conjugated anti-CD8 mAbs (Biolegend, USA, 1:200 dilution) and incubated for 15 min. Cells were then washed and resuspended in 400 μL PBS-1% BSA for flow cytometric analysis (LSRFortessa X-20 Analyzer).

Tumor RNA sequencing and bulk T cell receptor Vβ CDR3 sequencing analysis. Post-vaccination tumor samples were collected from Patients 12, 16, 23 and 24 after obtaining patient consent. RNA was isolated from patient tumor tissues using an RNA Extraction Kit (Qiagen, USA). Libraries were generated using the NEBNext® Ultra™ RNA Library Prep Kit (Illumina, USA) following manufacturer's instructions. Libraries were purified with AMPure XP system (Beckman Coulter, Germany) and samples were sequenced using an Illumina NovaSeq platform (Illumina, USA). For T cell receptor (TCR) diversity analysis, DNA samples were extracted from patient PBMC or pre- and post-treatment tumor biopsies from Patient 5 using a DNA extraction kit (Qiagen, USA), followed by library construction with two rounds of PCR-based amplification. CDR3 fragments were first amplified using specific primers for each V and J gene, and target fragments of multiplex-PCR products were purified using magnetic beads (A63882, Beckman, Germany). Next, PCR was performed using universal primers, and target fragments 200-350 bp were retrieved and purified by QIAquick Gel Purification Kit (Qiagen, USA). PCR products were then sequenced using the Illumina X10 platform. Single-read CDR3 sequences eliminated and the remaining sequences were analyzed to evaluate TCRVβ IMGT clonality of patients before and after treatment, as previously described (Tumeh et al., 2014).

Single-cell T cell receptor sequencing. HLA-A*1101/KITDFGRAK (SEQ ID NO: 4) Tetramer⁺ CD8 T cells from post-PPV PBMCs of Patient 5 were sorted using a flow-based cell sorter (BD FACSAria HI, USA) and imaged by confocal microscope (Leica SP8, Germany). Sorted Tet⁺ cells were adjusted to 1×10⁶ cells per ml in PBS, and loaded on a Chromium Single Cell Controller (10X Genomics, USA) to generate single-cell gel beads in emulsion (GEMs) using a Single Cell 5⁺ Library and Gel Bead Kit (10× Genomics, USA). Captured cells were lysed and released RNA was barcoded through reverse transcription to produce barcode-containing cDNA in individual GEMs as per the manufacturer's introductions. V(D)J sequences were enriched by nested PCR amplification with specific primers targeting conserved TCR sequences. Sequencing was performed on an Illumina NovaSeq platform with 150 bp (PE150) paired ends. Cellranger VDJ was used for analyzing V(D)J recombination, T cell diversity, and pairing of appropriate TCR a and R chain sequences for each individual T cell. Single-cell sequencing of Tet+ cells resulted in the identification of 639 distinct TCRα/β pairs. However, since tetramer-based cell sorting can result in contamination with non-antigen specific T cells, we chose to select TCR clones for which a minimum of 10 VβCDR3 reads were detected, resulting in 51 unique TCR clones. Four clones with Vβ-CDR3 sequences that showed little or no presence in any PBMC or TIL samples were eliminated from the analysis, and 5 additional TCR clones with 7 or 8 CDR3 reads were included based on their increasing frequencies in PBMC and/or TIL during PPV, as would be expected for a neoantigen-specific T cell clone.

TCR cloning and validation. The variable region sequences of TCR Vα-N1 and Vβ-N1 chains obtained by single cell sequencing were fused to an engineered human constant region to enhance α and β chain pairing (Cohen et al., 2007). These modified Vα and Vβ chain sequences were synthesized and inserted into an EF1a promoter based lentiviral expression vector pCDH to create lentivirus lenti-EF1a-TCR-N1. Healthy donor PBMC were prepared using lymphocyte separation medium (Stem Cell, Canada). T cells were isolated using the Dynabeads® Human T-Expander CD3/CD28 Kit (Thermofisher, USA), mixed with 3 ml X-Vivo15 serum-free medium (Lonza, Switzerland) and cultured at 37° C. in 5% CO₂ for 48 hours. Cell density was adjusted to 1×10⁶/mL and co-cultured with packaged lentivirus lenti-EF1a-TCR-N1 for 4 days at 37° C. in 5% CO₂. TCR-N1 expression and antigen specificity was confirmed by staining with the HLA-A*1101/KITDFGRAK (SEQ ID NO: 4)tetramer. A minigene encoding the KITDFGRAK peptide (SEQ ID NO: 4) linked to the HLA-A*l101 cDNA through an IRES sequence was synthesized and inserted into lentiviral vector pCDH with EF1a promoter to create lentivirus lenti-EF1a-KIT-A11. Control lentiviral constructs included vectors that expressed either the KITDFGRAK (SEQ ID NO: 4) minigene or HLA-A*1101 individually. Lentiviral-transduced A549 lung cancer cells stably expressing the gene(s) of interest were selected through purinomycin-based selection. HLA-A*1101 surface expression was confirmed by staining with an A*1101-specific mAb followed by flow cytometric analysis. Engineered TCR-N1-T cells were co-cultured with parental A549, A549-A11, A549-KIT, or A549-A11.KIT cells (20,000 target cells per well) at 37° C. in 5% CO₂ with effector-to-target ratios of 1:1, 2.5:1, 5:1 and 10:1. Non-transduced, expanded T cells from the same PBMC donor were used for a control. Supernatants were collected after 24 hours of co-culture to assess levels of IFN-γ secretion by ELISA.

Immunoprecipitation and Western Blot. H1975 (EGFR-L85R/T790M) and H1299 (EGFR-WT) lung cancer cell lines (ATCC, USA) were treated with different concentrations (0.1, 1, 2, and 5 μM) of EGFRi Osimertinib (LC Laboratories, USA) for different time periods to optimize EGFRi treatment conditions. Cells were washed with cold PBS and lysed using lysis buffer (1% Triton X-100, Sigma, USA) and Halt Protease Inhibitor Cocktail 100X and 0.5 μM EDTA 100X (Thermo Scientific, USA). Lysates were collected and centrifuged at high speed for 30 minutes at 4° C. prior to measuring protein concentration with a Bradford assay kit (BioRad, USA). Preclearing was performed using 10 μL Pierce Protein A/G Ultra Link resin (Thermo Scientific) per sample and incubating for 2 hours at 4° C. Immunoprecipitations were performed using the same amount of total protein and by incubating cell lysates for 18 hours at 4° C. with the following antibodies at a 500:1 dilution: anti-phospho-EGFR (EMD Millipore), anti-EGFR, Anti-p44/42 MAPK ERK1/2, anti-phospho-p44/42 MAPK ERK1/2, and anti-GAPDH (Cell Signaling Technology). Protein A/G crosslinking beads were added and incubated for 4 hours prior to washing with cold PBS. Samples were run using an SDS-PAGE gradient (8-16%) gel (Invitrogen, USA). Proteins were transferred to a PVDF membrane (Thermo Scientific, USA) and blots were blocked with 5% milk prior to incubation with specific antibodies overnight at a concentration of 1:1000. Blots were washed and incubated with peroxidase-conjugated anti-rabbit secondary antibody (1:10,000) (Jackson Immuno Research, USA). Blots were developed using the Super Signal West Pico PLUS Chemiluminescent enhanced horseradish peroxidase substrate (Thermo Scientific, USA) and visualized with X-ray film.

RNA sequencing of lung tumor cell lines. H1975 and H1299 lung cancer cells were treated with 1 μM of EGFRi Osimertinib (LC Laboratories, USA) for 0 h, 12 h, or 24 h; or 24 h with 1 μM of EGFRi Osimertinib (LC Laboratories, USA)+500 U/mL recombinant human IFNγ (R&D Systems, USA) added for the last 12 hours of culture. Cells were lysed and total RNA prepared using an RNeasy Mini Kit (Qiagen, USA) according to the manufacturer's protocol. RNAseq was performed by the Avera Institute for Human Genetics (South Dakota, USA) as follows: Total RNA was assessed for degradation on an RNA 6000 Nano chip ran on a 2100 Bioanalyzer (Agilent, USA) where the average RNA integrity score for the sample set was 9.7. Sequencing libraries were prepared using the TruSeq Stranded Total RNA Library Prep Kit (Illumina, Inc, USA) following the low sample procedure. Briefly, ribosomal RNA (rRNA) was depleted from total RNA and the remaining RNA was purified, fragmented appropriately, and primed for cDNA synthesis. Blunt-ended cDNA was generated after first and second strand synthesis. Adenylation of the 3′ blunt-ends was followed by adapter ligation prior to the enrichment of the cDNA fragments. Final library quality control was carried out by evaluating the fragment size on a DNA1000 chip ran on a 2100 BioAnalyzer (Agilent, USA). The average library yielded an insert bp size of 326. The concentration of each library was determined by quantitative PCR (qPCR) by the KAPA Library Quantification Kit for Next Generation Sequencing (KAPA Biosystems, USA) prior to sequencing. Libraries were normalized to 2 nM in 10 mM Tris-Cl, pH 8.5 with 0.1% Tween 20 then pooled evenly. The pooled libraries were denatured with 0.05 M NaOH and diluted to 20 μM. Cluster generation of the denatured libraries was performed according to the manufacturer's specifications (Illumina, Inc, USA) utilizing the HiSeq PE Cluster Kit v2 chemistry and flow cells. Libraries were clustered appropriately with a 1% PhiX spike-in. Sequencing-by-synthesis (SBS) was performed on a HiSeq2500 utilizing v2 chemistry with paired-end 101 bp reads resulting in an average of 52.4 million paired-end reads per sample. Sequence read data were processed and converted to FASTQ format for downstream analysis by Illumina BaseSpace analysis software, FASTQ Generation v1.0.0.

RNA sequencing data analysis. Quality control of patient and cell line RNAseq data was performed using FastQC v0.11.5, FastQ Screen v0.11.1, RSeQC v3.0.0, MultiQC v1.6 and proprietary algorithms of the BostonGene platform (Wingett et al., 2018). Four patient samples with the acceptable quality (good phred scores, good per base sequencing content along the read length, coding reads >50 M, adapter content <15%, <5% microbiome/mouse unique genomes contamination) were chosen for further analysis. RNAseq reads were pseudo-aligned using Kalisto v0.42.4 to GENCODE v23 transcripts (Bray et al., 2016). Transcripts with transcript type in [protein_coding, IG_C_gene, IG_D_gene, IG_J_gene, IG_V_gene, TR_C_gene, TR_V_gene, TR_D_gene, TR_J_gene] were selected, then non-coding RNA transcripts and histones and mitochondrial transcripts were removed resulting in 20,062 protein coding genes. Gene expressions were quantified as transcripts per million (TPM) and log 2 transformed (Conesa et al., 2016). Gene expression changes in cell lines treated with EGFRi were shown as relative (log) expression normalized to untreated control cells. Patients' tumor gene expression was median-centered within the 4 patient samples, with gene expression relative to the control median value shown on the heatmaps. PROGENy v1.4.1 was used to calculate 7 pathways activity scores (EGFR, MAPK, PI3K, TRAIL, TNFa, NFkB, JAK-STAT; Schubert et al., 2018). Other pathway signature scores were calculated using ssGSEA using in-house python implementation. The pathways activity were represented as gene signatures, downloaded from mSiGdB v6.2 (Subramanian et al., 2005), unless other specified: “Cell cycle signature”—HALLMARK_G2M_CHECKPOINT, “Apoptosis signature”—HALLMARK_APOPTOSIS, MYC HALLMARK_MYC_TARGETS_V2, “EMT signature” 788-HALLMARK_EPITHELIAL_MESENCHYMAL_TRANSITION, “iFN-0 signature” HALLMARK_INTERFERON_GAMMA_RESPONSE, “HLA expression”—gene set (HILA-A, HLA-B, HLA-C). Pathway score differences relative to the control were normalized to the maximum values in each pathway separately and displayed on the line plots. For schematic representation, the maximum absolute deviation of the pathway activity score change from the control point (0 h) to the 24 h time point was calculated within each pathway. Pathway colors on the schema corresponds to the percent of the maximum absolute deviation. Cell deconvolution was performed from RNAseq data using the quanTIseq approach (Finotello et al., 2018). Heatmaps, dot plots, line plots, bar plots were created using pandas v0.23.4, matplotlib v2.1.1 and seaborn v0.9.0 python packages (Liang et al., 2016).

Luminex assay. Duplicate samples of supernatants from untreated and 1 μM EGFRi-treated H1975 and H1299 cell lines were analyzed for the presence of CCL2, CXCL1, CXCL2, CXCL8, IP10, IL1RA, IL6, VEGFA, CSF2, and CSF3 proteins using a custom Luminex kit according to the manufacturer's instructions (R&D Systems. USA). Fifty microliters of test supernatant and the appropriate microparticle cocktails were added (1:10 dilution) to each well and incubated for 2 hours in a microplate shaker at room temperature. Plate was washed 3 times and 50 μl microliters of Biotin-Antibody cocktail (1:10 dilution) was added and incubated for 1 hour, followed by 3 washes. After incubating with 50 μl of Streptavidin (1:25 dilution) for 30 minutes, the plate was washed 3 times, microparticles were resuspended in buffer and the plate was read using a Luminex plate reader. EGFRi-induced changes were expressed as fold increase or fold decrease compared to measured baseline (0 h) concentrations. In cases where concentrations measured fell below the level of detection, the minimum threshold of detection according to the manufacturer was used.

T-cell functional assays. T-cell migration: EGFRi Osimertinib (LC laboratories, USA) at 1 μM concentration was used to treat H1975 cells for 24 hrs. DMSO only with media was used as a control. Cells were then washed and incubated in ImmunoCulut-XF T cell expansion medium (Stem Cell Technology, Canada) for 24 hrs, after which cell supernatants were collected and filtered using a Millex-GS filter. Healthy donor PBMC or expanded melanoma CD8⁺ tumor-infiltrating lymphocytes (TIL 3311 and TIL3329) were thawed ˜16 hours prior to performing the migration assay. 650 μL of H1975 cell supernatant was placed at the bottom of a transwell plate (Corning, USA) and incubated with 3×10⁵ healthy donor PBMCs in the top well for 6 hrs. Migrated cells at the well bottom were collected and stained for CD4, CD8, or CD14 (Biolegend) for 30 mins at 4° C., washed with PBS and fixed with 4% PFA. 50 μl of counting beads were added to each sample to obtain accurate cell counts. Samples were run on a Canto II flow cytometer and analyzed using FlowJo V10.

HLA class I quantitation and T-cell antigen recognition: H1299 and H1975 cells were treated with 1 μM EGFRi Osimertinib (LC laboratories) or DMSO control for 6 hrs or 20 hrs. Cells were collected and stained for total class I (W6/32-APC, Thermo-Fisher, USA), washed, and fixed in 4% PFA. Samples were run on a Canto II flow cytometer and analyzed using FlowJo V10. H1975 cells were seeded at 50,000 cells per well in 96-well plates and EGFRi was used to treat cells at concentrations of 0, 0.1, and 0.3 μM per well for 24 hrs. H1975 cells were then pulsed with 0, 10, or 100 nM of cognate HLA-A0101-restricted VGLL1 peptide LSELETPGKY (SEQ ID NO: 978) for one hour prior to washing. VGLL1 peptide antigen-specific CD8⁺ T cells were then added at a 1:1 effector-to-target cell ratio and co-cultured overnight. IFN-γ in 24 hour cell supernatants was analyzed using a human IFN-7 ELISA kit (Invitrogen, USA) and plates were read using SpectraMax® M5/M5e Multimode PlateReader.

Statistical analysis. All statistical analyses were performed using the GraphPad Prism 5 (GraphPad Software Inc., La Jolla, Calif.). Survival curves and rates were calculated using the Log-rank (Mantel-Cox) Test, and overall survival was measured from the date of enrollment up to Dec. 31, 2018 or the time of death. Two-tailed Student's t test or Mann-Whitney U test was used to analyze the statistical significance between groups. A P-value less than or equal to 0.05 was the threshold used to determine statistical significance.

Data Availability. Raw data of bulk RNA sequencing of patient tumors and cell lines, single cell TCR paired as chain sequencing, TCR VβCDR3 sequencing generated and analyzed during the current study are available through NCBI Sequence Read Archive (SRA) (code SRP188005). DNA sequencing data (for finding mutations) with potential patient identification was not include in the informed consent of enrolled patients, data without identifying information can be shared upon reasonable request. All other data are available from the corresponding author upon reasonable request.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims. The following references, to the extent that they provide exemplary procedural or other details supplementary to those set forth herein, are specifically incorporated herein by reference.
Andreatta and Nielsen, Bioinformatics, 32:511-517, 2015.
Bedognetti et al., J. Immunother. Cancer, 7:131, 2019.
Bradley et al., Cancer, Immunol. Res., 3:602-609, 2015.
Bray et al., Nat. Biotechnol., 34:525-527, 2016.
Cafri et al., Nat. Commun., 10:449, 2019.
Carreno et al., Science, 348:803-808, 2015.
Chen et al., J. Cin. Invest., 129(5):2056-2070, 2019.
Cohen et al., (Cancer. Rev., 67:3898-3903, 2007.
Conesa et al., Genome. Biol., 17:13-31, 2016.
El Kadi et al., Cancer Res., 78:6728-6735, 2018.
Finn and Rammensee, Cold Spring Harb. Perspect. Biol., 10:a028829, 2018.
Finotello and Trajanoski, Cancer. Immunol. Immun., 67:1031-1040, 2018.
Frederick et al., Clin. Cancer. Res., 19:1225-1231, 2013.
Galon and Bruni, Nat. Rev. Drug. Discov., 18:197-218, 2019.
GonzAlez-Galarza et al., Nucleic Acids Res., 43:D784-788, 2015.
Goodridge et al., J. Immunol., 191:1567-1577, 2013.
Griffin et al., Oncotarget, 8:78174-78192, 2017.
Hailemichael et al., Nat. Med., 19:465-472, 2013.
Harjanto et al., PLoS One, 9:e86655, 2014.
Hao et al., Nat. Genet., 48:1500-1507, 2016.
Hilf et al., Nature, 565:240-245, 2019.
Hu et al., Nat. Rev. Immunol., 18:168-182, 2018.
Iiizumi et al., Cancers, 11:266-279, 2019.
Im et al., PLoS One, 11:e0160004, 2016.
Jemal et al., CA. Cancer J. Clin., 61:69-90, 2011.
Jensen et al., Immunology, 154:394-406, 2018.
Jia et al., Int. J. Cancer. 145(5):1432-1444, 2019.
Keenan et al., Nat. Med., 25:389-402, 2019.
Keskin et al., Nature, 565, 234-239, 2019.
Khalili et al., Clin. Cancer. Res., 1:5329-5340, 2012.
Knight et al., J. Clin. Invest., 123:1371-1381, 2013.
Kobayashi et al., Cancer Sci., 107:1179-1186, 2016.
Kobayashi et al., N. Engl. J. Med., 352:786-792, 2005.
Kreiter et al., Nature, 520:692-696, 2015.
Lawrence et al., Nature, 499:214-218, 2013.
Levine et al., Cancer Cell, 35:10-15, 2019.
Li and Gillanders, Ann. Oncol., 28:xii11-xii17, 2017.
Li et al., Oncoimmunology, 5:e1238539, 2016.
Liang et al., Ieee. Acm. T. Comput. Bi., 13:549-556, 2016.
Liu and Mardis, Cell, 168:600-612, 2017.
Lu and Robbins, Semin. Immunol., 28:22-27, 2016.
Malekzadeh et al., J. Clin. Invest., 129:1109-1114, 2019.
McGranahan et al., Cell, 171:1259-1271, 2017.
Morgensztern et al., J. Thorac. Oncol., 7:1485-1489, 2012a.
Morgensztern et al., J. Thorac. Oncol., 7:1479-1484, 2012b.
Munn and Bronte, Curr. Opin. Immunol., 39:1-6, 2016.
Ott et al., Nature, 13:217-221, 2017.
Overwijk, Nat. Med., 21:12-14, 2015.
Parker et al., Adv. Cancer. Res., 128:95-139, 2015.
Pol et al., Oncoimmunology, 4:e974411, 2015.
Rizvi et al., Science, 348:124-128, 2015.
Robinson et al., Nucleic. Acids Re., 43:D423-D431, 2014.
Rosenthal et al., Nature, 567:479-485, 2019.
Sahin et al., Nature, 547:222-226, 2017.
Schoenborn and Wilson, Adv. Immunol., 96:41-101, 2007.
Schroder et al., J. Leukoc. Biol., 75:163-189, 2004.
Schubert et al., Nat. Commun., 9:20-30, 2018.
Schumacher and Schreiber, Science, 348:69-74, 2015.
Sidney et al., BMC Immunol., 22(9):12, 2008.
Siegel et al., CA: Cancer J. Clin., 67:7-30, 2016.
Subramanian et al., Proc. Natl. Acad. Sci., 102:15545-15550, 2005.
Sumitro et al., PLoS One, 27:e86655, 2014.
Tran et al., Science, 344:641-645, 2014.
Tumeh et al., Nature, 515:568-571, 2014.
Venugopalan et al., Oncotarget, 23:54137-54156, 2016.
Wang et al., Mol. Cancer, 17:168, 2018.
Watanabe et al., Cancer. Sci., 110:52-60, 2019.
Wilmott et al., Clin. Cancer. Res., 18:1386-1394, 2012.
Wingett and Andrews, F1000Res., 7:1338-1350, 2018.
Wirth and Kühnel, Front. Immunol., 8:1848, 2017.
Xu et al., Oncotarget, 8:90557-90578, 2017.
Yamaoka et al., Int. J. Mol. Sci., 18:E2420, 2017.
Yossef et al., JCI Insight, 3:122647, 2018.

1. A method of treating a subject having a EGFR-mutant cancer comprising administering to the subject at least a first HLA-binding peptide from EGFR and at least a first EGFR inhibitor, said HLA-binding peptide comprising a mutated amino acid sequence relative to wild type human EGFR that matches the mutation in the cancer of the subject.

2. The method of claim 1, wherein the HLA-binding peptide from EGFR binds to a HLA class I molecule.

3. The method of claim 2, wherein the HLA class I-binding peptide is 8, 9, 10, 11, 12 or 13 amino acids in length, optionally 9, 10 or 11 amino acids in length.

4. The method of claim 1, wherein the HLA-binding peptide from EGFR binds to a HLA class II molecule.

5. The method of claim 4, wherein the HILA class II-binding peptide is 13-30 amino acids in length, optionally 15-23 amino acids in length.

6. The method of claim 1, comprising administering at least a first and a second HLA-binding peptide from EGFR, wherein said first and second HLA-binding peptides each comprise a mutated amino acid sequence relative to wild type human EGFR that matches the mutation in the cancer of the subject.

7. The method of claim 6, wherein the first HLA-binding peptide from EGFR binds to a HLA class I molecule and the second HILA-binding peptide from EGFR binds to a HLA class II molecule.

8. The method of claim 6, comprising administering a plurality of HLA-binding peptides from EGFR, wherein said HLA-binding peptides each comprise a mutated amino acid sequence relative to wild type human EGFR that matches the mutation in the cancer of the subject.

9. The method of claim 8, wherein the plurality of HLA-binding peptides comprises peptides that bind to both HLA class I and HLA class II molecules.

10. The method of claim 8, comprising administering 2 to 30 different HLA-binding peptides to the subject, optionally 5 to 30 different HLA-binding peptides.

11-26. (canceled)

27. An immunogenic composition comprising at least a first and a second HLA-binding peptide from EGFR, said first and second HLA-binding peptides each comprising a mutated amino acid sequence relative to wild type human EGFR that matches a mutation in a human EGFR-mutant cancer, wherein the first HLA-binding peptide from EGFR binds to a HLA class I molecule and the second HLA-binding peptide from EGFR binds to a HLA class II molecule.

28. The composition of claim 27, wherein the HLA-binding peptides are formulated in a pharmaceutically acceptable carrier.

29. The composition of claim 28, wherein the pharmaceutically acceptable carrier is an aqueous carrier, a salt solution, a saline solution, and/or an isotonic saline solution.

30. The composition of claim 27, wherein the HLA class I-binding peptide is 8, 9, 10, 11, 12 or 13 amino acids in length, optionally 9, 10 or 11 amino acids in length.

31. The composition of claim 27, wherein the HLA class II-binding peptide is 13-30 amino acids in length, optionally 15-23 amino acids in length.

32. The composition of claim 27, comprising a plurality of HLA-binding peptides from EGFR wherein said HLA-binding peptides each comprise a mutated amino acid sequence relative to wild type human EGFR that matches a EGFR mutation in a human EGFR-mutant cancer.

33. The composition of claim 32, comprising 2 to 30 different HLA-binding peptides to the subject.

34. The composition of claim 32, comprising at least two HLA class I-binding peptides and at least one HLA class II-binding peptide.

35. The composition of claim 32, comprising at least one HLA class I-binding peptide and at least two HLA class II-binding peptides.

36. The composition of claim 33, comprising at least two HLA class I-binding peptides and at least two HLA class II-binding peptides, optionally at least 3 HLA class I-binding peptides and at least 3 HLA class II-binding peptides.

37-85. (canceled)


SEQUENCE LISTING (982 sequences)

SEQ ID NO: 1 - protein, 9 aa, Artificial Sequence
  Synthetic peptide
HVKITDFGR

SEQ ID NO: 2 - protein, 10 aa, Artificial Sequence
  Synthetic sequence
RAKLLGAEEK

SEQ ID NO: 3 - protein, 9 aa, Artificial Sequence
  Synthetic peptide
ITDFGRAKL

SEQ ID NO: 4 - protein, 9 aa, Artificial Sequence
  Synthetic peptide
KITDFGRAK

SEQ ID NO: 5 - protein, 10 aa, Artificial Sequence
  Synthetic peptide
QHVKITDFGR

SEQ ID NO: 6 - protein, 9 aa, Artificial Sequence
  Synthetic peptide
FGRAKLLGA

SEQ ID NO: 7 - protein, 11 aa, Artificial Sequence
  Synthetic peptide
GRAKLLGAEEK

SEQ ID NO: 8 - protein, 9 aa, Artificial Sequence
  Synthetic peptide
RAKLLGAEE

SEQ ID NO: 9 - protein, 11 aa, Artificial Sequence
  Synthetic peptide
HVKITDFGRAK

SEQ ID NO: 10 - protein, 10 aa, Artificial Sequence
  Synthetic peptide
KITDFGRAKL

SEQ ID NO: 11 - protein, 12 aa, Artificial Sequence
  Synthetic peptide
TPQHVKITDFGR

SEQ ID NO: 12 - protein, 10 aa, Artificial Sequence
  Synthetic peptide
ITDFGRAKLL

SEQ ID NO: 13 - protein, 12 aa, Artificial Sequence
  Synthetic peptide
RAKLLGAEEKEY

SEQ ID NO: 14 - protein, 10 aa, Artificial Sequence
  Synthetic peptide
HVKITDFGRA

SEQ ID NO: 15 - protein, 9 aa, Artificial Sequence
  Synthetic peptide
TDFGRAKLL

SEQ ID NO: 16 - protein, 10 aa, Artificial Sequence
  Synthetic peptide
VKITDFGRAK

SEQ ID NO: 17 - protein, 12 aa, Artificial Sequence
  Synthetic peptide
ITDFGRAKLLGA

SEQ ID NO: 18 - protein, 9 aa, Artificial Sequence
  Synthetic peptide
GRAKLLGAE

SEQ ID NO: 19 - protein, 10 aa, Artificial Sequence
  Synthetic peptide
IPVAIKTSPK

SEQ ID NO: 20 - protein, 10 aa, Artificial Sequence
  Synthetic peptide
AIKTSPKANK

SEQ ID NO: 21 - protein, 11 aa, Artificial Sequence
  Synthetic peptide
KIPVAIKTSPK

SEQ ID NO: 22 - protein, 10 aa, Artificial Sequence
  Synthetic peptide
KVKIPVAIKT

SEQ ID NO: 23 - protein, 9 aa, Artificial Sequence
  Synthetic peptide
PVAIKTSPK

SEQ ID NO: 24 - protein, 12 aa, Artificial Sequence
  Synthetic peptide
VKIPVAIKTSPK

SEQ ID NO: 25 - protein, 8 aa, Artificial Sequence
  Synthetic peptide
KTSPKANK

SEQ ID NO: 26 - protein, 11 aa, Artificial Sequence
  Synthetic peptide
IPVAIKTSPKA

SEQ ID NO: 27 - protein, 9 aa, Artificial Sequence
  Synthetic peptide
IPVAIKTSP

SEQ ID NO: 28 - protein, 11 aa, Artificial Sequence
  Synthetic peptide
VAIKTSPKANK

SEQ ID NO: 29 - protein, 10 aa, Artificial Sequence
  Synthetic peptide
KTSPKANKEI

SEQ ID NO: 30 - protein, 9 aa, Artificial Sequence
  Synthetic peptide
VAIKTSPKA

SEQ ID NO: 31 - protein, 11 aa, Artificial Sequence
  Synthetic peptide
KTSPKANKEIL

SEQ ID NO: 32 - protein, 10 aa, Artificial Sequence
  Synthetic peptide
AIKESPKANK

SEQ ID NO: 33 - protein, 10 aa, Artificial Sequence
  Synthetic peptide
IPVAIKESPK

SEQ ID NO: 34 - protein, 11 aa, Artificial Sequence
  Synthetic peptide
KIPVAIKESPK

SEQ ID NO: 35 - protein, 11 aa, Artificial Sequence
  Synthetic peptide
IPVAIKESPKA

SEQ ID NO: 36 - protein, 11 aa, Artificial Sequence
  Synthetic peptide
KVKIPVAIKES

SEQ ID NO: 37 - protein, 11 aa, Artificial Sequence
  Synthetic peptide
VAIKESPKANK

SEQ ID NO: 38 - protein, 10 aa, Artificial Sequence
  Synthetic peptide
KESPKANKEI

SEQ ID NO: 39 - protein, 9 aa, Artificial Sequence
  Synthetic peptide
IPVAIKESP

SEQ ID NO: 40 - protein, 11 aa, Artificial Sequence
  Synthetic peptide
KESPKANKEIL

SEQ ID NO: 41 - protein, 12 aa, Artificial Sequence
  Synthetic peptide
VKIPVAIKESPK

SEQ ID NO: 42 - protein, 10 aa, Artificial Sequence
  Synthetic peptide
ESPKANKEIL

SEQ ID NO: 43 - protein, 9 aa, Artificial Sequence
  Synthetic peptide
PVAIKESPK

SEQ ID NO: 44 - protein, 12 aa, Artificial Sequence
  Synthetic peptide
IKESPKANKEIL

SEQ ID NO: 45 - protein, 9 aa, Artificial Sequence
  Synthetic peptide
VAIKESPKA

SEQ ID NO: 46 - protein, 9 aa, Artificial Sequence
  Synthetic peptide
AIKESKANK

SEQ ID NO: 47 - protein, 12 aa, Artificial Sequence
  Synthetic peptide
KVKIPVAIKESK

SEQ ID NO: 48 - protein, 10 aa, Artificial Sequence
  Synthetic peptide
IPVAIKESKA

SEQ ID NO: 49 - protein, 10 aa, Artificial Sequence
  Synthetic peptide
KESKANKEIL

SEQ ID NO: 50 - protein, 9 aa, Artificial Sequence
  Synthetic peptide
KESKANKEI

SEQ ID NO: 51 - protein, 10 aa, Artificial Sequence
  Synthetic peptide
KIPVAIKESK

SEQ ID NO: 52 - protein, 10 aa, Artificial Sequence
  Synthetic peptide
VAIKESKANK

SEQ ID NO: 53 - protein, 11 aa, Artificial Sequence
  Synthetic peptide
KVKIPVAIKES

SEQ ID NO: 54 - protein, 12 aa, Artificial Sequence
  Synthetic peptide
SKANKEILDEAY

SEQ ID NO: 55 - protein, 9 aa, Artificial Sequence
  Synthetic peptide
IPVAIKESK

SEQ ID NO: 56 - protein, 11 aa, Artificial Sequence
  Synthetic peptide
AIKESKANKEI

SEQ ID NO: 57 - protein, 12 aa, Artificial Sequence
  Synthetic peptide
IPVAIKESKANK

SEQ ID NO: 58 - protein, 10 aa, Artificial Sequence
  Synthetic peptide
IKESKANKEI

SEQ ID NO: 59 - protein, 10 aa, Artificial Sequence
  Synthetic peptide
VQLIMQLMPF

SEQ ID NO: 60 - protein, 10 aa, Artificial Sequence
  Synthetic peptide
MQLMPFGCLL

SEQ ID NO: 61 - protein, 9 aa, Artificial Sequence
  Synthetic peptide
MQLMPFGCL

SEQ ID NO: 62 - protein, 9 aa, Artificial Sequence
  Synthetic peptide
QLIMQLMPF

SEQ ID NO: 63 - protein, 11 aa, Artificial Sequence
  Synthetic peptide
IMQLMPFGCLL

SEQ ID NO: 64 - protein, 12 aa, Artificial Sequence
  Synthetic peptide
MQLMPFGCLLDY

SEQ ID NO: 65 - protein, 9 aa, Artificial Sequence
  Synthetic peptide
LTSTVQLIM

SEQ ID NO: 66 - protein, 12 aa, Artificial Sequence
  Synthetic peptide
LIMQLMPFGCLL

SEQ ID NO: 67 - protein, 10 aa, Artificial Sequence
  Synthetic peptide
IMQLMPFGCL

SEQ ID NO: 68 - protein, 9 aa, Artificial Sequence
  Synthetic peptide
STVQLIMQL

SEQ ID NO: 69 - protein, 8 aa, Artificial Sequence
  Synthetic peptide
LIMQLMPF

SEQ ID NO: 70 - protein, 11 aa, Artificial Sequence
  Synthetic peptide
TVQLIMQLMPF

SEQ ID NO: 71 - protein, 8 aa, Artificial Sequence
  Synthetic peptide
VQLIMQLM

SEQ ID NO: 72 - protein, 10 aa, Artificial Sequence
  Synthetic peptide
CLTSTVQLIM

SEQ ID NO: 73 - protein, 12 aa, Artificial Sequence
  Synthetic peptide
STVQLIMQLMPF

SEQ ID NO: 74 - protein, 10 aa, Artificial Sequence
  Synthetic peptide
TSTVQLIMQL

SEQ ID NO: 75 - protein, 11 aa, Artificial Sequence
  Synthetic peptide
LIMQLMPFGCL

SEQ ID NO: 76 - protein, 10 aa, Artificial Sequence
  Synthetic peptide
STVQLIMQLM

SEQ ID NO: 77 - protein, 10 aa, Artificial Sequence
  Synthetic peptide
QLIMQLMPFG

SEQ ID NO: 78 - protein, 12 aa, Artificial Sequence
  Synthetic peptide
LTSTVQLIMQLM

SEQ ID NO: 79 - protein, 11 aa, Artificial Sequence
  Synthetic peptide
LTSTVQLIMQL

SEQ ID NO: 80 - protein, 11 aa, Artificial Sequence
  Synthetic peptide
ICLTSTVQLIM

SEQ ID NO: 81 - protein, 10 aa, Artificial Sequence
  Synthetic peptide
LTSTVQLIMQ

SEQ ID NO: 82 - protein, 9 aa, Artificial Sequence
  Synthetic peptide
TVQLIMQLM

SEQ ID NO: 83 - protein, 10 aa, Artificial Sequence
  Synthetic peptide
KQLGAEEKEY

SEQ ID NO: 84 - protein, 9 aa, Artificial Sequence
  Synthetic peptide
TDFGLAKQL

SEQ ID NO: 85 - protein, 10 aa, Artificial Sequence
  Synthetic peptide
KITDFGLAKQ

SEQ ID NO: 86 - protein, 12 aa, Artificial Sequence
  Synthetic peptide
LAKQLGAEEKEY

SEQ ID NO: 87 - protein, 10 aa, Artificial Sequence
  Synthetic peptide
ITDFGLAKQL

SEQ ID NO: 88 - protein, 10 aa, Artificial Sequence
  Synthetic peptide
LAKQLGAEEK

SEQ ID NO: 89 - protein, 11 aa, Artificial Sequence
  Synthetic peptide
GLAKQLGAEEK

SEQ ID NO: 90 - protein, 9 aa, Artificial Sequence
  Synthetic peptide
FGLAKQLGA

SEQ ID NO: 91 - protein, 11 aa, Artificial Sequence
  Synthetic peptide
KITDFGLAKQL

SEQ ID NO: 92 - protein, 9 aa, Artificial Sequence
  Synthetic peptide
QLGAEEKEY

SEQ ID NO: 93 - protein, 12 aa, Artificial Sequence
  Synthetic peptide
ITDFGLAKQLGA

SEQ ID NO: 94 - protein, 10 aa, Artificial Sequence
  Synthetic peptide
ILDEAYVMAI

SEQ ID NO: 95 - protein, 11 aa, Artificial Sequence
  Synthetic peptide
YVMAIVDNPHV

SEQ ID NO: 96 - protein, 10 aa, Artificial Sequence
  Synthetic peptide
VMAIVDNPHV

SEQ ID NO: 97 - protein, 11 aa, Artificial Sequence
  Synthetic peptide
ILDEAYVMAIV

SEQ ID NO: 98 - protein, 12 aa, Artificial Sequence
  Synthetic peptide
KEILDEAYVMAI

SEQ ID NO: 99 - protein, 11 aa, Artificial Sequence
  Synthetic peptide
MAIVDNPHVCR

SEQ ID NO: 100 - protein, 9 aa, Artificial Sequence
  Synthetic peptide
MAIVDNPHV

SEQ ID NO: 101 - protein, 12 aa, Artificial Sequence
  Synthetic peptide
AYVMAIVDNPHV

SEQ ID NO: 102 - protein, 10 aa, Artificial Sequence
  Synthetic peptide
AIVDNPHVCR

SEQ ID NO: 103 - protein, 12 aa, Artificial Sequence
  Synthetic peptide
EILDEAYVMAIV

SEQ ID NO: 104 - protein, 12 aa, Artificial Sequence
  Synthetic peptide
VMAIVDNPHVCR

SEQ ID NO: 105 - protein, 11 aa, Artificial Sequence
  Synthetic peptide
EILDEAYVMAI

SEQ ID NO: 106 - protein, 9 aa, Artificial Sequence
  Synthetic peptide
IVDNPHVCR

SEQ ID NO: 107 - protein, 9 aa, Artificial Sequence
  Synthetic peptide
DEAYVMAIV

SEQ ID NO: 108 - protein, 12 aa, Artificial Sequence
  Synthetic peptide
YVMAIVDNPHVC

SEQ ID NO: 109 - protein, 10 aa, Artificial Sequence
  Synthetic peptide
YVMAIVDNPH

SEQ ID NO: 110 - protein, 10 aa, Artificial Sequence
  Synthetic peptide
IVDNPHVCRL

SEQ ID NO: 111 - protein, 9 aa, Artificial Sequence
  Synthetic peptide
VMAIVDNPH

SEQ ID NO: 112 - protein, 10 aa, Artificial Sequence
  Synthetic peptide
LDEAYVMAIV

SEQ ID NO: 113 - protein, 8 aa, Artificial Sequence
  Synthetic peptide
DEAYVMAI

SEQ ID NO: 114 - protein, 8 aa, Artificial Sequence
  Synthetic peptide
EAYVMAIV

SEQ ID NO: 115 - protein, 11 aa, Artificial Sequence
  Synthetic peptide
IVDNPHVCRLL

SEQ ID NO: 116 - protein, 9 aa, Artificial Sequence
  Synthetic peptide
YVMAIVDNP

SEQ ID NO: 117 - protein, 11 aa, Artificial Sequence
  Synthetic peptide
AIVDNPHVCRL

SEQ ID NO: 118 - protein, 9 aa, Artificial Sequence
  Synthetic peptide
LDEAYVMAI

SEQ ID NO: 119 - protein, 12 aa, Artificial Sequence
  Synthetic peptide
MAIVDNPHVCRL

SEQ ID NO: 120 - protein, 8 aa, Artificial Sequence
  Synthetic peptide
MAIVDNPH

SEQ ID NO: 121 - protein, 12 aa, Artificial Sequence
  Synthetic peptide
AIVDNPHVCRLL

SEQ ID NO: 122 - protein, 9 aa, Artificial Sequence
  Synthetic peptide
IKVLASGAF

SEQ ID NO: 123 - protein, 10 aa, Artificial Sequence
  Synthetic peptide
ASGAFGTVYK

SEQ ID NO: 124 - protein, 10 aa, Artificial Sequence
  Synthetic peptide
VLASGAFGTV

SEQ ID NO: 125 - protein, 10 aa, Artificial Sequence
  Synthetic peptide
KIKVLASGAF

SEQ ID NO: 126 - protein, 10 aa, Artificial Sequence
  Synthetic peptide
LASGAFGTVY

SEQ ID NO: 127 - protein, 9 aa, Artificial Sequence
  Synthetic peptide
ASGAFGTVY

SEQ ID NO: 128 - protein, 9 aa, Artificial Sequence
  Synthetic peptide
KIKVLASGA

SEQ ID NO: 129 - protein, 9 aa, Artificial Sequence
  Synthetic peptide
VLASGAFGT

SEQ ID NO: 130 - protein, 9 aa, Artificial Sequence
  Synthetic peptide
LASGAFGTV

SEQ ID NO: 131 - protein, 11 aa, Artificial Sequence
  Synthetic peptide
KKIKVLASGAF

SEQ ID NO: 132 - protein, 12 aa, Artificial Sequence
  Synthetic peptide
VLASGAFGTVYK

SEQ ID NO: 133 - protein, 9 aa, Artificial Sequence
  Synthetic peptide
KVLASGAFG

SEQ ID NO: 134 - protein, 11 aa, Artificial Sequence
  Synthetic peptide
LASGAFGTVYK

SEQ ID NO: 135 - protein, 12 aa, Artificial Sequence
  Synthetic peptide
KVLASGAFGTVY

SEQ ID NO: 136 - protein, 11 aa, Artificial Sequence
  Synthetic peptide
VLASGAFGTVY

SEQ ID NO: 137 - protein, 11 aa, Artificial Sequence
  Synthetic peptide
KVLASGAFGTV

SEQ ID NO: 138 - protein, 12 aa, Artificial Sequence
  Synthetic peptide
FKKIKVLASGAF

SEQ ID NO: 139 - protein, 10 aa, Artificial Sequence
  Synthetic peptide
IKVLASGAFG

SEQ ID NO: 140 - protein, 8 aa, Artificial Sequence
  Synthetic peptide
KVLASGAF

SEQ ID NO: 141 - protein, 10 aa, Artificial Sequence
  Synthetic peptide
TEFKKIKVLA

SEQ ID NO: 142 - protein, 11 aa, Artificial Sequence
  Synthetic peptide
ASGAFGTVYKG

SEQ ID NO: 143 - protein, 10 aa, Artificial Sequence
  Synthetic peptide
KKIKVLASGA

SEQ ID NO: 144 - protein, 9 aa, Artificial Sequence
  Synthetic peptide
FKKIKVLAS

SEQ ID NO: 145 - protein, 12 aa, Artificial Sequence
  Synthetic peptide
KETEFKKIKVLA

SEQ ID NO: 146 - protein, 8 aa, Artificial Sequence
  Synthetic peptide
FKKIKVLA

SEQ ID NO: 147 - protein, 12 aa, Artificial Sequence
  Synthetic peptide
ASGAFGTVYKGL

SEQ ID NO: 148 - protein, 11 aa, Artificial Sequence
  Synthetic peptide
FKKIKVLASGA

SEQ ID NO: 149 - protein, 9 aa, Artificial Sequence
  Synthetic peptide
IKVLCSGAF

SEQ ID NO: 150 - protein, 10 aa, Artificial Sequence
  Synthetic peptide
VLCSGAFGTV

SEQ ID NO: 151 - protein, 10 aa, Artificial Sequence
  Synthetic peptide
CSGAFGTVYK

SEQ ID NO: 152 - protein, 10 aa, Artificial Sequence
  Synthetic peptide
KIKVLCSGAF

SEQ ID NO: 153 - protein, 11 aa, Artificial Sequence
  Synthetic peptide
KKIKVLCSGAF

SEQ ID NO: 154 - protein, 9 aa, Artificial Sequence
  Synthetic peptide
CSGAFGTVY

SEQ ID NO: 155 - protein, 9 aa, Artificial Sequence
  Synthetic peptide
KIKVLCSGA

SEQ ID NO: 156 - protein, 12 aa, Artificial Sequence
  Synthetic peptide
VLCSGAFGTVYK

SEQ ID NO: 157 - protein, 9 aa, Artificial Sequence
  Synthetic peptide
KVLCSGAFG

SEQ ID NO: 158 - protein, 12 aa, Artificial Sequence
  Synthetic peptide
KVLCSGAFGTVY

SEQ ID NO: 159 - protein, 9 aa, Artificial Sequence
  Synthetic peptide
VLCSGAFGT

SEQ ID NO: 160 - protein, 11 aa, Artificial Sequence
  Synthetic peptide
KVLCSGAFGTV

SEQ ID NO: 161 - protein, 10 aa, Artificial Sequence
  Synthetic peptide
LCSGAFGTVY

SEQ ID NO: 162 - protein, 8 aa, Artificial Sequence
  Synthetic peptide
KVLCSGAF

SEQ ID NO: 163 - protein, 12 aa, Artificial Sequence
  Synthetic peptide
FKKIKVLCSGAF

SEQ ID NO: 164 - protein, 11 aa, Artificial Sequence
  Synthetic peptide
VLCSGAFGTVY

SEQ ID NO: 165 - protein, 10 aa, Artificial Sequence
  Synthetic peptide
IKVLCSGAFG

SEQ ID NO: 166 - protein, 11 aa, Artificial Sequence
  Synthetic peptide
LCSGAFGTVYK

SEQ ID NO: 167 - protein, 10 aa, Artificial Sequence
  Synthetic peptide
KKIKVLCSGA

SEQ ID NO: 168 - protein, 10 aa, Artificial Sequence
  Synthetic peptide
TEFKKIKVLC

SEQ ID NO: 169 - protein, 11 aa, Artificial Sequence
  Synthetic peptide
KIKVLCSGAFG

SEQ ID NO: 170 - protein, 9 aa, Artificial Sequence
  Synthetic peptide
LCSGAFGTV

SEQ ID NO: 171 - protein, 9 aa, Artificial Sequence
  Synthetic peptide
IKVLSSGAF

SEQ ID NO: 172 - protein, 10 aa, Artificial Sequence
  Synthetic peptide
SSGAFGTVYK

SEQ ID NO: 173 - protein, 10 aa, Artificial Sequence
  Synthetic peptide
VLSSGAFGTV

SEQ ID NO: 174 - protein, 10 aa, Artificial Sequence
  Synthetic peptide
KIKVLSSGAF

SEQ ID NO: 175 - protein, 9 aa, Artificial Sequence
  Synthetic peptide
KIKVLSSGA

SEQ ID NO: 176 - protein, 10 aa, Artificial Sequence
  Synthetic peptide
LSSGAFGTVY

SEQ ID NO: 177 - protein, 9 aa, Artificial Sequence
  Synthetic peptide
SSGAFGTVY

SEQ ID NO: 178 - protein, 11 aa, Artificial Sequence
  Synthetic peptide
KKIKVLSSGAF

SEQ ID NO: 179 - protein, 9 aa, Artificial Sequence
  Synthetic peptide
LSSGAFGTV

SEQ ID NO: 180 - protein, 11 aa, Artificial Sequence
  Synthetic peptide
LSSGAFGTVYK

SEQ ID NO: 181 - protein, 12 aa, Artificial Sequence
  Synthetic peptide
VLSSGAFGTVYK

SEQ ID NO: 182 - protein, 9 aa, Artificial Sequence
  Synthetic peptide
KVLSSGAFG

SEQ ID NO: 183 - protein, 9 aa, Artificial Sequence
  Synthetic peptide
VLSSGAFGT

SEQ ID NO: 184 - protein, 12 aa, Artificial Sequence
  Synthetic peptide
KVLSSGAFGTVY

SEQ ID NO: 185 - protein, 12 aa, Artificial Sequence
  Synthetic peptide
FKKIKVLSSGAF

SEQ ID NO: 186 - protein, 10 aa, Artificial Sequence
  Synthetic peptide
IKVLSSGAFG

SEQ ID NO: 187 - protein, 11 aa, Artificial Sequence
  Synthetic peptide
KVLSSGAFGTV

SEQ ID NO: 188 - protein, 11 aa, Artificial Sequence
  Synthetic peptide
VLSSGAFGTVY

SEQ ID NO: 189 - protein, 8 aa, Artificial Sequence
  Synthetic peptide
KVLSSGAF

SEQ ID NO: 190 - protein, 10 aa, Artificial Sequence
  Synthetic peptide
KKIKVLSSGA

SEQ ID NO: 191 - protein, 11 aa, Artificial Sequence
  Synthetic peptide
SSGAFGTVYKG

SEQ ID NO: 192 - protein, 9 aa, Artificial Sequence
  Synthetic peptide
FKKIKVLSS

SEQ ID NO: 193 - protein, 10 aa, Artificial Sequence
  Synthetic peptide
TEFKKIKVLS

SEQ ID NO: 194 - protein, 10 aa, Artificial Sequence
  Synthetic peptide
IPVAIKVPKA

SEQ ID NO: 195 - protein, 12 aa, Artificial Sequence
  Synthetic peptide
KVKIPVAIKVPK

SEQ ID NO: 196 - protein, 9 aa, Artificial Sequence
  Synthetic peptide
VPKANKEIL

SEQ ID NO: 197 - protein, 9 aa, Artificial Sequence
  Synthetic peptide
AIKVPKANK

SEQ ID NO: 198 - protein, 10 aa, Artificial Sequence
  Synthetic peptide
KVKIPVAIKV

SEQ ID NO: 199 - protein, 10 aa, Artificial Sequence
  Synthetic peptide
KIPVAIKVPK

SEQ ID NO: 200 - protein, 10 aa, Artificial Sequence
  Synthetic peptide
KVPKANKEIL

SEQ ID NO: 201 - protein, 10 aa, Artificial Sequence
  Synthetic peptide
VAIKVPKANK

SEQ ID NO: 202 - protein, 9 aa, Artificial Sequence
  Synthetic peptide
IPVAIKVPK

SEQ ID NO: 203 - protein, 9 aa, Artificial Sequence
  Synthetic peptide
VKIPVAIKV

SEQ ID NO: 204 - protein, 12 aa, Artificial Sequence
  Synthetic peptide
GEKVKIPVAIKV

SEQ ID NO: 205 - protein, 11 aa, Artificial Sequence
  Synthetic peptide
VKIPVAIKVPK

SEQ ID NO: 206 - protein, 11 aa, Artificial Sequence
  Synthetic peptide
IKVPKANKEIL

SEQ ID NO: 207 - protein, 9 aa, Artificial Sequence
  Synthetic peptide
KVPKANKEI

SEQ ID NO: 208 - protein, 9 aa, Artificial Sequence
  Synthetic peptide
AIKEPTSPK

SEQ ID NO: 209 - protein, 10 aa, Artificial Sequence
  Synthetic peptide
VAIKEPTSPK

SEQ ID NO: 210 - protein, 9 aa, Artificial Sequence
  Synthetic peptide
IPVAIKEPT

SEQ ID NO: 211 - protein, 12 aa, Artificial Sequence
  Synthetic peptide
AIKEPTSPKANK

SEQ ID NO: 212 - protein, 12 aa, Artificial Sequence
  Synthetic peptide
IPVAIKEPTSPK

SEQ ID NO: 213 - protein, 10 aa, Artificial Sequence
  Synthetic peptide
AIKEPTSPKA

SEQ ID NO: 214 - protein, 10 aa, Artificial Sequence
  Synthetic peptide
IPVAIKEPTS

SEQ ID NO: 215 - protein, 11 aa, Artificial Sequence
  Synthetic peptide
PVAIKEPTSPK

SEQ ID NO: 216 - protein, 11 aa, Artificial Sequence
  Synthetic peptide
KVKIPVAIKEP

SEQ ID NO: 217 - protein, 10 aa, Artificial Sequence
  Synthetic peptide
KIPVAIKEPT

SEQ ID NO: 218 - protein, 9 aa, Artificial Sequence
  Synthetic peptide
VAIKEPSPK

SEQ ID NO: 219 - protein, 9 aa, Artificial Sequence
  Synthetic peptide
IPVAIKEPS

SEQ ID NO: 220 - protein, 9 aa, Artificial Sequence
  Synthetic peptide
AIKEPSPKA

SEQ ID NO: 221 - protein, 11 aa, Artificial Sequence
  Synthetic peptide
AIKEPSPKANK

SEQ ID NO: 222 - protein, 12 aa, Artificial Sequence
  Synthetic peptide
KIPVAIKEPSPK

SEQ ID NO: 223 - protein, 12 aa, Artificial Sequence
  Synthetic peptide
IPVAIKEPSPKA

SEQ ID NO: 224 - protein, 12 aa, Artificial Sequence
  Synthetic peptide
KVKIPVAIKEPS

SEQ ID NO: 225 - protein, 12 aa, Artificial Sequence
  Synthetic peptide
VAIKEPSPKANK

SEQ ID NO: 226 - protein, 10 aa, Artificial Sequence
  Synthetic peptide
KIPVAIKEPK

SEQ ID NO: 227 - protein, 10 aa, Artificial Sequence
  Synthetic peptide
KEPKANKEIL

SEQ ID NO: 228 - protein, 10 aa, Artificial Sequence
  Synthetic peptide
VAIKEPKANK

SEQ ID NO: 229 - protein, 9 aa, Artificial Sequence
  Synthetic peptide
EPKANKEIL

SEQ ID NO: 230 - protein, 9 aa, Artificial Sequence
  Synthetic peptide
IPVAIKEPK

SEQ ID NO: 231 - protein, 9 aa, Artificial Sequence
  Synthetic peptide
KEPKANKEI

SEQ ID NO: 232 - protein, 9 aa, Artificial Sequence
  Synthetic peptide
VAIKIPVAI

SEQ ID NO: 233 - protein, 9 aa, Artificial Sequence
  Synthetic peptide
IPVAIKIPV

SEQ ID NO: 234 - protein, 10 aa, Artificial Sequence
  Synthetic peptide
IPVAIKIPVA

SEQ ID NO: 235 - protein, 9 aa, Artificial Sequence
  Synthetic peptide
AIKIPVAIK

SEQ ID NO: 236 - protein, 10 aa, Artificial Sequence
  Synthetic peptide
KIPVAIKELR

SEQ ID NO: 237 - protein, 10 aa, Artificial Sequence
  Synthetic peptide
KVKIPVAIKI

SEQ ID NO: 238 - protein, 10 aa, Artificial Sequence
  Synthetic peptide
KIPVAIKIPV

SEQ ID NO: 239 - protein, 8 aa, Artificial Sequence
  Synthetic peptide
IPVAIKEL

SEQ ID NO: 240 - protein, 12 aa, Artificial Sequence
  Synthetic peptide
KVKIPVAIKIPV

SEQ ID NO: 241 - protein, 12 aa, Artificial Sequence
  Synthetic peptide
AIKELREATSPK

SEQ ID NO: 242 - protein, 11 aa, Artificial Sequence
  Synthetic peptide
IKELREATSPK

SEQ ID NO: 243 - protein, 10 aa, Artificial Sequence
  Synthetic peptide
VAIKIPVAIK

SEQ ID NO: 244 - protein, 12 aa, Artificial Sequence
  Synthetic peptide
AIKIPVAIKELR

SEQ ID NO: 245 - protein, 11 aa, Artificial Sequence
  Synthetic peptide
IPVAIKIPVAI

SEQ ID NO: 246 - protein, 9 aa, Artificial Sequence
  Synthetic peptide
IPVAIKELR

SEQ ID NO: 247 - protein, 10 aa, Artificial Sequence
  Synthetic peptide
IKIPVAIKEL

SEQ ID NO: 248 - protein, 9 aa, Artificial Sequence
  Synthetic peptide
KIPVAIKEL

SEQ ID NO: 249 - protein, 9 aa, Artificial Sequence
  Synthetic peptide
VKIPVAIKI

SEQ ID NO: 250 - protein, 11 aa, Artificial Sequence
  Synthetic peptide
IPVAIKELREA

SEQ ID NO: 251 - protein, 9 aa, Artificial Sequence
  Synthetic peptide
VAIKELREA

SEQ ID NO: 252 - protein, 11 aa, Artificial Sequence
  Synthetic peptide
VKIPVAIKIPV

SEQ ID NO: 253 - protein, 12 aa, Artificial Sequence
  Synthetic peptide
GEKVKIPVAIKI

SEQ ID NO: 254 - protein, 12 aa, Artificial Sequence
  Synthetic peptide
KIPVAIKIPVAI

SEQ ID NO: 255 - protein, 11 aa, Artificial Sequence
  Synthetic peptide
IKIPVAIKELR

SEQ ID NO: 256 - protein, 8 aa, Artificial Sequence
  Synthetic peptide
PVAIKIPV

SEQ ID NO: 257 - protein, 11 aa, Artificial Sequence
  Synthetic peptide
KIPVAIKIPVA

SEQ ID NO: 258 - protein, 10 aa, Artificial Sequence
  Synthetic peptide
PVAIKIPVAI

SEQ ID NO: 259 - protein, 8 aa, Artificial Sequence
  Synthetic peptide
AIKIPVAI

SEQ ID NO: 260 - protein, 12 aa, Artificial Sequence
  Synthetic peptide
VAIKIPVAIKEL

SEQ ID NO: 261 - protein, 10 aa, Artificial Sequence
  Synthetic peptide
FQEAYVMASV

SEQ ID NO: 262 - protein, 9 aa, Artificial Sequence
  Synthetic peptide
ILDEAFQEA

SEQ ID NO: 263 - protein, 11 aa, Artificial Sequence
  Synthetic peptide
ILDEAFQEAYV

SEQ ID NO: 264 - protein, 9 aa, Artificial Sequence
  Synthetic peptide
EAFQEAYVM

SEQ ID NO: 265 - protein, 12 aa, Artificial Sequence
  Synthetic peptide
EAFQEAYVMASV

SEQ ID NO: 266 - protein, 9 aa, Artificial Sequence
  Synthetic peptide
QEAYVMASV

SEQ ID NO: 267 - protein, 12 aa, Artificial Sequence
  Synthetic peptide
EILDEAFQEAYV

SEQ ID NO: 268 - protein, 10 aa, Artificial Sequence
  Synthetic peptide
DEAFQEAYVM

SEQ ID NO: 269 - protein, 12 aa, Artificial Sequence
  Synthetic peptide
ILDEAFQEAYVM

SEQ ID NO: 270 - protein, 8 aa, Artificial Sequence
  Synthetic peptide
DEAFQEAY

SEQ ID NO: 271 - protein, 11 aa, Artificial Sequence
  Synthetic peptide
AFQEAYVMASV

SEQ ID NO: 272 - protein, 10 aa, Artificial Sequence
  Synthetic peptide
ILDEAFQEAY

SEQ ID NO: 273 - protein, 10 aa, Artificial Sequence
  Synthetic peptide
EAFQEAYVMA

SEQ ID NO: 274 - protein, 12 aa, Artificial Sequence
  Synthetic peptide
AYVMASVDNPHV

SEQ ID NO: 275 - protein, 12 aa, Artificial Sequence
  Synthetic peptide
KEILDEAFQEAY

SEQ ID NO: 276 - protein, 11 aa, Artificial Sequence
  Synthetic peptide
KEILDEAFQEA

SEQ ID NO: 277 - protein, 10 aa, Artificial Sequence
  Synthetic peptide
EILDEAFQEA

SEQ ID NO: 278 - protein, 8 aa, Artificial Sequence
  Synthetic peptide
EAYVMASV

SEQ ID NO: 279 - protein, 11 aa, Artificial Sequence
  Synthetic peptide
KANKEILDEAF

SEQ ID NO: 280 - protein, 8 aa, Artificial Sequence
  Synthetic peptide
KEILDEAF

SEQ ID NO: 281 - protein, 8 aa, Artificial Sequence
  Synthetic peptide
AFQEAYVM

SEQ ID NO: 282 - protein, 8 aa, Artificial Sequence
  Synthetic peptide
EAFQEAYV

SEQ ID NO: 283 - protein, 11 aa, Artificial Sequence
  Synthetic peptide
LDEAFQEAYVM

SEQ ID NO: 284 - protein, 9 aa, Artificial Sequence
  Synthetic peptide
NKEILDEAF

SEQ ID NO: 285 - protein, 10 aa, Artificial Sequence
  Synthetic peptide
QEAYVMASVD

SEQ ID NO: 286 - protein, 9 aa, Artificial Sequence
  Synthetic peptide
LDEAFQEAY

SEQ ID NO: 287 - protein, 9 aa, Artificial Sequence
  Synthetic peptide
DEAFQEAYV

SEQ ID NO: 288 - protein, 9 aa, Artificial Sequence
  Synthetic peptide
AFQEAYVMA

SEQ ID NO: 289 - protein, 10 aa, Artificial Sequence
  Synthetic peptide
LDEAFQEAYV

SEQ ID NO: 290 - protein, 11 aa, Artificial Sequence
  Synthetic peptide
DEAFQEAYVMA

SEQ ID NO: 291 - protein, 9 aa, Artificial Sequence
  Synthetic peptide
YVMASVASV

SEQ ID NO: 292 - protein, 10 aa, Artificial Sequence
  Synthetic peptide
AYVMASVASV

SEQ ID NO: 293 - protein, 10 aa, Artificial Sequence
  Synthetic peptide
YVMASVASVD

SEQ ID NO: 294 - protein, 10 aa, Artificial Sequence
  Synthetic peptide
SVASVDNPHV

SEQ ID NO: 295 - protein, 11 aa, Artificial Sequence
  Synthetic peptide
EAYVMASVASV

SEQ ID NO: 296 - protein, 12 aa, Artificial Sequence
  Synthetic peptide
SVASVDNPHVCR

SEQ ID NO: 297 - protein, 8 aa, Artificial Sequence
  Synthetic peptide
VMASVASV

SEQ ID NO: 298 - protein, 12 aa, Artificial Sequence
  Synthetic peptide
DEAYVMASVASV

SEQ ID NO: 299 - protein, 10 aa, Artificial Sequence
  Synthetic peptide
ASVDNPHVCR

SEQ ID NO: 300 - protein, 9 aa, Artificial Sequence
  Synthetic peptide
SVDNPHVCR

SEQ ID NO: 301 - protein, 11 aa, Artificial Sequence
  Synthetic peptide
YVMASVASVDN

SEQ ID NO: 302 - protein, 9 aa, Artificial Sequence
  Synthetic peptide
EAYVMASVA

SEQ ID NO: 303 - protein, 10 aa, Artificial Sequence
  Synthetic peptide
DEAYVMASVA

SEQ ID NO: 304 - protein, 9 aa, Artificial Sequence
  Synthetic peptide
SVASVDNPH

SEQ ID NO: 305 - protein, 10 aa, Artificial Sequence
  Synthetic peptide
SVDNPHVCRL

SEQ ID NO: 306 - protein, 9 aa, Artificial Sequence
  Synthetic peptide
VDNPHVCRL

SEQ ID NO: 307 - protein, 9 aa, Artificial Sequence
  Synthetic peptide
VASVDNPHV

SEQ ID NO: 308 - protein, 10 aa, Artificial Sequence
  Synthetic peptide
EAYVMASVAS

SEQ ID NO: 309 - protein, 11 aa, Artificial Sequence
  Synthetic peptide
SVDNPHVCRLL

SEQ ID NO: 310 - protein, 10 aa, Artificial Sequence
  Synthetic peptide
MASVASVDNP

SEQ ID NO: 311 - protein, 10 aa, Artificial Sequence
  Synthetic peptide
VDNPHVCRLL

SEQ ID NO: 312 - protein, 11 aa, Artificial Sequence
  Synthetic peptide
DEAYVMASVAS

SEQ ID NO: 313 - protein, 9 aa, Artificial Sequence
  Synthetic peptide
YVMASVDSV

SEQ ID NO: 314 - protein, 10 aa, Artificial Sequence
  Synthetic peptide
AYVMASVDSV

SEQ ID NO: 315 - protein, 10 aa, Artificial Sequence
  Synthetic peptide
YVMASVDSVD

SEQ ID NO: 316 - protein, 11 aa, Artificial Sequence
  Synthetic peptide
EAYVMASVDSV

SEQ ID NO: 317 - protein, 10 aa, Artificial Sequence
  Synthetic peptide
DSVDNPHVCR

SEQ ID NO: 318 - protein, 12 aa, Artificial Sequence
  Synthetic peptide
DEAYVMASVDSV

SEQ ID NO: 319 - protein, 10 aa, Artificial Sequence
  Synthetic peptide
SVDSVDNPHV

SEQ ID NO: 320 - protein, 9 aa, Artificial Sequence
  Synthetic peptide
SVDSVDNPH

SEQ ID NO: 321 - protein, 9 aa, Artificial Sequence
  Synthetic peptide
MASVDNPPR

SEQ ID NO: 322 - protein, 11 aa, Artificial Sequence
  Synthetic peptide
YVMASVDNPPR

SEQ ID NO: 323 - protein, 12 aa, Artificial Sequence
  Synthetic peptide
VMASVDNPPRHV

SEQ ID NO: 324 - protein, 10 aa, Artificial Sequence
  Synthetic peptide
VMASVDNPPR

SEQ ID NO: 325 - protein, 10 aa, Artificial Sequence
  Synthetic peptide
NPPRHVCRLL

SEQ ID NO: 326 - protein, 9 aa, Artificial Sequence
  Synthetic peptide
NPPRHVCRL

SEQ ID NO: 327 - protein, 10 aa, Artificial Sequence
  Synthetic peptide
YVMASVDNPP

SEQ ID NO: 328 - protein, 9 aa, Artificial Sequence
  Synthetic peptide
RHVCRLLGI

SEQ ID NO: 329 - protein, 12 aa, Artificial Sequence
  Synthetic peptide
ASVDNPPRHVCR

SEQ ID NO: 330 - protein, 12 aa, Artificial Sequence
  Synthetic peptide
AYVMASVDNPPR

SEQ ID NO: 331 - protein, 9 aa, Artificial Sequence
  Synthetic peptide
DNPPRHVCR

SEQ ID NO: 332 - protein, 9 aa, Artificial Sequence
  Synthetic peptide
PPRHVCRLL

SEQ ID NO: 333 - protein, 12 aa, Artificial Sequence
  Synthetic peptide
RHVCRLLGICLT

SEQ ID NO: 334 - protein, 12 aa, Artificial Sequence
  Synthetic peptide
VDNPPRHVCRLL

SEQ ID NO: 335 - protein, 9 aa, Artificial Sequence
  Synthetic peptide
VMASVDNPP

SEQ ID NO: 336 - protein, 11 aa, Artificial Sequence
  Synthetic peptide
SVDNPPRHVCR

SEQ ID NO: 337 - protein, 9 aa, Artificial Sequence
  Synthetic peptide
SVDNPPRHV

SEQ ID NO: 338 - protein, 11 aa, Artificial Sequence
  Synthetic peptide
RHVCRLLGICL

SEQ ID NO: 339 - protein, 10 aa, Artificial Sequence
  Synthetic peptide
MASVDNPPRH

SEQ ID NO: 340 - protein, 10 aa, Artificial Sequence
  Synthetic peptide
ASVDNPPRHV

SEQ ID NO: 341 - protein, 11 aa, Artificial Sequence
  Synthetic peptide
MASVDNPPRHV

SEQ ID NO: 342 - protein, 12 aa, Artificial Sequence
  Synthetic peptide
YVMASVDNPHHV

SEQ ID NO: 343 - protein, 11 aa, Artificial Sequence
  Synthetic peptide
VMASVDNPHHV

SEQ ID NO: 344 - protein, 10 aa, Artificial Sequence
  Synthetic peptide
SVDNPHHVCR

SEQ ID NO: 345 - protein, 9 aa, Artificial Sequence
  Synthetic peptide
NPHHVCRLL

SEQ ID NO: 346 - protein, 10 aa, Artificial Sequence
  Synthetic peptide
MASVDNPHHV

SEQ ID NO: 347 - protein, 9 aa, Artificial Sequence
  Synthetic peptide
HHVCRLLGI

SEQ ID NO: 348 - protein, 11 aa, Artificial Sequence
  Synthetic peptide
ASVDNPHHVCR

SEQ ID NO: 349 - protein, 12 aa, Artificial Sequence
  Synthetic peptide
MASVDNPHHVCR

SEQ ID NO: 350 - protein, 9 aa, Artificial Sequence
  Synthetic peptide
ASVDNPHHV

SEQ ID NO: 351 - protein, 11 aa, Artificial Sequence
  Synthetic peptide
HHVCRLLGICL

SEQ ID NO: 352 - protein, 8 aa, Artificial Sequence
  Synthetic peptide
NPHHVCRL

SEQ ID NO: 353 - protein, 9 aa, Artificial Sequence
  Synthetic peptide
MASVDNPHH

SEQ ID NO: 354 - protein, 10 aa, Artificial Sequence
  Synthetic peptide
HVCRLLGICL

SEQ ID NO: 355 - protein, 12 aa, Artificial Sequence
  Synthetic peptide
SVDNPHHVCRLL

SEQ ID NO: 356 - protein, 11 aa, Artificial Sequence
  Synthetic peptide
VDNPHHVCRLL

SEQ ID NO: 357 - protein, 10 aa, Artificial Sequence
  Synthetic peptide
VMASVDNPHH

SEQ ID NO: 358 - protein, 8 aa, Artificial Sequence
  Synthetic peptide
DNPHHVCR

SEQ ID NO: 359 - protein, 10 aa, Artificial Sequence
  Synthetic peptide
DNPHHVCRLL

SEQ ID NO: 360 - protein, 11 aa, Artificial Sequence
  Synthetic peptide
NPHHVCRLLGI

SEQ ID NO: 361 - protein, 10 aa, Artificial Sequence
  Synthetic peptide
NPHHVCRLLG

SEQ ID NO: 362 - protein, 11 aa, Artificial Sequence
  Synthetic peptide
HVCRLLGICLT

SEQ ID NO: 363 - protein, 11 aa, Artificial Sequence
  Synthetic peptide
SVDNPHHVCRL

SEQ ID NO: 364 - protein, 10 aa, Artificial Sequence
  Synthetic peptide
VDNPHHVCRL

SEQ ID NO: 365 - protein, 21 aa, Artificial Sequence
  Synthetic peptide
EAYVMASVDNPHVCRLLGICL

SEQ ID NO: 366 - protein, 21 aa, Artificial Sequence
  Synthetic peptide
AYVMASVDNPHVCRLLGICLT

SEQ ID NO: 367 - protein, 21 aa, Artificial Sequence
  Synthetic peptide
QEAYVMASVDNPHVCRLLGIC

SEQ ID NO: 368 - protein, 21 aa, Artificial Sequence
  Synthetic peptide
FQEAYVMASVDNPHVCRLLGI

SEQ ID NO: 369 - protein, 21 aa, Artificial Sequence
  Synthetic peptide
ANKEILDEAFQEAYVMASVDN

SEQ ID NO: 370 - protein, 21 aa, Artificial Sequence
  Synthetic peptide
KANKEILDEAFQEAYVMASVD

SEQ ID NO: 371 - protein, 21 aa, Artificial Sequence
  Synthetic peptide
PKANKEILDEAFQEAYVMASV

SEQ ID NO: 372 - protein, 21 aa, Artificial Sequence
  Synthetic peptide
SPKANKEILDEAFQEAYVMAS

SEQ ID NO: 373 - protein, 21 aa, Artificial Sequence
  Synthetic peptide
TSPKANKEILDEAFQEAYVMA

SEQ ID NO: 374 - protein, 21 aa, Artificial Sequence
  Synthetic peptide
ATSPKANKEILDEAFQEAYVM

SEQ ID NO: 375 - protein, 21 aa, Artificial Sequence
  Synthetic peptide
EATSPKANKEILDEAFQEAYV

SEQ ID NO: 376 - protein, 21 aa, Artificial Sequence
  Synthetic peptide
REATSPKANKEILDEAFQEAY

SEQ ID NO: 377 - protein, 21 aa, Artificial Sequence
  Synthetic peptide
LREATSPKANKEILDEAFQEA

SEQ ID NO: 378 - protein, 21 aa, Artificial Sequence
  Synthetic peptide
ELREATSPKANKEILDEAFQE

SEQ ID NO: 379 - protein, 21 aa, Artificial Sequence
  Synthetic peptide
AFQEAYVMASVDNPHVCRLLG

SEQ ID NO: 380 - protein, 21 aa, Artificial Sequence
  Synthetic peptide
KELREATSPKANKEILDEAFQ

SEQ ID NO: 381 - protein, 21 aa, Artificial Sequence
  Synthetic peptide
IKELREATSPKANKEILDEAF

SEQ ID NO: 382 - protein, 21 aa, Artificial Sequence
  Synthetic peptide
EAFQEAYVMASVDNPHVCRLL

SEQ ID NO: 383 - protein, 21 aa, Artificial Sequence
  Synthetic peptide
DEAFQEAYVMASVDNPHVCRL

SEQ ID NO: 384 - protein, 21 aa, Artificial Sequence
  Synthetic peptide
LDEAFQEAYVMASVDNPHVCR

SEQ ID NO: 385 - protein, 21 aa, Artificial Sequence
  Synthetic peptide
ILDEAFQEAYVMASVDNPHVC

SEQ ID NO: 386 - protein, 21 aa, Artificial Sequence
  Synthetic peptide
EILDEAFQEAYVMASVDNPHV

SEQ ID NO: 387 - protein, 21 aa, Artificial Sequence
  Synthetic peptide
KEILDEAFQEAYVMASVDNPH

SEQ ID NO: 388 - protein, 21 aa, Artificial Sequence
  Synthetic peptide
NKEILDEAFQEAYVMASVDNP

SEQ ID NO: 389 - protein, 21 aa, Artificial Sequence
  Synthetic peptide
DNPHVCRLLGICLTSTVQLIT

SEQ ID NO: 390 - protein, 21 aa, Artificial Sequence
  Synthetic peptide
VDNPHVCRLLGICLTSTVQLI

SEQ ID NO: 391 - protein, 21 aa, Artificial Sequence
  Synthetic peptide
SVDNPHVCRLLGICLTSTVQL

SEQ ID NO: 392 - protein, 21 aa, Artificial Sequence
  Synthetic peptide
EAYVMASVDSVDNPHVCRLLG

SEQ ID NO: 393 - protein, 21 aa, Artificial Sequence
  Synthetic peptide
DEAYVMASVDSVDNPHVCRLL

SEQ ID NO: 394 - protein, 21 aa, Artificial Sequence
  Synthetic peptide
LDEAYVMASVDSVDNPHVCRL

SEQ ID NO: 395 - protein, 21 aa, Artificial Sequence
  Synthetic peptide
ILDEAYVMASVDSVDNPHVCR

SEQ ID NO: 396 - protein, 21 aa, Artificial Sequence
  Synthetic peptide
EILDEAYVMASVDSVDNPHVC

SEQ ID NO: 397 - protein, 21 aa, Artificial Sequence
  Synthetic peptide
KEILDEAYVMASVDSVDNPHV

SEQ ID NO: 398 - protein, 21 aa, Artificial Sequence
  Synthetic peptide
NKEILDEAYVMASVDSVDNPH

SEQ ID NO: 399 - protein, 21 aa, Artificial Sequence
  Synthetic peptide
ANKEILDEAYVMASVDSVDNP

SEQ ID NO: 400 - protein, 21 aa, Artificial Sequence
  Synthetic peptide
KANKEILDEAYVMASVDSVDN

SEQ ID NO: 401 - protein, 21 aa, Artificial Sequence
  Synthetic peptide
PKANKEILDEAYVMASVDSVD

SEQ ID NO: 402 - protein, 21 aa, Artificial Sequence
  Synthetic peptide
DSVDNPHVCRLLGICLTSTVQ

SEQ ID NO: 403 - protein, 21 aa, Artificial Sequence
  Synthetic peptide
SPKANKEILDEAYVMASVDSV

SEQ ID NO: 404 - protein, 21 aa, Artificial Sequence
  Synthetic peptide
TSPKANKEILDEAYVMASVDS

SEQ ID NO: 405 - protein, 21 aa, Artificial Sequence
  Synthetic peptide
VDSVDNPHVCRLLGICLTSTV

SEQ ID NO: 406 - protein, 21 aa, Artificial Sequence
  Synthetic peptide
SVDSVDNPHVCRLLGICLTST

SEQ ID NO: 407 - protein, 21 aa, Artificial Sequence
  Synthetic peptide
ASVDSVDNPHVCRLLGICLTS

SEQ ID NO: 408 - protein, 21 aa, Artificial Sequence
  Synthetic peptide
MASVDSVDNPHVCRLLGICLT

SEQ ID NO: 409 - protein, 21 aa, Artificial Sequence
  Synthetic peptide
VMASVDSVDNPHVCRLLGICL

SEQ ID NO: 410 - protein, 21 aa, Artificial Sequence
  Synthetic peptide
YVMASVDSVDNPHVCRLLGIC

SEQ ID NO: 411 - protein, 21 aa, Artificial Sequence
  Synthetic peptide
AYVMASVDSVDNPHVCRLLGI

SEQ ID NO: 412 - protein, 21 aa, Artificial Sequence
  Synthetic peptide
KVKIPVAIKTSPKANKEILDE

SEQ ID NO: 413 - protein, 21 aa, Artificial Sequence
  Synthetic peptide
EKVKIPVAIKTSPKANKEILD

SEQ ID NO: 414 - protein, 21 aa, Artificial Sequence
  Synthetic peptide
GEKVKIPVAIKTSPKANKEIL

SEQ ID NO: 415 - protein, 21 aa, Artificial Sequence
  Synthetic peptide
EGEKVKIPVAIKTSPKANKEI

SEQ ID NO: 416 - protein, 21 aa, Artificial Sequence
  Synthetic peptide
PEGEKVKIPVAIKTSPKANKE

SEQ ID NO: 417 - protein, 21 aa, Artificial Sequence
  Synthetic peptide
IPEGEKVKIPVAIKTSPKANK

SEQ ID NO: 418 - protein, 21 aa, Artificial Sequence
  Synthetic peptide
WIPEGEKVKIPVAIKTSPKAN

SEQ ID NO: 419 - protein, 21 aa, Artificial Sequence
  Synthetic peptide
LWIPEGEKVKIPVAIKTSPKA

SEQ ID NO: 420 - protein, 21 aa, Artificial Sequence
  Synthetic peptide
GLWIPEGEKVKIPVAIKTSPK

SEQ ID NO: 421 - protein, 21 aa, Artificial Sequence
  Synthetic peptide
KGLWIPEGEKVKIPVAIKTSP

SEQ ID NO: 422 - protein, 21 aa, Artificial Sequence
  Synthetic peptide
KTSPKANKEILDEAYVMASVD

SEQ ID NO: 423 - protein, 21 aa, Artificial Sequence
  Synthetic peptide
YKGLWIPEGEKVKIPVAIKTS

SEQ ID NO: 424 - protein, 21 aa, Artificial Sequence
  Synthetic peptide
VYKGLWIPEGEKVKIPVAIKT

SEQ ID NO: 425 - protein, 21 aa, Artificial Sequence
  Synthetic peptide
IKTSPKANKEILDEAYVMASV

SEQ ID NO: 426 - protein, 21 aa, Artificial Sequence
  Synthetic peptide
AIKTSPKANKEILDEAYVMAS

SEQ ID NO: 427 - protein, 21 aa, Artificial Sequence
  Synthetic peptide
VAIKTSPKANKEILDEAYVMA

SEQ ID NO: 428 - protein, 21 aa, Artificial Sequence
  Synthetic peptide
PVAIKTSPKANKEILDEAYVM

SEQ ID NO: 429 - protein, 21 aa, Artificial Sequence
  Synthetic peptide
IPVAIKTSPKANKEILDEAYV

SEQ ID NO: 430 - protein, 21 aa, Artificial Sequence
  Synthetic peptide
KIPVAIKTSPKANKEILDEAY

SEQ ID NO: 431 - protein, 21 aa, Artificial Sequence
  Synthetic peptide
VKIPVAIKTSPKANKEILDEA

SEQ ID NO: 432 - protein, 21 aa, Artificial Sequence
  Synthetic peptide
AIKVPKANKEILDEAYVMASV

SEQ ID NO: 433 - protein, 21 aa, Artificial Sequence
  Synthetic peptide
EGEKVKIPVAIKVPKANKEIL

SEQ ID NO: 434 - protein, 21 aa, Artificial Sequence
  Synthetic peptide
EKVKIPVAIKVPKANKEILDE

SEQ ID NO: 435 - protein, 21 aa, Artificial Sequence
  Synthetic peptide
GEKVKIPVAIKVPKANKEILD

SEQ ID NO: 436 - protein, 21 aa, Artificial Sequence
  Synthetic peptide
GLWIPEGEKVKIPVAIKVPKA

SEQ ID NO: 437 - protein, 21 aa, Artificial Sequence
  Synthetic peptide
IKVPKANKEILDEAYVMASVD

SEQ ID NO: 438 - protein, 21 aa, Artificial Sequence
  Synthetic peptide
IPEGEKVKIPVAIKVPKANKE

SEQ ID NO: 439 - protein, 21 aa, Artificial Sequence
  Synthetic peptide
IPVAIKVPKANKEILDEAYVM

SEQ ID NO: 440 - protein, 21 aa, Artificial Sequence
  Synthetic peptide
KGLWIPEGEKVKIPVAIKVPK

SEQ ID NO: 441 - protein, 21 aa, Artificial Sequence
  Synthetic peptide
KIPVAIKVPKANKEILDEAYV

SEQ ID NO: 442 - protein, 21 aa, Artificial Sequence
  Synthetic peptide
KVKIPVAIKVPKANKEILDEA

SEQ ID NO: 443 - protein, 21 aa, Artificial Sequence
  Synthetic peptide
KVPKANKEILDEAYVMASVDN

SEQ ID NO: 444 - protein, 21 aa, Artificial Sequence
  Synthetic peptide
LWIPEGEKVKIPVAIKVPKAN

SEQ ID NO: 445 - protein, 21 aa, Artificial Sequence
  Synthetic peptide
PEGEKVKIPVAIKVPKANKEI

SEQ ID NO: 446 - protein, 21 aa, Artificial Sequence
  Synthetic peptide
PVAIKVPKANKEILDEAYVMA

SEQ ID NO: 447 - protein, 21 aa, Artificial Sequence
  Synthetic peptide
VAIKVPKANKEILDEAYVMAS

SEQ ID NO: 448 - protein, 21 aa, Artificial Sequence
  Synthetic peptide
VKIPVAIKVPKANKEILDEAY

SEQ ID NO: 449 - protein, 21 aa, Artificial Sequence
  Synthetic peptide
VPKANKEILDEAYVMASVDNP

SEQ ID NO: 450 - protein, 21 aa, Artificial Sequence
  Synthetic peptide
VYKGLWIPEGEKVKIPVAIKV

SEQ ID NO: 451 - protein, 21 aa, Artificial Sequence
  Synthetic peptide
WIPEGEKVKIPVAIKVPKANK

SEQ ID NO: 452 - protein, 21 aa, Artificial Sequence
  Synthetic peptide
YKGLWIPEGEKVKIPVAIKVP

SEQ ID NO: 453 - protein, 21 aa, Artificial Sequence
  Synthetic peptide
ASGAFGTVYKGLWIPEGEKVK

SEQ ID NO: 454 - protein, 21 aa, Artificial Sequence
  Synthetic peptide
TEFKKIKVLASGAFGTVYKGL

SEQ ID NO: 455 - protein, 21 aa, Artificial Sequence
  Synthetic peptide
ETEFKKIKVLASGAFGTVYKG

SEQ ID NO: 456 - protein, 21 aa, Artificial Sequence
  Synthetic peptide
KETEFKKIKVLASGAFGTVYK

SEQ ID NO: 457 - protein, 21 aa, Artificial Sequence
  Synthetic peptide
LKETEFKKIKVLASGAFGTVY

SEQ ID NO: 458 - protein, 21 aa, Artificial Sequence
  Synthetic peptide
ILKETEFKKIKVLASGAFGTV

SEQ ID NO: 459 - protein, 21 aa, Artificial Sequence
  Synthetic peptide
RILKETEFKKIKVLASGAFGT

SEQ ID NO: 460 - protein, 21 aa, Artificial Sequence
  Synthetic peptide
LRILKETEFKKIKVLASGAFG

SEQ ID NO: 461 - protein, 21 aa, Artificial Sequence
  Synthetic peptide
LLRILKETEFKKIKVLASGAF

SEQ ID NO: 462 - protein, 21 aa, Artificial Sequence
  Synthetic peptide
ALLRILKETEFKKIKVLASGA

SEQ ID NO: 463 - protein, 21 aa, Artificial Sequence
  Synthetic peptide
QALLRILKETEFKKIKVLASG

SEQ ID NO: 464 - protein, 21 aa, Artificial Sequence
  Synthetic peptide
LASGAFGTVYKGLWIPEGEKV

SEQ ID NO: 465 - protein, 21 aa, Artificial Sequence
  Synthetic peptide
NQALLRILKETEFKKIKVLAS

SEQ ID NO: 466 - protein, 21 aa, Artificial Sequence
  Synthetic peptide
PNQALLRILKETEFKKIKVLA

SEQ ID NO: 467 - protein, 21 aa, Artificial Sequence
  Synthetic peptide
VLASGAFGTVYKGLWIPEGEK

SEQ ID NO: 468 - protein, 21 aa, Artificial Sequence
  Synthetic peptide
KVLASGAFGTVYKGLWIPEGE

SEQ ID NO: 469 - protein, 21 aa, Artificial Sequence
  Synthetic peptide
IKVLASGAFGTVYKGLWIPEG

SEQ ID NO: 470 - protein, 21 aa, Artificial Sequence
  Synthetic peptide
KIKVLASGAFGTVYKGLWIPE

SEQ ID NO: 471 - protein, 21 aa, Artificial Sequence
  Synthetic peptide
KKIKVLASGAFGTVYKGLWIP

SEQ ID NO: 472 - protein, 21 aa, Artificial Sequence
  Synthetic peptide
FKKIKVLASGAFGTVYKGLWI

SEQ ID NO: 473 - protein, 21 aa, Artificial Sequence
  Synthetic peptide
EFKKIKVLASGAFGTVYKGLW

SEQ ID NO: 474 - protein, 21 aa, Artificial Sequence
  Synthetic peptide
CSGAFGTVYKGLWIPEGEKVK

SEQ ID NO: 475 - protein, 21 aa, Artificial Sequence
  Synthetic peptide
TEFKKIKVLCSGAFGTVYKGL

SEQ ID NO: 476 - protein, 21 aa, Artificial Sequence
  Synthetic peptide
ETEFKKIKVLCSGAFGTVYKG

SEQ ID NO: 477 - protein, 21 aa, Artificial Sequence
  Synthetic peptide
KETEFKKIKVLCSGAFGTVYK

SEQ ID NO: 478 - protein, 21 aa, Artificial Sequence
  Synthetic peptide
LKETEFKKIKVLCSGAFGTVY

SEQ ID NO: 479 - protein, 21 aa, Artificial Sequence
  Synthetic peptide
ILKETEFKKIKVLCSGAFGTV

SEQ ID NO: 480 - protein, 21 aa, Artificial Sequence
  Synthetic peptide
RILKETEFKKIKVLCSGAFGT

SEQ ID NO: 481 - protein, 21 aa, Artificial Sequence
  Synthetic peptide
LRILKETEFKKIKVLCSGAFG

SEQ ID NO: 482 - protein, 21 aa, Artificial Sequence
  Synthetic peptide
LLRILKETEFKKIKVLCSGAF

SEQ ID NO: 483 - protein, 21 aa, Artificial Sequence
  Synthetic peptide
ALLRILKETEFKKIKVLCSGA

SEQ ID NO: 484 - protein, 21 aa, Artificial Sequence
  Synthetic peptide
QALLRILKETEFKKIKVLCSG

SEQ ID NO: 485 - protein, 21 aa, Artificial Sequence
  Synthetic peptide
LCSGAFGTVYKGLWIPEGEKV

SEQ ID NO: 486 - protein, 21 aa, Artificial Sequence
  Synthetic peptide
NQALLRILKETEFKKIKVLCS

SEQ ID NO: 487 - protein, 21 aa, Artificial Sequence
  Synthetic peptide
PNQALLRILKETEFKKIKVLC

SEQ ID NO: 488 - protein, 21 aa, Artificial Sequence
  Synthetic peptide
VLCSGAFGTVYKGLWIPEGEK

SEQ ID NO: 489 - protein, 21 aa, Artificial Sequence
  Synthetic peptide
KVLCSGAFGTVYKGLWIPEGE

SEQ ID NO: 490 - protein, 21 aa, Artificial Sequence
  Synthetic peptide
IKVLCSGAFGTVYKGLWIPEG

SEQ ID NO: 491 - protein, 21 aa, Artificial Sequence
  Synthetic peptide
KIKVLCSGAFGTVYKGLWIPE

SEQ ID NO: 492 - protein, 21 aa, Artificial Sequence
  Synthetic peptide
KKIKVLCSGAFGTVYKGLWIP

SEQ ID NO: 493 - protein, 21 aa, Artificial Sequence
  Synthetic peptide
FKKIKVLCSGAFGTVYKGLWI

SEQ ID NO: 494 - protein, 21 aa, Artificial Sequence
  Synthetic peptide
EFKKIKVLCSGAFGTVYKGLW

SEQ ID NO: 495 - protein, 21 aa, Artificial Sequence
  Synthetic peptide
SSGAFGTVYKGLWIPEGEKVK

SEQ ID NO: 496 - protein, 21 aa, Artificial Sequence
  Synthetic peptide
TEFKKIKVLSSGAFGTVYKGL

SEQ ID NO: 497 - protein, 21 aa, Artificial Sequence
  Synthetic peptide
ETEFKKIKVLSSGAFGTVYKG

SEQ ID NO: 498 - protein, 21 aa, Artificial Sequence
  Synthetic peptide
KETEFKKIKVLSSGAFGTVYK

SEQ ID NO: 499 - protein, 21 aa, Artificial Sequence
  Synthetic peptide
LKETEFKKIKVLSSGAFGTVY

SEQ ID NO: 500 - protein, 21 aa, Artificial Sequence
  Synthetic peptide
ILKETEFKKIKVLSSGAFGTV

SEQ ID NO: 501 - protein, 21 aa, Artificial Sequence
  Synthetic peptide
RILKETEFKKIKVLSSGAFGT

SEQ ID NO: 502 - protein, 21 aa, Artificial Sequence
  Synthetic peptide
LRILKETEFKKIKVLSSGAFG

SEQ ID NO: 503 - protein, 21 aa, Artificial Sequence
  Synthetic peptide
LLRILKETEFKKIKVLSSGAF

SEQ ID NO: 504 - protein, 21 aa, Artificial Sequence
  Synthetic peptide
ALLRILKETEFKKIKVLSSGA

SEQ ID NO: 505 - protein, 21 aa, Artificial Sequence
  Synthetic peptide
QALLRILKETEFKKIKVLSSG

SEQ ID NO: 506 - protein, 21 aa, Artificial Sequence
  Synthetic peptide
LSSGAFGTVYKGLWIPEGEKV

SEQ ID NO: 507 - protein, 21 aa, Artificial Sequence
  Synthetic peptide
NQALLRILKETEFKKIKVLSS

SEQ ID NO: 508 - protein, 21 aa, Artificial Sequence
  Synthetic peptide
PNQALLRILKETEFKKIKVLS

SEQ ID NO: 509 - protein, 21 aa, Artificial Sequence
  Synthetic peptide
VLSSGAFGTVYKGLWIPEGEK

SEQ ID NO: 510 - protein, 21 aa, Artificial Sequence
  Synthetic peptide
KVLSSGAFGTVYKGLWIPEGE

SEQ ID NO: 511 - protein, 21 aa, Artificial Sequence
  Synthetic peptide
IKVLSSGAFGTVYKGLWIPEG

SEQ ID NO: 512 - protein, 21 aa, Artificial Sequence
  Synthetic peptide
KIKVLSSGAFGTVYKGLWIPE

SEQ ID NO: 513 - protein, 21 aa, Artificial Sequence
  Synthetic peptide
KKIKVLSSGAFGTVYKGLWIP

SEQ ID NO: 514 - protein, 21 aa, Artificial Sequence
  Synthetic peptide
FKKIKVLSSGAFGTVYKGLWI

SEQ ID NO: 515 - protein, 21 aa, Artificial Sequence
  Synthetic peptide
EFKKIKVLSSGAFGTVYKGLW

SEQ ID NO: 516 - protein, 21 aa, Artificial Sequence
  Synthetic peptide
HVCRLLGICLTSTVQLITQLM

SEQ ID NO: 517 - protein, 21 aa, Artificial Sequence
  Synthetic peptide
VMASVDNPHHVCRLLGICLTS

SEQ ID NO: 518 - protein, 21 aa, Artificial Sequence
  Synthetic peptide
YVMASVDNPHHVCRLLGICLT

SEQ ID NO: 519 - protein, 21 aa, Artificial Sequence
  Synthetic peptide
AYVMASVDNPHHVCRLLGICL

SEQ ID NO: 520 - protein, 21 aa, Artificial Sequence
  Synthetic peptide
EAYVMASVDNPHHVCRLLGIC

SEQ ID NO: 521 - protein, 21 aa, Artificial Sequence
  Synthetic peptide
DEAYVMASVDNPHHVCRLLGI

SEQ ID NO: 522 - protein, 21 aa, Artificial Sequence
  Synthetic peptide
LDEAYVMASVDNPHHVCRLLG

SEQ ID NO: 523 - protein, 21 aa, Artificial Sequence
  Synthetic peptide
ILDEAYVMASVDNPHHVCRLL

SEQ ID NO: 524 - protein, 21 aa, Artificial Sequence
  Synthetic peptide
EILDEAYVMASVDNPHHVCRL

SEQ ID NO: 525 - protein, 21 aa, Artificial Sequence
  Synthetic peptide
KEILDEAYVMASVDNPHHVCR

SEQ ID NO: 526 - protein, 21 aa, Artificial Sequence
  Synthetic peptide
NKEILDEAYVMASVDNPHHVC

SEQ ID NO: 527 - protein, 21 aa, Artificial Sequence
  Synthetic peptide
HHVCRLLGICLTSTVQLITQL

SEQ ID NO: 528 - protein, 21 aa, Artificial Sequence
  Synthetic peptide
ANKEILDEAYVMASVDNPHHV

SEQ ID NO: 529 - protein, 21 aa, Artificial Sequence
  Synthetic peptide
KANKEILDEAYVMASVDNPHH

SEQ ID NO: 530 - protein, 21 aa, Artificial Sequence
  Synthetic peptide
PHHVCRLLGICLTSTVQLITQ

SEQ ID NO: 531 - protein, 21 aa, Artificial Sequence
  Synthetic peptide
NPHHVCRLLGICLTSTVQLIT

SEQ ID NO: 532 - protein, 21 aa, Artificial Sequence
  Synthetic peptide
DNPHHVCRLLGICLTSTVQLI

SEQ ID NO: 533 - protein, 21 aa, Artificial Sequence
  Synthetic peptide
VDNPHHVCRLLGICLTSTVQL

SEQ ID NO: 534 - protein, 21 aa, Artificial Sequence
  Synthetic peptide
SVDNPHHVCRLLGICLTSTVQ

SEQ ID NO: 535 - protein, 21 aa, Artificial Sequence
  Synthetic peptide
ASVDNPHHVCRLLGICLTSTV

SEQ ID NO: 536 - protein, 21 aa, Artificial Sequence
  Synthetic peptide
MASVDNPHHVCRLLGICLTST

SEQ ID NO: 537 - protein, 21 aa, Artificial Sequence
  Synthetic peptide
PVAIKELREATSPKANKEILD

SEQ ID NO: 538 - protein, 21 aa, Artificial Sequence
  Synthetic peptide
VAIKELREATSPKANKEILDE

SEQ ID NO: 539 - protein, 21 aa, Artificial Sequence
  Synthetic peptide
AIKELREATSPKANKEILDEA

SEQ ID NO: 540 - protein, 21 aa, Artificial Sequence
  Synthetic peptide
IKELREATSPKANKEILDEAY

SEQ ID NO: 541 - protein, 21 aa, Artificial Sequence
  Synthetic peptide
IPVAIKELREATSPKANKEIL

SEQ ID NO: 542 - protein, 21 aa, Artificial Sequence
  Synthetic peptide
KIPVAIKELREATSPKANKEI

SEQ ID NO: 543 - protein, 21 aa, Artificial Sequence
  Synthetic peptide
EKVKIPVAIKIPVAIKELREA

SEQ ID NO: 544 - protein, 21 aa, Artificial Sequence
  Synthetic peptide
GEKVKIPVAIKIPVAIKELRE

SEQ ID NO: 545 - protein, 21 aa, Artificial Sequence
  Synthetic peptide
EGEKVKIPVAIKIPVAIKELR

SEQ ID NO: 546 - protein, 21 aa, Artificial Sequence
  Synthetic peptide
PEGEKVKIPVAIKIPVAIKEL

SEQ ID NO: 547 - protein, 21 aa, Artificial Sequence
  Synthetic peptide
IPEGEKVKIPVAIKIPVAIKE

SEQ ID NO: 548 - protein, 21 aa, Artificial Sequence
  Synthetic peptide
WIPEGEKVKIPVAIKIPVAIK

SEQ ID NO: 549 - protein, 21 aa, Artificial Sequence
  Synthetic peptide
LWIPEGEKVKIPVAIKIPVAI

SEQ ID NO: 550 - protein, 21 aa, Artificial Sequence
  Synthetic peptide
GLWIPEGEKVKIPVAIKIPVA

SEQ ID NO: 551 - protein, 21 aa, Artificial Sequence
  Synthetic peptide
KGLWIPEGEKVKIPVAIKIPV

SEQ ID NO: 552 - protein, 21 aa, Artificial Sequence
  Synthetic peptide
YKGLWIPEGEKVKIPVAIKIP

SEQ ID NO: 553 - protein, 21 aa, Artificial Sequence
  Synthetic peptide
IKIPVAIKELREATSPKANKE

SEQ ID NO: 554 - protein, 21 aa, Artificial Sequence
  Synthetic peptide
VYKGLWIPEGEKVKIPVAIKI

SEQ ID NO: 555 - protein, 21 aa, Artificial Sequence
  Synthetic peptide
AIKIPVAIKELREATSPKANK

SEQ ID NO: 556 - protein, 21 aa, Artificial Sequence
  Synthetic peptide
VAIKIPVAIKELREATSPKAN

SEQ ID NO: 557 - protein, 21 aa, Artificial Sequence
  Synthetic peptide
PVAIKIPVAIKELREATSPKA

SEQ ID NO: 558 - protein, 21 aa, Artificial Sequence
  Synthetic peptide
IPVAIKIPVAIKELREATSPK

SEQ ID NO: 559 - protein, 21 aa, Artificial Sequence
  Synthetic peptide
KIPVAIKIPVAIKELREATSP

SEQ ID NO: 560 - protein, 21 aa, Artificial Sequence
  Synthetic peptide
VKIPVAIKIPVAIKELREATS

SEQ ID NO: 561 - protein, 21 aa, Artificial Sequence
  Synthetic peptide
KVKIPVAIKIPVAIKELREAT

SEQ ID NO: 562 - protein, 21 aa, Artificial Sequence
  Synthetic peptide
AIKEPTSPKANKEILDEAYVM

SEQ ID NO: 563 - protein, 21 aa, Artificial Sequence
  Synthetic peptide
EGEKVKIPVAIKEPTSPKANK

SEQ ID NO: 564 - protein, 21 aa, Artificial Sequence
  Synthetic peptide
EKVKIPVAIKEPTSPKANKEI

SEQ ID NO: 565 - protein, 21 aa, Artificial Sequence
  Synthetic peptide
EPTSPKANKEILDEAYVMASV

SEQ ID NO: 566 - protein, 21 aa, Artificial Sequence
  Synthetic peptide
GEKVKIPVAIKEPTSPKANKE

SEQ ID NO: 567 - protein, 21 aa, Artificial Sequence
  Synthetic peptide
GLWIPEGEKVKIPVAIKEPTS

SEQ ID NO: 568 - protein, 21 aa, Artificial Sequence
  Synthetic peptide
IKEPTSPKANKEILDEAYVMA

SEQ ID NO: 569 - protein, 21 aa, Artificial Sequence
  Synthetic peptide
IPEGEKVKIPVAIKEPTSPKA

SEQ ID NO: 570 - protein, 21 aa, Artificial Sequence
  Synthetic peptide
IPVAIKEPTSPKANKEILDEA

SEQ ID NO: 571 - protein, 21 aa, Artificial Sequence
  Synthetic peptide
KEPTSPKANKEILDEAYVMAS

SEQ ID NO: 572 - protein, 21 aa, Artificial Sequence
  Synthetic peptide
KGLWIPEGEKVKIPVAIKEPT

SEQ ID NO: 573 - protein, 21 aa, Artificial Sequence
  Synthetic peptide
KIPVAIKEPTSPKANKEILDE

SEQ ID NO: 574 - protein, 21 aa, Artificial Sequence
  Synthetic peptide
KVKIPVAIKEPTSPKANKEIL

SEQ ID NO: 575 - protein, 21 aa, Artificial Sequence
  Synthetic peptide
LWIPEGEKVKIPVAIKEPTSP

SEQ ID NO: 576 - protein, 21 aa, Artificial Sequence
  Synthetic peptide
PEGEKVKIPVAIKEPTSPKAN

SEQ ID NO: 577 - protein, 21 aa, Artificial Sequence
  Synthetic peptide
PTSPKANKEILDEAYVMASVD

SEQ ID NO: 578 - protein, 21 aa, Artificial Sequence
  Synthetic peptide
PVAIKEPTSPKANKEILDEAY

SEQ ID NO: 579 - protein, 21 aa, Artificial Sequence
  Synthetic peptide
VAIKEPTSPKANKEILDEAYV

SEQ ID NO: 580 - protein, 21 aa, Artificial Sequence
  Synthetic peptide
VKIPVAIKEPTSPKANKEILD

SEQ ID NO: 581 - protein, 21 aa, Artificial Sequence
  Synthetic peptide
WIPEGEKVKIPVAIKEPTSPK

SEQ ID NO: 582 - protein, 21 aa, Artificial Sequence
  Synthetic peptide
YKGLWIPEGEKVKIPVAIKEP

SEQ ID NO: 583 - protein, 21 aa, Artificial Sequence
  Synthetic peptide
AIKESKANKEILDEAYVMASV

SEQ ID NO: 584 - protein, 21 aa, Artificial Sequence
  Synthetic peptide
EGEKVKIPVAIKESKANKEIL

SEQ ID NO: 585 - protein, 21 aa, Artificial Sequence
  Synthetic peptide
EKVKIPVAIKESKANKEILDE

SEQ ID NO: 586 - protein, 21 aa, Artificial Sequence
  Synthetic peptide
ESKANKEILDEAYVMASVDNP

SEQ ID NO: 587 - protein, 21 aa, Artificial Sequence
  Synthetic peptide
GEKVKIPVAIKESKANKEILD

SEQ ID NO: 588 - protein, 21 aa, Artificial Sequence
  Synthetic peptide
GLWIPEGEKVKIPVAIKESKA

SEQ ID NO: 589 - protein, 21 aa, Artificial Sequence
  Synthetic peptide
IKESKANKEILDEAYVMASVD

SEQ ID NO: 590 - protein, 21 aa, Artificial Sequence
  Synthetic peptide
IPEGEKVKIPVAIKESKANKE

SEQ ID NO: 591 - protein, 21 aa, Artificial Sequence
  Synthetic peptide
IPVAIKESKANKEILDEAYVM

SEQ ID NO: 592 - protein, 21 aa, Artificial Sequence
  Synthetic peptide
KESKANKEILDEAYVMASVDN

SEQ ID NO: 593 - protein, 21 aa, Artificial Sequence
  Synthetic peptide
KGLWIPEGEKVKIPVAIKESK

SEQ ID NO: 594 - protein, 21 aa, Artificial Sequence
  Synthetic peptide
KIPVAIKESKANKEILDEAYV

SEQ ID NO: 595 - protein, 21 aa, Artificial Sequence
  Synthetic peptide
KVKIPVAIKESKANKEILDEA

SEQ ID NO: 596 - protein, 21 aa, Artificial Sequence
  Synthetic peptide
LWIPEGEKVKIPVAIKESKAN

SEQ ID NO: 597 - protein, 21 aa, Artificial Sequence
  Synthetic peptide
PEGEKVKIPVAIKESKANKEI

SEQ ID NO: 598 - protein, 21 aa, Artificial Sequence
  Synthetic peptide
PVAIKESKANKEILDEAYVMA

SEQ ID NO: 599 - protein, 21 aa, Artificial Sequence
  Synthetic peptide
SKANKEILDEAYVMASVDNPH

SEQ ID NO: 600 - protein, 21 aa, Artificial Sequence
  Synthetic peptide
VAIKESKANKEILDEAYVMAS

SEQ ID NO: 601 - protein, 21 aa, Artificial Sequence
  Synthetic peptide
VKIPVAIKESKANKEILDEAY

SEQ ID NO: 602 - protein, 21 aa, Artificial Sequence
  Synthetic peptide
WIPEGEKVKIPVAIKESKANK

SEQ ID NO: 603 - protein, 21 aa, Artificial Sequence
  Synthetic peptide
YKGLWIPEGEKVKIPVAIKES

SEQ ID NO: 604 - protein, 21 aa, Artificial Sequence
  Synthetic peptide
VKIPVAIKEPKANKEILDEAY

SEQ ID NO: 605 - protein, 21 aa, Artificial Sequence
  Synthetic peptide
KVKIPVAIKEPKANKEILDEA

SEQ ID NO: 606 - protein, 21 aa, Artificial Sequence
  Synthetic peptide
EKVKIPVAIKEPKANKEILDE

SEQ ID NO: 607 - protein, 21 aa, Artificial Sequence
  Synthetic peptide
GEKVKIPVAIKEPKANKEILD

SEQ ID NO: 608 - protein, 21 aa, Artificial Sequence
  Synthetic peptide
EGEKVKIPVAIKEPKANKEIL

SEQ ID NO: 609 - protein, 21 aa, Artificial Sequence
  Synthetic peptide
PEGEKVKIPVAIKEPKANKEI

SEQ ID NO: 610 - protein, 21 aa, Artificial Sequence
  Synthetic peptide
IPEGEKVKIPVAIKEPKANKE

SEQ ID NO: 611 - protein, 21 aa, Artificial Sequence
  Synthetic peptide
WIPEGEKVKIPVAIKEPKANK

SEQ ID NO: 612 - protein, 21 aa, Artificial Sequence
  Synthetic peptide
LWIPEGEKVKIPVAIKEPKAN

SEQ ID NO: 613 - protein, 21 aa, Artificial Sequence
  Synthetic peptide
GLWIPEGEKVKIPVAIKEPKA

SEQ ID NO: 614 - protein, 21 aa, Artificial Sequence
  Synthetic peptide
EPKANKEILDEAYVMASVDNP

SEQ ID NO: 615 - protein, 21 aa, Artificial Sequence
  Synthetic peptide
KGLWIPEGEKVKIPVAIKEPK

SEQ ID NO: 616 - protein, 21 aa, Artificial Sequence
  Synthetic peptide
YKGLWIPEGEKVKIPVAIKEP

SEQ ID NO: 617 - protein, 21 aa, Artificial Sequence
  Synthetic peptide
KEPKANKEILDEAYVMASVDN

SEQ ID NO: 618 - protein, 21 aa, Artificial Sequence
  Synthetic peptide
IKEPKANKEILDEAYVMASVD

SEQ ID NO: 619 - protein, 21 aa, Artificial Sequence
  Synthetic peptide
AIKEPKANKEILDEAYVMASV

SEQ ID NO: 620 - protein, 21 aa, Artificial Sequence
  Synthetic peptide
VAIKEPKANKEILDEAYVMAS

SEQ ID NO: 621 - protein, 21 aa, Artificial Sequence
  Synthetic peptide
PVAIKEPKANKEILDEAYVMA

SEQ ID NO: 622 - protein, 21 aa, Artificial Sequence
  Synthetic peptide
IPVAIKEPKANKEILDEAYVM

SEQ ID NO: 623 - protein, 21 aa, Artificial Sequence
  Synthetic peptide
KIPVAIKEPKANKEILDEAYV

SEQ ID NO: 624 - protein, 21 aa, Artificial Sequence
  Synthetic peptide
VKIPVAIKESPKANKEILDEA

SEQ ID NO: 625 - protein, 21 aa, Artificial Sequence
  Synthetic peptide
KVKIPVAIKESPKANKEILDE

SEQ ID NO: 626 - protein, 21 aa, Artificial Sequence
  Synthetic peptide
EKVKIPVAIKESPKANKEILD

SEQ ID NO: 627 - protein, 21 aa, Artificial Sequence
  Synthetic peptide
GEKVKIPVAIKESPKANKEIL

SEQ ID NO: 628 - protein, 21 aa, Artificial Sequence
  Synthetic peptide
EGEKVKIPVAIKESPKANKEI

SEQ ID NO: 629 - protein, 21 aa, Artificial Sequence
  Synthetic peptide
PEGEKVKIPVAIKESPKANKE

SEQ ID NO: 630 - protein, 21 aa, Artificial Sequence
  Synthetic peptide
IPEGEKVKIPVAIKESPKANK

SEQ ID NO: 631 - protein, 21 aa, Artificial Sequence
  Synthetic peptide
WIPEGEKVKIPVAIKESPKAN

SEQ ID NO: 632 - protein, 21 aa, Artificial Sequence
  Synthetic peptide
LWIPEGEKVKIPVAIKESPKA

SEQ ID NO: 633 - protein, 21 aa, Artificial Sequence
  Synthetic peptide
GLWIPEGEKVKIPVAIKESPK

SEQ ID NO: 634 - protein, 21 aa, Artificial Sequence
  Synthetic peptide
ESPKANKEILDEAYVMASVDN

SEQ ID NO: 635 - protein, 21 aa, Artificial Sequence
  Synthetic peptide
KGLWIPEGEKVKIPVAIKESP

SEQ ID NO: 636 - protein, 21 aa, Artificial Sequence
  Synthetic peptide
YKGLWIPEGEKVKIPVAIKES

SEQ ID NO: 637 - protein, 21 aa, Artificial Sequence
  Synthetic peptide
KESPKANKEILDEAYVMASVD

SEQ ID NO: 638 - protein, 21 aa, Artificial Sequence
  Synthetic peptide
IKESPKANKEILDEAYVMASV

SEQ ID NO: 639 - protein, 21 aa, Artificial Sequence
  Synthetic peptide
AIKESPKANKEILDEAYVMAS

SEQ ID NO: 640 - protein, 21 aa, Artificial Sequence
  Synthetic peptide
VAIKESPKANKEILDEAYVMA

SEQ ID NO: 641 - protein, 21 aa, Artificial Sequence
  Synthetic peptide
PVAIKESPKANKEILDEAYVM

SEQ ID NO: 642 - protein, 21 aa, Artificial Sequence
  Synthetic peptide
IPVAIKESPKANKEILDEAYV

SEQ ID NO: 643 - protein, 21 aa, Artificial Sequence
  Synthetic peptide
KIPVAIKESPKANKEILDEAY

SEQ ID NO: 644 - protein, 21 aa, Artificial Sequence
  Synthetic peptide
AIKEPSPKANKEILDEAYVMA

SEQ ID NO: 645 - protein, 21 aa, Artificial Sequence
  Synthetic peptide
EGEKVKIPVAIKEPSPKANKE

SEQ ID NO: 646 - protein, 21 aa, Artificial Sequence
  Synthetic peptide
EKVKIPVAIKEPSPKANKEIL

SEQ ID NO: 647 - protein, 21 aa, Artificial Sequence
  Synthetic peptide
EPSPKANKEILDEAYVMASVD

SEQ ID NO: 648 - protein, 21 aa, Artificial Sequence
  Synthetic peptide
GEKVKIPVAIKEPSPKANKEI

SEQ ID NO: 649 - protein, 21 aa, Artificial Sequence
  Synthetic peptide
GLWIPEGEKVKIPVAIKEPSP

SEQ ID NO: 650 - protein, 21 aa, Artificial Sequence
  Synthetic peptide
IKEPSPKANKEILDEAYVMAS

SEQ ID NO: 651 - protein, 21 aa, Artificial Sequence
  Synthetic peptide
IPEGEKVKIPVAIKEPSPKAN

SEQ ID NO: 652 - protein, 21 aa, Artificial Sequence
  Synthetic peptide
IPVAIKEPSPKANKEILDEAY

SEQ ID NO: 653 - protein, 21 aa, Artificial Sequence
  Synthetic peptide
KEPSPKANKEILDEAYVMASV

SEQ ID NO: 654 - protein, 21 aa, Artificial Sequence
  Synthetic peptide
KGLWIPEGEKVKIPVAIKEPS

SEQ ID NO: 655 - protein, 21 aa, Artificial Sequence
  Synthetic peptide
KIPVAIKEPSPKANKEILDEA

SEQ ID NO: 656 - protein, 21 aa, Artificial Sequence
  Synthetic peptide
KVKIPVAIKEPSPKANKEILD

SEQ ID NO: 657 - protein, 21 aa, Artificial Sequence
  Synthetic peptide
LWIPEGEKVKIPVAIKEPSPK

SEQ ID NO: 658 - protein, 21 aa, Artificial Sequence
  Synthetic peptide
PEGEKVKIPVAIKEPSPKANK

SEQ ID NO: 659 - protein, 21 aa, Artificial Sequence
  Synthetic peptide
PSPKANKEILDEAYVMASVDN

SEQ ID NO: 660 - protein, 21 aa, Artificial Sequence
  Synthetic peptide
PVAIKEPSPKANKEILDEAYV

SEQ ID NO: 661 - protein, 21 aa, Artificial Sequence
  Synthetic peptide
VAIKEPSPKANKEILDEAYVM

SEQ ID NO: 662 - protein, 21 aa, Artificial Sequence
  Synthetic peptide
VKIPVAIKEPSPKANKEILDE

SEQ ID NO: 663 - protein, 21 aa, Artificial Sequence
  Synthetic peptide
WIPEGEKVKIPVAIKEPSPKA

SEQ ID NO: 664 - protein, 21 aa, Artificial Sequence
  Synthetic peptide
YKGLWIPEGEKVKIPVAIKEP

SEQ ID NO: 665 - protein, 21 aa, Artificial Sequence
  Synthetic peptide
RAKLLGAEEKEYHAEGGKVPI

SEQ ID NO: 666 - protein, 21 aa, Artificial Sequence
  Synthetic peptide
QHVKITDFGRAKLLGAEEKEY

SEQ ID NO: 667 - protein, 21 aa, Artificial Sequence
  Synthetic peptide
PQHVKITDFGRAKLLGAEEKE

SEQ ID NO: 668 - protein, 21 aa, Artificial Sequence
  Synthetic peptide
TPQHVKITDFGRAKLLGAEEK

SEQ ID NO: 669 - protein, 21 aa, Artificial Sequence
  Synthetic peptide
KTPQHVKITDFGRAKLLGAEE

SEQ ID NO: 670 - protein, 21 aa, Artificial Sequence
  Synthetic peptide
VKTPQHVKITDFGRAKLLGAE

SEQ ID NO: 671 - protein, 21 aa, Artificial Sequence
  Synthetic peptide
LVKTPQHVKITDFGRAKLLGA

SEQ ID NO: 672 - protein, 21 aa, Artificial Sequence
  Synthetic peptide
VLVKTPQHVKITDFGRAKLLG

SEQ ID NO: 673 - protein, 21 aa, Artificial Sequence
  Synthetic peptide
NVLVKTPQHVKITDFGRAKLL

SEQ ID NO: 674 - protein, 21 aa, Artificial Sequence
  Synthetic peptide
RNVLVKTPQHVKITDFGRAKL

SEQ ID NO: 675 - protein, 21 aa, Artificial Sequence
  Synthetic peptide
ARNVLVKTPQHVKITDFGRAK

SEQ ID NO: 676 - protein, 21 aa, Artificial Sequence
  Synthetic peptide
GRAKLLGAEEKEYHAEGGKVP

SEQ ID NO: 677 - protein, 21 aa, Artificial Sequence
  Synthetic peptide
AARNVLVKTPQHVKITDFGRA

SEQ ID NO: 678 - protein, 21 aa, Artificial Sequence
  Synthetic peptide
LAARNVLVKTPQHVKITDFGR

SEQ ID NO: 679 - protein, 21 aa, Artificial Sequence
  Synthetic peptide
FGRAKLLGAEEKEYHAEGGKV

SEQ ID NO: 680 - protein, 21 aa, Artificial Sequence
  Synthetic peptide
DFGRAKLLGAEEKEYHAEGGK

SEQ ID NO: 681 - protein, 21 aa, Artificial Sequence
  Synthetic peptide
TDFGRAKLLGAEEKEYHAEGG

SEQ ID NO: 682 - protein, 21 aa, Artificial Sequence
  Synthetic peptide
ITDFGRAKLLGAEEKEYHAEG

SEQ ID NO: 683 - protein, 21 aa, Artificial Sequence
  Synthetic peptide
KITDFGRAKLLGAEEKEYHAE

SEQ ID NO: 684 - protein, 21 aa, Artificial Sequence
  Synthetic peptide
VKITDFGRAKLLGAEEKEYHA

SEQ ID NO: 685 - protein, 21 aa, Artificial Sequence
  Synthetic peptide
HVKITDFGRAKLLGAEEKEYH

SEQ ID NO: 686 - protein, 21 aa, Artificial Sequence
  Synthetic peptide
QLGAEEKEYHAEGGKVPIKWM

SEQ ID NO: 687 - protein, 21 aa, Artificial Sequence
  Synthetic peptide
KITDFGLAKQLGAEEKEYHAE

SEQ ID NO: 688 - protein, 21 aa, Artificial Sequence
  Synthetic peptide
VKITDFGLAKQLGAEEKEYHA

SEQ ID NO: 689 - protein, 21 aa, Artificial Sequence
  Synthetic peptide
HVKITDFGLAKQLGAEEKEYH

SEQ ID NO: 690 - protein, 21 aa, Artificial Sequence
  Synthetic peptide
QHVKITDFGLAKQLGAEEKEY

SEQ ID NO: 691 - protein, 21 aa, Artificial Sequence
  Synthetic peptide
PQHVKITDFGLAKQLGAEEKE

SEQ ID NO: 692 - protein, 21 aa, Artificial Sequence
  Synthetic peptide
TPQHVKITDFGLAKQLGAEEK

SEQ ID NO: 693 - protein, 21 aa, Artificial Sequence
  Synthetic peptide
KTPQHVKITDFGLAKQLGAEE

SEQ ID NO: 694 - protein, 21 aa, Artificial Sequence
  Synthetic peptide
VKTPQHVKITDFGLAKQLGAE

SEQ ID NO: 695 - protein, 21 aa, Artificial Sequence
  Synthetic peptide
LVKTPQHVKITDFGLAKQLGA

SEQ ID NO: 696 - protein, 21 aa, Artificial Sequence
  Synthetic peptide
VLVKTPQHVKITDFGLAKQLG

SEQ ID NO: 697 - protein, 21 aa, Artificial Sequence
  Synthetic peptide
KQLGAEEKEYHAEGGKVPIKW

SEQ ID NO: 698 - protein, 21 aa, Artificial Sequence
  Synthetic peptide
NVLVKTPQHVKITDFGLAKQL

SEQ ID NO: 699 - protein, 21 aa, Artificial Sequence
  Synthetic peptide
RNVLVKTPQHVKITDFGLAKQ

SEQ ID NO: 700 - protein, 21 aa, Artificial Sequence
  Synthetic peptide
AKQLGAEEKEYHAEGGKVPIK

SEQ ID NO: 701 - protein, 21 aa, Artificial Sequence
  Synthetic peptide
LAKQLGAEEKEYHAEGGKVPI

SEQ ID NO: 702 - protein, 21 aa, Artificial Sequence
  Synthetic peptide
GLAKQLGAEEKEYHAEGGKVP

SEQ ID NO: 703 - protein, 21 aa, Artificial Sequence
  Synthetic peptide
FGLAKQLGAEEKEYHAEGGKV

SEQ ID NO: 704 - protein, 21 aa, Artificial Sequence
  Synthetic peptide
DFGLAKQLGAEEKEYHAEGGK

SEQ ID NO: 705 - protein, 21 aa, Artificial Sequence
  Synthetic peptide
TDFGLAKQLGAEEKEYHAEGG

SEQ ID NO: 706 - protein, 21 aa, Artificial Sequence
  Synthetic peptide
ITDFGLAKQLGAEEKEYHAEG

SEQ ID NO: 707 - protein, 21 aa, Artificial Sequence
  Synthetic peptide
RHVCRLLGICLTSTVQLITQL

SEQ ID NO: 708 - protein, 21 aa, Artificial Sequence
  Synthetic peptide
PRHVCRLLGICLTSTVQLITQ

SEQ ID NO: 709 - protein, 21 aa, Artificial Sequence
  Synthetic peptide
YVMASVDNPPRHVCRLLGICL

SEQ ID NO: 710 - protein, 21 aa, Artificial Sequence
  Synthetic peptide
AYVMASVDNPPRHVCRLLGIC

SEQ ID NO: 711 - protein, 21 aa, Artificial Sequence
  Synthetic peptide
EAYVMASVDNPPRHVCRLLGI

SEQ ID NO: 712 - protein, 21 aa, Artificial Sequence
  Synthetic peptide
DEAYVMASVDNPPRHVCRLLG

SEQ ID NO: 713 - protein, 21 aa, Artificial Sequence
  Synthetic peptide
LDEAYVMASVDNPPRHVCRLL

SEQ ID NO: 714 - protein, 21 aa, Artificial Sequence
  Synthetic peptide
ILDEAYVMASVDNPPRHVCRL

SEQ ID NO: 715 - protein, 21 aa, Artificial Sequence
  Synthetic peptide
EILDEAYVMASVDNPPRHVCR

SEQ ID NO: 716 - protein, 21 aa, Artificial Sequence
  Synthetic peptide
KEILDEAYVMASVDNPPRHVC

SEQ ID NO: 717 - protein, 21 aa, Artificial Sequence
  Synthetic peptide
NKEILDEAYVMASVDNPPRHV

SEQ ID NO: 718 - protein, 21 aa, Artificial Sequence
  Synthetic peptide
ANKEILDEAYVMASVDNPPRH

SEQ ID NO: 719 - protein, 21 aa, Artificial Sequence
  Synthetic peptide
PPRHVCRLLGICLTSTVQLIT

SEQ ID NO: 720 - protein, 21 aa, Artificial Sequence
  Synthetic peptide
KANKEILDEAYVMASVDNPPR

SEQ ID NO: 721 - protein, 21 aa, Artificial Sequence
  Synthetic peptide
PKANKEILDEAYVMASVDNPP

SEQ ID NO: 722 - protein, 21 aa, Artificial Sequence
  Synthetic peptide
NPPRHVCRLLGICLTSTVQLI

SEQ ID NO: 723 - protein, 21 aa, Artificial Sequence
  Synthetic peptide
DNPPRHVCRLLGICLTSTVQL

SEQ ID NO: 724 - protein, 21 aa, Artificial Sequence
  Synthetic peptide
VDNPPRHVCRLLGICLTSTVQ

SEQ ID NO: 725 - protein, 21 aa, Artificial Sequence
  Synthetic peptide
SVDNPPRHVCRLLGICLTSTV

SEQ ID NO: 726 - protein, 21 aa, Artificial Sequence
  Synthetic peptide
ASVDNPPRHVCRLLGICLTST

SEQ ID NO: 727 - protein, 21 aa, Artificial Sequence
  Synthetic peptide
MASVDNPPRHVCRLLGICLTS

SEQ ID NO: 728 - protein, 21 aa, Artificial Sequence
  Synthetic peptide
VMASVDNPPRHVCRLLGICLT

SEQ ID NO: 729 - protein, 21 aa, Artificial Sequence
  Synthetic peptide
IVDNPHVCRLLGICLTSTVQL

SEQ ID NO: 730 - protein, 21 aa, Artificial Sequence
  Synthetic peptide
ILDEAYVMAIVDNPHVCRLLG

SEQ ID NO: 731 - protein, 21 aa, Artificial Sequence
  Synthetic peptide
EILDEAYVMAIVDNPHVCRLL

SEQ ID NO: 732 - protein, 21 aa, Artificial Sequence
  Synthetic peptide
KEILDEAYVMAIVDNPHVCRL

SEQ ID NO: 733 - protein, 21 aa, Artificial Sequence
  Synthetic peptide
NKEILDEAYVMAIVDNPHVCR

SEQ ID NO: 734 - protein, 21 aa, Artificial Sequence
  Synthetic peptide
ANKEILDEAYVMAIVDNPHVC

SEQ ID NO: 735 - protein, 21 aa, Artificial Sequence
  Synthetic peptide
KANKEILDEAYVMAIVDNPHV

SEQ ID NO: 736 - protein, 21 aa, Artificial Sequence
  Synthetic peptide
PKANKEILDEAYVMAIVDNPH

SEQ ID NO: 737 - protein, 21 aa, Artificial Sequence
  Synthetic peptide
SPKANKEILDEAYVMAIVDNP

SEQ ID NO: 738 - protein, 21 aa, Artificial Sequence
  Synthetic peptide
TSPKANKEILDEAYVMAIVDN

SEQ ID NO: 739 - protein, 21 aa, Artificial Sequence
  Synthetic peptide
ATSPKANKEILDEAYVMAIVD

SEQ ID NO: 740 - protein, 21 aa, Artificial Sequence
  Synthetic peptide
AIVDNPHVCRLLGICLTSTVQ

SEQ ID NO: 741 - protein, 21 aa, Artificial Sequence
  Synthetic peptide
EATSPKANKEILDEAYVMAIV

SEQ ID NO: 742 - protein, 21 aa, Artificial Sequence
  Synthetic peptide
REATSPKANKEILDEAYVMAI

SEQ ID NO: 743 - protein, 21 aa, Artificial Sequence
  Synthetic peptide
MAIVDNPHVCRLLGICLTSTV

SEQ ID NO: 744 - protein, 21 aa, Artificial Sequence
  Synthetic peptide
VMAIVDNPHVCRLLGICLTST

SEQ ID NO: 745 - protein, 21 aa, Artificial Sequence
  Synthetic peptide
YVMAIVDNPHVCRLLGICLTS

SEQ ID NO: 746 - protein, 21 aa, Artificial Sequence
  Synthetic peptide
AYVMAIVDNPHVCRLLGICLT

SEQ ID NO: 747 - protein, 21 aa, Artificial Sequence
  Synthetic peptide
EAYVMAIVDNPHVCRLLGICL

SEQ ID NO: 748 - protein, 21 aa, Artificial Sequence
  Synthetic peptide
DEAYVMAIVDNPHVCRLLGIC

SEQ ID NO: 749 - protein, 21 aa, Artificial Sequence
  Synthetic peptide
LDEAYVMAIVDNPHVCRLLGI

SEQ ID NO: 750 - protein, 21 aa, Artificial Sequence
  Synthetic peptide
MQLMPFGCLLDYVREHKDNIG

SEQ ID NO: 751 - protein, 21 aa, Artificial Sequence
  Synthetic peptide
CLTSTVQLIMQLMPFGCLLDY

SEQ ID NO: 752 - protein, 21 aa, Artificial Sequence
  Synthetic peptide
ICLTSTVQLIMQLMPFGCLLD

SEQ ID NO: 753 - protein, 21 aa, Artificial Sequence
  Synthetic peptide
GICLTSTVQLIMQLMPFGCLL

SEQ ID NO: 754 - protein, 21 aa, Artificial Sequence
  Synthetic peptide
LGICLTSTVQLIMQLMPFGCL

SEQ ID NO: 755 - protein, 21 aa, Artificial Sequence
  Synthetic peptide
LLGICLTSTVQLIMQLMPFGC

SEQ ID NO: 756 - protein, 21 aa, Artificial Sequence
  Synthetic peptide
RLLGICLTSTVQLIMQLMPFG

SEQ ID NO: 757 - protein, 21 aa, Artificial Sequence
  Synthetic peptide
CRLLGICLTSTVQLIMQLMPF

SEQ ID NO: 758 - protein, 21 aa, Artificial Sequence
  Synthetic peptide
VCRLLGICLTSTVQLIMQLMP

SEQ ID NO: 759 - protein, 21 aa, Artificial Sequence
  Synthetic peptide
HVCRLLGICLTSTVQLIMQLM

SEQ ID NO: 760 - protein, 21 aa, Artificial Sequence
  Synthetic peptide
PHVCRLLGICLTSTVQLIMQL

SEQ ID NO: 761 - protein, 21 aa, Artificial Sequence
  Synthetic peptide
IMQLMPFGCLLDYVREHKDNI

SEQ ID NO: 762 - protein, 21 aa, Artificial Sequence
  Synthetic peptide
NPHVCRLLGICLTSTVQLIMQ

SEQ ID NO: 763 - protein, 21 aa, Artificial Sequence
  Synthetic peptide
DNPHVCRLLGICLTSTVQLIM

SEQ ID NO: 764 - protein, 21 aa, Artificial Sequence
  Synthetic peptide
LIMQLMPFGCLLDYVREHKDN

SEQ ID NO: 765 - protein, 21 aa, Artificial Sequence
  Synthetic peptide
QLIMQLMPFGCLLDYVREHKD

SEQ ID NO: 766 - protein, 21 aa, Artificial Sequence
  Synthetic peptide
VQLIMQLMPFGCLLDYVREHK

SEQ ID NO: 767 - protein, 21 aa, Artificial Sequence
  Synthetic peptide
TVQLIMQLMPFGCLLDYVREH

SEQ ID NO: 768 - protein, 21 aa, Artificial Sequence
  Synthetic peptide
STVQLIMQLMPFGCLLDYVRE

SEQ ID NO: 769 - protein, 21 aa, Artificial Sequence
  Synthetic peptide
TSTVQLIMQLMPFGCLLDYVR

SEQ ID NO: 770 - protein, 21 aa, Artificial Sequence
  Synthetic peptide
LTSTVQLIMQLMPFGCLLDYV

SEQ ID NO: 771 - protein, 21 aa, Artificial Sequence
  Synthetic peptide
VDNPHVCRLLGICLTSTVQLI

SEQ ID NO: 772 - protein, 21 aa, Artificial Sequence
  Synthetic peptide
SVDNPHVCRLLGICLTSTVQL

SEQ ID NO: 773 - protein, 21 aa, Artificial Sequence
  Synthetic peptide
ASVDNPHVCRLLGICLTSTVQ

SEQ ID NO: 774 - protein, 21 aa, Artificial Sequence
  Synthetic peptide
DEAYVMASVASVDNPHVCRLL

SEQ ID NO: 775 - protein, 21 aa, Artificial Sequence
  Synthetic peptide
LDEAYVMASVASVDNPHVCRL

SEQ ID NO: 776 - protein, 21 aa, Artificial Sequence
  Synthetic peptide
ILDEAYVMASVASVDNPHVCR

SEQ ID NO: 777 - protein, 21 aa, Artificial Sequence
  Synthetic peptide
EILDEAYVMASVASVDNPHVC

SEQ ID NO: 778 - protein, 21 aa, Artificial Sequence
  Synthetic peptide
KEILDEAYVMASVASVDNPHV

SEQ ID NO: 779 - protein, 21 aa, Artificial Sequence
  Synthetic peptide
NKEILDEAYVMASVASVDNPH

SEQ ID NO: 780 - protein, 21 aa, Artificial Sequence
  Synthetic peptide
ANKEILDEAYVMASVASVDNP

SEQ ID NO: 781 - protein, 21 aa, Artificial Sequence
  Synthetic peptide
KANKEILDEAYVMASVASVDN

SEQ ID NO: 782 - protein, 21 aa, Artificial Sequence
  Synthetic peptide
PKANKEILDEAYVMASVASVD

SEQ ID NO: 783 - protein, 21 aa, Artificial Sequence
  Synthetic peptide
SPKANKEILDEAYVMASVASV

SEQ ID NO: 784 - protein, 21 aa, Artificial Sequence
  Synthetic peptide
VASVDNPHVCRLLGICLTSTV

SEQ ID NO: 785 - protein, 21 aa, Artificial Sequence
  Synthetic peptide
TSPKANKEILDEAYVMASVAS

SEQ ID NO: 786 - protein, 21 aa, Artificial Sequence
  Synthetic peptide
ATSPKANKEILDEAYVMASVA

SEQ ID NO: 787 - protein, 21 aa, Artificial Sequence
  Synthetic peptide
SVASVDNPHVCRLLGICLTST

SEQ ID NO: 788 - protein, 21 aa, Artificial Sequence
  Synthetic peptide
ASVASVDNPHVCRLLGICLTS

SEQ ID NO: 789 - protein, 21 aa, Artificial Sequence
  Synthetic peptide
MASVASVDNPHVCRLLGICLT

SEQ ID NO: 790 - protein, 21 aa, Artificial Sequence
  Synthetic peptide
VMASVASVDNPHVCRLLGICL

SEQ ID NO: 791 - protein, 21 aa, Artificial Sequence
  Synthetic peptide
YVMASVASVDNPHVCRLLGIC

SEQ ID NO: 792 - protein, 21 aa, Artificial Sequence
  Synthetic peptide
AYVMASVASVDNPHVCRLLGI

SEQ ID NO: 793 - protein, 21 aa, Artificial Sequence
  Synthetic peptide
EAYVMASVASVDNPHVCRLLG

SEQ ID NO: 794 - protein, 17 aa, Artificial Sequence
  Synthetic peptide
WTIISDPGRRIHLSFND

SEQ ID NO: 795 - protein, 9 aa, Artificial Sequence
  Synthetic peptide
STYMIQKAV

SEQ ID NO: 796 - protein, 17 aa, Artificial Sequence
  Synthetic peptide
PFSTYMIQKAVKNYYSD

SEQ ID NO: 797 - protein, 9 aa, Artificial Sequence
  Synthetic peptide
MNLRPILTI

SEQ ID NO: 798 - protein, 10 aa, Artificial Sequence
  Synthetic peptide
MNLRPILTII

SEQ ID NO: 799 - protein, 17 aa, Artificial Sequence
  Synthetic peptide
SSCMGGMNLRPILTIIT

SEQ ID NO: 800 - protein, 17 aa, Artificial Sequence
  Synthetic peptide
TLVVLHRAVARLDVRDA

SEQ ID NO: 801 - protein, 9 aa, Artificial Sequence
  Synthetic peptide
TQGSAAELI

SEQ ID NO: 802 - protein, 17 aa, Artificial Sequence
  Synthetic peptide
RCAFCKHLVATIKCCEE

SEQ ID NO: 803 - protein, 9 aa, Artificial Sequence
  Synthetic peptide
KWIHCFNDV

SEQ ID NO: 804 - protein, 9 aa, Artificial Sequence
  Synthetic peptide
HCFNDVTAI

SEQ ID NO: 805 - protein, 17 aa, Artificial Sequence
  Synthetic peptide
TVLPSVSQMLSHHHIVS

SEQ ID NO: 806 - protein, 9 aa, Artificial Sequence
  Synthetic peptide
EASGITYEY

SEQ ID NO: 807 - protein, 9 aa, Artificial Sequence
  Synthetic peptide
ITYEYRPSK

SEQ ID NO: 808 - protein, 10 aa, Artificial Sequence
  Synthetic peptide
GITYEYRPSK

SEQ ID NO: 809 - protein, 10 aa, Artificial Sequence
  Synthetic peptide
ITYEYRPSKA

SEQ ID NO: 810 - protein, 11 aa, Artificial Sequence
  Synthetic peptide
YEYRPSKAPSF

SEQ ID NO: 811 - protein, 17 aa, Artificial Sequence
  Synthetic peptide
EASGITYEYRPSKAPSF

SEQ ID NO: 812 - protein, 9 aa, Artificial Sequence
  Synthetic peptide
LLKHPFTLM

SEQ ID NO: 813 - protein, 10 aa, Artificial Sequence
  Synthetic peptide
KELLKHPFTL

SEQ ID NO: 814 - protein, 10 aa, Artificial Sequence
  Synthetic peptide
FTLMYEERAV

SEQ ID NO: 815 - protein, 17 aa, Artificial Sequence
  Synthetic peptide
KELLKHPFTLMYEERAV

SEQ ID NO: 816 - protein, 9 aa, Artificial Sequence
  Synthetic peptide
LKHPFTLMY

SEQ ID NO: 817 - protein, 17 aa, Artificial Sequence
  Synthetic peptide
VKIPVAIKTSPKANKEI

SEQ ID NO: 818 - protein, 9 aa, Artificial Sequence
  Synthetic peptide
GEYFTHQIR

SEQ ID NO: 819 - protein, 17 aa, Artificial Sequence
  Synthetic peptide
LDGEYFTHQIRGRERFE

SEQ ID NO: 820 - protein, 9 aa, Artificial Sequence
  Synthetic peptide
DEVFGSSRL

SEQ ID NO: 821 - protein, 11 aa, Artificial Sequence
  Synthetic peptide
RLDPRMSTLQK

SEQ ID NO: 822 - protein, 17 aa, Artificial Sequence
  Synthetic peptide
RGNQCGCPAGLSRPRRT

SEQ ID NO: 823 - protein, 9 aa, Artificial Sequence
  Synthetic peptide
LEFENKQAL

SEQ ID NO: 824 - protein, 10 aa, Artificial Sequence
  Synthetic peptide
YMKSLEFENK

SEQ ID NO: 825 - protein, 10 aa, Artificial Sequence
  Synthetic peptide
SLEFENKQAL

SEQ ID NO: 826 - protein, 17 aa, Artificial Sequence
  Synthetic peptide
HVKITDFGRAKLLGAEE

SEQ ID NO: 827 - protein, 10 aa, Artificial Sequence
  Synthetic peptide
SSCMGGMNLR

SEQ ID NO: 828 - protein, 17 aa, Artificial Sequence
  Synthetic peptide
LDGEYFTLEIRGRERFE

SEQ ID NO: 829 - protein, 17 aa, Artificial Sequence
  Synthetic peptide
IFHAENDDDQFTKMFTL

SEQ ID NO: 830 - protein, 9 aa, Artificial Sequence
  Synthetic peptide
FEIRARTAA

SEQ ID NO: 831 - protein, 10 aa, Artificial Sequence
  Synthetic peptide
TIYVFEIRAR

SEQ ID NO: 832 - protein, 17 aa, Artificial Sequence
  Synthetic peptide
KPDTIYVFEIRARTAAG

SEQ ID NO: 833 - protein, 9 aa, Artificial Sequence
  Synthetic peptide
QEECLWMAV

SEQ ID NO: 834 - protein, 9 aa, Artificial Sequence
  Synthetic peptide
EECLWMAVL

SEQ ID NO: 835 - protein, 9 aa, Artificial Sequence
  Synthetic peptide
LLGGNSFEV

SEQ ID NO: 836 - protein, 11 aa, Artificial Sequence
  Synthetic peptide
NLLGGNSFEVR

SEQ ID NO: 837 - protein, 20 aa, Artificial Sequence
  Synthetic peptide
TLEDSSGNLLGGNSFEVRVC

SEQ ID NO: 838 - protein, 10 aa, Artificial Sequence
  Synthetic peptide
YMPGGDLVKL

SEQ ID NO: 839 - protein, 20 aa, Artificial Sequence
  Synthetic peptide
EYMPGGDLVKLMSNYDVPEK

SEQ ID NO: 840 - protein, 9 aa, Artificial Sequence
  Synthetic peptide
GEQDHTFRV

SEQ ID NO: 841 - protein, 9 aa, Artificial Sequence
  Synthetic peptide
RFKDIFQEM

SEQ ID NO: 842 - protein, 11 aa, Artificial Sequence
  Synthetic peptide
IFQEMFDKHYK

SEQ ID NO: 843 - protein, 10 aa, Artificial Sequence
  Synthetic peptide
SEITKQEKDF

SEQ ID NO: 844 - protein, 10 aa, Artificial Sequence
  Synthetic peptide
AEVDQDGVVM

SEQ ID NO: 845 - protein, 20 aa, Artificial Sequence
  Synthetic peptide
ALKPKAEVDQDGVVMCSGPE

SEQ ID NO: 846 - protein, 20 aa, Artificial Sequence
  Synthetic peptide
AAIHCKAGKGGTGVMICAYL

SEQ ID NO: 847 - protein, 9 aa, Artificial Sequence
  Synthetic peptide
QLWVDSTPL

SEQ ID NO: 848 - protein, 10 aa, Artificial Sequence
  Synthetic peptide
STPLPGTRVR

SEQ ID NO: 849 - protein, 10 aa, Artificial Sequence
  Synthetic peptide
TPLPGTRVRA

SEQ ID NO: 850 - protein, 17 aa, Artificial Sequence
  Synthetic peptide
QLWVDSTPLPGTRVRAM

SEQ ID NO: 851 - protein, 9 aa, Artificial Sequence
  Synthetic peptide
NYICNSSCM

SEQ ID NO: 852 - protein, 17 aa, Artificial Sequence
  Synthetic peptide
CTTIHYNYICNSSCMGG

SEQ ID NO: 853 - protein, 11 aa, Artificial Sequence
  Synthetic peptide
LPEPDKMLVAV

SEQ ID NO: 854 - protein, 10 aa, Artificial Sequence
  Synthetic peptide
IAAISEQVRF

SEQ ID NO: 855 - protein, 10 aa, Artificial Sequence
  Synthetic peptide
KAEDLGMLLK

SEQ ID NO: 856 - protein, 9 aa, Artificial Sequence
  Synthetic peptide
YTAPGIVEI

SEQ ID NO: 857 - protein, 10 aa, Artificial Sequence
  Synthetic peptide
LVRRSRTYSL

SEQ ID NO: 858 - protein, 18 aa, Artificial Sequence
  Synthetic peptide
SQTGLVRRSRTYSLAATP

SEQ ID NO: 859 - protein, 9 aa, Artificial Sequence
  Synthetic peptide
FRHSLVVPY

SEQ ID NO: 860 - protein, 9 aa, Artificial Sequence
  Synthetic peptide
QVVKGDPPL

SEQ ID NO: 861 - protein, 11 aa, Artificial Sequence
  Synthetic peptide
KPPKAKPSPTY

SEQ ID NO: 862 - protein, 9 aa, Artificial Sequence
  Synthetic peptide
TPITMTLPF

SEQ ID NO: 863 - protein, 18 aa, Artificial Sequence
  Synthetic peptide
EVLLKVLDKAHKLFRVFL

SEQ ID NO: 864 - protein, 10 aa, Artificial Sequence
  Synthetic peptide
SSADHPGASK

SEQ ID NO: 865 - protein, 18 aa, Artificial Sequence
  Synthetic peptide
SIMGQFDHSNIIHLEGVV

SEQ ID NO: 866 - protein, 9 aa, Artificial Sequence
  Synthetic peptide
KTKPVHSSL

SEQ ID NO: 867 - protein, 9 aa, Artificial Sequence
  Synthetic peptide
LAECKDSLL

SEQ ID NO: 868 - protein, 9 aa, Artificial Sequence
  Synthetic peptide
CVVTEVFLL

SEQ ID NO: 869 - protein, 9 aa, Artificial Sequence
  Synthetic peptide
VVTEVFLLA

SEQ ID NO: 870 - protein, 10 aa, Artificial Sequence
  Synthetic peptide
QINITAVAHV

SEQ ID NO: 871 - protein, 17 aa, Artificial Sequence
  Synthetic peptide
QINITAVAHVEIRGLEP

SEQ ID NO: 872 - protein, 9 aa, Artificial Sequence
  Synthetic peptide
FAKNPHLHY

SEQ ID NO: 873 - protein, 10 aa, Artificial Sequence
  Synthetic peptide
FAKNPHLHYI

SEQ ID NO: 874 - protein, 9 aa, Artificial Sequence
  Synthetic peptide
RLPVGAYEV

SEQ ID NO: 875 - protein, 9 aa, Artificial Sequence
  Synthetic peptide
KARLPVGAY

SEQ ID NO: 876 - protein, 9 aa, Artificial Sequence
  Synthetic peptide
RASKARLPV

SEQ ID NO: 877 - protein, 9 aa, Artificial Sequence
  Synthetic peptide
RRASKARLP

SEQ ID NO: 878 - protein, 9 aa, Artificial Sequence
  Synthetic peptide
ARRASKARL

SEQ ID NO: 879 - protein, 10 aa, Artificial Sequence
  Synthetic peptide
ARLPVGAYEV

SEQ ID NO: 880 - protein, 9 aa, Artificial Sequence
  Synthetic peptide
VMASVDNPL

SEQ ID NO: 881 - protein, 9 aa, Artificial Sequence
  Synthetic peptide
RLSISFENL

SEQ ID NO: 882 - protein, 9 aa, Artificial Sequence
  Synthetic peptide
ISFENLDTA

SEQ ID NO: 883 - protein, 10 aa, Artificial Sequence
  Synthetic peptide
SISFENLDTA

SEQ ID NO: 884 - protein, 17 aa, Artificial Sequence
  Synthetic peptide
RLSISFENLDTAKKKLP

SEQ ID NO: 885 - protein, 10 aa, Artificial Sequence
  Synthetic peptide
EPLSYSLLQR

SEQ ID NO: 886 - protein, 18 aa, Artificial Sequence
  Synthetic peptide
GHYEDTILKSKNSLNQPG

SEQ ID NO: 887 - protein, 10 aa, Artificial Sequence
  Synthetic peptide
HPLCGYHEQV

SEQ ID NO: 888 - protein, 9 aa, Artificial Sequence
  Synthetic peptide
GRMRSLEAL

SEQ ID NO: 889 - protein, 18 aa, Artificial Sequence
  Synthetic peptide
ELMLGRMRSLEALGEWGQ

SEQ ID NO: 890 - protein, 9 aa, Artificial Sequence
  Synthetic peptide
FHLKGHEDL

SEQ ID NO: 891 - protein, 9 aa, Artificial Sequence
  Synthetic peptide
HRGCSIWPR

SEQ ID NO: 892 - protein, 9 aa, Artificial Sequence
  Synthetic peptide
SPWATSSHL

SEQ ID NO: 893 - protein, 18 aa, Artificial Sequence
  Synthetic peptide
DGPLLLIVSTPNTAPCGA

SEQ ID NO: 894 - protein, 18 aa, Artificial Sequence
  Synthetic peptide
VAQYPFEDHNPPLLELIK

SEQ ID NO: 895 - protein, 11 aa, Artificial Sequence
  Synthetic peptide
DVSDNEPDLYR

SEQ ID NO: 896 - protein, 11 aa, Artificial Sequence
  Synthetic peptide
YYILDKKEHFK

SEQ ID NO: 897 - protein, 9 aa, Artificial Sequence
  Synthetic peptide
RILKVTEFK

SEQ ID NO: 898 - protein, 10 aa, Artificial Sequence
  Synthetic peptide
RILKVTEFKK

SEQ ID NO: 899 - protein, 10 aa, Artificial Sequence
  Synthetic peptide
LRILKVTEFK

SEQ ID NO: 900 - protein, 17 aa, Artificial Sequence
  Synthetic peptide
QALLRILKVTEFKKIKV

SEQ ID NO: 901 - protein, 9 aa, Artificial Sequence
  Synthetic peptide
TSSGLYVFR

SEQ ID NO: 902 - protein, 9 aa, Artificial Sequence
  Synthetic peptide
GLYVFRNER

SEQ ID NO: 903 - protein, 10 aa, Artificial Sequence
  Synthetic peptide
KTSSGLYVFR

SEQ ID NO: 904 - protein, 10 aa, Artificial Sequence
  Synthetic peptide
SGLYVFRNER

SEQ ID NO: 905 - protein, 11 aa, Artificial Sequence
  Synthetic peptide
EKTSSGLYVFR

SEQ ID NO: 906 - protein, 17 aa, Artificial Sequence
  Synthetic peptide
EKTSSGLYVFRNEREEE

SEQ ID NO: 907 - protein, 17 aa, Artificial Sequence
  Synthetic peptide
LTSTVQLIMQLMPFGCL

SEQ ID NO: 908 - protein, 9 aa, Artificial Sequence
  Synthetic peptide
RLSSSSSSK

SEQ ID NO: 909 - protein, 9 aa, Artificial Sequence
  Synthetic peptide
AQPAVQSAF

SEQ ID NO: 910 - protein, 10 aa, Artificial Sequence
  Synthetic peptide
VETDAAQPAV

SEQ ID NO: 911 - protein, 17 aa, Artificial Sequence
  Synthetic peptide
KVETDAAQPAVQSAFAV

SEQ ID NO: 912 - protein, 9 aa, Artificial Sequence
  Synthetic peptide
NLNKHRRYK

SEQ ID NO: 913 - protein, 17 aa, Artificial Sequence
  Synthetic peptide
NLNKHRRYKIRMSVYNA

SEQ ID NO: 914 - protein, 17 aa, Artificial Sequence
  Synthetic peptide
KIPVAIKESPKANKEIL

SEQ ID NO: 915 - protein, 17 aa, Artificial Sequence
  Synthetic peptide
LLGRNSFELRVCACPGR

SEQ ID NO: 916 - protein, 17 aa, Artificial Sequence
  Synthetic peptide
IKLIPDVSTTENYLEIE

SEQ ID NO: 917 - protein, 9 aa, Artificial Sequence
  Synthetic peptide
KLIPGVSTT

SEQ ID NO: 918 - protein, 10 aa, Artificial Sequence
  Synthetic peptide
IKLIPGVSTT

SEQ ID NO: 919 - protein, 11 aa, Artificial Sequence
  Synthetic peptide
EVVRHCPHHER

SEQ ID NO: 920 - protein, 10 aa, Artificial Sequence
  Synthetic peptide
VVRHCPHHER

SEQ ID NO: 921 - protein, 17 aa, Artificial Sequence
  Synthetic peptide
AIYKQSQHMTEVVRHCP

SEQ ID NO: 922 - protein, 9 aa, Artificial Sequence
  Synthetic peptide
SVSFGTVYK

SEQ ID NO: 923 - protein, 9 aa, Artificial Sequence
  Synthetic peptide
GSVSFGTVY

SEQ ID NO: 924 - protein, 16 aa, Artificial Sequence
  Synthetic peptide
PDGQITVGRIGSVSFG

SEQ ID NO: 925 - protein, 10 aa, Artificial Sequence
  Synthetic peptide
KLVVVGACGV

SEQ ID NO: 926 - protein, 17 aa, Artificial Sequence
  Synthetic peptide
EYKLVVVGACGVGKSAL

SEQ ID NO: 927 - protein, 10 aa, Artificial Sequence
  Synthetic peptide
VLMGAVALGA

SEQ ID NO: 928 - protein, 11 aa, Artificial Sequence
  Synthetic peptide
VLMGAVALGAL

SEQ ID NO: 929 - protein, 17 aa, Artificial Sequence
  Synthetic peptide
PHEGPLHALPPPAPHQP

SEQ ID NO: 930 - protein, 9 aa, Artificial Sequence
  Synthetic peptide
CMSCTVNLL

SEQ ID NO: 931 - protein, 9 aa, Artificial Sequence
  Synthetic peptide
AILLGAEEK

SEQ ID NO: 932 - protein, 10 aa, Artificial Sequence
  Synthetic peptide
RAILLGAEEK

SEQ ID NO: 933 - protein, 9 aa, Artificial Sequence
  Synthetic peptide
TDFGRAILL

SEQ ID NO: 934 - protein, 17 aa, Artificial Sequence
  Synthetic peptide
HVKITDFGRAILLGAEE

SEQ ID NO: 935 - protein, 17 aa, Artificial Sequence
  Synthetic peptide
VKITDFGRAILLGAEEK

SEQ ID NO: 936 - protein, 9 aa, Artificial Sequence
  Synthetic peptide
GSLHSGTAK

SEQ ID NO: 937 - protein, 10 aa, Artificial Sequence
  Synthetic peptide
LGSLHSGTAK

SEQ ID NO: 938 - protein, 17 aa, Artificial Sequence
  Synthetic peptide
GSYGFRLGSLHSGTAKS

SEQ ID NO: 939 - protein, 9 aa, Artificial Sequence
  Synthetic peptide
GDFGLATEK

SEQ ID NO: 940 - protein, 10 aa, Artificial Sequence
  Synthetic peptide
IGDFGLATEK

SEQ ID NO: 941 - protein, 17 aa, Artificial Sequence
  Synthetic peptide
IGDFGLATEKSRWSGSH

SEQ ID NO: 942 - protein, 9 aa, Artificial Sequence
  Synthetic peptide
AITSPKANK

SEQ ID NO: 943 - protein, 10 aa, Artificial Sequence
  Synthetic peptide
KIPVAITSPK

SEQ ID NO: 944 - protein, 17 aa, Artificial Sequence
  Synthetic peptide
KVKIPVAITSPKANKEI

SEQ ID NO: 945 - protein, 10 aa, Artificial Sequence
  Synthetic peptide
LGPMGGSLTL

SEQ ID NO: 946 - protein, 17 aa, Artificial Sequence
  Synthetic peptide
SVLGPMGGSLTLTTGLN

SEQ ID NO: 947 - protein, 9 aa, Artificial Sequence
  Synthetic peptide
KSSPFTAAA

SEQ ID NO: 948 - protein, 9 aa, Artificial Sequence
  Synthetic peptide
FTAAALAGH

SEQ ID NO: 949 - protein, 17 aa, Artificial Sequence
  Synthetic peptide
MQEKSSPFTAAALAGHM

SEQ ID NO: 950 - protein, 9 aa, Artificial Sequence
  Synthetic peptide
QEIQILKAL

SEQ ID NO: 951 - protein, 10 aa, Artificial Sequence
  Synthetic peptide
RDFQQEIQIL

SEQ ID NO: 952 - protein, 10 aa, Artificial Sequence
  Synthetic peptide
QQEIQILKAL

SEQ ID NO: 953 - protein, 9 aa, Artificial Sequence
  Synthetic peptide
FQQEIQILK

SEQ ID NO: 954 - protein, 17 aa, Artificial Sequence
  Synthetic peptide
QQRDFQQEIQILKALHS

SEQ ID NO: 955 - protein, 9 aa, Artificial Sequence
  Synthetic peptide
MASVDNPLM

SEQ ID NO: 956 - protein, 10 aa, Artificial Sequence
  Synthetic peptide
NPLMCRLLGI

SEQ ID NO: 957 - protein, 9 aa, Artificial Sequence
  Synthetic peptide
VDNPLMCRL

SEQ ID NO: 958 - protein, 17 aa, Artificial Sequence
  Synthetic peptide
MASVDNPLMCRLLGICL

SEQ ID NO: 959 - protein, 17 aa, Artificial Sequence
  Synthetic peptide
TNELYMMMWDCWHAVPS

SEQ ID NO: 960 - protein, 9 aa, Artificial Sequence
  Synthetic peptide
MMMWDCWHA

SEQ ID NO: 961 - protein, 9 aa, Artificial Sequence
  Synthetic peptide
MMWDCWHAV

SEQ ID NO: 962 - protein, 10 aa, Artificial Sequence
  Synthetic peptide
YMMMWDCWHA

SEQ ID NO: 963 - protein, 10 aa, Artificial Sequence
  Synthetic peptide
MMMWDCWHAV

SEQ ID NO: 964 - protein, 11 aa, Artificial Sequence
  Synthetic peptide
YMMMWDCWHAV

SEQ ID NO: 965 - protein, 9 aa, Artificial Sequence
  Synthetic peptide
TYQVSYGFR

SEQ ID NO: 966 - protein, 9 aa, Artificial Sequence
  Synthetic peptide
YQVSYGFRL

SEQ ID NO: 967 - protein, 10 aa, Artificial Sequence
  Synthetic peptide
KTYQVSYGFR

SEQ ID NO: 968 - protein, 11 aa, Artificial Sequence
  Synthetic peptide
QKTYQVSYGFR

SEQ ID NO: 969 - protein, 17 aa, Artificial Sequence
  Synthetic peptide
VPSQKTYQVSYGFRLGF

SEQ ID NO: 970 - protein, 9 aa, Artificial Sequence
  Synthetic peptide
HSHFWDNSK

SEQ ID NO: 971 - protein, 17 aa, Artificial Sequence
  Synthetic peptide
ERIVGYPAHSHFWDNSK

SEQ ID NO: 972 - protein, 17 aa, Artificial Sequence
  Synthetic peptide
RAETDDEDNEESDEEEE

SEQ ID NO: 973 - protein, 11 aa, Artificial Sequence
  Synthetic peptide
KSAVLASNSQF

SEQ ID NO: 974 - protein, 17 aa, Artificial Sequence
  Synthetic peptide
KYEKSAVLASNSQFLVK

SEQ ID NO: 975 - protein, 17 aa, Artificial Sequence
  Synthetic peptide
DLVPFALLRLLHCLLSK

SEQ ID NO: 976 - protein, 17 aa, Artificial Sequence
  Synthetic peptide
RKRKFINRGLAHDSEEL

SEQ ID NO: 977 - protein, 17 aa, Artificial Sequence
  Synthetic peptide
QDFRRNILSVCKEAVDL

SEQ ID NO: 978 - protein, 10 aa, Artificial Sequence
  Synthetic peptide
LSELETPGKY

SEQ ID NO: 979 - protein, 23 aa, Artificial Sequence
  Synthetic peptide
KTPQHVKITDFGLAKLLGAEEKE

SEQ ID NO: 980 - protein, 23 aa, Artificial Sequence
  Synthetic peptide
KTPQHVKITDFGRAKLLGAEEKE

SEQ ID NO: 981 - protein, 23 aa, Artificial Sequence
  Synthetic peptide
CRLLGICLTSTVQLITQLMPFGC

SEQ ID NO: 982 - protein, 23 aa, Artificial Sequence
  Synthetic peptide
CRLLGICLTSTVQLIMQLMPFGC